SEQUENCE LISTING (590 sequences)

SEQ ID NO: 1 - DNA, 2241 bp, Homo sapiens
atctttgctgcaaaggctgggtatcggctgtgctcagcaaagcgtcaactcgtgcaagaacttagcaggaatagttctggctaaggttaggaggctgccaccaaagtctcttttttgttcctctgcttctcccgtttgcctccttatcatgagatctttttgctaagctggcagaaagattgcatagtcagtgcttccagctctgctcccacctgatcctgcactgtcctctggtccctgaatgaatgaactctgatacccaatcttgtctcgagccttctctatgccactcatggctcctcttctgctctttccatctttttgctgagagttctgagctctgtacttcctcttggcccatctcacttcctgaaacacccctgaagagggttgcttatcttgatggaactcaaaaagccaaaaagctgcaggcagaggcgttgaggacatctgtttggggaactaagagcagcagcactttcagattcagtccatatagagctgtcctacagcattctggaaacttgaggatgtgcggtgcataaaggggctggaagtgacccacctgtgatgagccctttctaaggagaagggtttccaagagatcaccccaccagaaaagggtaggaatgagcaagttgggaattttagactgtcactgcacatggacctctgggaagacgtctggcgagagctaggcccactggccctacagacggatcttgctggctcacctgtccctgtggaggttcccctgggaaggcaagatgcccaacaacagcactgctctgtcattggccaatgttacctacatcaccatggaaattttcattggactctgcgccatagtgggcaacgtgctggtcatctgcgtggtcaagctgaaccccagcctgcagaccaccaccttctatttcattgtctctctagccctggctgacattgctgttggggtgctggtcatgcctttggccattgttgtcagcctgggcatcacaatccacttctacagctgcctttttatgacttgcctactgcttatctttacccacgcctccatcatgtccttgctggccatcgctgtggaccgatacttgcgggtcaagcttaccgtcagatacaagagggtcaccactcacagaagaatatggctggccctgggcctttgctggctggtgtcattcctggtgggattgacccccatgtttggctggaacatgaaactgacctcagagtaccacagaaatgtcaccttcctttcatgccaatttgtttccgtcatgagaatggactacatggtatacttcagcttcctcacctggattttcatccccctggttgtcatgtgcgccatctatcttgacatcttttacatcattcggaacaaactcagtctgaacttatctaactccaaagagacaggtgcattttatggacgggagttcaagacggctaagtccttgtttctggttcttttcttgtttgctctgtcatggctgcctttatctatcatcaactgcatcatctactttaatggtgaggtaccacagcttgtgctgtacatgggcatcctgctgtcccatgccaactccatgatgaaccctatcgtctatgcctataaaataaagaagttcaaggaaacctaccttttgatcctcaaagcctgtgtggtctgccatccctctgattctttggacacaagcattgagaagaattctgagtagttatccatcagagatgactctgtctcattgaccttcagattccccatcaacaaacacttgagggcctgtatgcctgggccaagggatttttacatccttgattacttccactgaggtgggagcatctccagtgctccccaattatatctcccccactccactactctcttcctccacttcatttttcctttgtcctttctctctaattcagtgttttggaggcctgacttggggacaacgtattattgatattattgtctgttttccttcttcccaatagaagaataagtcatggagcctgaagggtgcctagttgacttactgacaaaaggctctagttgggctgaacatgtgtgtggtggtgactcatttccatgccattgtggaattgagcagagaacctgctctcggaggatgcctagaagatgttgggaacagaagaaataaactgagtttaagggggacttaaactgctgaattcacctgtggatgtttttgagtaaataaaagctaatag

SEQ ID NO: 2 - DNA, 1755 bp, Homo sapiens
ggggactacggagagctctgcagggagccgaggcccccgcccgggccaagggagcttctgtcccgaggaccaggggatgcgaagggattgccccctgtgggtcactttctcagtcattttgagctcagcctaatcaaagactgaggttatgaagtcgatcctagatggccttgcagataccaccttccgcaccatcaccactgacctcctgtacgtgggctcaaatgacattcagtacgaagacatcaaaggtgacatggcatccaaattagggtacttcccacagaaattccctttaacttcctttaggggaagtcccttccaagagaagatgactgcgggagacaacccccagctagtcccagcagaccaggtgaacattacagaattttacaacaagtctctctcgtccttcaaggagaatgaggagaacatccagtgtggggagaacttcatggacatagagtgtttcatggtcctgaaccccagccagcagctggccattgcagtcctgtccctcacgctgggcaccttcacggtcctggagaacctcctggtgctgtgcgtcatcctccactcccgcagcctccgctgcaggccttcctaccacttcatcggcagcctggcggtggcagacctcctggggagtgtcatttttgtctacagcttcattgacttccacgtgttccaccgcaaagatagccgcaacgtgtttctgttcaaactgggtggggtcacggcctccttcactgcctccgtgggcagcctgttcctcacagccatcgacaggtacatatccattcacaggcccctggcctataagaggattgtcaccaggcccaaggccgtggtggcgttttgcctgatgtggaccatagccattgtgatcgccgtgctgcctctcctgggctggaactgcgagaaactgcaatctgtttgctcagacattttcccacacattgatgaaacctacctgatgttctggatcggggtcaccagcgtactgcttctgttcatcgtgtatgcgtacatgtatattctctggaaggctcacagccacgccgtccgcatgattcagcgtggcacccagaagagcatcatcatccacacgtctgaggatgggaaggtacaggtgacccggccagaccaagcccgcatggacattaggttagccaagaccctggtcctgatcctggtggtgttgatcatctgctggggccctctgcttgcaatcatggtgtatgatgtctttgggaagatgaacaagctcattaagacggtgtttgcattctgcagtatgctctgcctgctgaactccaccgtgaaccccatcatctatgctctgaggagtaaggacctgcgacacgctttccggagcatgtttccctcttgtgaaggcactgcgcagcctctggataacagcatgggggactcggactgcctgcacaaacacgcaaacaatgcagccagtgttcacagggccgcagaaagctgcatcaagagcacggtcaagattgccaaggtaaccatgtctgtgtccacagacacgtctgccgaggctctgtgagcctgatgcctccctggcagcacaggaaaagaatttttttttttaagctcaaaatctagaagagtctattgtctccttggttatatttttttaactttaccatgctcaatgaaaaggtgattgccacatgtcacttatttgcttagtttccgtttgggctaatcttccggggttcgtaggaaaccttt

SEQ ID NO: 3 - DNA, 4039 bp, Homo sapiens
agagccgcgggggccgtaggaagccaaccttccctgcttctccggggccctcgccccctcctccccacaaaatcagggatggaggcgcctccccggcaccctcttagcagccctccccaggaaaagtgtcccccctgagctcctaacgctccccaacagctacccctgccccccacgccatggggcccggggccccttttgcccgggtggggtggccactgccgcttctggttgtgatggcggcaggggtggctccggtgtgggcctcccactccccccatctcccgcggcctcactcgcgggtccccccgcacccctcctcagaacggcgcgcagtgtacatcggggcactgtttcccatgagcgggggctggccagggggccaggcctgccagcccgcggtggagatggcgctggaggacgtgaatagccgcagggacatcctgccggactatgagctcaagctcatccaccacgacagcaagtgtgatccaggccaagccaccaagtacctatatgagctgctctacaacgaccctatcaagatcatccttatgcctggctgcagctctgtctccacgctggtggctgaggctgctaggatgtggaacctcattgtgctttcctatggctccagctcaccagccctgtcaaaccggcagcgtttccccactttcttccgaacgcacccatcagccacactccacaaccctacccgcgtgaaactctttgaaaagtggggctggaagaagattgctaccatccagcagaccactgaggtcttcacttcgactctggacgacctggaggaacgagtgaaggaggctggaattgagattactttccgccagagtttcttctcagatccagctgtgcccgtcaaaaacctgaagcgccaggatgcccgaatcatcgtgggacttttctatgagactgaagcccggaaagttttttgtgaggtgtacaaggagcgtctctttgggaagaagtacgtctggttcctcattgggtggtatgctgacaattggttcaagatctacgacccttctatcaactgcacagtggatgagatgactgaggcggtggagggccacatcacaactgagattgtcatgctgaatcctgccaatacccgcagcatttccaacatgacatcccaggaatttgtggagaaactaaccaagcgactgaaaagacaccctgaggagacaggaggcttccaggaggcaccgctggcctatgatgccatctgggccttggcactggccctgaacaagacatctggaggaggcggccgttctggtgtgcgcctggaggacttcaactacaacaaccagaccattaccgaccaaatctaccgggcaatgaactcttcgtcctttgagggtgtctctggccatgtggtgtttgatgccagcggctctcggatggcatggacgcttatcgagcagcttcagggtggcagctacaagaagattggctactatgacagcaccaaggatgatctttcctggtccaaaacagataaatggattggagggtcccccccagctgaccagaccctggtcatcaagacattccgcttcctgtcacagaaactctttatctccgtctcagttctctccagcctgggcattgtcctagctgttgtctgtctgtcctttaacatctacaactcacatgtccgttatatccagaactcacagcccaacctgaacaacctgactgctgtgggctgctcactggctttagctgctgtcttccccctggggctcgatggttaccacattgggaggaaccagtttcctttcgtctgccaggcccgcctctggctcctgggcctgggctttagtctgggctacggttccatgttcaccaagatttggtgggtccacacggtcttcacaaagaaggaagaaaagaaggagtggaggaagactctggaaccctggaagctgtatgccacagtgggcctgctggtgggcatggatgtcctcactctcgccatctggcagatcgtggaccctctgcaccggaccattgagacatttgccaaggaggaacctaaggaagatattgacgtctctattctgccccagctggagcattgcagctccaggaagatgaatacatggcttggcattttctatggttacaaggggctgctgctgctgctgggaatcttccttgcttatgagaccaagagtgtgtccactgagaagatcaatgatcaccgggctgtgggcatggctatctacaatgtggcagtcctgtgcctcatcactgctcctgtcaccatgattctgtccagccagcaggatgcagcctttgcctttgcctctcttgccatagttttctcctcctatatcactcttgttgtgctctttgtgcccaagatgcgcaggctgatcacccgaggggaatggcagtcggaggcgcaggacaccatgaagacagggtcatcgaccaacaacaacgaggaggagaagtcccggctgttggagaaggagaaccgtgaactggaaaagatcattgctgagaaagaggagcgtgtctctgaactgcgccatcaactccagtctcggcagcagctccgctcccggcgccacccaccgacacccccagaaccctctgggggcctgcccaggggaccccctgagccccccgaccggcttagctgtgatgggagtcgagtgcatttgctttataagtgagggtagggtgagggaggacaggccagtagggggagggaaagggagaggggaagggcaggggactcaggaagcagggggtccccatccccagctgggaagaacatgctatccaatctcatctcttgtaaatacatgtccccctgtgagttctgggctgatttgggtctctcatacctctgggaaacagacctttttctctcttactgcttcatgtaattttgtatcacctcttcacaatttagttcgtacctggcttgaagctgctcactgctcacacgctgcctcctcagcagcctcactgcatctttctcttcccatgcaacaccctcttctagttaccacggcaacccctgcagctcctctgcctttgtgctctgttcctgtccagcaggggtctcccaacaagtgctctttccaccccaaaggggcctctccttttctccactgtcataatctctttccatcttacttgcccttctatactttctcacatgtggctccccctgaattttgcttcctttgggagctcattcttttcgccaaggctcacatgctccttgcctctgctctgtgcactcacgctcagcacacatgcatcctcccctctcctgcgtgtgcccactgaacatgctcatgtgtacacacgcttttcccgtatgctttcttcatgttcagtcacatgtgctctcgggtgccctgcattcacagctacgtgtgcccctctcatggtcatgggtctgcccttgagcgtgtttgggtaggcatgtgcaatttgtctagcatgctgagtcatgtctttcctatttgcacacgtccatgtttatccatgtactttccctgtgtaccctccatgtaccttgtgtactttcttcccttaaatcatggtattcttctgacagagccatatgtaccctaccctgcacattgttatgcacttttccccaattcatgtttggtggggccatccacaccctctccttgtcacagaatctccatttctgctcagattccccccatctccattgcattcatgtactaccctcagtctacactcacaatcatcttctcccaagactgctcccttttgttttgtgtttttttgaggggaattaaggaaaaataagtgggggcaggtttggagagctgcttccagtggatagttgatgagaatcctgaccaaaggaaggcacccttgactgttgggatagacagatggacctatggggtgggaggtggtgtccctttcacactgtggtgtctcttggggaaggatctccccgaatctcaataaaccagtgaacagtgtgactcggcaaaaaaaaaaa

SEQ ID NO: 4 - DNA, 1401 bp, Homo sapiens
atgaataactcaacaaactcctctaacaatagcctggctcttacaagtccttataagacatttgaagtggtgtttattgtcctggtggctggatccctcagtttggtgaccattatcgggaacatcctagtcatggtttccattaaagtcaaccgccacctccagaccgtcaacaattactttttattcagcttggcctgtgctgaccttatcataggtgttttctccatgaacttgtacaccctctacactgtgattggttactggcctttgggacctgtggtgtgtgacctttggctagccctggactatgtggtcagcaatgcctcagttatgaatctgctcatcatcagctttgacaggtacttctgtgtcacaaaacctctgacctacccagtcaagcggaccacaaaaatggcaggtatgatgattgcagctgcctgggtcctctctttcatcctctgggctccagccattctcttctggcagttcattgtaggggtgagaactgtggaggatggggagtgctacattcagtttttttccaatgctgctgtcacctttggtacggctattgcagccttctatttgccagtgatcatcatgactgtgctatattggcacatatcccgagccagcaagagcaggataaagaaggacaagaaggagcctgttgccaaccaagaccccgtttctccaagtctggtacaaggaaggatagtgaagccaaacaataacaacatgcccagcagtgacgatggcctggagcacaacaaaatccagaatggcaaagcccccagggatcctgtgactgaaaactgtgttcagggagaggagaaggagagctccaatgactccacctcagtcagtgctgttgcctctaatatgagagatgatgaaataacccaggatgaaaacacagtttccacttccctgggccattccaaagatgagaactctaagcaaacatgcatcagaattggcaccaagaccccaaaaagtgactcatgtaccccaactaataccaccgtggaggtagtggggtcttcaggtcagaatggagatgaaaagcagaatattgtagcccgcaagattgtgaagatgactaagcagcctgcaaaaaagaagcctcctccttcccgggaaaagaaagtcaccaggacaatcttggctattctgttggctttcatcatcacttgggccccatacaatgtcatggtgctcattaacaccttttgtgcaccttgcatccccaacactgtgtggacaattggttactggctttgttacatcaacagcactatcaaccctgcctgctatgcactttgcaatgccaccttcaagaagacctttaaacaccttctcatgtgtcattataagaacataggcgctacaaggtaa

SEQ ID NO: 5 - DNA, 2699 bp, Homo sapiens
agtggtacaagatgtggaggtggaagacagtgatattgtgtccggaatttattccttcttgtgggttctcggtctcactaacttcaagaatgaagccgcagaccgtcgcggtgtgttacagttcttaaagatggtgtgtccagagtttgttccttcagatgttcagatgtgtcgagtttcttccttccggtgggttcgtggtctccctgacttcagaagtgaagccacataccttcgcaatgctggccaagaagagctgaaatagaaaacagcctagaacctaacactatttactgtaaaatttttgcaccaggatggaaggggattcttaccacaatgcaaccaccgtcaatggcaccccagtaaatcaccagcctttggaacgccacaggttgtgggaagtcatcaccattgcagctgtgactgctgtggtaagcctgatcaccattgtgggcaatgtcttggtcatgatctccttcaaagtcaacagccagctcaagacagttaacaactattacctgctcagcttagcctgtgcagatctcatcattggaatcttctccatgaacctctacaccacctacatcctcatgggacgctgggctctcgggagtctggcttgtgacctttggcttgcactggactacgtggccagcaacgcttctgtcatgaaccttctggtgatcagttttgaccgttacttttccatcacaagacccttgacatatcgggccaagcgtactccgaaaagggctggcatcatgattggcttggcctggctgatctccttcatcctctgggccccagcaatcctctgctggcagtacttggttgggaagcggacagttccactggatgagtgccagatccagtttctctctgagcccaccatcacttttggcactgccattgctgccttctacatccctgtttctgtcatgaccatcctctactgtcgaatctaccgggaaacagagaagcgaaccaaggacctggctgacctccagggttctgactctgtgaccaaagctgagaagagaaagccagctcatagggctctgttcagatcctgcttgcgctgtcctcgacccaccctggcccagcgggaaaggaaccaggcctcctggtcatcctcccgcaggagcacctccaccactgggaagccatcccaagccactggcccaagcgccaattgggccaaagctgagcagctcaccacctgtagcagctacccttcctcagaggatgaggacaagcccgccactgaccctgtcctccaagtggtctacaagagtcagggtaaggaaagcccaggggaagaattcagtgctgaagagactgaggaaacttttgtgaaagctgaaactgaaaaaagtgactatgacaccccaaactaccttctgtctccagcagctgctcatagacccaagagtcagaaatgtgtggcctataagttccgattggtggtaaaagctgacgggaaccaggagaccaacaatggctgtcacaaggtgaaaatcatgccctgccccttcccagtggccaaggaaccttcaacgaaaggcctcaatcccaaccccagccatcaaatgaccaaacgaaagagagtggtcctagtcaaagagaggaaagcagcccagacactgagtgccattctcctggccttcatcatcacatggaccccgtataacatcatggtcctggtttctaccttctgtgacaagtgtgtcccagtcaccctgtggcacttgggctattggttgtgctatgtcaatagcactgtcaaccccatctgctatgccctctgcaacagaaccttcaggaagacctttaagatgctgcttctctgccgatggaaaaagaaaaaagtggaagagaagttgtactggcaggggaacagcaagctaccctgaaaagtcaacaactcctctcgaaagaacaatgaccacagtcaacatcctctgaggatgagcaagctgattctggtttgtatattttcaaaaagaagacatctcattttgagtccttgaagatttttgtaaaggctcaagtttggttgccaaatggaaggggccatagctgcagcaattgctgacatattaaatgactcttgcctatgaccaaggccatttgatgccaggggagtttgccaatgaagtaaagggataggctcatggcccttcacaagaggaagcacactgggtaacaatgaacagtgactcagggaacttatgccccttctgtaggaaacagcagagaccaggtggaaaccttttcctgtggaaacctgtcatagaattttgtgcaatatgtatgtgtctatgaagctgtcttgtgccagtgagaaccagcaggagaatgtacaacagtgtcacttgttaacgagactgatattcaacaggcttctaagaatatgtatcttcataaactgatcactatctattatgcagctattatgtggtctatactatactgtggtttgttttcctgtccccacatctgagtgaaggtcttgctcttccctttcatatccaatgtcaattccttgtactcactgaacccatgctgatctccagggaaccatccttcctctcagaatccagagtctggaaggactgaaacctggtcatacccagtcctttcaaggggcctcttttctactaataaagatggatcaagtctcaaaaaaaaaaaaaaaa

SEQ ID NO: 6 - DNA, 2921 bp, Homo sapiens
agcctgtggagacgggacagccctgtcccactcactctttcccctgccgctcctgccggcagctccaaccatgggaggccgcgtctttctcgcattctgtgtctggctgactctgccgggagctgaaacccaggactccaggggctgtgcccggtggtgccctcagaactcctcgtgtgtcaatgccaccgcctgtcgctgcaatccagggttcagctctttttctgagatcatcaccaccccgacggagacttgtgacgacatcaacgagtgtgcaacaccgtcgaaagtgtcatgcggaaaattctcggactgctggaacacagaggggagctacgactgcgtgtgcagcccgggatatgagcctgtttctggggcaaaaacattcaagaatgagagcgagaacacctgtcaagatgtggacgagtgcagctccgggcagcatcagtgtgacagctccaccgtctgcttcaacaccgtgggttcatacagctgccgctgccgcccaggctggaagcccagacacggaatcccgaataaccaaaaggacactgtctgtgaagatatgactttctccacctggaccccgccccctggagtccacagccagacgctttcccgattcttcgacaaagtccaggacctgggcagagactccaagacaagctcagccgaggtcaccatccagaatgtcatcaaattggtggatgaactgatggaagctcctggagacgtagaggccctggcgccacctgtccggcacctcatagccacccagctgctctcaaaccttgaagatatcatgaggatcctggccaagagcctgcctaaaggccccttcacctacatttccccttcgaacacagagctgaccctgatgatccaggagcggggggacaagaacgtcactatgggtcagagcagcgcacgcatgaagctgaattgggctgtggcagctggagccgaggatccaggccccgccgtggcgggcatcctctccatccagaacatgacgacattgctggccaatgcctccttgaacctgcattccaagaagcaagccgaactggaggagatatatgaaagcagcatccgtggtgtccaactcagacgcctctctgccgtcaactccatctttctgagccacaacaacaccaaggaactcaactcccccatccttttcgccttctcccaccttgagtcctccgatggggaggcgggaagagaccctcctgccaaggacgtgatgcctgggccacggcaggagctgctctgtgccttctggaagagtgacagcgacaggggagggcactgggccaccgagggctgccaggtgctgggcagcaagaacggcagcaccacctgccaatgcagccacctgagcagctttgcgatccttatggctcattatgacgtggaggactggaagctgaccctgatcaccagggtgggactggcgctgtcactcttctgcctgctgctgtgcatcctcactttcctgctggtgcggcccatccagggctcgcgcaccaccatacacctgcacctctgcatctgcctcttcgtgggctccaccatcttcctggccggcatcgagaacgaaggcggccaggtggggctgcgctgccgcctggtggccgggctgctgcactactgtttcctggccgccttctgctggatgagcctcgaaggcctggagctctactttcttgtggtgcgcgtgttccaaggccagggcctgagtacgcgctggctctgcctgatcggctatggcgtgcccctgctcatcgtgggcgtctcggctgccatctacagcaagggctacggccgccccagatactgctggttggactttgagcagggcttcctctggagcttcttgggacctgtgaccttcatcattttgtgcaatgctgtcattttcgtgactaccgtctggaagctcactcagaagttttctgaaatcaatccagacatgaagaaattaaagaaggcgagggcgctgaccatcacggccatcgcgcagctcttcctgttgggctgcacctgggtctttggcctgttcatcttcgacgatcggagcttggtgctgacctatgtgtttaccatcctcaactgcctgcagggcgccttcctctacctgctgcactgcctgctcaacaagaaggttcgggaagaataccggaagtgggcctgcctagttgctggggggagcaagtactcagaattcacctccaccacgtctggcactggccacaatcagacccgggccctcagggcatcagagtccggcatatgaaggcgcatggttctggacggcccagcagctcctgtggccacagcagctttgtacacgaagaccatccatcctcccttcgtccaccactctactccctccaccctccctccctgatcccgtgtgccaccaggagggagtggcagctatagtctggcaccaaagtccaggacacccagtggggtggagtcggagccactggtcctgctgctggctgcctctctgctccaccttgtgacccagggtggggacaggggctggcccagggctgcaatgcagcatgttgccctggcacctgtggccagtactcgggacagactaagggcgcttgtcccatcctggacttttcctctcatgtctttgctgcagaactgaagagactaggcgctggggctcagcttccctcttaagctaagactgatgtcagaggccccatggcgaggccccttggggccactgcctgaggctcacggtacagaggcctgccctgcctggccgggcaggaggttctcactgttgtgaaggttgtagacgttgtgtaatgtgtttttatctgttaaaatttttcagtgttgacacttaaaattaaacacatgcatacagaaaaaaaaaaaaaaaaaaaaaa

SEQ ID NO: 7 - DNA, 1726 bp, Homo sapiens
ccagattctaaatatcaggaaagacgctgtgggaaaatagcaggccaaaagttcttagtaaactgcagccagggagactcagactagaatggaggtagaaagaactgatgcagagtgggtttaattctaagcctttttgtggctaagttttgttgttgttaacttattgaatttagagttgtattgcactggtcatgtgaaagccagagcagcaccagtgtcaaaatagtgacagagagttttgaataccatagttagtatatatgtactcagagtatttttattaaagaaggcaaagagcccggcatagatcttatcttcatcttcactcggttgcaaaatcaatagttaagaaatagcatctaagggaacttttaggtgggaaaaaaaatctagagatggctctaaatgactgtttccttctgaacttggaggtggaccatttcatgcactgcaacatctccagtcacagtgcggatctccccgtgaacgatgactggtcccacccggggatcctctatgtcatccctgcagtttatggggttatcattctgataggcctcattggcaacatcactttgatcaagatcttctgtacagtcaagtccatgcgaaacgttccaaacctgttcatttccagtctggctttgggagacctgctcctcctaataacgtgtgctccagtggatgccagcaggtacctggctgacagatggctatttggcaggattggctgcaaactgatcccctttatacagcttacctctgttggggtgtctgtcttcacactcacggcgctctcggcagacagatacaaagccattgtccggccaatggatatccaggcctcccatgccctgatgaagatctgcctcaaagccgcctttatctggatcatctccatgctgctggccattccagaggccgtgttttctgacctccatcccttccatgaggaaagcaccaaccagaccttcattagctgtgccccatacccacactctaatgagcttcaccccaaaatccattctatggcttcctttctggtcttctacgtcatcccactgtcgatcatctctgtttactactacttcattgctaaaaatctgatccagagtgcttacaatcttcccgtggaagggaatatacatgtcaagaagcagattgaatcccggaagcgacttgccaagacagtgctggtgtttgtgggcctgttcgccttctgctggctccccaatcatgtcatctacctgtaccgctcctaccactactctgaggtggacacctccatgctccactttgtcaccagcatctgtgcccgcctcctggccttcaccaactcctgcgtgaacccctttgccctctacctgctgagcaagagtttcaggaaacagttcaacactcagctgctctgttgccagcctggcctgatcatccggtctcacagcactggaaggagtacaacctgcatgacctccctcaagagtaccaacccctccgtggccacctttagcctcatcaatggaaacatctgtcacgagcggtatgtctagattgacccttgattttgccccctgagggacggttttgctttatggctagacaggaacccttgcatccattgttgtgtctgtgccctccaaagagccttcagaatgctcctgagtggtgtaggtgggggtggggaggcccaaatgatggatcaccattatattttgaaagaagc

SEQ ID NO: 8 - DNA, 2057 bp, Homo sapiens
cactcactctctcccagccttccttacgaagcctgtgactttcgtgactgctttctcttttttgttttccttttttctttttttttttttttcctggctcagcttgaaacagagcctcgtaccaggggaggctcaggccttggattttaatgtcagggatggaaaaacttcagaatgcttcctggatctaccagcagaaactagaagatccattccagaaacacctgaacagcaccgaggagtatctggccttcctctgcggacctcggcgcagccacttcttcctccccgtgtctgtggtgtatgtgccaatttttgtggtgggggtcattggcaatgtcctggtgtgcctggtgattctgcagcaccaggctatgaagacgcccaccaactactacctcttcagcctggcggtctctgacctcctggtcctgctccttggaatgcccctggaggtctatgagatgtggcgcaactaccctttcttgttcgggcccgtgggctgctacttcaagacggccctctttgagaccgtgtgcttcgcctccatcctcagcatcaccaccgtcagcgtggagcgctacgtggccatcctacacccgttccgcgccaaactgcagagcacccggcgccgggccctcaggatcctcggcatcgtctggggcttctccgtgctcttctccctgcccaacaccagcatccatggcatcaagttccactacttccccaatgggtccctggtcccaggttcggccacctttacggtcatcaagcccatgtggatctacaatttcatcatccaggtcacctccttcctattctacctcctccccatgactgtcatcagtgtcctctactacctcatggcactcagactaaagaaagacaaatctcttgaggcagatgaagggaatgcaaatattcaaagaccctgcagaaaatcagtcaacaagatgctgtttgtcttggtcttagtgtttgctatctgttgggccccgttccacattgaccgactcttcttcagctttgtggaggagtggagtgaatccctggctgctgtgttcaacctcgtccatgtggtgtcaggtgtcttcttctacctgagctcagctgtcaaccccattatctataacctactgtctcgccgcttccaggcagcattccagaatgtgatctcttctttccacaaacagtggcactcccagcatgacccacagttgccacctgcccagcggaacatcttcctgacagaatgccactttgtggagctgaccgaagatataggtccccaattcccatgtcagtcatccatgcacaactctcacctcccaacagccctctctagtgaacagatgtcaagaacaaactatcaaagcttccactttaacaaaacctgaattctttcagagctgactctcctctatgcctcaaaacttcagagaggaacatcccataatgtatgccttctcatatgatattagagaggtagaatggctcttacaactcatgtacccattgctagtttttttttttttaataaacgtgaaaactgagagttagatctggtttcaaaacccaagactgcctgatttttagttatctttccactatcctaactgcctcatgccccttcactagttcatgccaagaacgtgactggaaaggcatggcacctataccttgattaatttccattaatggaaatggttcgtcctgagtcatctacgttccgagtcaggctgtcactcctactaccaatggccactgtgagccacagaaggaagacatgtacgtgctgttctactttatatgatgtgagaagccaacatcagtgtcccttggcagcagtctacaggcataacttgttttattgtgctgaattttattgtgcttctcaaatattgcacttttaaaaaaattgaaagtttgtggcaatgctgttgagcaaatctgctggcatctttcttcccaaaggcatgtgctcacttcatgtctctgtatcaattgtcataattttcgcaataaacttgattattattaaaaaaaaaaaaaaaaaa

SEQ ID NO: 9 - DNA, 3122 bp, Homo sapiens
tcggcggagacctgctccccagaagacgcctcctgcttcccactgcgccctggaggacgcgggctggctgctgggcgagctcggcggaggcacgcccctcgcctccccgcggagtgcggactcgccccggtgcccaaactccgcccaccctctagggagctccgctctcccgcctaaccccggcactccggacagagctgggcctggggaaggggttcctgaactacgcggacgccgaacgggacgcgctgcagaagcgcacgagtctgcggccacgcgcgctccgatggctgccaggagctgagctcagggtgggcggaggaagcggttagacgccccgaaactgagctgcacgtttctaaggtagggaggaggaagatgcccccaattaagttgatctttgagccaaggaggctggggagcagcctccccaagctagagccctgcagagcgagtttcccttgacctcgctgcgcctctggcgcgctctgcagcgcggacccgcggcccctcgggaaagcgcagtcggaaagttatccgcggcggttccctgcgcgccctgttgtgtaagctcggcgttgccagcggacggagaagttgctggcttgcccgatagcccagttcggtggcggcccggggcggatttcatggcccgcggcgaacgcggggccagagctggcgtgggcgagcccctgcgcgccccctcccgcggggatccagttcgcctgctcccttccgctcgctggcttttccgatgcttgctgcgcccctggccgccgctgccctctcgccgcctcctacccctcggagccgccgcctaagtcgaggaggagagaatgaccgaggtgctgtggccggctgtccccaacgggacggacgctgccttcctggccggtccgggttcgtcctgggggaacagcacggtcgcctccactgccgccgtctcctcgtcgttcaaatgcgccttgaccaagacgggcttccagttttactacctgccggctgtctacatcttggtattcatcatcggcttcctgggcaacagcgtggccatctggatgttcgtcttccacatgaagccctggagcggcatctccgtgtacatgttcaatttggctctggccgacttcttgtacgtgctgactctgccagccctgatcttctactacttcaataaaacagactggatcttcggggatgccatgtgtaaactgcagaggttcatctttcatgtgaacctctatggcagcatcttgtttctgacatgcatcagtgcccaccggtacagcggtgtggtgtaccccctcaagtccctgggccggctcaaaaagaagaatgcgatctgtatcagcgtgctggtgtggctcattgtggtggtggcgatctcccccatcctcttctactcaggtaccggggtccgcaaaaacaaaaccatcacctgttacgacaccacctcagacgagtacctgcgaagttatttcatctacagcatgtgcacgaccgtggccatgttctgtgtccccttggtgctgattctgggctgttacggattaattgtgagagctttgatttacaaagatctggacaactctcctctgaggagaaaatcgatttacctggtaatcattgtactgactgtttttgctgtgtcttacatccctttccatgtgatgaaaacgatgaacttgagggcccggcttgattttcagaccccagcaatgtgtgctttcaatgacagggtttatgccacgtatcaggtgacaagaggtctagcaagtctcaacagttgtgtggaccccattctctatttcttggcgggagatactttcagaaggagactctcccgagccacaaggaaagcttctagaagaagtgaggcaaatttgcaatccaagagtgaagacatgaccctcaatattttacctgagttcaagcagaatggagatacaagcctgtgaaggcacaagaatctccaaacacctctctgttgtaatatggtaggatgcttaacagaatcaagtacttttcccctctttaactttctagtttagaaaaaaatcaaaccaagaaaatagtgagttaaaaaaataatagaagtagaaatgcccacatccacacttagcttgtttgggtttgctttcacagtctctcttccttctgactagaagtatgtataataaaacaatactacctagttaaacatttactttctcttttgcctttaaaatgtgcaggcttttctgtttaaagtgtgtgtgcacatgagtactggggctgtttttgatattagtaatttctctaagaaaactagccccctgcaacttgagtttgtggtttatctagcctttattgtttttttaaaatccacagtaggaataaaaaatctatattctcagaaatatctagcatggtatataacaaaacactaaactcatcagttcatccggcatcagatcaatggatctctgagcggggtgtttttttcagtgtcttataagcatagatgatagttgactgagtttctttagggcattgaatagacaagtaaagctaatgaatttaaaagcctgaaaagtgattgttttccagttatttctggaaaaggtctcattatatattgggtgctaaatgtttgatggggaaagcctgcatatattatcgtactggtaaaatgcattcaaaataattaaagtgcatgtattttccttgtaaacaccatgagctctcttagacatcttgtgataaagagcatttacttgccccactgctgtgcaatgccttaggactttgtttgtgttccaggacaagtgttcactcacatctgtaaaaacaattttaagaattgcaaataaattacagaccaaagattgagtaaagtcaaataactgttagtaagttgaaggatattggacaggaggacagtatttcagaaaaggagaggttgacagtcatccacaaggcatagcctccaagtatactctcaaatgtatgaagcaactggggtgggcagaagacattttagaatgagggctttagtttaaattaaagtcatggtggagaagactcttgcttcctccaagtgtttgaaaacacaaaatgcgatatgaaaaaaaaaaaaaaaa

SEQ ID NO: 10 - DNA, 2395 bp, Homo sapiens
gccgccgtcggcgcgctgggtgcgggaagggggctctggatttcggtccctcccctttttcctctgagtctcggaacgctccagctctcagaccctcttcctcccaggtaaaggccgggagaggagggcgcatctcttttccaggcaccccaccatgggcaatgcctccaatgactcccagtctgaggactgcgagacgcgacagtggcttcccccaggcgaaagcccagccatcagctccgtcatgttctcggccggggtgctggggaacctcatagcactggcgctgctggcgcgccgctggcggggggacgtggggtgcagcgccggccgcaggagctccctctccttgttccacgtgctggtgaccgagctggtgttcaccgacctgctcgggacctgcctcatcagcccagtggtactggcttcgtacgcgcggaaccagaccctggtggcactggcgcccgagagccgcgcgtgcacctacttcgctttcgccatgaccttcttcagcctggccacgatgctcatgctcttcgccatggccctggagcgctacctctcgatcgggcacccctacttctaccagcgccgcgtctcgcgctccgggggcctggccgtgctgcctgtcatctatgcagtctccctgctcttctgctcgctgccgctgctggactatgggcagtacgtccagtactgccccgggacctggtgcttcatccggcacgggcggaccgcttacctgcagctgtacgccaccctgctgctgcttctcattgtctcggtgctcgcctgcaacttcagtgtcattctcaacctcatccgcatgcaccgccgaagccggagaagccgctgcggaccttccctgggcagtggccggggcggccccggggcccgcaggagaggggaaagggtgtccatggcggaggagacggaccacctcattctcctggctatcatgaccatcaccttcgccgtctgctccttgcctttcacgatttttgcatatatgaatgaaacctcttcccgaaaggaaaaatgggacctccaagctcttaggtttttatcaattaattcaataattgacccttgggtctttgccatccttaggcctcctgttctgagactaatgcgttcagtcctctgttgtcggatttcattaagaacacaagatgcaacacaaacttcctgttctacacagtcagatgccagtaaacaggctgacctttgaggtcagtagtttaaaagttcttagttatatagcatctggaagatcattttgaaattgttccttggagaaatgaaaacagtgtgtaaacaaaatgaagctgccctaataaaaaggagtatacaaacatttaagctgtggtcaaggctacagatgtgctgacaaggcacttcatgtaaagtgtcagaaggagctacaaaacctaccctcagtgagcatggtacttggcctttggaggaacaatcggctgcattgaagatccagctgcctattgatttaagctttcctgttgaatgacaaagtatgtggttttgtaatttgtttgaaaccccaaacagtgactgtactttctattttaatcttgctactaccgttatacacatatagtgtacagccagaccagattaaacttcatatgtaatctctaggaagtcaatatgtggaagcaaccaagcctgctgtcttgtgatcacttagcgaaccctttatttgaacaatgaagttgaaaatcataggcaccttttactgtgatgtttgtgtatgtgggagtactctcatcactacagtattactcttacaagagtggactcagtgggttaacatcagttttgtttactcatcctccaggaactgcaggtcaagttgtcaggttatttattttataatgtccatatgctaatagtgatcaagaagactttaggaatggttctctcaacaagaaataatagaaatgtctcaaggcagttaattctcattaatactcttattatcctatttctgggggaggatgtacgtggccatgtatgaagccaaatattaggcttaaaaactgaaaaatctggttcattcttcagatatactggaacccttttaaagttgatattggggccatgagtaaaatagattttataagatgactgtgttgtaccaaaattcatctgtctatattttatttagggaacatggtttgactcatcttatatgggaaaccatgtagcagtgagtcatatcttaatatatttctaaatgtttggcatgtaaatgtaaactcagcatcaaaatatttcagtgaatttgcactgtttaatcatagttactgtgtaaactcatctgaaatgttacaaaaataaactataaaacaaaaatttgaaaaaaaaaaaaaaaaaa

SEQ ID NO: 11 - DNA, 1047 bp, Homo sapiens
atggtgaataatttctcccaagctgaggctgtggagctgtgttacaagaacgtgaacgaatcctgcattaaaactccttactcgccaggtcctcgatctatcctctacgccgtccttggttttggggctgtgctggcagcgtttggaaacttactggtcatgattgctatccttcacttcaaacaactgcacacacctacaaactttctgattgcgtcgctggcctgtgctgacttcttggtgggagtcactgtgatgcccttcagcacagtgaggtctgtggagagctgttggtactttggggacagttactgtaaattccatacatgttttgacacatccttctgttttgcttctttatttcatttatgctgtatctctgttgatagatacattgctgttactgatcctctgacctatccaaccaagtttactgtgtcagtttcagggatatgcattgttctttcctggttcttttctgtcacatacagcttttcgatcttttacacgggagccaacgaagaaggaattgaggaattagtagttgctctaacctgtgtaggaggctgccaggctccactgaatcaaaactgggtcctactttgttttcttctattctttatacccaatgtcgccatggtgtttatatacagtaagatatttttggtggccaagcatcaggctaggaagatagaaagtacagccagccaagctcagtcctcctcagagagttacaaggaaagagtagcaaaaagagagagaaaggctgccaaaaccttgggaattgctatggcagcatttcttgtctcttggctaccatacctcgttgatgcagtgattgatgcttatatgaattttataactcctccttatgtttatgagattttagtttggtgtgtttattataattcagctatgaaccccttgatttatgctttcttttaccaatggtttgggaaggcaataaaacttattgtaagcggcaaggtcttaaggactgattcgtcaacaactaatttattttctgaagaagtagagacagattaa

SEQ ID NO: 12 - DNA, 1020 bp, Homo sapiens
atgatgcccttttgccacaatataattaatatttcctgtgtgaaaaacaactggtcaaatgatgtccgtgcttccctgtacagtttaatggtgctcataattctgaccacactcgttggcaatctgatagttattgtttctatatcacacttcaaacaacttcataccccaacaaattggctcattcattccatggccactgtggactttcttctggggtgtctggtcatgccttacagtatggtgagatctgctgagcactgttggtattttggagaagtcttctgtaaaattcacacaagcaccgacattatgctgagctcagcctccattttccatttgtctttcatctccattgaccgctactatgctgtgtgtgatccactgagatataaagccaagatgaatatcttggttatttgtgtgatgatcttcattagttggagtgtccctgctgtttttgcatttggaatgatctttctggagctaaacttcaaaggcgctgaagagatatattacaaacatgttcactgcagaggaggttgctctgtcttctttagcaaaatatctggggtactgacctttatgacttctttttatatacctggatctattatgttatgtgtctattacagaatatatcttatcgctaaagaacaggcaagattaattagtgatgccaatcagaagctccaaattggattggaaatgaaaaatggaatttcacaaagcaaagaaaggaaagctgtgaagacattggggattgtgatgggagttttcctaatatgctggtgccctttctttatctgtacagtcatggacccttttcttcactacattattccacctactttgaatgatgtattgatttggtttggctacttgaactctacatttaatccaatggtttatgcatttttctatccttggtttagaaaagcactgaagatgatgctgtttggtaaaattttccaaaaagattcatccaggtgtaaattatttttggaattgagttcatag

SEQ ID NO: 13 - DNA, 2290 bp, Homo sapiens
gaattccgaatcatgtgcagaatgctgaatcttcccccagccaggacgaataagacagcgcggaaaagcagattctcgtaattctggaattgcatgttgcaaggagtctcctggatcttcgcacccagcttcgggtagggagggagtccgggtcccgggctaggccagcccggcaggtggagagggtccccggcagccccgcgcgcccctggccatgtctttaatgccctgccccttcatgtggccttctgagggttcccagggctggccagggttgtttcccacccgcgcgcgcgctctcacccccagccaaacccacctggcagggctccctccagccgagaccttttgattcccggctcccgcgctcccgcctccgcgccagcccgggaggtggccctggacagccggacctcgcccggccccggctgggaccatggtgtttctctcgggaaatgcttccgacagctccaactgcacccaaccgccggcaccggtgaacatttccaaggccattctgctcggggtgatcttggggggcctcattcttttcggggtgctgggtaacatcctagtgatcctctccgtagcctgtcaccgacacctgcactcagtcacgcactactacatcgtcaacctggcggtggccgacctcctgctcacctccacggtgctgcccttctccgccatcttcgaggtcctaggctactgggccttcggcagggtcttctgcaacatctgggcggcagtggatgtgctgtgctgcaccgcgtccatcatgggcctctgcatcatctccatcgaccgctacatcggcgtgagctacccgctgcgctacccaaccatcgtcacccagaggaggggtctcatggctctgctctgcgtctgggcactctccctggtcatatccattggacccctgttcggctggaggcagccggcccccgaggacgagaccatctgccagatcaacgaggagccgggctacgtgctcttctcagcgctgggctccttctacctgcctctggccatcatcctggtcatgtactgccgcgtctacgtggtggccaagagggagagccggggcctcaagtctggcctcaagaccgacaagtcggactcggagcaagtgacgctccgcatccatcggaaaaacgccccggcaggaggcagcgggatggccagcgccaagaccaagacgcacttctcagtgaggctcctcaagttctcccgggagaagaaagcggccaaaacgctgggcatcgtggtcggctgcttcgtcctctgctggctgccttttttcttagtcatgcccattgggtctttcttccctgatttcaagccctctgaaacagtttttaaaatagtattttggctcggatatctaaacagctgcatcaaccccatcatatacccatgctccagccaagagttcaaaaaggcctttcagaatgtcttgagaatccagtgtctccgcagaaagcagtcttccaaacatgccctgggctacaccctgcacccgcccagccaggccgtggaagggcaacacaaggacatggtgcgcatccccgtgggatcaagagagaccttctacaggatctccaagacggatggcgtttgtgaatggaaatttttctcttccatgccccgtggatctgccaggattacagtgtccaaagaccaatcctcctgtaccacagcccgggtgagaagtaaaagctttttggaggtctgctgctgtgtagggccctcaacccccagccttgacaagaaccatcaagttccaaccattaaggtccacaccatctccctcagtgagaacggggaggaagtctaggacaggaaagatgcagaggaaaggggaataatcttaggtacccaccccacttccttctcggaaggccagctcttcttggaggacaagacaggaccaatcaaagaggggacctgctgggaatggggtgggtggtagacccaactcatcaggcagcgggtagggcacagggaagagggagggtgtctcacaaccaaccagttcagaatgatacggaacagcatttccctgcagctaatgctttcttggtcactctgtgcccacttcaacgaaaaccaccatgggaaacagaatttcatgcacaatccaaaagactataaatataggattatgatttcatcatgaatattttgagcacacactctaagtttggagctatttcttgatggaagtgaggggattttattttcaggctcaacctactgacagccacatttgacatttatgccggaattc

SEQ ID NO: 14 - DNA, 2089 bp, Homo sapiens
gaattccgaatcatgtgcagaatgctgaatcttcccccagccaggacgaataagacagcgcggaaaagcagattctcgtaattctggaattgcatgttgcaaggagtctcctggatcttcgcacccagcttcgggtagggagggagtccgggtcccgggctaggccagcccggcaggtggagagggtccccggcagccccgcgcgcccctggccatgtctttaatgccctgccccttcatgtggccttctgagggttcccagggctggccagggttgtttcccacccgcgcgcgcgctctcacccccagccaaacccacctggcagggctccctccagccgagaccttttgattcccggctcccgcgctcccgcctccgcgccagcccgggaggtggccctggacagccggacctcgcccggccccggctgggaccatggtgtttctctcgggaaatgcttccgacagctccaactgcacccaaccgccggcaccggtgaacatttccaaggccattctgctcggggtgatcttggggggcctcattcttttcggggtgctgggtaacatcctagtgatcctctccgtagcctgtcaccgacacctgcactcagtcacgcactactacatcgtcaacctggcggtggccgacctcctgctcacctccacggtgctgcccttctccgccatcttcgaggtcctaggctactgggccttcggcagggtcttctgcaacatctgggcggcagtggatgtgctgtgctgcaccgcgtccatcatgggcctctgcatcatctccatcgaccgctacatcggcgtgagctacccgctgcgctacccaaccatcgtcacccagaggaggggtctcatggctctgctctgcgtctgggcactctccctggtcatatccattggacccctgttcggctggaggcagccggcccccgaggacgagaccatctgccagatcaacgaggagccgggctacgtgctcttctcagcgctgggctccttctacctgcctctggccatcatcctggtcatgtactgccgcgtctacgtggtggccaagagggagagccggggcctcaagtctggcctcaagaccgacaagtcggactcggagcaagtgacgctccgcatccatcggaaaaacgccccggcaggaggcagcgggatggccagcgccaagaccaagacgcacttctcagtgaggctcctcaagttctcccgggagaagaaagcggccaaaacgctgggcatcgtggtcggctgcttcgtcctctgctggctgccttttttcttagtcatgcccattgggtctttcttccctgatttcaagccctctgaaacagtttttaaaatagtattttggctcggatatctaaacagctgcatcaaccccatcatatacccatgctccagccaagagttcaaaaaggcctttcagaatgtcttgagaatccagtgtctccgcagaaagcagtcttccaaacatgccctgggctacaccctgcacccgcccagccaggccgtggaagggcaacacaaggacatggtgcgcatccccgtgggatcaagagagaccttctacaggatctccaagacggatggcgtttgtgaatggaaatttttctcttccatgccccgtggatctgccaggattacagtgtccaaagaccaatcctcctgtaccacagcccggggacacacacccatgacatgaagccagcttcccgtccacgactgttgtccttactgcccaaggaaggggagcatgaaacccaccactggtcctgcgacccactgtctttggaatccaccccaggagcccaggagccttgcctgacacttggatttacttctttatcaagcatccatctgactaaggcacaaatccaacatgttactgttactgatacaggaaaaacagtaacttaaggaatgatcatgaatgcaaagggaaagaggaaaagagccttcagggacaaatagctcgattttttgtaaatcagtttcatacaacctccctcccccatttcattcttaaaagttaattgagaatcatcagccacgtgtagggtgtgag

SEQ ID NO: 15 - DNA, 4267 bp, Homo sapiens
ctccgaggaggggtggggacggtcctgacggtggggacatcaggctgccccgcagtaccagggagcgacttgaagtgcccatgccgcttgctccgggagaagcccaggtgtggcctcactcacatcccactctgagtccaaatgttctctccctggaagatatcaatgtttctgtctgttcgtgaggactccgtgcccaccacggcctctttcagcgccgacatgctcaatgtcaccttgcaagggcccactcttaacgggacctttgcccagagcaaatgcccccaagtggagtggctgggctggctcaacaccatccagccccccttcctctgggtgctgttcgtgctggccaccctagagaacatctttgtcctcagcgtcttctgcctgcacaagagcagctgcacggtggcagagatctacctggggaacctggccgcagcagacctgatcctggcctgcgggctgcccttctgggccatcaccatctccaacaacttcgactggctctttggggagacgctctgccgcgtggtgaatgccattatctccatgaacctgtacagcagcatctgtttcctgatgctggtgagcatcgaccgctacctggccctggtgaaaaccatgtccatgggccggatgcgcggcgtgcgctgggccaagctctacagcttggtgatctgggggtgtacgctgctcctgagctcacccatgctggtgttccggaccatgaaggagtacagcgatgagggccacaacgtcaccgcttgtgtcatcagctacccatccctcatctgggaagtgttcaccaacatgctcctgaatgtcgtgggcttcctgctgcccctgagtgtcatcaccttctgcacgatgcagatcatgcaggtgctgcggaacaacgagatgcagaagttcaaggagatccagacggagaggagggccacggtgctagtcctggttgtgctgctgctattcatcatctgctggctgcccttccagatcagcaccttcctggatacgctgcatcgcctcggcatcctctccagctgccaggacgagcgcatcatcgatgtaatcacacagatcgcctccttcatggcctacagcaacagctgcctcaacccactggtgtacgtgatcgtgggcaagcgcttccgaaagaagtcttgggaggtgtaccagggagtgtgccagaaagggggctgcaggtcagaacccattcagatggagaactccatgggcacactgcggacctccatctccgtggaacgccagattcacaaactgcaggactgggcagggagcagacagtgagcaaacgccagcagggctgctgtgaatttgtgtaaggattgagggacagttgcttttcagcatgggcccaggaatgccaaggagacatctatgcacgaccttgggaaatgagttgatgtctccggtaaaacaccggagactaattcctgccctgcccaattttgcagggagcatggctgtgaggatggggtgaactcacgcacagccaaggactccaaaatcacaacagcattactgttcttatttgctgccacacctgagccagcctgctccttcccaggagtggaggaggcctgggggcagggagaggagtgactgagcttccctcccgtgtgttctccgtccctgccccagcaagacaacttagatctccaggagaactgccatccagctttggtgcaatggctgagtgcacaagtgagttgttgccctgggtttctttaatctattcagctagaactttgaaggacaatttcttgcattaataaaggttaagccctgaggggtccctgataacaacctggagaccaggattttatggctcccctcactgatggacaaggaggtctgtgccaaagaagaatccaataagcacatattgagcacttgctgtatatgcagtattgagcactgtaggcaagagggaagaaagagaaggagccatctccatcttgaaggaactcaaagactcaagtgggaacgactgggcactgccaccaccagaaagctgttcgacgagacggtcgagcagggtgctgtgggtgatatggacagcagaagggggagaccaaggttccagctcaaccaataactattgcacaaccacctgtccctgcctcagttccctcttctgtaacatgaagtcgttgtgagggttaaaggcagtaacaggtataaagtacttagaaaagcaaagggtgctacgtacatgtgaggcatcattacgcagacgtaactgggatatgtttactataaggaaaagacactgaggtctagaaatagctccgtggagcagaatcagtattgggagccggtggcggtgtgaagcaccagtgtctggcacacagtaggtgctcattggctcccttccacctgtcattcccaccaccctgaggccccaaccgccacacacacaggagcatttggagagaaggccatgtcttcaaagtctgatttgtgatgaggcagaggaagatatttctaatcggtcttgcccagaggatcacagtgctgagaccccccaccaccagccggtacctgggaagggggagagtgcaggcctgctcagggactgttcctgtctcagcaaccaagggattgttcctgtcaatcaatggtttattggaaggtggcccagtatgagccctagaagagtgtgaaaaggaatggcaatggtgttcaccatcggcagtgccagggcagcactcattcacttgataaatgaatatttattagctggttggagagctagaacctggagaggctagaacctggagaactagaacctggagggctagaacctggagaggctagaaccaagaagggctagaacctggaggggctagaacctagagaagctaaaacctgagctagaagctggaggactagaacctggagggctggaatctggagagctagaacctggagggctagaacctggagggctagaatctggagagctagaacctggagggctagaatctggagagctagaacctggagggctagaacctggagagctagaacctagaagggctagaacctggagggctggaatctggagagctagaacctggagggctagaacctggagggctagaacctagaagggctagaacctggagggctggaatctggagagctagaacctggagggctagaacctggagggctagaacctagaagggctagaacctggagggctagaacctggcaggttagaacctagaagggctagaacctggagagccagaacctggagggctagaacctggaagggctagaacctgtagagctagaacatggagagctagaacccggcaggctagaacctggcaagctagaacctggagggaatgaacctggagggctagaacctggagaatgagaaaaatttacatggcaaagagcccataaatcctgaccaatccaactctgaattttaaagcaaaagcgtcaaaaaaaagattccctccttacccccaacccactcttttttcccaccacccactctcctctgcctcagtaagtatctggaggaagaaaacaggtgaaagaagaagtaaaaaccatttagtattagtattagaatgaagtcaaactgtgccacacatggtgaatgaaaaaaaaaaaaagaggctgtgttttgtcacacagggcagtcattcagcaccagagcacgtgatggtctgagactctcttaggagcagagctctgccgcaatggccatgtggggatccacacctggtctgaggggcaactgagtctgcgggagaagagcggccctatgcatggtgtagatgccctgataaagaacatctgtcctgtgaaagactcaatgagctgttatgttgtaaacaggaagcatttcacatccaaacgagaaaatcatgtaaacatgtgtcttttctgtagagcataataaatggatgaggtttttgcatagctctagcatttgttacaactcccgaaacccccgagtttggtccctggggtaccgccttgcacactcagaagcctttgggaaggggtgctattcatttctgctcaatctgttaacaggcttctggcatgtagatcagtggtctccaagcttttgtgattgtatattcctataggaaaaaaagaattgattatgcatacccagtatgtatacttattaatctgtatgaagatgtacattctaaaatataatcaaccagtagaaatttaagaaagaagatgtaaaaaa

SEQ ID NO: 16 - DNA, 1330 bp, Homo sapiens
cggccgccggcagggttcgcgaggcacccacgctcctaaaaagagcacgacgcacccgatgctcggattggatgaagtgcaaagctttaatccctggaaaggccacgaacaatgaatccatttcatgcatcttgttggaacacctctgccgaacttttaaacaaatcctggaataaagagtttgcttatcaaactgccagtgtggtggatacagtcatcctcccttccatgattgggattatctgttcaacagggctggttggcaacatcctcattgtattcactataataagatccaggaaaaaaacagtccctgacatctatatctgcaacctggctgtggctgatttggtccacatagttggaatgccttttcttattcaccaatgggcccgagggggagagtgggtgtttggggggcctctctgcaccatcatcacatccctggatacttgtaaccaatttgcctgtagtgccatcatgactgtaatgagtgtggacaggtactttgccctcgtccaaccatttcgactgacacgttggagaacaaggtacaagaccatccggatcaatttgggcctttgggcagcttcctttatcctggcattgcctgtctgggtctactcgaaggtcatcaaatttaaagacggtgttgagagttgtgcttttgatttgacatcccctgacgatgtactctggtatacactttatttgacgataacaactttttttttccctctacccttgattttggtgtgctatattttaattttatgctatacttgggagatgtatcaacagaataaggatgccagatgctgcaatcccagtgtaccaaaacagaragtgatgaagttgacaaagatggtgctggtgctggtggtagtctttatcctgagtgctgccccttatcatgtgatacaactggtgaacttacagatggaacagcccacactggccttctatgtgggttattacctctccatctgtctcagctatgccagcagcagcattaacccttttctctacatcctgctgagtggaaatttccagaaacgtctgcctcaaatccaaagaagagcgactgagaaggaaatcaacaatatgggaaacactctgaaatcacacttttaggaaagtacatggatcaccatgagtctagacatgattgtctatcttactggtattattagaaagggcaggtgtaccgatatgtttatgcccattcttcttgtgtacttgtgactcttagcagcatggaagagaagtgtaaccatgcaaatacaatgagcttaatatgctaactgtaaaaaaaaaaaaaaaaaaaaaa

SEQ ID NO: 17 - DNA, 1773 bp, Homo sapiens
atgaccttgcacaataacagtacaacctcgcctttgtttccaaacatcagctcctcctggatacacagcccctccgatgcagggctgcccccgggaaccgtcactcatttcggcagctacaatgtttctcgagcagctggcaatttctcctctccagacggtaccaccgatgaccctctgggaggtcataccgtctggcaagtggtcttcatcgctttcttaacgggcatcctggccttggtgaccatcatcggcaacatcctggtaattgtgtcatttaaggtcaacaagcagctgaagacggtcaacaactacttcctcttaagcctggcctgtgccgatctgattatcggggtcatttcaatgaatctgtttacgacctacatcatcatgaatcgatgggccttagggaacttggcctgtgacctctggcttgccattgactacgtagccagcaatgcctctgttatgaatcttctggtcatcagctttgacagatacttttccatcacgaggccgctcacgtaccgagccaaacgaacaacaaagagagccggtgtgatgatcggtctggcttgggtcatctcctttgtcctttgggctcctgccatcttgttctggcaatactttgttggaaagagaactgtgcctccgggagagtgcttcattcagttcctcagtgagcccaccattacttttggcacagccatcgctgctttttatatgcctgtcaccattatgactattttatactggaggatctataaggaaactgaaaagcgtaccaaagagcttgctggcctgcaagcctctgggacagaggcagagacagaaaactttgtccaccccacgggcagttctcgaagctgcagcagttacgaacttcaacagcaaagcatgaaacgctccaacaggaggaagtatggccgctgccacttctggttcacaaccaagagctggaaacccagctccgagcagatggaccaagaccacagcagcagtgacagttggaacaacaatgatgctgctgcctccctggagaactccgcctcctccgacgaggaggacattggctccgagacgagagccatctactccatcgtgctcaagcttccgggtcacagcaccatcctcaactccaccaagttaccctcatcggacaacctgcaggtgcctgaggaggagctggggatggtggacttggagaggaaagccgacaagctgcaggcccagaagagcgtggacgatggaggcagttttccaaaaagcttctccaagcttcccatccagctagagtcagccgtggacacagctaagacttctgacgtcaactcctcagtgggtaagagcacggccactctacctctgtccttcaaggaagccactctggccaagaggtttgctctgaagaccagaagtcagatcactaagcggaaaaggatgtccctggtcaaggagaagaaagcggcccagaccctcagtgcgatcttgcttgccttcatcatcacttggaccccatacaacatcatggttctggtgaacaccttttgtgacagctgcatacccaaaaccttttggaatctgggctactggctgtgctacatcaacagcaccgtgaaccccgtgtgctatgctctgtgcaacaaaacattcagaaccactttcaagatgctgctgctgtgccagtgtgacaaaaaaaagaggcgcaagcagcagtaccagcagagacagtcggtcatttttcacaagcgcgcacccgagcaggccttgtag

SEQ ID NO: 18 - DNA, 2162 bp, Homo sapiens
  misc_feature (2063)..(2063): n is a, c, g, or t
ggaattccggctataggcagaggagaatgtcagatgctcagctcggtcccctccgcctgacgctcctctctgtctcagccaggactggtttctgtaagaaacagcaggagctgtggcagcggcgaaaggaagcggctgaggcgcttggaacccgaaaagtctcggtgctcctggctacctcgcacagcggtgcccgcccggccgtcagtaccatggacagcagcgctgcccccacgaacgccagcaattgcactgatgccttggcgtactcaagttgctccccagcacccagccccggttcctgggtcaacttgtcccacttagatggcaacctgtccgacccatgcggtccgaaccgcaccaacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctcgtgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcagctagaaaatctggaagcagaaactgctccgttgccctaacagggtctcatgccattccgaccttcaccaagcttagaagccaccatgtatgtggaagcaggttgcttcaagaatgtgtaggaggctctaattctctaggaaagtgcctacttttaggtcatccaacctctttcctctctggccactctgctctgcacattagagggacagccaaaagtaagtggagcatttggaaggaaaggaatataccacaccgaggagtccagtttgtgcaagacacccagtggaaccaaaacccatcgtggtatgtgaattgaagtcatcataaaaggtgacccttctgtctgtaagattttattttcaagcaaatatttatgacctcaacaaagaagaaccatcttttgttaagttcaccgtagtaacacataaagtaaatgctacctctgatcaaagcaccttgaatggaaggtccgagtctttttagtgtttttgcaagggaatgaatccattattctattttagacttttaacttcaacttaaaattagcatctggctaaggcatcattttcacctccatttcttggttttgtattgtttaaaaaaaataacatctctttcatctagctccataattgcaagggaagagattagcatgaaaggtaatctgaaacacagtcatgtgtcanctgtagaaaggttgattctcatgcactncaaatacttccaaagagtcatcatgggggatttttcattcttaggctttcagtggtttgttcctggaattc

SEQ ID NO: 19 - DNA, 1766 bp, Homo sapiens
aattcagagccaccgcgggcaggcgggcagtgcatccagaagcgtttatattctgagcgccagttcagctttcaaaaagagtgctgcccataaaaagccttccaccctcctgtctgctttagaaggaccctgagccccaggcgccagccacaggactctgctgcagaggggggttgtgtacagatagtaggctttacgcctagcttcgaaatggataacgtcctcccggtggactcagacctctccccaaacatctccactaacacctcggaacccaatcagttcgtgcaaccagcctggcaaattgtcctttgggcagctgcctacacggtcattgtggtgacctctgtggtgggcaacgtggtagtgatgtggatcatcttagcccacaaaagaatgaggacagtgacgaactattttctggtgaacctggccttcgcggaggcctccatggctgcattcaatacagtggtgaacttcacctatgctgtccacaacgaatggtactacggcctgttctactgcaagttccacaacttctttcccatcgccgctgtcttcgccagtatctactccatgacggctgtggcctttgataggtacatggccatcatacatcccctccagccccggctgtcagccacagccaccaaagtggtcatctgtgtcatctgggtcctggctctcctgctggccttcccccagggctactactcaaccacagagaccatgcccagcagagtcgtgtgcatgatcgaatggccagagcatccgaacaagatttatgagaaagtgtaccacatctgtgtgactgtgctgatctacttcctccccctgctggtgattggctatgcatacaccgtagtgggaatcacactatgggccagtgagatccccggggactcctctgaccgctaccacgagcaagtctctgccaagcgcaaggtggtcaaaatgatgattgtcgtggtgtgcaccttcgccatctgctggctgcccttccacatcttcttcctcctgccctacatcaacccagatctctacctgaagaagtttatccagcaggtctacctggccatcatgtggctggccatgagctccaccatgtacaaccccatcatctactgctgcctcaatgacaggttccgtctgggcttcaagcatgccttccggtgctgccccttcatcagcgccggcgactatgaggggctggaaatgaaatccacccggtatctccagacccagggcagtgtgtacaaagtcagccgcctggagaccaccatctccacagtggtgggggcccacgaggaggagccagaggacggccccaaggccacaccctcgtccctggacctgacctccaactgctcttcacgaagtgactccaagaccatgacagagagcttcagcttctcctccaatgtgctctcctaggccacagggcctttggcaggtgcagcccccactgcctttgacctgcctcccttcatgcatggaaattcccttcatctggaaccatcagaaacaccctcacactgggacttgcaaaaagggtcagtatgggttagggaaaacattccatccttgagtcaaaaaatctcaattcttccctatctttgccaccctcatgctgtgtgactcaaaccaaatcactgaactttgctgagcctgtaaaataaaaggtcggaccagcttttcctcaagagcccaatgcattccatttctggaagtgactttggctgcatgcgagtgctcatttcaggatg

SEQ ID NO: 20 - DNA, 1002 bp, Homo sapiens
atggagtcctcaggcaacccagagagcaccacctttttttactatgaccttcagagccagccgtgtgagaaccaggcctgggtctttgctaccctcgccaccactgtcctgtactgcctggtgtttctcctcagcctagtgggcaacagcctggtcctgtgggtcctggtgaagtatgagagcctggagtccctcaccaacatcttcatcctcaacctgtgcctctcagacctggtgttcgcctgcttgttgcctgtgtggatctccccataccactggggctgggtgctgggagacttcctctgcaaactcctcaatatgatcttctccatcagcctctacagcagcatcttcttcctgaccatcatgaccatccaccgctacctgtcggtagtgagccccctctccaccctgcgcgtccccaccctccgctgccgggtgctggtgaccatggctgtgtgggtagccagcatcctgtcctccatcctcgacaccatcttccacaaggtgctttcttcgggctgtgattattccgaactcacgtggtacctcacctccgtctaccagcacaacctcttcttcctgctgtccctggggattatcctgttctgctacgtggagatcctcaggaccctgttccgctcacgctccaagcggcgccaccgcacggtcaagctcatcttcgccatcgtggtggcctacttcctcagctggggtccctacaacttcaccctgtttctgcagacgctgtttcggacccagatcatccggagctgcgaggccaaacagcagctagaatacgccctgctcatctgccgcaacctcgccttctcccactgctgctttaacccggtgctctatgtcttcgtgggggtcaagttccgcacacacctgaaacatgttctccggcagttctggttctgccggctgcaggcacccagcccagcctcgatcccccactcccctggtgccttcgcctatgagggcgcctccttctactga

SEQ ID NO: 21 - DNA, 2753 bp, Homo sapiens
gtcgggggcagcagcaagatgcgaagcgagccgtacagatcccgggctctccgaacgcaacttcgccctgcttgagcgaggctgcggtttccgaggccctctccagccaaggaaaagctacacaaaaagcctggatcactcatcgaaccacccctgaagccagtgaaggctctctcgcctcgccctctagcgttcgtctggagtagcgccaccccggcttcctggggacacagggttggcaccatggggcccaccagcgtcccgctggtcaaggcccaccgcagctcggtctctgactacgtcaactatgatatcatcgtccggcattacaactacacgggaaagctgaatatcagcgcggacaaggagaacagcattaaactgacctcggtggtgttcattctcatctgctgctttatcatcctggagaacatctttgtcttgctgaccatttggaaaaccaagaaattccaccgacccatgtactattttattggcaatctggccctctcagacctgttggcaggagtagcctacacagctaacctgctcttgtctggggccaccacctacaagctcactcccgcccagtggtttctgcgggaagggagtatgtttgtggccctgtcagcctccgtgttcagtctcctcgccatcgccattgagcgctatatcacaatgctgaaaatgaaactccacaacgggagcaataacttccgcctcttcctgctaatcagcgcctgctgggtcatctccctcatcctgggtggcctgcctatcatgggctggaactgcatcagtgcgctgtccagctgctccaccgtgctgccgctctaccacaagcactatatcctcttctgcaccacggtcttcactctgcttctgctctccatcgtcattctgtactgcagaatctactccttggtcaggactcggagccgccgcctgacgttccgcaagaacatttccaaggccagccgcagctctgagaagtcgctggcgctgctcaagaccgtaattatcgtcctgagcgtcttcatcgcctgctgggcaccgctcttcatcctgctcctgctggatgtgggctgcaaggtgaagacctgtgacatcctcttcagagcggagtacttcctggtgttagctgtgctcaactccggcaccaaccccatcatttacactctgaccaacaaggagatgcgtcgggccttcatccggatcatgtcctgctgcaagtgcccgagcggagactctgctggcaaattcaagcgacccatcatcgccggcatggaattcagccgcagcaaatcggacaattcctcccacccccagaaagacgaaggggacaacccagagaccattatgtcttctggaaacgtcaactcttcttcctagaactggaagctgtccacccaccggaagcgctctttacttggtcgctggccaccccagtgtttggaaaaaaatctctgggcttcgactgctgccagggaggagctgctgcaagccagagggaggaagggggagaatacgaacagcctggtggtgtcgggtgttggtgggtagagttagttcctgtgaacaatgcactgggaagggtggagatcaggtcccggcctggaatatatattctacccccctggagctttgattttgcactgagccaaaggtctagcattgtcaagctcctaaagggttcatttggcccctcctcaaagactaatgtccccatgtgaaagcgtctctttgtctggagctttgaggagatgttttccttcactttagtttcaaacccaagtgagtgtgtgcacttctgcttctttagggatgccctgtacatcccacaccccaccctcccttcccttcatacccctcctcaacgttcttttactttatactttaactacctgagagttatcagagctggggttgtggaatgatcgatcatctatagcaaataggctatgttgagtacgtaggctgtgggaagatgaagatggtttggaggtgtaaaacaatgtccttcgctgaggccaaagtttccatgtaagcgggatccgttttttggaatttggttgaagtcactttgatttctttaaaaaacatcttttcaatgaaatgtgttaccatttcatatccattgaagccgaaatctgcataaggaagcccactttatctaaatgatattagccaggatccttggtgtcctaggagaaacagacaagcaaaacaaagtgaaaaccgaatggattaacttttgcaaaccaagggagatttcttagcaaatgagtctaacaaatatgacatccgtctttcccacttttgttgatgtttatttcagaatcttgtgtgattcatttcaagcaacaacatgttgtattttgttgtgttaaaagtacttttcttgatttttgaatgtatttgtttcaggaagaagtcattttatggatttttctaacccgtgttaacttttctagaatccaccctcttgtgcccttaagcattactttaactggtagggaacgccagaacttttaagtccagctattcattagatagtaattgaagatatgtataaatattacaaagaataaaaatatattactgtctctttagtatggttttcagtgcaattaaaccgagagatgtcttgtttttttaaaaagaatagtatttaataggtttctgacttttgtggatcattttgcacatagctttatcaacttttaaacattaataaactgatttttttaaag

SEQ ID NO: 22 - DNA, 3023 bp, Homo sapiens
gccattctctcacatcccgtgcggtcaggaagcccttcctgaactctgacttcagttcttgctgcggtttctgcccatttttttcatatcctctgacagctgcgaggtcatctctgctctggcttttctccaagcagaacaagtgggggctctggaaaggttaagggacctcagtggccaccattatactttgcatctttcctgagaagtgagagttgaaagggaagcaggaaggcccatggtcagattgaaggaaggactttttagtttcttttttttttttttgagatggagtctcgctctgtcattcaggctggagtgcagtggtgcgatctcagctcactgcagcctccacttcctgggttcacatgattctcctgcctcagcctcccaagtagctgagactacaggcacatgccactacacccagctaacttttgtatttttagtagagacggggtttcaccatgttggccaggctggtctcaaactgctaacatcaagtgatctgctcccctcagcctcccaaagtgctgggattaccggtatgaaccaccacaacctgccaggaatttttagtttttagcttttgcaggagacttcaaggaaaggagacattcctctgtccaggaaacgggtaaggggaccatttctgcattgctggtttcccctcttggcagggtgggcatgaggcatcactgttcctgctccctcactcctgctcctcatgctcagcctgccagctcggcctcaactttgtgtgtctaaagtggaactgaatagtaggctgtgagaagataggaaagaggtagtgccaatctccttgcccagatcataaatccagactcagcagggtaaccacatgggcaagcacaaggtaggtgcttggggaaaggggaagtaattggcattctgtgtgataccaaggagaccatttggattttggcttctaccaaagagaatggagaattggttgacctaaatggaaccagtccctttaagtaaggggaggaaagggggtgctggaagatggccctcttcccaccacctagatcatagcttgaactgaagccaaggacagagtgctgcccccttcggcatttactgatgtgccctctttaaatcatgatgttatctaacccaaacccagacccaggacctagtcacagctccaacctacacttcctattaatcttaaaacaaagcaaaacaaaacaaaaagatatcagcattgtagcctccaatctgagcccatttcccttctctggctaccatacctccttctcctatatgataccattcactactttgttcaattatccagtctagacctgcatcttgaggccacacccagccttctcactccccacacccctctttcctctctcactgctccttcctggtctcttctcatctggccccacctctaaggagtcctcctgccttctgggttgccctggaaaacagactatcccccctcctagtgaagggagtgggtaggggtttcagccccaccctcaggaagatgcgtcttccctgtcctctgctctgtggtacttcctctctggctgatttagcaaacagcacctagacctggggccaggcctttggcagtgggacagatccagggataggctacaccaccctgccctgaccctgggattggcatcagcttccaaccagttcctgccaaagcttgtaagtcctcccgacggccatgaacactacatcttctgcagcacccccctcactaggtgtagagttcatctctctgctggctatcatcctgctgtcagtggcgctggctgtggggcttcccggcaacagctttgtggtgtggagtatcctgaaaaggatgcagaagcgctctgtcactgccctgatggtgctgaacctggccctggccgacctggccgtattgctcactgctccctttttccttcacttcctggcccaaggcacctggagttttggactggctggttgccgcctgtgtcactatgtctgcggagtcagcatgtacgccagcgtcctgcttatcacggccatgagtctagaccgctcactggcggtggcccgcccctttgtgtcccagaagctacgcaccaaggcgatggcccggcgggtgctggcaggcatctgggtgttgtcctttctgctggccacacccgtcctcgcgtaccgcacagtagtgccctggaaaacgaacatgagcctgtgcttcccgcggtaccccagcgaagggcaccgggccttccatctaatcttcgaggctgtcacgggcttcctgctgcccttcctggctgtggtggccagctactcggacatagggcgtcggctacaggcccggcgcttccgccgcagccgccgcaccggccgcctggtggtgctcatcatcctgaccttcgccgccttctggctgccctaccacgtggtgaacctggctgaggcgggccgcgcgctggccggccaggccgccgggttagggctcgtggggaagcggctgagcctggcccgcaacgtgctcatcgcactcgccttcctgagcagcagcgtgaaccccgtgctgtacgcgtgcgccggcggcggcctgctgcgctcggcgggcgtgggcttcgtcgccaagctgctggagggcacgggctccgaggcgtccagcacgcgccgcgggggcagcctgggccagaccgctaggagcggccccgccgctctggagcccggcccttccgagagcctcactgcctccagccctctcaagttaaacgaactgaactaggcctggtggaaggaggcgcactttcctcctggcagaatgctagctctgagccagttcagtacctggaggaggagcaggggcgtggagggcgtggagggcgtgggagcgtgggaggcgggagtggagtggaagaagagggagaggtggagcaaagtgagggccgagtgagagcgtgctccagcctggctcccacaggcagctttaaccattaaaactgaagtctgaaatttggtcaaaaaaaaaaaaa

SEQ ID NO: 23 - DNA, 2273 bp, Homo sapiens
caggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataacattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctagagacagaaatgacagatctctgctttggaaatcacacgtctggcttcacagatgtgtgattcacagtgtgaatcttggtgtctacgttaccaggcaggaaggctgagaggagagagactccagctgggttggaaaacagtattttccaaactaccttccagttcctcatttttgaatacaggcatagagttcagactttttttaaatagtaaaaataaaattaaagctgaaaactgcaacttgtaaatgtggtaaagagttagtttgagttgctatcatgtcaaacgtgaaaatgctgtattagtcacagagataattctagctttgagcttaagaattttgagcaggtggtatgtttgggagactgctgagtcaacccaatagttgttgattggcaggagttggaagtgtgtgatctgtgggcacattagcctatgtgcatgcagcatctaagtaatgatgtcgtttgaatcacagtatacgctccatcgctgtcatctcagctggatctccattctctcaggcttgctgccaaaagccttttgtgttttgttttgtatcattatgaagtcatgcgtttaatcacattcgagtgtttcagtgcttcgcagatgtccttgatgctcatattgttccctaatttgccagtgggaactcctaaatcaaattggcttctaatcaaagcttttaaaccctattggtaaagaatggaaggtggagaagctccctgaagtaagcaaagactttcctcttagtcgagccaagttaagaatgttcttatgttgcccagtgtgtttctgatctgatgcaagcaagaaacactgggcttctagaaccaggcaacttgggaactagactcccaagctggactatggctctactttcaggccacatggctaaagaaggtttcagaaagaagtggggacagagcagaactttcaccttcatatatttgtatgatcctaatgaatgcataaaatgttaagttgatggtgatgaaatgtaaatactgtttttaacaactatgatttggaaaataaatcaatgctataactatgttgataaaag

SEQ ID NO: 24 - DNA, 3655 bp, Homo sapiens
cttcagatagattatatctggagtgaaggatcctgccacctacgtatctggcatagtattctgtgtagtgggatgagcagagaacaaaaacaaaataatccagtgagaaaagcccgtaaataaaccttcagaccagagatctattctccagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttggtttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatataggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctttctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttcta

SEQ ID NO: 25 - DNA, 3177 bp, Homo sapiens
tgcaaacgttcccaaatcttcccagtcggcttgcagagactccttgctcccaggagataaccagaagctgcatcttattgacagatggtcatcacattggtgagctggagtcatcagattgtggggcccggagtgaggctgaagggagtggatcagagcactgcctgagagtcacctctactttcctgctaccgctgcctgtgagctgaaggggctgaaccatacactcctttttctacaaccagcttgcattttttctgcccacaatgagcggggaatcaatgaatttcagcgatgttttcgactccagtgaagattattttgtgtcagtcaatacttcatattactcagttgattctgagatgttactgtgctccttgcaggaggtcaggcagttctccaggctatttgtaccgattgcctactccttgatctgtgtctttggcctcctggggaatattctggtggtgatcacctttgctttttataagaaggccaggtctatgacagacgtctatctcttgaacatggccattgcagacatcctctttgttcttactctcccattctgggcagtgagtcatgccaccggtgcgtgggttttcagcaatgccacgtgcaagttgctaaaaggcatctatgccatcaactttaactgcgggatgctgctcctgacttgcattagcatggaccggtacatcgccattgtacaggcgactaagtcattccggctccgatccagaacactaccgcgcagcaaaatcatctgccttgttgtgtgggggctgtcagtcatcatctccagctcaacttttgtcttcaaccaaaaatacaacacccaaggcagcgatgtctgtgaacccaagtaccagactgtctcggagcccatcaggtggaagctgctgatgttggggcttgagctactctttggtttctttatccctttgatgttcatgatattttgttacacgttcattgtcaaaaccttggtgcaagctcagaattctaaaaggcacaaagccatccgtgtaatcatagctgtggtgcttgtgtttctggcttgtcagattcctcataacatggtcctgcttgtgacggctgcaaatttgggtaaaatgaaccgatcctgccagagcgaaaagctaattggctatacgaaaactgtcacagaagtcctggctttcctgcactgctgcctgaaccctgtgctctacgcttttattgggcagaagttcagaaactactttctgaagatcttgaaggacctgtggtgtgtgagaaggaagtacaagtcctcaggcttctcctgtgccgggaggtactcagaaaacatttctcggcagaccagtgagaccgcagataacgacaatgcgtcgtccttcactatgtgatagaaagctgagtctccctaaggcatgtgtgaaacatactcatagatgttatgcaaaaaaaagtctatggccaggtatgcatggaaaatgtgggaattaagcaaaatcaagcaagcctctctcctgcgggacttaacgtgctcatgggctgtgtgatctcttcagggtggggtggtctctgataggtagcattttccagcactttgcaaggaatgttttgtagctctagggtatatatccgcctggcatttcacaaaacagcctttgggaaatgctgaattaaagtgaattgttgacaaatgtaaacattttcagaaatattcatgaagcggtcacagatcacagtgtcttttggttacagcacaaaatgatggcagtggtttgaaaaactaaaacagaaaaaaaaatggaagccaacacatcactcattttaggcaaatgtttaaacatttttatctatcagaatgtttattgttgctggttataagcagcaggattggccggctagtgtttcctctcatttccctttgatacagtcaacaagcctgaccctgtaaaatggaggtggaaagacaagctcaagtgttcacaacctggaagtgcttcgggaagaaggggacaatggcagaacaggtgttggtgacaattgtcaccaattggataaagcagctcaggttgtagtgggccattaggaaactgtcggtttgctttgatttccctgggagctgttctctgtcgtgagtgtctcttgtctaaacgtccattaagctgagagtgctatgaagacaggatctagaataatcttgctcacagctgtgctctgagtgcctagcggagttccagcaaacaaaatggactcaagagagatttgattaatgaatcgtaatgaagttggggtttattgtacagtttaaaatgttagatgtttttaattttttaaataaatggaatactttttttttttttttaaagaaagcaactttactgagacaatgtagaaagaagttttgttccgtttctttaatgtggttgaagagcaatgtgtggctgaagacttttgttatgaggagctgcagattagctaggggacagctggaattatgctggcttctgataattattttaaaggggtctgaaatttgtgatggaatcagattttaacagctctcttcaatgacatagaaagttcatggaactcatgtttttaaagggctatgtaaatatatgaacattagaaaaatagcaacttgtgttacaaaaatacaaacacatgttaggaaggtactgtcatgggctaggcatggtggctcacacctgtaatcccagcattttgggaagctaagatgggtggatcacttgaggtcaggagtttgagaccagcctggccaacatggcgaaacccctctctactaaaaatacaaaaatttgccaggcgtggtggcgggtgcctgtaatcccagctacttgggaggctgaggcaagagaatcgcttgaacccaggaggcagaggttgcagtgagccgagatcgtgccattgcactccagcctgggtgacaaagcgagactccatctcaaaaaaaaaaaaaaaaaaaaaggaaagaactgtcatgtaaacataccgacatgtttaaacctgacaatggtgttatttgaaactttatattgttcttgtaagctttaactatatctctctttaaaatgcaaaataatgtcttaagattcaaagtctgtatttttaaagcatggctttggctttgcaaaataaaaaatgtgttttgtacatgaa

SEQ ID NO: 26 - DNA, 2561 bp, Homo sapiens
tacccagcctgtcgctaaactttccgggcgccagcccggctctgagtcgcgcttctcagcggagtgacccagggacggaggacccaggctggctggggactgtctgctcttctcggcgggatccgtggagagtcctttccctggaatccgagccctaaccgtctctccccagccctatccggcgaggagcggagcgctgccagcggaggcagcgccttcccgaagcagtttatctttggacggttttctttaaaggaaaaagcaaccaacaggttgccagccccggcgccacacacgagacgccggagggagaagccccggcccggattcctctgcctgtgtgcgtccctcgcgggctgctggaggcgaggggagggagggggcgatggctcggcctgacccatccgcgccgccctcgctgttgctgctgctcctggcgcagctggtgggccgggcggccgccgcgtccaaggccccggtgtgccaggaaatcacggtgcccatgtgccgcggcatcggctacaacctgacgcacatgcccaaccagttcaaccacgacacgcaggacgaggcgggcctggaggtgcaccagttctggccgctggtggagatccaatgctcgccggacctgcgcttcttcctatgctctatgtacacgcccatctgtctgcccgactaccacaagccgctgccgccctgccgctcggtgtgcgagcgcgccaaggccggctgctcgccgctgatgcgccagtacggcttcgcctggcccgagcgcatgagctgcgaccgcctcccggtgctgggccgcgacgccgaggtcctctgcatggattacaaccgcagcgaggccaccacggcgccccccaggcctttcccagccaagcccacccttccaggcccgccaggggcgccggcctcggggggcgaatgccccgctgggggcccgttcgtgtgcaagtgtcgcgagcccttcgtgcccattctgaaggagtcacacccgctctacaacaaggtgcggacgggccaggtgcccaactgcgcggtaccctgctaccagccgtccttcagtgccgacgagcgcacgttcgccaccttctggataggcctgtggtcggtgctgtgcttcatctccacgtccaccacagtggccaccttcctcatcgacatggaacgcttccgctatcctgagcgccccatcatcttcctgtcagcctgctacctgtgcgtgtcgctgggcttcctggtgcgtctggtcgtgggccatgccagcgtggcctgcagccgcgagcacaaccacatccactacgagaccacgggccctgcactgtgcaccatcgtcttcctcctggtctacttcttcggcatggccagctccatctggtgggtcatcctgtcgctcacctggttcctggccgccggcatgaagtggggcaacgaggccatcgcgggctacgcgcagtacttccacctggctgcgtggctcatccccagcgtcaagtccatcacggcactggcgctgagctccgtggacggggacccagtggccggcatctgctacgtgggcaaccagaacctgaactcgctgcgcggcttcgtgctgggcccgctggtgctctacctgctggtgggcacgctcttcctgctggcgggcttcgtgtcgctcttccgcatccgcagcgtcatcaagcagggcggcaccaagacggacaagctggagaagctcatgatccgcatcggcatcttcacgctgctctacacggtccccgccagcattgtggtggcctgctacctgtacgagcagcactaccgcgagagctgggaggcggcgctcacctgcgcctgcccgggccacgacaccggccagccgcgcgccaagcccgagtactgggtgctcatgctcaagtacttcatgtgcctggtggtgggcatcacgtcgggcgtctggatctggtcgggcaagacggtggagtcgtggcggcgtttcaccagccgctgctgctgccgcccgcggcgcggccacaagagcgggggcgccatggccgcaggggactaccccgaggcgagcgccgcgctcacaggcaggaccgggccgccgggccccgccgccacctaccacaagcaggtgtccctgtcgcacgtgtaggaggctgccgccgagggactcggccggagagctgaggggaggggggcgttttgtttggtagttttgccaaggtcacttccgtttaccttcatggtgctgttgccccctcccgcggcgacttggagagagggaagaggggcgttttcgaggaagaacctgtcccaggtcttctccaaggggcccagctcacgtgtattctattttgcgtttcttactgccttctttatgggaaccctctttttaatttatatgtatttttcttaatttgtaactttgttgcattttggcaacaatttacctttgctttgggggctttacaatcctaaggttggcgttgtaatgaagttccacttggttcaggtttctttgaactgtgtggtctcaattgggaaaatatatttcctatacgtgtgtcttt

SEQ ID NO: 27 - DNA, 1945 bp, Homo sapiens
agcccctaaagcagcactaattgcaaggatttctcaggtgactggttagtatgtggtgtcaatcatgaaaaagaagaactgcacgaaagtatctttctgaaaacttgcaaaactgagaagctagtctgtttaaaacaggaagttatatacttacattgtttactactttactaatgtctgtgatctgatggtatctctgtttcaggagtggtgacgcctaagctatcactggacatatcaaggacttcactaaattagcaggtaccactggtcttcttgtgcttatccgggcaagaacttatcgaaatacaatagaagtttttacttagaagagattttcagcagatgagaagctggtaacagagaccaaaatagtttggagactaaagaatcattgcacatttcactgctgagttgtattggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagctt

SEQ ID NO: 28 - DNA, 2347 bp, Homo sapiens
attcggagctgcctcctcgccaatgattccagcgcctgacagccaggaccccaggcagcagcgagtgacaggacgtctggaccggcgcgccgctagcagctctgccgggccgcggcggtgatcgatggggagcggctggagcggacccagcgagtgagggcgcacagccgggacgccgaggcggcgggcgggagacccgcaccagcgcagccggccctcggcgggacgtgacgcagcgcccggggcgcgggtttgatatttgacaaattgatctaaaatggctgggtttttatctgaataactcactgatgccatcccagaaagtcggcaccaggtgtatttgatatagtgtttgcaacaaattcgacccaggtgatcaaaatgattctcaactcttctactgaagatggtattaaaagaatccaagatgattgtcccaaagctggaaggcataattacatatttgtcatgattcctactttatacagtatcatctttgtggtgggaatatttggaaacagcttggtggtgatagtcatttacttttatatgaagctgaagactgtggccagtgtttttcttttgaatttagcactggctgacttatgctttttactgactttgccactatgggctgtctacacagctatggaataccgctggccctttggcaattacctatgtaagattgcttcagccagcgtcagtttcaacctgtacgctagtgtgtttctactcacgtgtctcagcattgatcgatacctggctattgttcacccaatgaagtcccgccttcgacgcacaatgcttgtagccaaagtcacctgcatcatcatttggctgctggcaggcttggccagtttgccagctataatccatcgaaatgtatttttcattgagaacaccaatattacagtttgtgctttccattatgagtcccaaaattcaacccttccgatagggctgggcctgaccaaaaatatactgggtttcctgtttccttttctgatcattcttacaagttatactcttatttggaaggccctaaagaaggcttatgaaattcagaagaacaaaccaagaaatgatgatatttttaagataattatggcaattgtgcttttctttttcttttcctggattccccaccaaatattcacttttctggatgtattgattcaactaggcatcatacgtgactgtagaattgcagatattgtggacacggccatgcctatcaccatttgtatagcttattttaacaattgcctgaatcctcttttttatggctttctggggaaaaaatttaaaagatattttctccagcttctaaaatatattcccccaaaagccaaatcccactcaaacctttcaacaaaaatgagcacgctttcctaccgcccctcagataatgtaagctcatccaccaagaagcctgcaccatgttttgaggttgagtgacatgttcgaaacctgtccataaagtaattttgtgaaagaaggagcaagagaacattcctctgcagcacttcactaccaaatgagcattagctacttttcagaattgaaggagaaaatgcattatgtggactgaaccgacttttctaaagctctgaacaaaagcttttctttccttttgcaacaagacaaagcaaagccacattttgcattagacagatgacggctgctcgaagaacaatgtcagaaactcgatgaatgtgttgatttgagaaattttactgacagaaatgcaatctccctagcctgcttttgtcctgttattttttatttccacataaaggtatttagaatatattaaatcgttagaggagcaacaggagatgagagttccagattgttctgtccagtttccaaagggcagtaaagttttcgtgccggttttcagctattagcaactgtgctacacttgcacctggtactgcacattttgtacaaagatatgctaagcagtagtcgtcaagttgcagatctttttgtgaaattcaacctgtgtcttataggtttacactgccaaaacaatgcccgtaagatggcttatttgtataatggtgttactaaagtcacatataaaagttaaactacttgtaaaggtgctgcactggtcccaagtagtagtgtcctcctagtatattagtttgatttaatatctgagaagtgtatatagtttgtggtaaaaagattatatatcataaagtatgccttcctgtttaaaaaaagtatatattctacacatatatatatatgtatatctatatctctaaactgctgttaattgattaaaatctggcaaagttatatttactttaaaataaaataattttattgc

SEQ ID NO: 29 - DNA, 1077 bp, Homo sapiens
atgccctctgtgtctccagcggggccctcggccggggcagtccccaatgccaccgcagtgacaacagtgcggaccaatgccagcgggctggaggtgcccctgttccacctgtttgcccggctggacgaggagctgcatggcaccttcccaggcctgtgcgtggcgctgatggcggtgcacggagccatcttcctggcagggctggtgctcaacgggctggcgctgtacgtcttctgctgccgcacccgggccaagacaccctcagtcatctacaccatcaacctggtggtgaccgatctactggtagggctgtccctgcccacgcgcttcgctgtgtactacggcgccaggggctgcctgcgctgtgccttcccgcacgtcctcggttacttcctcaacatgcactgctccatcctcttcctcacctgcatctgcgtggaccgctacctggccatcgtgcggcccgaagctcccgccgcctgccgccagcctgcctgtgccagggccgtgtgcgccttcgtgtggctggccgccggtgccgtcaccctgtcggtgctgggcgtgacaggcagccggccctgctgccgtgtctttgcgctgactgtcctggagttcctgctgcccctgctggtcatcagcgtgtttaccggccgcatcatgtgtgcactgtcgcggccgggtctgctccaccagggtcgccagcgccgcgtgcgggccatgcagctcctgctcacggtgctcatcatctttctcgtctgcttcacgcccttccacgcccgccaagtggccgtggcgctgtggcccgacatgccacaccacacgagcctcgtggtctaccacgtggccgtgaccctcagcagcctcaacagctgcatggaccccatcgtctactgcttcgtcaccagtggcttccaggccaccgtccgaggcctcttcggccagcacggagagcgtgagcccagcagcggtgacgtggtcagcatgcacaggagctccaagggctcaggccgtcatcacatcctcagtgccggccctcacgccctcacccaggccctggctaatgggcccgaggcttag

SEQ ID NO: 30 - DNA, 1523 bp, Homo sapiens
gtcagcgtgaaaggcagggcatggaccctcactcagggctgcgtgtgcccgacgtgggtggggggggcgtggaatcggagcacaacttggccctgcgttcccaggaagggacccgaagacctccccaggccaccccgccatctgcgtgcgcgctggcaagaggaggggcgcggggtaatggtggccggcgccacagccccgcgccgcgcagggtcttgcccaccaccagcgatgcccagcccttggtagagcttgaaccaccttctataaacaggatggcggtggagagacaggcccagtccctgagcccatgaggagtgtggccccttcaggcccaaagatggggaacatcactgcagacaactcctcgatgagctgtaccatcgaccataccatccaccagacgctggccccggtggtctatgttaccgtgctggtggtgggcttcccggccaactgcctgtccctctacttcggctacctgcagatcaaggcccggaacgagctgggcgtgtacctgtgcaacctgacggtggccgacctcttctacatctgctcgctgcccttctggctgcagtacgtgctgcagcacgacaactggtctcacggcgacctgtcctgccaggtgtgcggcatcctcctgtacgagaacatctacatcagcgtgggcttcctctgctgcatctccgtggaccgctacctggctgtggcccatcccttccgcttccaccagttccggaccctgaaggcggccgtcggcgtcagcgtggtcatctgggccaaggagctgctgaccagcatctacttcctgatgcacgaggaggtcatcgaggacgagaaccagcaccgcgtgtgctttgagcactaccccatccaggcatggcagcgcgccatcaactactaccgcttcctggtgggcttcctcttccccatctgcctgctgctggcgtcctaccagggcatcctgcgcgccgtgcgccggagccacggcacccagaagagccgcaaggaccagatccagcggctggtgctcagcaccgtggtcatcttcctggcctgcttcctgccctaccacgtgttgctgctggtgcgcagcgtctgggaggccagctgcgacttcgccaagggcgttttcaacgcctaccacttctccctcctgctcaccagcttcaactgcgtcgccgaccccgtgctctactgcttcgtcagcgagaccacccaccgggacctggcccgcctccgcggggcctgcctggccttcctcacctgctccaggaccggccgggccagggaggcctacccgctgggtgcccccgaggcctccgggaaaagcggggcccagggtgaggagcccgagctgttgaccaagctccacccggccttccagacccctaactcgccagggtcgggcgggttccccacgggcaggttggcctagcctgggtcctccgcgggtggctccacgtgaggcctgagccttcagcccacgggcctcagggcctgccgcctcctgcttccctcgc

SEQ ID NO: 31 - DNA, 2074 bp, Homo sapiens
tagggggtggccctgaactggggcctggccctggctggcctctcccgccgcctcactgggggacaggtccagcctgtggtgtccacaatgccccaggcctctgagcaccgcctgggccgtacccgagagccacctgttaatatccagccccgagtgggatccaagctaccatttgcccccagggcccgcagcaaggagcgcagaaacccagcctctgggccaaaccccatgttacgacctctgcctccccggccaggtctgcctgatgaacggctcaagaaactggagctgggacggggacggacctcaggccctcgtcccagaggcccccttcgagcagatcatggggttcccctgcctggctcaccacccccaacagtggctttgcctctcccatctcggaccaacttagcccgttccaagtctgtgagcagtggggacttgcgtccaatggggattgccttgggagggcaccgtggcaccggagagcttggggctgcactgagccgcttggccctccggcctgagccacccactttgagacgtagcacttctctccgccgcctagggggctttcctggaccccctaccctgttcagcatacggacagagccccctgcttcccatggctccttccacatgatatccgcccggtcctctgagcctttctactctgatgacaagatggctcatcacacactccttctgggctctggtcatgttggccttcgaaacctgggaaacacgtgcttcctgaatgctgtgctgcagtgtctgagcagcactcgacctcttcgggacttctgtctgagaagggacttccggcaagaggtgcctggaggaggccgagcccaagagctcactgaagcctttgcagatgtgattggtgccctctggcaccctgactcctgcgaagctgtgaatcctactcgattccgagctgtcttccagaaatatgttccctccttctctggatacagccagcaggatgcccaagagttcctgaagctcctcatggagcggctacaccttgaaatcaaccgccgaggccgccgggctccaccgatacttgccaatggtccagttccctctccaccccgccgaggaggggctctgctagaagaacctgagttaagtgatgatgaccgagccaacctaatgtggaaacgttacctggagcgagaggacagcaagattgtggacctgtttgtgggccagttgaaaagttgtctcaagtgccaggcctgtgggtatcgctccacgaccttcgaggttttttgtgacctgtccctgcccatccccaagaaaggatttgctgggggcaaggtgtctctgcgggattgtttcaaccttttcactaaggaagaagagctagagtcggagaatgccccagtgtgtgaccgatgtcggcagaaaactcgaagtaccaaaaagttgacagtacaaagattccctcgaatcctcgtgctccatctgaatcgattttctgcctcccgaggctccatcaaaaaaagttcagtaggtgtagactttccactgcagcgactgagcctaggggactttgccagtgacaaagccggaagtcctgtataccagctgtatgccctttgcaaccactcaggcagcgtccactatggccactacacagccctgtgccggtgccagactggttggcatgtctacaatgactctcgtgtctcccctgtcagtgaaaaccaggtggcatccagcgagggctacgtgctgttctaccaactgatgcaggagccaccccggtgcctgtgacacctctaagctctggcacctgtgaagccctttaaacacccttaagccccaggctccccgtttacctcagagacgtctatttttgtgtctttttaatcggggaggggggagggggtggttgtagctccattattttttttattaaaaaatacccttccacctggaggctcccttgtctcccagccccatgtacaaagctcaccaagcccctgcccatgtacagcccccagaccctctgcaatatcactttttgtgaataaatttattaagaaaaaaaaaaaaaaaaaaa

SEQ ID NO: 32 - DNA, 2924 bp, Homo sapiens
ggcttctggttcggcccacctctgaaggttccagaatcgatagtgaattcgtggttccaagtttggagcttttagctgccagccctggcccatcatgtagctgcagcacagccttccctaacgttgcaactgggggaaaaatcactttccagtctgttttgcaaggtgtgcatttccatcttgattccctgaaagtccatctgctgcatcggtcaagagaaactccacttgcatgaagattgcacgcctgcagcttgcatctttgttgcaaaactagctacagaagagaagcaaggcaaagtcttttgtgctcccctcccccatcaaaggaaaggggaaaatgtctcagtcgaaaggcaagaagcgaaaccctggccttaaaattccaaaagaagcatttgaacaacctcagaccagttccacaccacctcgagatttagactccaaggcttgcatttctattggaaatcagaactttgaggtgaaggcagatgacctggagcctataatggaactgggacgaggtgcgtacggggtggtggagaagatgcggcacgtgcccagcgggcagatcatggcagtgaagcggatccgagccacagtaaatagccaggaacagaaacggctactgatggatttggatatttccatgaggacggtggactgtccattcactgtcaccttttatggcgcactgtttcgggagggtgatgtgtggatctgcatggagctcatggatacatcactagataaattctacaaacaagttattgataaaggccagacaattccagaggacatcttagggaaaatagcagtttctattgtaaaagcattagaacatttacatagtaagctgtctgtcattcacagagacgtcaagccttctaatgtactcatcaatgctctcggtcaagtgaagatgtgcgattttggaatcagtggctacttggtggactctgttgctaaaacaattgatgcaggttgcaaaccatacatggcccctgaaagaataaacccagagctcaaccagaagggatacagtgtgaagtctgacatttggagtctgggcatcacgatgattgagttggccatccttcgatttccctatgattcatggggaactccatttcagcagctcaaacaggtggtagaggagccatcgccacaactcccagcagacaagttctctgcagagtttgttgactttacctcacagtgcttaaagaagaattccaaagaacggcctacatacccagagctaatgcaacatccatttttcaccctacatgaatccaaaggaacagatgtggcatcttttgtaaaactgattcttggagactaaaaagcagtggacttaatcggttgaccctactgtggattggtgggtttcggggtgaagcaagttcactacagcatcaatagaaagtcatctttgagataatttaaccctgcctctcagagggttttctctcccaattttctttttactccccctcttaagggggccttggaatctatagtatagaatgaactgtctagatggatgaattatgataaaggcttaggacttcaaaaggtgattaaatatttaatgatgtgtcatatgagtcctcaagcttctcagacttctcttattctttacaaaatgaatgcattggccctgacaaaaaggtgctacggtagtgatgaaattataagtagatttgtagtttgtcccatttattattttaatatttatgtttaagtgcttggttgaaaagattccattttatacaagaagggagattcaaaaaaaaaatataaggttgggttagcaatatttatagggcttttattttttaagttcaattgtgtctgtggtccagaagaaattatttaatatgcatctttgagaatattataaaaatatcaaaaaggagctcttcttgtgaaatgtctgttccagctgttgtgactgctgccatttttggaaacatctgcccaatcctgggtgatcaccacatcttttaggggaagtgacaagatgctctggtcatactctttttcccaactttggaaaacataaaaatcactcatataacagctcaaagagtaaaacatttggttcttctgacacttgtggtatagtattagtggaaagtgatttgtaatatgattttatatccacctacctattcatctacctgtgtgtatgtgtgtgtttgtgtgtctatttggcaattcacaagtcctgccaagtggtttctatgagcatctctgtttggtaaggaggacaattgtcagttttgagggggacatgtgttaaatcacagaaaaaaatggtgccttcttctgcgtttgtccctcctgccatgtgtaagttgtaaggattgcctttgtagttaatgtactctttggctttgtttgtttgttttcttcttcagtgaagcagccttactattcatagaagggctagaataggagaaaatgaaaggtagtgagtaattctttgataagatgaggaaataatgggaaaggttgaattaattcctgggcatggactaccagatgaccacaagttgcgttgaggccgcatctttcttcagcagcgtgcaatagctggctcctctataggagatgagcttcattgggagttcctagcaagttgactaaacagcaaaagttctttctcgtgggtaaatatacccacaggttctatgatttgtagctctaggtttcttgatgatcaaggagtgaagtaattgacagggaaaatatagacctatgataaataaccaggaagcattgcttttggacaaggaagaacagagggttttgattttaaaaagaagaaaaaaaaaccttattttttctttcttggcctcaagttcaatatggagaggattgcttccctgaatcctctcttccttccccttttagag

SEQ ID NO: 33 - DNA, 947 bp, Homo sapiens
ggctcggggccggggccagcacccacactgggtctccacagcggcatggaggcctgcgtgtcttcactgctggtgctggccctgggggccctgtcagtaggcagctcctttgggacccagatcatcgggggccgggaggtgatcccccactcgcgcccgtacatggcctcactgcagagaaatggctcccacctgtgcgggggtgtcctggtgcacccaaagtgggtgctgacggctgcccactgcctggcccagcggatggcccagctgaggctggtgctggggctccacaccctggacagccccggtctcaccttccacatcaaggcagccatccagcaccctcgctacaagcccgtccctgccctggagaacgacctcgcgctgcttcagctggacgggaaagtgaagcccagccggaccatccggccgttggccctgcccagtaagcgccaggtggtggcagcagggactcggtgcagcatggccggctgggggctgacccaccagggcgggcgcctgtcccgggtgctgcgggagctggacctccaagtgctggacacccgcatgtgtaacaacagccgcttctggaacggcagcctctcccccagcatggtctgcctggcggccgactccaaggaccaggctccctgcaagggtgactcgggcgggcccctggtgtgtggcaaaggccgggtgttggccggagtcctgtccttcagctccagggtctgcactgacatcttcaagcctcccgtggccaccgctgtggcgccttacgtgtcctggatcaggaaggtcaccggccgatcggcctgatgccctggggtgatggggaccccctcgctgtctccacaggacccttcccctccaggggtgcagtggggtgggtgaggacgggtgggagggacagggagggaccaataaatcataatgaagaaacgctc

SEQ ID NO: 34 - DNA, 2633 bp, Homo sapiens
gatttcccggggaggtcccttctgggcccccggcggaggtgggagagagtcaggcaggagccgaggccggggagccctcttcgtcagctggtgctcactgcgccgcgccagcgccagccgggactcacccgcagctccatgcttgtgcccggttcgactcgtccatcctccaagaagaggcagcccatgaggctcccagtccccactgagtgccaccctgaaggatgtcccagctctcctccaccctgaagcgctacacagaatcggcccgctacacagatgcccactatgccaagtcgggctatggtgcctacaccccgtcctcctatggggccaatctggctgcctccttactggagaaggagaaacttggtttcaagccggtccccaccagcagcttcctcacccgtccccgtacctatggcccctcctccctcctggactatgaccggggccgccccctgctgagacccgacatcactgggggtggtaagcgggcagagagccagacccggggtactgagcggcctttaggcagtggcctcagcgggggcagcggattcccttatggagtgaccaacaactgcctcagctacctgcccatcaatgcctatgaccagggggtgaccctaacccagaagctggacagccaatcagacctggcccgggatttctccagcctccggacctcagatagctaccggatagaccccaggaacctgggccgcagccccatgctggcccggacgcgcaaggagctctgcaccctgcaggggctctaccagacagccagctgccctgaatacctggtcgactacctggagaactatggtcgcaagggcagtgcatctcaggtgccctcccaggcccctccctcacgagtccctgaaatcatcagcccaacctaccgacccattggccgctacacgctgtgggagacgggaaagggtcaggcccctgggcccagccgctccagctccccgggaagagacggcatgaattctaagagtgcccagggtctggctggtcttcgaaaccttgggaacacgtgcttcatgaactcaattctgcagtgcctgagcaacactcgggagttgagagattactgcctccagaggctctacatgcgggacctgcaccacggcagcaatgcacacacagccctcgtggaagagtttgcaaaactaattcagaccatatggacttcatcccccaatgatgtggtgagcccatctgagttcaagacccagatccagagatacgcaccgcgctttgttggctataatcagcaggatgctcaggagttccttcgctttcttctggatgggctccataacgaggtgaaccgagtgacactgagacctaagtccaaccctgagaacctcgatcatcttcctgatgacgagaaaggccgacagatgtggagaaaatatctagaacgggaagacagtaggatcggggatctctttgttgggcagctaaagggctcgctgacgtgtacagattgtggttactgttctacggtcttcgaccccttctgggacctctcactgcccattgctaagcgaggttatcctgaggtgacattaatggactgcatgaggctcttcaccaaagaggatgtgcttgatggagatgaaaagccaacatgctgtcgctgccgaggcagaaaacggtgtataaagaagttctccatccagaggttcccaaagatcttggtgctccgtctgaagcggttctcagaatccaggatccgaaccagcaagctcacaacatttgtgaacttccccctaagagacctggacttaagagaatttgcctcagaaaacaccaaccatgctgtttacaacctgtacgctgtgtccaatcactccggaaccaccatgggtggccactatacagcctactgtcgcagtccagggacaggagaatggcacactttcaacgactccagcgtcactcccatgtcctccagccaagtgcgcaccagcgacgcctacctgctcttctacgaactggccagcccgccctcccgaatgtagcgccaggagccacgtcccttctcccttccccgtggtggccccgctccctaaattttttaaaaagacaaaaacaaaacaacaacaacaacacacaaacctgacaagagaaaaacaaacctgaagctgccgagcaggagtggatgcagcctgatcagggtctggagcaaggagccgggctttcctgagctgtggcccggcagggaagatcgcctggacgtggagccagcatcgccccgtgccctcggcgtttgcatttgtaaacttgtggtcttcctatgtgtcagaaacaactgtgtcttgggggggaagaccctcgctgcgccgcttcccgccgcagcgcccgcgcctccgaggggacagcgccctctggagctcgctgggagcatcaccgcctggacgcccgcgccgcggaggagccggcgcccatctccacccgcacggctcgccggtccagagccatgagccaagagccctcttcacgctgctaactccaggggacagacgaagggacatctttggaaaacgctggttttggtttttaaaaagcccaacttttttttttaatttccataactaaagtgttcagactgg

SEQ ID NO: 35 - DNA, 4673 bp, Homo sapiens
agccagaaggatggggtggctcccactcctgctgcttctgactcaatgcttaggggtccctggtgagtgcccccaaccttgatccccatctgccttcaggaggggcttggccccattctcctattctgggatgagaaaaaagtcagggagccagaggctcagtgggcatggggcagtgaccttggcctcttgagcacagctgggaagccctaggaacacatagacatggcccacttaggcctctattagcacgtctgctctagcactgaagcagtgttaggaccacacagatgcacgcacacagcaggcagtgacccctcctgagcctgatctacccctctaacctagcgtatgcctttgtgcaggtgagagcccagctttggagtctgaatgcctagccagggcccctggctgggtaatgtgatggctctgagccttagcattctcatttgagagatgagatggggcaagctccatcacccactgctctcacagagcgtgtgtgttagatctgagcccggtgcctgggccactacacagaggcaccggtgataactaccaagtctgggcctgcttcccaggggaaattttttgacaagtatctgtgcaggggggctagactggcccttgaaagtgcatacagggtccatcccagaagccttgtagctttgatcccctgaatgaacaaactgtggacatgccaatacacattactgacatgtatgcccacctgacctgcacccactctgcagggcagcgctcgccattgaatgacttctaggtgctccggggcacagagctacagcgccgctacaagcggtggtgcccgggccttggcaggaggatgtggcagatgctgaagagtgtgctggtcgctgtgggcccttaatggactgccggtgagtggccactgggcatagataagactgggggcaggggagcctgggccgtggcgttaccttgtgccttcttctctccagggcgttccactacaatgtgagcagccatggttgccaactgctgccatggactcaacactcaccccacacgaggctgcggcattctgggcgctgtgacctcttccaggagaaaggcgagtgggggtggagaggggcagggtgggagacaggggacctcagcccaagttgatcttctgtctcttgctcccagactacatacggacctgcatcatgaacaatggggttgggtaccggggcaccatggccacgaccgtgggtggcctgtcctgccaggcttggagccacaagttcccgaacgatcaccagtgggacaaacaccttccctccgtcccggcctgggaccttcccccagcacacactatagtgatgctctgggccctcaggtacatgcccacgctccggaatggcctggaagagaacttctgccgtaaccctgatggcgaccccggaggtccttggtgccacacaacagaccctgccgtgcgcttccagagctgcggcatcaaatcctgccgggtgggtaagcggcgccgggtcaagctgggagagtggagggacaagcccacgcccatccacgaacccactggctctttgtctccagccgcgtgtgtctggtgcaatggcgaggaataccgcggcgcggtagaccgcaccgagtcagggcgcgagtgccagcgctgggatcttcagcacccgcaccagcaccccttcgagccgggcaagtacgcgtaggcggtatcggcgccctgggggccgggctagggaaggtccaggactccaggggcagggctccgtgtagggcaactgggcggggccagataagccagagtcccagggtcttcttcacgccccattaccgcccccaggttcctcgaccaaggtctggacgacaactattgccggaatcctgacggctccgagcggccatggtgctacactacggatccgcagatcgagcgagaattctgtgacctcccccgctgcggtaggcggcggggaccaggcctgggagggtacctgggaaccttggggaggggcgtggcttggccggggaggtcagaggggctgggcgtgacctgagagcatatcccgtggagtaccgtacacctgggaaaggcgggtttggtcccagccccagagggatctcagctgtcgctcggggccggacctatctcggtccatctaagggtccgaggcacagccccgccaagaggccacaagtgtcagctgcttccgcgggaagggtgagggctaccggggcacagccaataccaccaccgcgggcgtaccttgccagcgttgggacgcgcaaatcccgcatcagcaccgatttacgccagaaaaatacgcgtgcaagtgaggtgggggggcgggcgttgggacgtgctgctgcgggtgagacgggaggagggtagtcacgggcttagggctggaggctggcgggctagggctgagtgcagcgcctgcttagagaccttcgggagaacttctgccggaaccccgacggctcagaggcgccctggtgcttcacactgcggcccggcatgcgcgtgggcttttgctaccagatccggcgttgtacagacgacgtgcggccccagggtgaggcccaagcttgggggctacagagccggggctggaagcctggaaccggagggccggggcggggtctcggcctgatggctgcccgcaccggccgcagactgctaccacggcgcgggggagcagtaccgcggcacggtcagcaagacccgcaagggtgtccagtgccagcgcgggtccgctgagacgccgcacaagccgcagtgaatccctggtgctcccggccccgccagggccctaaccctggggcggcatgctttgatgtctgggaccagagcctggaaatggttgagactaccctgccacgacttcgctcccgctcccgcctcggttcacgtttacctccgaaccgcatgcacaactggaggagaacttctgccagacccagatggggatagccatgggccctggtgctacacgatggacccaaggaccccattcgactactgtgccctgcgacgctgcggtgagcactagtgacgcttgccccatgaccctgcctcagccctcaccaccaaaggctggctcccttaaccgcagtgaactttgtctttcagctgatgaccagccgccatcaatcctggacccccccaggttaggagttgggccagttatgggtcaggccctttagcccacgacatccacacagtctgggtttcatccagcccaccccatcctacagaccaggtgcagtttgagaagtgtggcaagagggtggatcggctggatcagcgttgttccaagctgcgcgtggctgggggccatccgggcaactcaccctggacagtcagcttgcggaattggtgaggcacaactgcctgtctcccacagagaggagctgaggttgtgtcctctgtggttatgccactgggggctgggaatctatccctgcccccagaggtcctagccagaagatggcaggtctagcatctgtcccaggagtctgttccctgtcctaattccccactcctctaggcagggccagcatttctgcggggggtctctagtgaaggagcagtggatactgactgcccggcagtgcttctcctcctggtgagcctcccttgtgtttggggacccagtctcatcccaccttcccctttccccaggcaagctaacaagtgagccttggggcaacggactgagagtcacaaatgacctagcagagcttctctcccagccatatgcctctcacgggctatgaggtatggttgggcaccctgttccagaacccacaacatggagagccaggcctacagcgggtcccagtagccaagatgctgtgtgggccctcaggctctcagcttgtcctgctcaagctggagaggtatgtggacaacctgggagggtgtgaggtggggctgagccttgtggcctcagaccctgagtgcccccattcttgctaaagatctgtgaccctgaaccagcgtgtggccctgatctgcctgccgcctgaatgatatgtggtgcctccagggaccaagtgtgagattgcaggccggggtgagaccaaaggtaagagcatagtgcacaggactgctggtggccaggaggcccagccctggatcttcctccaggaccgtctccttctccccattcccctcactgcaggtacgggtaatgacacagtcctaaatgtggccttgctgaatgtcatctccaaccaggagtgtaacatcaagcaccgaggacatgtgcgggagagcgagatgtgcactgagggactgttggcccctgtgggggcctgtgaggttggtggcagggccctgggccagccctggaagggtatggggggctagaaatgaactattttatcatgaagcaggctagtcatggctgtggcccagggccctcatcagttctcctacctgccagggtgactacgggggcccacttgcctgctttacccacaactgctgggtcctgaaaggaattagaatccccaaccgagtatgcgcaaggtcgcgctggccagccgtcttcacgcgtgtctctgtgtttgtggactggattcacaaggtcatgagactgggttaggcccagccttgacgccatatgctttggggaggacaaaacttgtaagtacagtca

SEQ ID NO: 36 - DNA, 2851 bp, Homo sapiens
gcttcacgctaaagccccagagcccgacgcggcagccgcggtagcggagaagactggagctccgaggagctgcatctgcggcaacctgtgtgctgacgctacgtgcctcctggctccgacgtagctcgcagctccccagtctcactccattccttccccacctggcgcgcacctgctcaagaccagggtcctgccaagcgctaggagggcgcgtgccaggggcgctagggaactgcggagcgcgcgcgccatggggccgccgcctggggccggggtctcctgccgcggtggctgcggcttttccagattgctggcatggtgcttcctgctggccctgagtccgcaggcacccggttcccggggggctgaagcagtgtggaccgcgtacctcaacgtgtcctggcgggttccgcacacgggagtgaaccgtacggtgtgggagctgagcgaggagggcgtgtacggccaggactcgccgctggagcctgtggctggggtcctggtaccgcccgacgggcccggggcgcttaacgcctgtaacccgcacacgaatttcacggtgcccacggtttggggaagcaccgtgcaagtctcttggttggccctcatccaacgcggcgggggctgcaccttcgcagacaagatccatctggcttatgagagaggggcgtctggagccgtcatctttaacttccccgggacccgcaatgaggtcatccccatgtctcacccgggtgcagtagacattgttgcaatcatgatcggcaatctgaaaggcacaaaaattctgcaatctattcaaagaggcatacaagtgacaatggtcatagaagtagggaaaaaacatggcccttgggtgaatcactattcaatttttttcgtttctgtgtccttttttattattacggcggcaactgtgggctattttatcttttattctgctcgaaggctacggaatgcaagagctcaaagcaggaagcagaggcaattaaaggcagatgctaaaaaagctattggaaggcttcaactacgcacactgaaacaaggagacaaggaaattggccctgatggagatagttgtgctgtgtgcattgaattgtataaaccaaatgatttggtacgcatcttaacgtgcaaccatattttccataagacatgtgttgacccatggctgttagaacacaggacttgccccatgtgcaaatgtgacatactcaaagctttgggaattgaggtggatgttgaagatggatcagtgtctttacaagtccctgtatccaatgaaatatctaatagtgcctcctcccatgaagaggataatcgcagcgagaccgcatcatctggatatgcttcagtacagggaacagatgaaccgcctctggaggaacacgtgcagtcaacaaatgaaagtctacagctggtaaaccatgaagcaaattctgtggcagtggatgttattcctcatgttgacaacccaacctttgaagaagacgaaactcctaatcaagagactgctgttcgagaaattaaatcttaaaatctgtgtaaatagaaaacttgaaccattagtaataacagaactgccaatcagggcctagtttctattaataaattggataaatttaataaaataagagtgatactgaaagtgctcagatgactaatattatgctatagttaaatggcttaaaatatttaacctgttaacttttttccacaaactcattataatatttttcataggcaagtttcctctcagtagtgataacaacatttttagacattcaaaactgtcttcaagaagtcacgtttttcatttataacaattttcttataaaaacatgttgcttttaaaatgtggagtagctgtaatcactttattttatgatagtatcttaatgaaaaatactacttctttagcttgggctacatgtgtcagggtttttctccaggtgcttatattgatctggaattgtaatgtaaaaagcaatgcaaacttaggcgagtacttcttgaaatgtctatttaagctgctttaagttaatagaaaagattaaagcaaaatattcatttttactttttcttatttttaaaattaggctgaatgtacttcatgtgatttgtcaaccatagtttatcagagattatggacttaattgattggtatattagtgacatcaacttgacacaagattagacaaaaaattccttacaaaaatactgtgtaactatttctcaaacttgtgggatttttcaaaagctcagtatatgaatcatcatactgtttgaaattgctaatgacagagtaagtaacactaatattggtcattgatcttcgttcatgaattagtctacagaaaaaaaatgttctgtaaaattagtctgttgaaaatgttttccaaacaatgttactttgaaaattgagtttatgtttgacctaaatgggctaaaattacattagataaactaaaattctgtccgtgtaactataaattttgtgaatgcattttcctggtgtttgaaaaagaagggggggagaattccaggtgccttaatataaagtttgaagcttcatccaccaaagttaaatagagctatttaaaaatgcactttatttgtactctgtgtggcttttgttttagaattttgttcaaattatagcagaatttaggcaaaaataaaacagacatgtatttttgtttgctgaatggatgaaaccattgcattcttgtacactgatttgaaatgctgtaaatatgtcccaatttgtattgattctctttaaatataaaatgtaaataaaatattccaataaaagtttgtgtctggtgttagtttaaaaaaaaaaaaa

SEQ ID NO: 37 - DNA, 4307 bp, Homo sapiens
cacagacacatatgcacgagagagacagaggaggaaagagacagagacaaaggcacagcggaagaaggcagagacagggcaggcacagaagcggcccagacagagtcctacagagggagaggccagagaagctgcagaagacacaggcagggagagacaaagatccaggaaaggagggctcaggaggagagtttggagaagccagacccctgggcacctctcccaagcccaaggactaagttttctccatttcctttaacggtcctcagcccttctgaaaactttgcctctgaccttggcaggagtccaagcccccaggctacagagaggagctttccaaagctagggtgtggaggacttggtgccctagacggcctcagtccctcccagctgcagtaccagtgccatgtcccagacaggctcgcatcccgggaggggcttggcagggcgctggctgtggggagcccaaccctgcctcctgctccccattgtgccgctctcctggctggtgtggctgcttctgctactgctggcctctctcctgccctcagcccggctggccagccccctcccccgggaggaggagatcgtgtttccagagaagctcaacggcagcgtcctgcctggctcgggcacccctgccaggctgttgtgccgcttgcaggcctttggggagacgctgctactagagctggagcaggactccggtgtgcaggtcgaggggctgacagtgcagtacctgggccaggcgcctgagctgctgggtggagcagagcctggcacctacctgactggcaccatcaatggagatccggagtcggtggcatctctgcactgggatgggggagccctgttaggcgtgttacaatatcggggggctgaactccacctccagcccctggagggaggcacccctaactctgctgggggacctggggctcacatcctacgccggaagagtcctgccagcggtcaaggtcccatgtgcaacgtcaaggctcctcttggaagccccagccccagaccccgaagagccaagcgctttgcttcactgagtagatttgtggagacactggtggtggcagatgacaagatggccgcattccacggtgcggggctaaagcgctacctgctaacagtgatggcagcagcagccaaggccttcaagcacccaagcatccgcaatcctgtcagcttggtggtgactcggctagtgatcctggggtcaggcgaggaggggccccaagtggggcccagtgctgcccagaccctgcgcagcttctgtgcctggcagcggggcctcaacacccctgaggactcggaccctgaccactttgacacagccattctgtttacccgtcaggacctgtgtggagtctccacttgcgacacgctgggtatggctgatgtgggcaccgtctgtgacccggctcggagctgtgccattgtggaggatgatgggctccagtcagccttcactgctgctcatgaactgggtcatgtcttcaacatgctccatgacaactccaagccatgcatcagtttgaatgggcctttgagcacctctcgccatgtcatggcccctgtgatggctcatgtggatcctgaggagccctggtccccctgcagtgcccgcttcatcactgacttcctggacaatggctatgggcactgtctcttagacaaaccagaggctccattgcatctgcctgtgactttccctggcaaggactatgatgctgaccgccagtgccagctgaccttcgggcccgactcacgccattgtccacagctgccgccgccctgtgctgccctctggtgctctggccacctcaatggccatgccatgtgccagaccaaacactcgccctgggccgatggcacaccctgcgggcccgcacaggcctgcatgggtggtcgctgcctccacatggaccagctccaggacttcaatattccacaggctggtggctggggtccttggggaccatggggtgactgctctcggacctgtgggggtggtgtccagttctcctcccgagactgcacgaggcctgtcccccggaatggtggcaagtactgtgagggccgccgtacccgcttccgctcctgcaacactgaggactgcccaactggctcagccctgaccttccgcgaggagcagtgtgctgcctacaaccaccgcaccgacctcttcaagagcttcccagggcccatggactgggttcctcgctacacaggcgtggccccccaggaccagtgcaaactcacctgccaggcccgggcactgggctactactatgtgctggagccacgggtggtagatgggaccccctgttccccggacagctcctcggtctgtgtccagggccgatgcatccatgctggctgtgatcgcatcattggctccaagaagaagtttgacaagtgcatggtgtgcggaggggacggttctggttgcagcaagcagtcaggctccttcaggaaattcaggtacggatacaacaatgtggtcactatccccgcgggggccacccacattcttgtccggcagcagggaaaccctggccaccggagcatctacttggccctgaagctgccagatggctcctatgccctcaatggtgaatacacgctgatgccctcccccacagatgtggtactgcctggggcagtcagcttgcgctacagcggggccactgcagcctcagagacactgtcaggccatgggccactggcccagcctttgacactgcaagtcctagtggctggcaacccccaggacacacgcctccgatacagcttcttcgtgccccggccgaccccttcaacgccacgccccactccccaggactggctgcaccgaagagcacagattctggagatccttcggcggcgcccctgggcgggcaggaaataacctcactatcccggctgccctttctgggcaccggggcctcggacttagctgggagaaagagagagcttctgttgctgcctcatgctaagactcagtggggaggggctgtgggcgtgagacctgcccctcctctctgccctaatgcgcaggctggccctgccctggtttcctgccctgggaggcagtgatgggttagtggatggaaggggctgacagacagccctccatctaaactgccccctctgccctgcgggtcacaggagggagggggaaggcagggagggcctgggccccagttgtatttatttagtatttattcacttttatttagcaccagggaaggggacaaggactagggtcctggggaacctgacccctgacccctcatagccctcaccctggggctaggaaatccagggtggtggtgataggtataagtggtgtgtgtatgcgtgtgtgtgtgtgtgtgaaaatgtgtgtgtgcttatgtatgaggtacaacctgttctgctttcctcttcctgaattttattttttgggaaaagaaaagtcaagggtagggtgggccttcagggagtgagggattatctttttttttttttctttctttctttcttttttttttttgagacagaatctcgctctgtcgcccaggctggagtgcaatggcacaatctcggctcactgcatcctccgcctcccgggttcaagtgattctcatgcctcagcctcctgagtagctgggattacaggctcctgccaccacgcccagctaatttttgttttgttttgtttggagacagagtctcgctattgtcaccagggctggaatgatttcagctcactgcaaccttcgccacctgggttccagcaattctcctgcctcagcctcccgagtagctgagattataggcacctaccaccacgcccggctaatttttgtatttttagtagagacggggtttcaccatgttggccaggctggtctcgaactcctgaccttaggtgatccactcgccttcatctcccaaagtgctgggattacaggcgtgagccaccgtgcctggccacgcccaactaatttttgtatttttagtagagacagggtttcaccatgttggccaggctgctcttgaactcctgacctcaggtaatcgacctgcctcggcctcccaaagtgctgggattacaggtgtgagccaccacgcccggtacatattttttaaattgaattctactatttatgtgatccttttggagtcagacagatgtggttgcatcctaactccatgtctctgagcattagatttctcatttgccaataataatacctcccttagaagtttgttgtgaggattaaataatgtaaataaagaactagcataac

SEQ ID NO: 38 - protein, 318 aa, Homo sapiens
MPNNSTALSLANVTYITMEIFIGLCAIVGNVLVICVVKLNPSLQTTTFYFIVSLALADIAVGVLVMPLAIVVSLGITIHFYSCLFMTCLLLIFTHASIMSLLAIAVDRYLRVKLTVRYKRVTTHRRIWLALGLCWLVSFLVGLTPMFGWNMKLTSEYHRNVTFLSCQFVSVMRMDYMVYFSFLTWIFIPLVVMCAIYLDIFYIIRNKLSLNLSNSKETGAFYGREFKTAKSLFLVLFLFALSWLPLSIINCIIYFNGEVPQLVLYMGILLSHANSMMNPIVYAYKIKKFKETYLLILKACVVCHPSDSLDTSIEKNSE

SEQ ID NO: 39 - protein, 472 aa, Homo sapiens
MKSILDGLADTTFRTITTDLLYVGSNDIQYEDIKGDMASKLGYFPQKFPLTSFRGSPFQEKMTAGDNPQLVPADQVNITEFYNKSLSSFKENEENIQCGENFMDIECFMVLNPSQQLAIAVLSLTLGTFTVLENLLVLCVILHSRSLRCRPSYHFIGSLAVADLLGSVIFVYSFIDFHVFHRKDSRNVFLFKLGGVTASFTASVGSLFLTAIDRYISIHRPLAYKRIVTRPKAVVAFCLMWTIAIVIAVLPLLGWNCEKLQSVCSDIFPHIDETYLMFWIGVTSVLLLFIVYAYMYILWKAHSHAVRMIQRGTQKSIIIHTSEDGKVQVTRPDQARMDIRLAKTLVLILVVLIICWGPLLAIMVYDVFGKMNKLIKTVFAFCSMLCLLNSTVNPIIYALRSKDLRHAFRSMFPSCEGTAQPLDNSMGDSDCLHKHANNAASVHRAAESCIKSTVKIAKVTMSVSTDTSAEAL

SEQ ID NO: 40 - protein, 844 aa, Homo sapiens
MGPGAPFARVGWPLPLLVVMAAGVAPVWASHSPHLPRPHSRVPPHPSSERRAVYIGALFPMSGGWPGGQACQPAVEMALEDVNSRRDILPDYELKLIHHDSKCDPGQATKYLYELLYNDPIKIILMPGCSSVSTLVAEAARMWNLIVLSYGSSSPALSNRQRFPTFFRTHPSATLHNPTRVKLFEKWGWKKIATIQQTTEVFTSTLDDLEERVKEAGIEITFRQSFFSDPAVPVKNLKRQDARIIVGLFYETEARKVFCEVYKERLFGKKYVWFLIGWYADNWFKIYDPSINCTVDEMTEAVEGHITTEIVMLNPANTRSISNMTSQEFVEKLTKRLKRHPEETGGFQEAPLAYDAIWALALALNKTSGGGGRSGVRLEDFNYNNQTITDQIYRAMNSSSFEGVSGHVVFDASGSRMAWTLIEQLQGGSYKKIGYYDSTKDDLSWSKTDKWIGGSPPADQTLVIKTFRFLSQKLFISVSVLSSLGIVLAVVCLSFNIYNSHVRYIQNSQPNLNNLTAVGCSLALAAVFPLGLDGYHIGRNQFPFVCQARLWLLGLGFSLGYGSMFTKIWWVHTVFTKKEEKKEWRKTLEPWKLYATVGLLVGMDVLTLAIWQIVDPLHRTIETFAKEEPKEDIDVSILPQLEHCSSRKMNTWLGIFYGYKGLLLLLGIFLAYETKSVSTEKINDHRAVGMAIYNVAVLCLITAPVTMILSSQQDAAFAFASLAIVFSSYITLVVLFVPKMRRLITRGEWQSEAQDTMKTGSSTNNNEEEKSRLLEKENRELEKIIAEKEERVSELRHQLQSRQQLRSRRHPPTPPEPSGGLPRGPPEPPDRLSCDGSRVHLLYK

SEQ ID NO: 41 - protein, 466 aa, Homo sapiens
MNNSTNSSNNSLALTSPYKTFEVVFIVLVAGSLSLVTIIGNILVMVSIKVNRHLQTVNNYFLFSLACADLIIGVFSMNLYTLYTVIGYWPLGPVVCDLWLALDYVVSNASVMNLLIISFDRYFCVTKPLTYPVKRTTKMAGMMIAAAWVLSFILWAPAILFWQFIVGVRTVEDGECYIQFFSNAAVTFGTAIAAFYLPVIIMTVLYWHISRASKSRIKKDKKEPVANQDPVSPSLVQGRIVKPNNNNMPSSDDGLEHNKIQNGKAPRDPVTENCVQGEEKESSNDSTSVSAVASNMRDDEITQDENTVSTSLGHSKDENSKQTCIRIGTKTPKSDSCTPTNTTVEVVGSSGQNGDEKQNIVARKIVKMTKQPAKKKPPPSREKKVTRTILAILLAFIITWAPYNVMVLINTFCAPCIPNTVWTIGYWLCYINSTINPACYALCNATFKKTFKHLLMCHYKNIGATR

SEQ ID NO: 42 - protein, 532 aa, Homo sapiens
MEGDSYHNATTVNGTPVNHQPLERHRLWEVITIAAVTAVVSLITIVGNVLVMISFKVNSQLKTVNNYYLLSLACADLIIGIFSMNLYTTYILMGRWALGSLACDLWLALDYVASNASVMNLLVISFDRYFSITRPLTYRAKRTPKRAGIMIGLAWLISFILWAPAILCWQYLVGKRTVPLDECQIQFLSEPTITFGTAIAAFYIPVSVMTILYCRIYRETEKRTKDLADLQGSDSVTKAEKRKPAHRALFRSCLRCPRPTLAQRERNQASWSSSRRSTSTTGKPSQATGPSANWAKAEQLTTCSSYPSSEDEDKPATDPVLQVVYKSQGKESPGEEFSAEETEETFVKAETEKSDYDTPNYLLSPAAAHRPKSQKCVAYKFRLVVKADGNQETNNGCHKVKIMPCPFPVAKEPSTKGLNPNPSHQMTKRKRVVLVKERKAAQTLSAILLAFIITWTPYNIMVLVSTFCDKCVPVTLWHLGYWLCYVNSTVNPICYALCNRTFRKTFKMLLLCRWKKKKVEEKLYWQGNSKLP

SEQ ID NO: 43 - protein, 742 aa, Homo sapiens
MGGRVFLAFCVWLTLPGAETQDSRGCARWCPQNSSCVNATACRCNPGFSSFSEIITTPTETCDDINECATPSKVSCGKFSDCWNTEGSYDCVCSPGYEPVSGAKTFKNESENTCQDVDECSSGQHQCDSSTVCFNTVGSYSCRCRPGWKPRHGIPNNQKDTVCEDMTFSTWTPPPGVHSQTLSRFFDKVQDLGRDSKTSSAEVTIQNVIKLVDELMEAPGDVEALAPPVRHLIATQLLSNLEDIMRILAKSLPKGPFTYISPSNTELTLMIQERGDKNVTMGQSSARMKLNWAVAAGAEDPGPAVAGILSIQNMTTLLANASLNLHSKKQAELEEIYESSIRGVQLRRLSAVNSIFLSHNNTKELNSPILFAFSHLESSDGEAGRDPPAKDVMPGPRQELLCAFWKSDSDRGGHWATEGCQVLGSKNGSTTCQCSHLSSFAILMAHYDVEDWKLTLITRVGLALSLFCLLLCILTFLLVRPIQGSRTTIHLHLCICLFVGSTIFLAGIENEGGQVGLRCRLVAGLLHYCFLAAFCWMSLEGLELYFLVVRVFQGQGLSTRWLCLIGYGVPLLIVGVSAAIYSKGYGRPRYCWLDFEQGFLWSFLGPVTFIILCNAVIFVTTVWKLTQKFSEINPDMKKLKKARALTITAIAQLFLLGCTWVFGLFIFDDRSLVLTYVFTILNCLQGAFLYLLHCLLNKKVREEYRKWACLVAGGSKYSEFTSTTSGTGHNQTRALRASESGI

SEQ ID NO: 44 - protein, 384 aa, Homo sapiens
MALNDCFLLNLEVDHFMHCNISSHSADLPVNDDWSHPGILYVIPAVYGVIILIGLIGNITLIKIFCTVKSMRNVPNLFISSLALGDLLLLITCAPVDASRYLADRWLFGRIGCKLIPFIQLTSVGVSVFTLTALSADRYKAIVRPMDIQASHALMKICLKAAFIWIISMLLAIPEAVFSDLHPFHEESTNQTFISCAPYPHSNELHPKIHSMASFLVFYVIPLSIISVYYYFIAKNLIQSAYNLPVEGNIHVKKQIESRKRLAKTVLVFVGLFAFCWLPNHVIYLYRSYHYSEVDTSMLHFVTSICARLLAFTNSCVNPFALYLLSKSFRKQFNTQLLCCQPGLIIRSHSTGRSTTCMTSLKSTNPSVATFSLINGNICHERYV

SEQ ID NO: 45 - protein, 415 aa, Homo sapiens
MSGMEKLQNASWIYQQKLEDPFQKHLNSTEEYLAFLCGPRRSHFFLPVSVVYVPIFVVGVIGNVLVCLVILQHQAMKTPTNYYLFSLAVSDLLVLLLGMPLEVYEMWRNYPFLFGPVGCYFKTALFETVCFASILSITTVSVERYVAILHPFRAKLQSTRRRALRILGIVWGFSVLFSLPNTSIHGIKFHYFPNGSLVPGSATFTVIKPMWIYNFIIQVTSFLFYLLPMTVISVLYYLMALRLKKDKSLEADEGNANIQRPCRKSVNKMLFVLVLVFAICWAPFHIDRLFFSFVEEWSESLAAVFNLVHVVSGVFFYLSSAVNPIIYNLLSRRFQAAFQNVISSFHKQWHSQHDPQLPPAQRNIFLTECHFVELTEDIGPQFPCQSSMHNSHLPTALSSEQMSRTNYQSFHFNKT

SEQ ID NO: 46 - protein, 373 aa, Homo sapiens
MTEVLWPAVPNGTDAAFLAGPGSSWGNSTVASTAAVSSSFKCALTKTGFQFYYLPAVYILVFIIGFLGNSVAIWMFVFHMKPWSGISVYMFNLALADFLYVLTLPALIFYYFNKTDWIFGDAMCKLQRFIFHVNLYGSILFLTCISAHRYSGVVYPLKSLGRLKKKNAICISVLVWLIVVVAISPILFYSGTGVRKNKTITCYDTTSDEYLRSYFIYSMCTTVAMFCVPLVLILGCYGLIVRALIYKDLDNSPLRRKSIYLVIIVLTVFAVSYIPFHVMKTMNLRARLDFQTPAMCAFNDRVYATYQVTRGLASLNSCVDPILYFLAGDTFRRRLSRATRKASRRSEANLQSKSEDMTLNILPEFKQNGDTSL

SEQ ID NO: 47 - protein, 358 aa, Homo sapiens
MGNASNDSQSEDCETRQWLPPGESPAISSVMFSAGVLGNLIALALLARRWRGDVGCSAGRRSSLSLFHVLVTELVFTDLLGTCLISPVVLASYARNQTLVALAPESRACTYFAFAMTFFSLATMLMLFAMALERYLSIGHPYFYQRRVSRSGGLAVLPVIYAVSLLFCSLPLLDYGQYVQYCPGTWCFIRHGRTAYLQLYATLLLLLIVSVLACNFSVILNLIRMHRRSRRSRCGPSLGSGRGGPGARRRGERVSMAEETDHLILLAIMTITFAVCSLPFTIFAYMNETSSRKEKWDLQALRFLSINSIIDPWVFAILRPPVLRLMRSVLCCRISLRTQDATQTSCSTQSDASKQADL

SEQ ID NO: 48 - protein, 348 aa, Homo sapiens
MVNNFSQAEAVELCYKNVNESCIKTPYSPGPRSILYAVLGFGAVLAAFGNLLVMIAILHFKQLHTPTNFLIASLACADFLVGVTVMPFSTVRSVESCWYFGDSYCKFHTCFDTSFCFASLFHLCCISVDRYIAVTDPLTYPTKFTVSVSGICIVLSWFFSVTYSFSIFYTGANEEGIEELVVALTCVGGCQAPLNQNWVLLCFLLFFIPNVAMVFIYSKIFLVAKHQARKIESTASQAQSSSESYKERVAKRERKAAKTLGIAMAAFLVSWLPYLVDAVIDAYMNFITPPYVYEILVWCVYYNSAMNPLIYAFFYQWFGKAIKLIVSGKVLRTDSSTTNLFSEEVETD

SEQ ID NO: 49 - protein, 339 aa, Homo sapiens
MMPFCHNIINISCVKNNWSNDVRASLYSLMVLIILTTLVGNLIVIVSISHFKQLHTPTNWLIHSMATVDFLLGCLVMPYSMVRSAEHCWYFGEVFCKIHTSTDIMLSSASIFHLSFISIDRYYAVCDPLRYKAKMNILVICVMIFISWSVPAVFAFGMIFLELNFKGAEEIYYKHVHCRGGCSVFFSKISGVLTFMTSFYIPGSIMLCVYYRIYLIAKEQARLISDANQKLQIGLEMKNGISQSKERKAVKTLGIVMGVFLICWCPFFICTVMDPFLHYIIPPTLNDVLIWFGYLNSTFNPMVYAFFYPWFRKALKMMLFGKIFQKDSSRCKLFLELSS

SEQ ID NO: 50 - protein, 466 aa, Homo sapiens
MVFLSGNASDSSNCTQPPAPVNISKAILLGVILGGLILFGVLGNILVILSVACHRHLHSVTHYYIVNLAVADLLLTSTVLPFSAIFEVLGYWAFGRVFCNIWAAVDVLCCTASIMGLCIISIDRYIGVSYPLRYPTIVTQRRGLMALLCVWALSLVISIGPLFGWRQPAPEDETICQINEEPGYVLFSALGSFYLPLAIILVMYCRVYVVAKRESRGLKSGLKTDKSDSEQVTLRIHRKNAPAGGSGMASAKTKTHFSVRLLKFSREKKAAKTLGIVVGCFVLCWLPFFLVMPIGSFFPDFKPSETVFKIVFWLGYLNSCINPIIYPCSSQEFKKAFQNVLRIQCLRRKQSSKHALGYTLHPPSQAVEGQHKDMVRIPVGSRETFYRISKTDGVCEWKFFSSMPRGSARITVSKDQSSCTTARVRSKSFLEVCCCVGPSTPSLDKNHQVPTIKVHTISLSENGEEV

SEQ ID NO: 51 - protein, 429 aa, Homo sapiens
MVFLSGNASDSSNCTQPPAPVNISKAILLGVILGGLILFGVLGNILVILSVACHRHLHSVTHYYIVNLAVADLLLTSTVLPFSAIFEVLGYWAFGRVFCNIWAAVDVLCCTASIMGLCIISIDRYIGVSYPLRYPTIVTQRRGLMALLCVWALSLVISIGPLFGWRQPAPEDETICQINEEPGYVLFSALGSFYLPLAIILVMYCRVYVVAKRESRGLKSGLKTDKSDSEQVTLRIHRKNAPAGGSGMASAKTKTHFSVRLLKFSREKKAAKTLGIVVGCFVLCWLPFFLVMPIGSFFPDFKPSETVFKIVFWLGYLNSCINPIIYPCSSQEFKKAFQNVLRIQCLRRKQSSKHALGYTLHPPSQAVEGQHKDMVRIPVGSRETFYRISKTDGVCEWKFFSSMPRGSARITVSKDQSSCTTARGHTPMT

SEQ ID NO: 52 - protein, 391 aa, Homo sapiens
MFSPWKISMFLSVREDSVPTTASFSADMLNVTLQGPTLNGTFAQSKCPQVEWLGWLNTIQPPFLWVLFVLATLENIFVLSVFCLHKSSCTVAEIYLGNLAAADLILACGLPFWAITISNNFDWLFGETLCRVVNAIISMNLYSSICFLMLVSIDRYLALVKTMSMGRMRGVRWAKLYSLVIWGCTLLLSSPMLVFRTMKEYSDEGHNVTACVISYPSLIWEVFTNMLLNVVGFLLPLSVITFCTMQIMQVLRNNEMQKFKEIQTERRATVLVLVVLLLFIICWLPFQISTFLDTLHRLGILSSCQDERIIDVITQIASFMAYSNSCLNPLVYVIVGKRFRKKSWEVYQGVCQKGGCRSEPIQMENSMGTLRTSISVERQIHKLQDWAGSRQ

SEQ ID NO: 53 - protein, 340 aa, Homo sapiens
  misc_feature (245)..(245): Xaa can be any naturally occurring amino acid
MNPFHASCWNTSAELLNKSWNKEFAYQTASVVDTVILPSMIGIICSTGLVGNILIVFTIIRSRKKTVPDIYICNLAVADLVHIVGMPFLIHQWARGGEWVFGGPLCTIITSLDTCNQFACSAIMTVMSVDRYFALVQPFRLTRWRTRYKTIRINLGLWAASFILALPVWVYSKVIKFKDGVESCAFDLTSPDDVLWYTLYLTITTFFFPLPLILVCYILILCYTWEMYQQNKDARCCNPSVPKQXVMKLTKMVLVLVVVFILSAAPYHVIQLVNLQMEQPTLAFYVGYYLSICLSYASSSINPFLYILLSGNFQKRLPQIQRRATEKEINNMGNTLKSHF

SEQ ID NO: 54 - protein, 590 aa, Homo sapiens
MTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDYVASNASVMNLLVISFDRYFSITRPLTYRAKRTTKRAGVMIGLAWVISFVLWAPAILFWQYFVGKRTVPPGECFIQFLSEPTITFGTAIAAFYMPVTIMTILYWRIYKETEKRTKELAGLQASGTEAETENFVHPTGSSRSCSSYELQQQSMKRSNRRKYGRCHFWFTTKSWKPSSEQMDQDHSSSDSWNNNDAAASLENSASSDEEDIGSETRAIYSIVLKLPGHSTILNSTKLPSSDNLQVPEEELGMVDLERKADKLQAQKSVDDGGSFPKSFSKLPIQLESAVDTAKTSDVNSSVGKSTATLPLSFKEATLAKRFALKTRSQITKRKRMSLVKEKKAAQTLSAILLAFIITWTPYNIMVLVNTFCDSCIPKTFWNLGYWLCYINSTVNPVCYALCNKTFRTTFKMLLLCQCDKKKRRKQQYQQRQSVIFHKRAPEQAL

SEQ ID NO: 55 - protein, 400 aa, Homo sapiens
MDSSAAPTNASNCTDALAYSSCSPAPSPGSWVNLSHLDGNLSDPCGPNRTNLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLVKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

SEQ ID NO: 56 - protein, 407 aa, Homo sapiens
MDNVLPVDSDLSPNISTNTSEPNQFVQPAWQIVLWAAAYTVIVVTSVVGNVVVMWIILAHKRMRTVTNYFLVNLAFAEASMAAFNTVVNFTYAVHNEWYYGLFYCKFHNFFPIAAVFASIYSMTAVAFDRYMAIIHPLQPRLSATATKVVICVIWVLALLLAFPQGYYSTTETMPSRVVCMIEWPEHPNKIYEKVYHICVTVLIYFLPLLVIGYAYTVVGITLWASEIPGDSSDRYHEQVSAKRKVVKMMIVVVCTFAICWLPFHIFFLLPYINPDLYLKKFIQQVYLAIMWLAMSSTMYNPIIYCCLNDRFRLGFKHAFRCCPFISAGDYEGLEMKSTRYLQTQGSVYKVSRLETTISTVVGAHEEEPEDGPKATPSSLDLTSNCSSRSDSKTMTESFSFSSNVLS

SEQ ID NO: 57 - protein, 333 aa, Homo sapiens
MESSGNPESTTFFYYDLQSQPCENQAWVFATLATTVLYCLVFLLSLVGNSLVLWVLVKYESLESLTNIFILNLCLSDLVFACLLPVWISPYHWGWVLGDFLCKLLNMIFSISLYSSIFFLTIMTIHRYLSVVSPLSTLRVPTLRCRVLVTMAVWVASILSSILDTIFHKVLSSGCDYSELTWYLTSVYQHNLFFLLSLGIILFCYVEILRTLFRSRSKRRHRTVKLIFAIVVAYFLSWGPYNFTLFLQTLFRTQIIRSCEAKQQLEYALLICRNLAFSHCCFNPVLYVFVGVKFRTHLKHVLRQFWFCRLQAPSPASIPHSPGAFAYEGASFY

SEQ ID NO: 58 - protein, 382 aa, Homo sapiens
MGPTSVPLVKAHRSSVSDYVNYDIIVRHYNYTGKLNISADKENSIKLTSVVFILICCFIILENIFVLLTIWKTKKFHRPMYYFIGNLALSDLLAGVAYTANLLLSGATTYKLTPAQWFLREGSMFVALSASVFSLLAIAIERYITMLKMKLHNGSNNFRLFLLISACWVISLILGGLPIMGWNCISALSSCSTVLPLYHKHYILFCTTVFTLLLLSIVILYCRIYSLVRTRSRRLTFRKNISKASRSSEKSLALLKTVIIVLSVFIACWAPLFILLLLDVGCKVKTCDILFRAEYFLVLAVLNSGTNPIIYTLTNKEMRRAFIRIMSCCKCPSGDSAGKFKRPIIAGMEFSRSKSDNSSHPQKDEGDNPETIMSSGNVNSSS

SEQ ID NO: 59 - protein, 352 aa, Homo sapiens
MNTTSSAAPPSLGVEFISLLAIILLSVALAVGLPGNSFVVWSILKRMQKRSVTALMVLNLALADLAVLLTAPFFLHFLAQGTWSFGLAGCRLCHYVCGVSMYASVLLITAMSLDRSLAVARPFVSQKLRTKAMARRVLAGIWVLSFLLATPVLAYRTVVPWKTNMSLCFPRYPSEGHRAFHLIFEAVTGFLLPFLAVVASYSDIGRRLQARRFRRSRRTGRLVVLIILTFAAFWLPYHVVNLAEAGRALAGQAAGLGLVGKRLSLARNVLIALAFLSSSVNPVLYACAGGGLLRSAGVGFVAKLLEGTGSEASSTRRGGSLGQTARSGPAALEPGPSESLTASSPLKLNELN

SEQ ID NO: 60 - protein, 374 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 61 - protein, 352 aa, Homo sapiens [sequence omitted]

SEQ ID NO: 62 - protein, 374 aa, Homo sapiens
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 63 - protein, 585 aa, Homo sapiens
MARPDPSAPPSLLLLLLAQLVGRAAAASKAPVCQEITVPMCRGIGYNLTHMPNQFNHDTQDEAGLEVHQFWPLVEIQCSPDLRFFLCSMYTPICLPDYHKPLPPCRSVCERAKAGCSPLMRQYGFAWPERMSCDRLPVLGRDAEVLCMDYNRSEATTAPPRPFPAKPTLPGPPGAPASGGECPAGGPFVCKCREPFVPILKESHPLYNKVRTGQVPNCAVPCYQPSFSADERTFATFWIGLWSVLCFISTSTTVATFLIDMERFRYPERPIIFLSACYLCVSLGFLVRLVVGHASVACSREHNHIHYETTGPALCTIVFLLVYFFGMASSIWWVILSLTWFLAAGMKWGNEAIAGYAQYFHLAAWLIPSVKSITALALSSVDGDPVAGICYVGNQNLNSLRGFVLGPLVLYLLVGTLFLLAGFVSLFRIRSVIKQGGTKTDKLEKLMIRIGIFTLLYTVPASIVVACYLYEQHYRESWEAALTCACPGHDTGQPRAKPEYWVLMLKYFMCLVVGITSGVWIWSGKTVESWRRFTSRCCCRPRRGHKSGGAMAAGDYPEASAALTGRTGPPGPAATYHKQVSLSHV

SEQ ID NO: 64 - protein, 355 aa, Homo sapiens
MTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 65 - protein, 359 aa, Homo sapiens [sequence omitted]

SEQ ID NO: 66 - protein, 358 aa, Homo sapiens
MPSVSPAGPSAGAVPNATAVTTVRTNASGLEVPLFHLFARLDEELHGTFPGLCVALMAVHGAIFLAGLVLNGLALYVFCCRTRAKTPSVIYTINLVVTDLLVGLSLPTRFAVYYGARGCLRCAFPHVLGYFLNMHCSILFLTCICVDRYLAIVRPEAPAACRQPACARAVCAFVWLAAGAVTLSVLGVTGSRPCCRVFALTVLEFLLPLLVISVFTGRIMCALSRPGLLHQGRQRRVRAMQLLLTVLIIFLVCFTPFHARQVAVALWPDMPHHTSLVVYHVAVTLSSLNSCMDPIVYCFVTSGFQATVRGLFGQHGEREPSSGDVVSMHRSSKGSGRHHILSAGPHALTQALANGPEA

SEQ ID NO: 67 - protein, 375 aa, Homo sapiens
MRSVAPSGPKMGNITADNSSMSCTIDHTIHQTLAPVVYVTVLVVGFPANCLSLYFGYLQIKARNELGVYLCNLTVADLFYICSLPFWLQYVLQHDNWSHGDLSCQVCGILLYENIYISVGFLCCISVDRYLAVAHPFRFHQFRTLKAAVGVSVVIWAKELLTSIYFLMHEEVIEDENQHRVCFEHYPIQAWQRAINYYRFLVGFLFPICLLLASYQGILRAVRRSHGTQKSRKDQIQRLVLSTVVIFLACFLPYHVLLLVRSVWEASCDFAKGVFNAYHFSLLLTSFNCVADPVLYCFVSETTHRDLARLRGACLAFLTCSRTGRAREAYPLGAPEASGKSGAQGEEPELLTKLHPAFQTPNSPGSGGFPTGRLA

SEQ ID NO: 68 - protein, 565 aa, Homo sapiens
MPQASEHRLGRTREPPVNIQPRVGSKLPFAPRARSKERRNPASGPNPMLRPLPPRPGLPDERLKKLELGRGRTSGPRPRGPLRADHGVPLPGSPPPTVALPLPSRTNLARSKSVSSGDLRPMGIALGGHRGTGELGAALSRLALRPEPPTLRRSTSLRRLGGFPGPPTLFSIRTEPPASHGSFHMISARSSEPFYSDDKMAHHTLLLGSGHVGLRNLGNTCFLNAVLQCLSSTRPLRDFCLRRDFRQEVPGGGRAQELTEAFADVIGALWHPDSCEAVNPTRFRAVFQKYVPSFSGYSQQDAQEFLKLLMERLHLEINRRGRRAPPILANGPVPSPPRRGGALLEEPELSDDDRANLMWKRYLEREDSKIVDLFVGQLKSCLKCQACGYRSTTFEVFCDLSLPIPKKGFAGGKVSLRDCFNLFTKEEELESENAPVCDRCRQKTRSTKKLTVQRFPRILVLHLNRFSASRGSIKKSSVGVDFPLQRLSLGDFASDKAGSPVYQLYALCNHSGSVHYGHYTALCRCQTGWHVYNDSRVSPVSENQVASSEGYVLFYQLMQEPPRCL

SEQ ID NO: 69 - protein, 334 aa, Homo sapiens
MSQSKGKKRNPGLKIPKEAFEQPQTSSTPPRDLDSKACISIGNQNFEVKADDLEPIMELGRGAYGVVEKMRHVPSGQIMAVKRIRATVNSQEQKRLLMDLDISMRTVDCPFTVTFYGALFREGDVWICMELMDTSLDKFYKQVIDKGQTIPEDILGKIAVSIVKALEHLHSKLSVIHRDVKPSNVLINALGQVKMCDFGISGYLVDSVAKTIDAGCKPYMAPERINPELNQKGYSVKSDIWSLGITMIELAILRFPYDSWGTPFQQLKQVVEEPSPQLPADKFSAEFVDFTSQCLKKNSKERPTYPELMQHPFFTLHESKGTDVASFVKLILGD

SEQ ID NO: 70 - protein, 257 aa, Homo sapiens
MEACVSSLLVLALGALSVGSSFGTQIIGGREVIPHSRPYMASLQRNGSHLCGGVLVHPKWVLTAAHCLAQRMAQLRLVLGLHTLDSPGLTFHIKAAIQHPRYKPVPALENDLALLQLDGKVKPSRTIRPLALPSKRQVVAAGTRCSMAGWGLTHQGGRLSRVLRELDLQVLDTRMCNNSRFWNGSLSPSMVCLAADSKDQAPCKGDSGGPLVCGKGRVLAGVLSFSSRVCTDIFKPPVATAVAPYVSWIRKVTGRSA

SEQ ID NO: 71 - protein, 605 aa, Homo sapiens
MSQLSSTLKRYTESARYTDAHYAKSGYGAYTPSSYGANLAASLLEKEKLGFKPVPTSSFLTRPRTYGPSSLLDYDRGRPLLRPDITGGGKRAESQTRGTERPLGSGLSGGSGFPYGVTNNCLSYLPINAYDQGVTLTQKLDSQSDLARDFSSLRTSDSYRIDPRNLGRSPMLARTRKELCTLQGLYQTASCPEYLVDYLENYGRKGSASQVPSQAPPSRVPEIISPTYRPIGRYTLWETGKGQAPGPSRSSSPGRDGMNSKSAQGLAGLRNLGNTCFMNSILQCLSNTRELRDYCLQRLYMRDLHHGSNAHTALVEEFAKLIQTIWTSSPNDVVSPSEFKTQIQRYAPRFVGYNQQDAQEFLRFLLDGLHNEVNRVTLRPKSNPENLDHLPDDEKGRQMWRKYLEREDSRIGDLFVGQLKGSLTCTDCGYCSTVFDPFWDLSLPIAKRGYPEVTLMDCMRLFTKEDVLDGDEKPTCCRCRGRKRCIKKFSIQRFPKILVLRLKRFSESRIRTSKLTTFVNFPLRDLDLREFASENTNHAVYNLYAVSNHSGTTMGGHYTAYCRSPGTGEWHTFNDSSVTPMSSSQVRTSDAYLLFYELASPPSRM

SEQ ID NO: 72 - protein, 648 aa, Homo sapiens
MGWLPLLLLLTQCLGVPGAPGHRATAPLQAVVPGPWQEDVADAEECAGRCGPLMDCRAFHYNVSSHGCQLLPWTQHSPHTRLRHSGRCDLFQEKGEWGYMPTLRNGLEENFCRNPDGDPGGPWCHTTDPAVRFQSCGIKSCRVAACVWCNGEEYRGAVDRTESGRECQRWDLQHPHQHPFEPGKFLDQGLDDNYCRNPDGSERPWCYTTDPQIEREFCDLPRCGSEAQPRQEATSVSCFRGKGEGYRGTANTTTAGVPCQRWDAQIPHQHRFTPEKYACKDLRENFCRNPDGSEAPWCFTLRPGMRVGFCYQIRRCTDDVRPQDCYHGAGEQYRGTVSKTRKGVQCQRGAWKWLRLPCHDFAPAPASVHVYLRTACTTGGELLPDPDGDSHGPWCYTMDPRTPFDYCALRRCDQVQFEKCGKRVDRLDQRCSKLRVAGGHPGNSPWTVSLRNWQGQHFCGGSLVKEQWILTARQCFSSCHMPLTGYEVWLGTLFQNPQHGEPGLQRVPVAKMLCGPSGSQLVLLKLERYVDNLGGWTKCEIAGRGETKGTGNDTVLNVALLNVISNQECNIKHRGHVRESEMCTEGLLAPVGACEGDYGGPLACFTHNCWVLKGIRIPNRVCARSRWPAVFTRVSVFVDWIHKVMRLG

SEQ ID NO: 73 - protein, 428 aa, Homo sapiens
MGPPPGAGVSCRGGCGFSRLLAWCFLLALSPQAPGSRGAEAVWTAYLNVSWRVPHTGVNRTVWELSEEGVYGQDSPLEPVAGVLVPPDGPGALNACNPHTNFTVPTVWGSTVQVSWLALIQRGGGCTFADKIHLAYERGASGAVIFNFPGTRNEVIPMSHPGAVDIVAIMIGNLKGTKILQSIQRGIQVTMVIEVGKKHGPWVNHYSIFFVSVSFFIITAATVGYFIFYSARRLRNARAQSRKQRQLKADAKKAIGRLQLRTLKQGDKEIGPDGDSCAVCIELYKPNDLVRILTCNHIFHKTCVDPWLLEHRTCPMCKCDILKALGIEVDVEDGSVSLQVPVSNEISNSASSHEEDNRSETASSGYASVQGTDEPPLEEHVQSTNESLQLVNHEANSVAVDVIPHVDNPTFEEDETPNQETAVREIKS

SEQ ID NO: 74 - protein, 837 aa, Homo sapiens
MSQTGSHPGRGLAGRWLWGAQPCLLLPIVPLSWLVWLLLLLLASLLPSARLASPLPREEEIVFPEKLNGSVLPGSGTPARLLCRLQAFGETLLLELEQDSGVQVEGLTVQYLGQAPELLGGAEPGTYLTGTINGDPESVASLHWDGGALLGVLQYRGAELHLQPLEGGTPNSAGGPGAHILRRKSPASGQGPMCNVKAPLGSPSPRPRRAKRFASLSRFVETLVVADDKMAAFHGAGLKRYLLTVMAAAAKAFKHPSIRNPVSLVVTRLVILGSGEEGPQVGPSAAQTLRSFCAWQRGLNTPEDSDPDHFDTAILFTRQDLCGVSTCDTLGMADVGTVCDPARSCAIVEDDGLQSAFTAAHELGHVFNMLHDNSKPCISLNGPLSTSRHVMAPVMAHVDPEEPWSPCSARFITDFLDNGYGHCLLDKPEAPLHLPVTFPGKDYDADRQCQLTFGPDSRHCPQLPPPCAALWCSGHLNGHAMCQTKHSPWADGTPCGPAQACMGGRCLHMDQLQDFNIPQAGGWGPWGPWGDCSRTCGGGVQFSSRDCTRPVPRNGGKYCEGRRTRFRSCNTEDCPTGSALTFREEQCAAYNHRTDLFKSFPGPMDWVPRYTGVAPQDQCKLTCQARALGYYYVLEPRVVDGTPCSPDSSSVCVQGRCIHAGCDRIIGSKKKFDKCMVCGGDGSGCSKQSGSFRKFRYGYNNVVTIPAGATHILVRQQGNPGHRSIYLALKLPDGSYALNGEYTLMPSPTDVVLPGAVSLRYSGATAASETLSGHGPLAQPLTLQVLVAGNPQDTRLRYSFFVPRPTPSTPRPTPQDWLHRRAQILEILRRRPWAGRK

SEQ ID NO: 75 - DNA, 19 bp, Homo sapiens
actgacctcagagtaccac

SEQ ID NO: 76 - DNA, 19 bp, Homo sapiens
atggtatacttcagcttcc

SEQ ID NO: 77 - DNA, 19 bp, Homo sapiens
ctccatgatgaaccctatc

SEQ ID NO: 78 - DNA, 19 bp, Homo sapiens
ggagaatgaggagaacatc

SEQ ID NO: 79 - DNA, 19 bp, Homo sapiens
gatagccgcaacgtgtttc

SEQ ID NO: 80 - DNA, 19 bp, Homo sapiens
acacgcaaacaatgcagcc

SEQ ID NO: 81 - DNA, 19 bp, Homo sapiens
gagcacggtcaagattgcc

SEQ ID NO: 82 - DNA, 19 bp, Homo sapiens
ggtaaccatgtctgtgtcc

SEQ ID NO: 83 - DNA, 19 bp, Homo sapiens
gaagtacgtctggttcctc

SEQ ID NO: 84 - DNA, 19 bp, Homo sapiens
ggatgatctttcctggtcc

SEQ ID NO: 85 - DNA, 19 bp, Homo sapiens
gacagggtcatcgaccaac

SEQ ID NO: 86 - DNA, 19 bp, Homo sapiens
catcctagtcatggtttcc

SEQ ID NO: 87 - DNA, 19 bp, Homo sapiens
agaaggacaagaaggagcc

SEQ ID NO: 88 - DNA, 19 bp, Homo sapiens
gcagaatattgtagcccgc

SEQ ID NO: 89 - DNA, 19 bp, Homo sapiens
cagcactatcaaccctgcc

SEQ ID NO: 90 - DNA, 19 bp, Homo sapiens
cctctacaccacctacatc

SEQ ID NO: 91 - DNA, 19 bp, Homo sapiens
ctacatcctcatgggacgc

SEQ ID NO: 92 - DNA, 19 bp, Homo sapiens
gagtcagggtaaggaaagc

SEQ ID NO: 93 - DNA, 19 bp, Homo sapiens
actaccttctgtctccagc

SEQ ID NO: 94 - DNA, 19 bp, Homo sapiens
ggaaccttcaacgaaaggc

SEQ ID NO: 95 - DNA, 19 bp, Homo sapiens
gcatgaagctgaattgggc

SEQ ID NO: 96 - DNA, 19 bp, Homo sapiens
ctccatctttctgagccac

SEQ ID NO: 97 - DNA, 19 bp, Homo sapiens
ttaaagaaggcgagggcgc

SEQ ID NO: 98 - DNA, 19 bp, Homo sapiens
cgtgtgctccagtggatgc

SEQ ID NO: 99 - DNA, 19 bp, Homo sapiens
agccgcctttatctggatc

SEQ ID NO: 100 - DNA, 19 bp, Homo sapiens
ggagtacaacctgcatgac

SEQ ID NO: 101 - DNA, 19 bp, Homo sapiens
gacgcccaccaactactac

SEQ ID NO: 102 - DNA, 19 bp, Homo sapiens
cctactgtctcgccgcttc

SEQ ID NO: 103 - DNA, 19 bp, Homo sapiens
ctctcacctcccaacagcc

SEQ ID NO: 104 - DNA, 19 bp, Homo sapiens
tttggctctggccgacttc

SEQ ID NO: 105 - DNA, 19 bp, Homo sapiens
ccatcacctgttacgacac

SEQ ID NO: 106 - DNA, 19 bp, Homo sapiens
acgatgaacttgagggccc

SEQ ID NO: 107 - DNA, 19 bp, Homo sapiens
gagtgaagacatgaccctc

SEQ ID NO: 108 - DNA, 19 bp, Homo sapiens
gcagaatggagatacaagc

SEQ ID NO: 109 - DNA, 19 bp, Homo sapiens
gatgctcatgctcttcgcc

SEQ ID NO: 110 - DNA, 19 bp, Homo sapiens
ttgacccttgggtctttgc

SEQ ID NO: 111 - DNA, 19 bp, Homo sapiens
ccttgggtctttgccatcc

SEQ ID NO: 112 - DNA, 19 bp, Homo sapiens
ttcaaacaactgcacacac

SEQ ID NO: 113 - DNA, 19 bp, Homo sapiens
agtgaggtctgtggagagc

SEQ ID NO: 114 - DNA, 19 bp, Homo sapiens
tctaacctgtgtaggaggc

SEQ ID NO: 115 - DNA, 19 bp, Homo sapiens
ccttgggaattgctatggc

SEQ ID NO: 116 - DNA, 19 bp, Homo sapiens
tatggcagcatttcttgtc

SEQ ID NO: 117 - DNA, 19 bp, Homo sapiens
taccatacctcgttgatgc

SEQ ID NO: 118 - DNA, 19 bp, Homo sapiens
gcggcaaggtcttaaggac

SEQ ID NO: 119 - DNA, 19 bp, Homo sapiens
tcattcattccatggccac

SEQ ID NO: 120 - DNA, 19 bp, Homo sapiens
tactatgctgtgtgtgatc

SEQ ID NO: 121 - DNA, 19 bp, Homo sapiens
actgcagaggaggttgctc

SEQ ID NO: 122 - DNA, 19 bp, Homo sapiens
catcctagtgatcctctcc

SEQ ID NO: 123 - DNA, 19 bp, Homo sapiens
gtctggcctcaagaccgac

SEQ ID NO: 124 - DNA, 19 bp, Homo sapiens
gtgacgctccgcatccatc

SEQ ID NO: 125 - DNA, 19 bp, Homo sapiens
gcagtcttccaaacatgcc

SEQ ID NO: 126 - DNA, 19 bp, Homo sapiens
agaccaatcctcctgtacc

SEQ ID NO: 127 - DNA, 19 bp, Homo sapiens
gaaccatcaagttccaacc

SEQ ID NO: 128 - DNA, 19 bp, Homo sapiens
catcctagtgatcctctcc

SEQ ID NO: 129 - DNA, 19 bp, Homo sapiens
gtctggcctcaagaccgac

SEQ ID NO: 130 - DNA, 19 bp, Homo sapiens
gtgacgctccgcatccatc

SEQ ID NO: 131 - DNA, 19 bp, Homo sapiens
gcagtcttccaaacatgcc

SEQ ID NO: 132 - DNA, 19 bp, Homo sapiens
agaccaatcctcctgtacc

SEQ ID NO: 133 - DNA, 19 bp, Homo sapiens
gcatccatctgactaaggc

SEQ ID NO: 134 - DNA, 19 bp, Homo sapiens
catctttgtcctcagcgtc

SEQ ID NO: 135 - DNA, 19 bp, Homo sapiens
gctctacagcttggtgatc

SEQ ID NO: 136 - DNA, 19 bp, Homo sapiens
ggagtacagcgatgagggc

SEQ ID NO: 137 - DNA, 19 bp, Homo sapiens
cgtcaccgcttgtgtcatc

SEQ ID NO: 138 - DNA, 19 bp, Homo sapiens
gtgttcaccaacatgctcc

SEQ ID NO: 139 - DNA, 19 bp, Homo sapiens
caacgagatgcagaagttc

SEQ ID NO: 140 - DNA, 19 bp, Homo sapiens
cccactggtgtacgtgatc

SEQ ID NO: 141 - DNA, 19 bp, Homo sapiens
ctgccagtgtggtggatac

SEQ ID NO: 142 - DNA, 19 bp, Homo sapiens
ccaatttgcctgtagtgcc

SEQ ID NO: 143 - DNA, 19 bp, Homo sapiens
atttccagaaacgtctgcc

SEQ ID NO: 144 - DNA, 19 bp, Homo sapiens
gcagctgaagacggtcaac

SEQ ID NO: 145 - DNA, 19 bp, Homo sapiens
gagagccggtgtgatgatc

SEQ ID NO: 146 - DNA, 19 bp, Homo sapiens
caacaatgatgctgctgcc

SEQ ID NO: 147 - DNA, 19 bp, Homo sapiens
tctgggctactggctgtgc

SEQ ID NO: 148 - DNA, 19 bp, Homo sapiens
cttgtcccacttagatggc

SEQ ID NO: 149 - DNA, 19 bp, Homo sapiens
gactgccaccaacatctac

SEQ ID NO: 150 - DNA, 19 bp, Homo sapiens
cagcatattcaccctctgc

SEQ ID NO: 151 - DNA, 19 bp, Homo sapiens
tgtctgcaactggatcctc

SEQ ID NO: 152 - DNA, 19 bp, Homo sapiens
ctacgttccagactgtttc

SEQ ID NO: 153 - DNA, 19 bp, Homo sapiens
gttccacaacttctttccc

SEQ ID NO: 154 - DNA, 19 bp, Homo sapiens
cacatctgtgtgactgtgc

SEQ ID NO: 155 - DNA, 19 bp, Homo sapiens
atcaacccagatctctacc

SEQ ID NO: 156 - DNA, 19 bp, Homo sapiens
tgacaggttccgtctgggc

SEQ ID NO: 157 - DNA, 19 bp, Homo sapiens
gtatgagagcctggagtcc

SEQ ID NO: 158 - DNA, 19 bp, Homo sapiens
catcatgaccatccaccgc

SEQ ID NO: 159 - DNA, 19 bp, Homo sapiens
cagcagctagaatacgccc

SEQ ID NO: 160 - DNA, 19 bp, Homo sapiens
cccggtgctctatgtcttc

SEQ ID NO: 161 - DNA, 19 bp, Homo sapiens
acatgttctccggcagttc

SEQ ID NO: 162 - DNA, 19 bp, Homo sapiens
gctgaatatcagcgcggac

SEQ ID NO: 163 - DNA, 19 bp, Homo sapiens
gggagtatgtttgtggccc

SEQ ID NO: 164 - DNA, 19 bp, Homo sapiens
tctactccttggtcaggac

SEQ ID NO: 165 - DNA, 19 bp, Homo sapiens
ggtgaagacctgtgacatc

SEQ ID NO: 166 - DNA, 19 bp, Homo sapiens
attcaagcgacccatcatc

SEQ ID NO: 167 - DNA, 19 bp, Homo sapiens
tgccctgatggtgctgaac

SEQ ID NO: 168 - DNA, 19 bp, Homo sapiens
gaacatgagcctgtgcttc

SEQ ID NO: 169 - DNA, 19 bp, Homo sapiens
gaagagggagagatggagc

SEQ ID NO: 170 - DNA, 19 bp, Homo sapiens
catgctggtcgtcctcatc

SEQ ID NO: 171 - DNA, 19 bp, Homo sapiens
tcccatcatctatgccttc

SEQ ID NO: 172 - DNA, 19 bp, Homo sapiens
gtgactacacaaggactcc

SEQ ID NO: 173 - DNA, 19 bp, Homo sapiens
catgctggtcatcctcatc

SEQ ID NO: 174 - DNA, 19 bp, Homo sapiens
gagcatgactgacatctac

SEQ ID NO: 175 - DNA, 19 bp, Homo sapiens
cattgtccttctcctgaac

SEQ ID NO: 176 - DNA, 19 bp, Homo sapiens
gcacattgccaaacgcttc

SEQ ID NO: 177 - DNA, 19 bp, Homo sapiens
gctcagtttacacccgatc

SEQ ID NO: 178 - DNA, 19 bp, Homo sapiens
ggccaggtctatgacagac

SEQ ID NO: 179 - DNA, 19 bp, Homo sapiens
atacaacacccaaggcagc

SEQ ID NO: 180 - DNA, 19 bp, Homo sapiens
gtacaagtcctcaggcttc

SEQ ID NO: 181 - DNA, 19 bp, Homo sapiens
ctcctgacctcaagtgatc

SEQ ID NO: 182 - DNA, 19 bp, Homo sapiens
tcgcttgaacccaggaggc

SEQ ID NO: 183 - DNA, 19 bp, Homo sapiens
atcacggtgcccatgtgcc

SEQ ID NO: 184 - DNA, 19 bp, Homo sapiens
ggagtcacacccgctctac

SEQ ID NO: 185 - DNA, 19 bp, Homo sapiens
gctcatgatccgcatcggc

SEQ ID NO: 186 - DNA, 19 bp, Homo sapiens
ctaccacaagcaggtgtcc

SEQ ID NO: 187 - DNA, 19 bp, Homo sapiens
tgtggtggtggtgatgatc

SEQ ID NO: 188 - DNA, 19 bp, Homo sapiens
gagactctttgcagtgctc

SEQ ID NO: 189 - DNA, 19 bp, Homo sapiens
gtacctgcgccacttcttc

SEQ ID NO: 190 - DNA, 19 bp, Homo sapiens
cagcttggtggtgatagtc

SEQ ID NO: 191 - DNA, 19 bp, Homo sapiens
gattgcttcagccagcgtc

SEQ ID NO: 192 - DNA, 19 bp, Homo sapiens
gccaaatcccactcaaacc

SEQ ID NO: 193 - DNA, 19 bp, Homo sapiens
gacaccctcagtcatctac

SEQ ID NO: 194 - DNA, 19 bp, Homo sapiens
accctcagtcatctacacc

SEQ ID NO: 195 - DNA, 19 bp, Homo sapiens
ggtgctcatcatctttctc

SEQ ID NO: 196 - DNA, 19 bp, Homo sapiens
ctgcctgtccctctacttc

SEQ ID NO: 197 - DNA, 19 bp, Homo sapiens
catctacatcagcgtgggc

SEQ ID NO: 198 - DNA, 19 bp, Homo sapiens
ggagctgctgaccagcatc

SEQ ID NO: 199 - DNA, 19 bp, Homo sapiens
gagccgcaaggaccagatc

SEQ ID NO: 200 - DNA, 19 bp, Homo sapiens
cgcctaccacttctccctc

SEQ ID NO: 201 - DNA, 19 bp, Homo sapiens
cggctcaagaaactggagc

SEQ ID NO: 202 - DNA, 19 bp, Homo sapiens
gatggctcatcacacactc

SEQ ID NO: 203 - DNA, 19 bp, Homo sapiens
cctgggaaacacgtgcttc

SEQ ID NO: 204 - DNA, 19 bp, Homo sapiens
acacgtgcttcctgaatgc

SEQ ID NO: 205 - DNA, 19 bp, Homo sapiens
tcgattccgagctgtcttc

SEQ ID NO: 206 - DNA, 19 bp, Homo sapiens
gttgtctcaagtgccaggc

SEQ ID NO: 207 - DNA, 19 bp, Homo sapiens
agccggaagtcctgtatac

SEQ ID NO: 208 - DNA, 19 bp, Homo sapiens
gccggaagtcctgtatacc

SEQ ID NO: 209 - DNA, 19 bp, Homo sapiens
tggaactgggacgaggtgc

SEQ ID NO: 210 - DNA, 19 bp, Homo sapiens
ttgatgcaggttgcaaacc

SEQ ID NO: 211 - DNA, 19 bp, Homo sapiens
cggcctacatacccagagc

SEQ ID NO: 212 - DNA, 19 bp, Homo sapiens
gcaagttcactacagcatc

SEQ ID NO: 213 - DNA, 19 bp, Homo sapiens
cttccacatcaaggcagcc

SEQ ID NO: 214 - DNA, 19 bp, Homo sapiens
atcaaggcagccatccagc

SEQ ID NO: 215 - DNA, 19 bp, Homo sapiens
ccgcatgtgtaacaacagc

SEQ ID NO: 216 - DNA, 19 bp, Homo sapiens
caacaactgcctcagctac

SEQ ID NO: 217 - DNA, 19 bp, Homo sapiens
actgagacctaagtccaac

SEQ ID NO: 218 - DNA, 19 bp, Homo sapiens
caaccatgctgtttacaac

SEQ ID NO: 219 - DNA, 19 bp, Homo sapiens
agagcgtgtgtgttagatc

SEQ ID NO: 220 - DNA, 19 bp, Homo sapiens
cggtgataactaccaagtc

SEQ ID NO: 221 - DNA, 19 bp, Homo sapiens
aagttcccgaacgatcacc

SEQ ID NO: 222 - DNA, 19 bp, Homo sapiens
aagtgtcagctgcttccgc

SEQ ID NO: 223 - DNA, 19 bp, Homo sapiens
ctagcagagcttctctccc

SEQ ID NO: 224 - DNA, 19 bp, Homo sapiens
catggcccttgggtgaatc

SEQ ID NO: 225 - DNA, 19 bp, Homo sapiens
ggagacaaggaaattggcc

SEQ ID NO: 226 - DNA, 19 bp, Homo sapiens
agctggtaaaccatgaagc

SEQ ID NO: 227 - DNA, 19 bp, Homo sapiens
tagagctggagcaggactc

SEQ ID NO: 228 - DNA, 19 bp, Homo sapiens
tgacttcctggacaatggc

SEQ ID NO: 229 - DNA, 19 bp, Homo sapiens
acgcctccgatacagcttc

SEQ ID NO: 230 - DNA, 19 bp, Homo sapiens
ttctcctgcctcagcctcc

SEQ ID NO: 231 - RNA, 7 nt, Homo sapiens
uugcuau

SEQ ID NO: 232 - DNA, 21 bp, Homo sapiens
acagtcaagtccatgcgaaac

SEQ ID NO: 233 - DNA, 21 bp, Homo sapiens
aacgtgtgctccagtggatgc

SEQ ID NO: 234 - DNA, 21 bp, Homo sapiens
aattggctgcaaactgatccc

SEQ ID NO: 235 - DNA, 21 bp, Homo sapiens
acagatacaaagccattgtcc

SEQ ID NO: 236 - DNA, 21 bp, Homo sapiens
acggccaatggatatccaggc

SEQ ID NO: 237 - DNA, 21 bp, Homo sapiens
tcccatgccctgatgaagatc

SEQ ID NO: 238 - DNA, 21 bp, Homo sapiens
aagatctgcctcaaagccgcc

SEQ ID NO: 239 - DNA, 21 bp, Homo sapiens
aaagccgcctttatctggatc

SEQ ID NO: 240 - DNA, 21 bp, Homo sapiens
accaaccagaccttcattagc

SEQ ID NO: 241 - DNA, 21 bp, Homo sapiens
acccacactctaatgagcttc

SEQ ID NO: 242 - DNA, 21 bp, Homo sapiens
acactctaatgagcttcaccc

SEQ ID NO: 243 - DNA, 21 bp, Homo sapiens
aatctgatccagagtgcttac

SEQ ID NO: 244 - DNA, 21 bp, Homo sapiens
acagtgctggtgtttgtgggc

SEQ ID NO: 245 - DNA, 21 bp, Homo sapiens
accatgtcatctacctgtacc

SEQ ID NO: 246 - DNA, 21 bp, Homo sapiens
aagtggacacctccatgctcc

SEQ ID NO: 247 - DNA, 21 bp, Homo sapiens
acctccatgctccactttgtc

SEQ ID NO: 248 - DNA, 21 bp, Homo sapiens
aacagttcaacactcagctgc

SEQ ID NO: 249 - DNA, 21 bp, Homo sapiens
aacactcagctgctctgttgc

SEQ ID NO: 250 - DNA, 21 bp, Homo sapiens
acactcagctgctctgttgcc

SEQ ID NO: 251 - DNA, 21 bp, Homo sapiens
actggaaggagtacaacctgc

SEQ ID NO: 252 - DNA, 21 bp, Homo sapiens
aaggagtacaacctgcatgac

SEQ ID NO: 253 - DNA, 21 bp, Homo sapiens
acaacctgcatgacctccctc

SEQ ID NO: 254 - DNA, 21 bp, Homo sapiens
acctccctcaagagtaccaac

SEQ ID NO: 255 - DNA, 21 bp, Homo sapiens
aaagagccttcagaatgctcc

SEQ ID NO: 256 - DNA, 19 bp, Homo sapiens
agtcaagtccatgcgaaac

SEQ ID NO: 257 - DNA, 19 bp, Homo sapiens
cgtgtgctccagtggatgc

SEQ ID NO: 258 - DNA, 19 bp, Homo sapiens
ttggctgcaaactgatccc

SEQ ID NO: 259 - DNA, 19 bp, Homo sapiens
agatacaaagccattgtcc

SEQ ID NO: 260 - DNA, 19 bp, Homo sapiens
ggccaatggatatccaggc

SEQ ID NO: 261 - DNA, 19 bp, Homo sapiens
ccatgccctgatgaagatc

SEQ ID NO: 262 - DNA, 19 bp, Homo sapiens
gatctgcctcaaagccgcc

SEQ ID NO: 263 - DNA, 19 bp, Homo sapiens
agccgcctttatctggatc

SEQ ID NO: 264 - DNA, 19 bp, Homo sapiens
caaccagaccttcattagc

SEQ ID NO: 265 - DNA, 19 bp, Homo sapiens
ccacactctaatgagcttc

SEQ ID NO: 266 - DNA, 19 bp, Homo sapiens
actctaatgagcttcaccc

SEQ ID NO: 267 - DNA, 19 bp, Homo sapiens
tctgatccagagtgcttac

SEQ ID NO: 268 - DNA, 19 bp, Homo sapiens
agtgctggtgtttgtgggc

SEQ ID NO: 269 - DNA, 19 bp, Homo sapiens
catgtcatctacctgtacc

SEQ ID NO: 270 - DNA, 19 bp, Homo sapiens
gtggacacctccatgctcc

SEQ ID NO: 271 - DNA, 19 bp, Homo sapiens
ctccatgctccactttgtc

SEQ ID NO: 272 - DNA, 19 bp, Homo sapiens
cagttcaacactcagctgc

SEQ ID NO: 273 - DNA, 19 bp, Homo sapiens
cactcagctgctctgttgc

SEQ ID NO: 274 - DNA, 19 bp, Homo sapiens
actcagctgctctgttgcc

SEQ ID NO: 275 - DNA, 19 bp, Homo sapiens
tggaaggagtacaacctgc

SEQ ID NO: 276 - DNA, 19 bp, Homo sapiens
ggagtacaacctgcatgac

SEQ ID NO: 277 - DNA, 19 bp, Homo sapiens
aacctgcatgacctccctc

SEQ ID NO: 278 - DNA, 19 bp, Homo sapiens
ctccctcaagagtaccaac

SEQ ID NO: 279 - DNA, 19 bp, Homo sapiens
agagccttcagaatgctcc

SEQ ID NO: 280 - DNA, 21 bp, Homo sapiens
aacatcctagtgatcctctcc

SEQ ID NO: 281 - DNA, 21 bp, Homo sapiens
acgcactactacatcgtcaac

SEQ ID NO: 282 - DNA, 21 bp, Homo sapiens
aagtctggcctcaagaccgac

SEQ ID NO: 283 - DNA, 21 bp, Homo sapiens
aagtgacgctccgcatccatc

SEQ ID NO: 284 - DNA, 21 bp, Homo sapiens
acgcacttctcagtgaggctc

SEQ ID NO: 285 - DNA, 21 bp, Homo sapiens
aaagcagtcttccaaacatgc

SEQ ID NO: 286 - DNA, 21 bp, Homo sapiens
aagcagtcttccaaacatgcc

SEQ ID NO: 287 - DNA, 21 bp, Homo sapiens
aaacatgccctgggctacacc

SEQ ID NO: 288 - DNA, 21 bp, Homo sapiens
acaaggacatggtgcgcatcc

SEQ ID NO: 289 - DNA, 21 bp, Homo sapiens
acagtgtccaaagaccaatcc

SEQ ID NO: 290 - DNA, 21 bp, Homo sapiens
aaagaccaatcctcctgtacc

SEQ ID NO: 291 - DNA, 21 bp, Homo sapiens
aatcctcctgtaccacagccc

SEQ ID NO: 292 - DNA, 21 bp, Homo sapiens
aagaaccatcaagttccaacc

SEQ ID NO: 293 - DNA, 21 bp, Homo sapiens
aagttccaaccattaaggtcc

SEQ ID NO: 294 - DNA, 21 bp, Homo sapiens
accattaaggtccacaccatc

SEQ ID NO: 295 - DNA, 21 bp, Homo sapiens
aaggtccacaccatctccctc

SEQ ID NO: 296 - DNA, 21 bp, Homo sapiens
accatctccctcagtgagaac

SEQ ID NO: 297 - DNA, 21 bp, Homo sapiens
aataatcttaggtacccaccc

SEQ ID NO: 298 - DNA, 21 bp, Homo sapiens
aacatcctagtgatcctctcc

SEQ ID NO: 299 - DNA, 21 bp, Homo sapiens
acgcactactacatcgtcaac

SEQ ID NO: 300 - DNA, 21 bp, Homo sapiens
aagtctggcctcaagaccgac

SEQ ID NO: 301 - DNA, 21 bp, Homo sapiens
aagtgacgctccgcatccatc

SEQ ID NO: 302 - DNA, 21 bp, Homo sapiens
acgcacttctcagtgaggctc

SEQ ID NO: 303 - DNA, 21 bp, Homo sapiens
aaagcagtcttccaaacatgc

SEQ ID NO: 304 - DNA, 21 bp, Homo sapiens
aagcagtcttccaaacatgcc

SEQ ID NO: 305 - DNA, 21 bp, Homo sapiens
aaacatgccctgggctacacc

SEQ ID NO: 306 - DNA, 21 bp, Homo sapiens
acaaggacatggtgcgcatcc

SEQ ID NO: 307 - DNA, 21 bp, Homo sapiens
acagtgtccaaagaccaatcc

SEQ ID NO: 308 - DNA, 21 bp, Homo sapiens
aaagaccaatcctcctgtacc

SEQ ID NO: 309 - DNA, 21 bp, Homo sapiens
aatcctcctgtaccacagccc

SEQ ID NO: 310 - DNA, 21 bp, Homo sapiens
acgaagtctcgctctgtcacc

SEQ ID NO: 311 - DNA, 21 bp, Homo sapiens
aatggcatgatcttggctcac

SEQ ID NO: 312 - DNA, 21 bp, Homo sapiens
acgatcttggctcactgcaac

SEQ ID NO: 313 - DNA, 21 bp, Homo sapiens
aagagattctcctgcctcagc

SEQ ID NO: 314 - DNA, 21 bp, Homo sapiens
accatgttggccaggatgatc

SEQ ID NO: 315 - DNA, 21 bp, Homo sapiens
acctcatgatctgcctgcctc

SEQ ID NO: 316 - DNA, 21 bp, Homo sapiens
aacacacacacacattctctc

SEQ ID NO: 317 - DNA, 21 bp, Homo sapiens
acacacacacacattctctcc

SEQ ID NO: 318 - DNA, 21 bp, Homo sapiens
acacattctctccatggtgac

SEQ ID NO: 319 - DNA, 21 bp, Homo sapiens
acatagtacaccatggagcac

SEQ ID NO: 320 - DNA, 21 bp, Homo sapiens
accatggagcacggtttaagc

SEQ ID NO: 321 - DNA, 21 bp, Homo sapiens
acggtttaagcaccactggac

SEQ ID NO: 322 - DNA, 21 bp, Homo sapiens
accttcccatagacacccagc

SEQ ID NO: 323 - DNA, 21 bp, Homo sapiens
aacatcctagtgatcctctcc

SEQ ID NO: 324 - DNA, 21 bp, Homo sapiens
acgcactactacatcgtcaac

SEQ ID NO: 325 - DNA, 21 bp, Homo sapiens
aagtctggcctcaagaccgac

SEQ ID NO: 326 - DNA, 21 bp, Homo sapiens
aagtgacgctccgcatccatc

SEQ ID NO: 327 - DNA, 21 bp, Homo sapiens
acgcacttctcagtgaggctc

SEQ ID NO: 328 - DNA, 21 bp, Homo sapiens
aaagcagtcttccaaacatgc

SEQ ID NO: 329 - DNA, 21 bp, Homo sapiens
aagcagtcttccaaacatgcc

SEQ ID NO: 330 - DNA, 21 bp, Homo sapiens
aaacatgccctgggctacacc

SEQ ID NO: 331 - DNA, 21 bp, Homo sapiens
acaaggacatggtgcgcatcc

SEQ ID NO: 332 - DNA, 21 bp, Homo sapiens
acagtgtccaaagaccaatcc

SEQ ID NO: 333 - DNA, 21 bp, Homo sapiens
aaagaccaatcctcctgtacc

SEQ ID NO: 334 - DNA, 21 bp, Homo sapiens
aatcctcctgtaccacagccc

SEQ ID NO: 335 - DNA, 21 bp, Homo sapiens
acacacccatgacatgaagcc

SEQ ID NO: 336 - DNA, 21 bp, Homo sapiens
acatgaagccagcttcccgtc

SEQ ID NO: 337 - DNA, 21 bp, Homo sapiens
acgactgttgtccttactgcc

SEQ ID NO: 338 - DNA, 21 bp, Homo sapiens
aagcatccatctgactaaggc

SEQ ID NO: 339 - DNA, 21 bp, Homo sapiens
aacatcctagtgatcctctcc

SEQ ID NO: 340 - DNA, 21 bp, Homo sapiens
acgcactactacatcgtcaac

SEQ ID NO: 341 - DNA, 21 bp, Homo sapiens
aagtctggcctcaagaccgac

SEQ ID NO: 342 - DNA, 21 bp, Homo sapiens
aagtgacgctccgcatccatc

SEQ ID NO: 343 - DNA, 21 bp, Homo sapiens
acgcacttctcagtgaggctc

SEQ ID NO: 344 - DNA, 21 bp, Homo sapiens
aaagcagtcttccaaacatgc

SEQ ID NO: 345 - DNA, 21 bp, Homo sapiens
aagcagtcttccaaacatgcc

SEQ ID NO: 346 - DNA, 21 bp, Homo sapiens
aaacatgccctgggctacacc

SEQ ID NO: 347 - DNA, 21 bp, Homo sapiens
acaaggacatggtgcgcatcc

SEQ ID NO: 348 - DNA, 21 bp, Homo sapiens
acagtgtccaaagaccaatcc

SEQ ID NO: 349 - DNA, 21 bp, Homo sapiens
aaagaccaatcctcctgtacc

SEQ ID NO: 350 - DNA, 21 bp, Homo sapiens
aatcctcctgtaccacagccc

SEQ ID NO: 351 - DNA, 21 bp, Homo sapiens
aaagggtctagaatgctgatc

SEQ ID NO: 352 - DNA, 21 bp, Homo sapiens
aatgaggagtcagctggaagc

SEQ ID NO: 353 - DNA, 21 bp, Homo sapiens
aaactggatatcccaaccttc

SEQ ID NO: 354 - DNA, 21 bp, Homo sapiens
accagtaggtttcatggttac

SEQ ID NO: 355 - DNA, 19 bp, Homo sapiens
catcctagtgatcctctcc

SEQ ID NO: 356 - DNA, 19 bp, Homo sapiens
gcactactacatcgtcaac

SEQ ID NO: 357 - DNA, 19 bp, Homo sapiens
gtctggcctcaagaccgac

SEQ ID NO: 358 - DNA, 19 bp, Homo sapiens
gtgacgctccgcatccatc

SEQ ID NO: 359 - DNA, 19 bp, Homo sapiens
gcacttctcagtgaggctc

SEQ ID NO: 360 - DNA, 19 bp, Homo sapiens
agcagtcttccaaacatgc

SEQ ID NO: 361 - DNA, 19 bp, Homo sapiens
gcagtcttccaaacatgcc

SEQ ID NO: 362 - DNA, 19 bp, Homo sapiens
acatgccctgggctacacc

SEQ ID NO: 363 - DNA, 19 bp, Homo sapiens
aaggacatggtgcgcatcc

SEQ ID NO: 364 - DNA, 19 bp, Homo sapiens
agtgtccaaagaccaatcc

SEQ ID NO: 365 - DNA, 19 bp, Homo sapiens
agaccaatcctcctgtacc

SEQ ID NO: 366 - DNA, 19 bp, Homo sapiens
tcctcctgtaccacagccc

SEQ ID NO: 367 - DNA, 19 bp, Homo sapiens
gaaccatcaagttccaacc

SEQ ID NO: 368 - DNA, 19 bp, Homo sapiens
gttccaaccattaaggtcc

SEQ ID NO: 369 - DNA, 19 bp, Homo sapiens
cattaaggtccacaccatc

SEQ ID NO: 370 - DNA, 19 bp, Homo sapiens
ggtccacaccatctccctc

SEQ ID NO: 371 - DNA, 19 bp, Homo sapiens
catctccctcagtgagaac

SEQ ID NO: 372 - DNA, 19 bp, Homo sapiens
taatcttaggtacccaccc

SEQ ID NO: 373 - DNA, 19 bp, Homo sapiens
catcctagtgatcctctcc

SEQ ID NO: 374 - DNA, 19 bp, Homo sapiens
gcactactacatcgtcaac

SEQ ID NO: 375 - DNA, 19 bp, Homo sapiens
gtctggcctcaagaccgac

SEQ ID NO: 376 - DNA, 19 bp, Homo sapiens
gtgacgctccgcatccatc

SEQ ID NO: 377 - DNA, 19 bp, Homo sapiens
gcacttctcagtgaggctc

SEQ ID NO: 378 - DNA, 19 bp, Homo sapiens
agcagtcttccaaacatgc

SEQ ID NO: 379 - DNA, 19 bp, Homo sapiens
gcagtcttccaaacatgcc

SEQ ID NO: 380 - DNA, 19 bp, Homo sapiens
acatgccctgggctacacc

SEQ ID NO: 381 - DNA, 19 bp, Homo sapiens
aaggacatggtgcgcatcc

SEQ ID NO: 382 - DNA, 19 bp, Homo sapiens
agtgtccaaagaccaatcc

SEQ ID NO: 383 - DNA, 19 bp, Homo sapiens
agaccaatcctcctgtacc

SEQ ID NO: 384 - DNA, 19 bp, Homo sapiens
tcctcctgtaccacagccc

SEQ ID NO: 385 - DNA, 19 bp, Homo sapiens
gaagtctcgctctgtcacc

SEQ ID NO: 386 - DNA, 19 bp, Homo sapiens
tggcatgatcttggctcac

SEQ ID NO: 387 - DNA, 19 bp, Homo sapiens
gatcttggctcactgcaac

SEQ ID NO: 388 - DNA, 19 bp, Homo sapiens
gagattctcctgcctcagc

SEQ ID NO: 389 - DNA, 19 bp, Homo sapiens
catgttggccaggatgatc

SEQ ID NO: 390 - DNA, 19 bp, Homo sapiens
ctcatgatctgcctgcctc

SEQ ID NO: 391 - DNA, 19 bp, Homo sapiens
cacacacacacattctctc

SEQ ID NO: 392 - DNA, 19 bp, Homo sapiens
acacacacacattctctcc

SEQ ID NO: 393 - DNA, 19 bp, Homo sapiens
acattctctccatggtgac

SEQ ID NO: 394 - DNA, 19 bp, Homo sapiens
atagtacaccatggagcac

SEQ ID NO: 395 - DNA, 19 bp, Homo sapiens
catggagcacggtttaagc

SEQ ID NO: 396 - DNA, 19 bp, Homo sapiens
ggtttaagcaccactggac

SEQ ID NO: 397 - DNA, 19 bp, Homo sapiens
cttcccatagacacccagc

SEQ ID NO: 398 - DNA, 19 bp, Homo sapiens
catcctagtgatcctctcc

SEQ ID NO: 399 - DNA, 19 bp, Homo sapiens
gcactactacatcgtcaac

SEQ ID NO: 400 - DNA, 19 bp, Homo sapiens
gtctggcctcaagaccgac

SEQ ID NO: 401 - DNA, 19 bp, Homo sapiens
gtgacgctccgcatccatc

SEQ ID NO: 402 - DNA, 19 bp, Homo sapiens
gcacttctcagtgaggctc

SEQ ID NO: 403 - DNA, 19 bp, Homo sapiens
agcagtcttccaaacatgc

SEQ ID NO: 404 - DNA, 19 bp, Homo sapiens
gcagtcttccaaacatgcc

SEQ ID NO: 405 - DNA, 19 bp, Homo sapiens
acatgccctgggctacacc

SEQ ID NO: 406 - DNA, 19 bp, Homo sapiens
aaggacatggtgcgcatcc

SEQ ID NO: 407 - DNA, 19 bp, Homo sapiens
agtgtccaaagaccaatcc

SEQ ID NO: 408 - DNA, 19 bp, Homo sapiens
agaccaatcctcctgtacc

SEQ ID NO: 409 - DNA, 19 bp, Homo sapiens
tcctcctgtaccacagccc

SEQ ID NO: 410 - DNA, 19 bp, Homo sapiens
acacccatgacatgaagcc

SEQ ID NO: 411 - DNA, 19 bp, Homo sapiens
atgaagccagcttcccgtc

SEQ ID NO: 412 - DNA, 19 bp, Homo sapiens
gactgttgtccttactgcc

SEQ ID NO: 413 - DNA, 19 bp, Homo sapiens
gcatccatctgactaaggc

SEQ ID NO: 414 - DNA, 19 bp, Homo sapiens
catcctagtgatcctctcc

SEQ ID NO: 415 - DNA, 19 bp, Homo sapiens
gcactactacatcgtcaac

SEQ ID NO: 416 - DNA, 19 bp, Homo sapiens
gtctggcctcaagaccgac

SEQ ID NO: 417 - DNA, 19 bp, Homo sapiens
gtgacgctccgcatccatc

SEQ ID NO: 418 - DNA, 19 bp, Homo sapiens
gcacttctcagtgaggctc

SEQ ID NO: 419 - DNA, 19 bp, Homo sapiens
agcagtcttccaaacatgc

SEQ ID NO: 420 - DNA, 19 bp, Homo sapiens
gcagtcttccaaacatgcc

SEQ ID NO: 421 - DNA, 19 bp, Homo sapiens
acatgccctgggctacacc

SEQ ID NO: 422 - DNA, 19 bp, Homo sapiens
aaggacatggtgcgcatcc

SEQ ID NO: 423 - DNA, 19 bp, Homo sapiens
agtgtccaaagaccaatcc

SEQ ID NO: 424 - DNA, 19 bp, Homo sapiens
agaccaatcctcctgtacc

SEQ ID NO: 425 - DNA, 19 bp, Homo sapiens
tcctcctgtaccacagccc

SEQ ID NO: 426 - DNA, 19 bp, Homo sapiens
agggtctagaatgctgatc

SEQ ID NO: 427 - DNA, 19 bp, Homo sapiens
tgaggagtcagctggaagc

SEQ ID NO: 428 - DNA, 19 bp, Homo sapiens
actggatatcccaaccttc

SEQ ID NO: 429 - DNA, 19 bp, Homo sapiens
cagtaggtttcatggttac

SEQ ID NO: 430 - DNA, 21 bp, Homo sapiens
aaccagcctggcaaattgtcc

SEQ ID NO: 431 - DNA, 21 bp, Homo sapiens
aattgtcctttgggcagctgc

SEQ ID NO: 432 - DNA, 21 bp, Homo sapiens
aacgtggtagtgatgtggatc

SEQ ID NO: 433 - DNA, 21 bp, Homo sapiens
aatacagtggtgaacttcacc

SEQ ID NO: 434 - DNA, 21 bp, Homo sapiens
aacttcacctatgctgtccac

SEQ ID NO: 435 - DNA, 21 bp, Homo sapiens
acaacgaatggtactacggcc

SEQ ID NO: 436 - DNA, 21 bp, Homo sapiens
aagttccacaacttctttccc

SEQ ID NO: 437 - DNA, 21 bp, Homo sapiens
acagccaccaaagtggtcatc

SEQ ID NO: 438 - DNA, 21 bp, Homo sapiens
accaaagtggtcatctgtgtc

SEQ ID NO: 439 - DNA, 21 bp, Homo sapiens
aaagtggtcatctgtgtcatc

SEQ ID NO: 440 - DNA, 21 bp, Homo sapiens
actcaaccacagagaccatgc

SEQ ID NO: 441 - DNA, 21 bp, Homo sapiens
accacatctgtgtgactgtgc

SEQ ID NO: 442 - DNA, 21 bp, Homo sapiens
actgtgctgatctacttcctc

SEQ ID NO: 443 - DNA, 21 bp, Homo sapiens
acaccgtagtgggaatcacac

SEQ ID NO: 444 - DNA, 21 bp, Homo sapiens
acactatgggccagtgagatc

SEQ ID NO: 445 - DNA, 21 bp, Homo sapiens
actatgggccagtgagatccc

SEQ ID NO: 446 - DNA, 21 bp, Homo sapiens
acgagcaagtctctgccaagc

SEQ ID NO: 447 - DNA, 21 bp, Homo sapiens
acatcaacccagatctctacc

SEQ ID NO: 448 - DNA, 21 bp, Homo sapiens
acctgaagaagtttatccagc

SEQ ID NO: 449 - DNA, 21 bp, Homo sapiens
aagaagtttatccagcaggtc

SEQ ID NO: 450 - DNA, 21 bp, Homo sapiens
aagtttatccagcaggtctac

SEQ ID NO: 451 - DNA, 21 bp, Homo sapiens
aatgacaggttccgtctgggc

SEQ ID NO: 452 - DNA, 21 bp, Homo sapiens
aaatgaaatccacccggtatc

SEQ ID NO: 453 - DNA, 21 bp, Homo sapiens
aactgctcttcacgaagtgac

SEQ ID NO: 454 - DNA, 21 bp, Homo sapiens
aagtgactccaagaccatgac

SEQ ID NO: 455 - DNA, 21 bp, Homo sapiens
aagaccatgacagagagcttc

SEQ ID NO: 456 - DNA, 21 bp, Homo sapiens
accatgacagagagcttcagc

SEQ ID NO: 457 - DNA, 21 bp, Homo sapiens
acagagagcttcagcttctcc

SEQ ID NO: 458 - DNA, 21 bp, Homo sapiens
aaattcccttcatctggaacc

SEQ ID NO: 459 - DNA, 21 bp, Homo sapiens
aaccatcagaaacaccctcac

SEQ ID NO: 460 - DNA, 21 bp, Homo sapiens
aatcactgaactttgctgagc

SEQ ID NO: 461 - DNA, 21 bp, Homo sapiens
actttggctgcatgcgagtgc

SEQ ID NO: 462 - DNA, 21 bp, Homo sapiens
aaccagcctggcaaattgtcc

SEQ ID NO: 463 - DNA, 21 bp, Homo sapiens
aattgtcctttgggcagctgc

SEQ ID NO: 464 - DNA, 21 bp, Homo sapiens
aacgtggtagtgatgtggatc

SEQ ID NO: 465 - DNA, 21 bp, Homo sapiens
aatacagtggtgaacttcacc

SEQ ID NO: 466 - DNA, 21 bp, Homo sapiens
aacttcacctatgctgtccac

SEQ ID NO: 467 - DNA, 21 bp, Homo sapiens
acaacgaatggtactacggcc

SEQ ID NO: 468 - DNA, 21 bp, Homo sapiens
acagccaccaaagtggtcatc

SEQ ID NO: 469 - DNA, 21 bp, Homo sapiens
accaaagtggtcatctgtgtc

SEQ ID NO: 470 - DNA, 21 bp, Homo sapiens
aaagtggtcatctgtgtcatc

SEQ ID NO: 471 - DNA, 21 bp, Homo sapiens
actcaaccacagagaccatgc

SEQ ID NO: 472 - DNA, 21 bp, Homo sapiens
accacatctgtgtgactgtgc

SEQ ID NO: 473 - DNA, 21 bp, Homo sapiens
actgtgctgatctacttcctc

SEQ ID NO: 474 - DNA, 19 bp, Homo sapiens
acaccgtagtgggaatcac

SEQ ID NO: 475 - DNA, 21 bp, Homo sapiens
acactatgggccagtgagatc

SEQ ID NO: 476 - DNA, 21 bp, Homo sapiens
actatgggccagtgagatccc

SEQ ID NO: 477 - DNA, 21 bp, Homo sapiens
acgagcaagtctctgccaagc

SEQ ID NO: 478 - DNA, 21 bp, Homo sapiens
acatcaacccagatctctacc

SEQ ID NO: 479 - DNA, 21 bp, Homo sapiens
acctgaagaagtttatccagc

SEQ ID NO: 480 - DNA, 21 bp, Homo sapiens
aagaagtttatccagcaggtc

SEQ ID NO: 481 - DNA, 21 bp, Homo sapiens
aagtttatccagcaggtctac

SEQ ID NO: 482 - DNA, 21 bp, Homo sapiens
accatctacatacacagtggc

SEQ ID NO: 483 - DNA, 21 bp, Homo sapiens
aactcagcctggctgattatc

SEQ ID NO: 484 - DNA, 21 bp, Homo sapiens
aaccagcctggcaaattgtcc

SEQ ID NO: 485 - DNA, 21 bp, Homo sapiens
aattgtcctttgggcagctgc

SEQ ID NO: 486 - DNA, 21 bp, Homo sapiens
aacgtggtagtgatgtggatc

SEQ ID NO: 487 - DNA, 21 bp, Homo sapiens
aatacagtggtgaacttcacc

SEQ ID NO: 488 - DNA, 21 bp, Homo sapiens
aacttcacctatgctgtccac

SEQ ID NO: 489 - DNA, 21 bp, Homo sapiens
acaacgaatggtactacggcc

SEQ ID NO: 490 - DNA, 21 bp, Homo sapiens
aagttccacaacttctttccc

SEQ ID NO: 491 - DNA, 21 bp, Homo sapiens
acagccaccaaagtggtcatc

SEQ ID NO: 492 - DNA, 21 bp, Homo sapiens
accaaagtggtcatctgtgtc

SEQ ID NO: 493 - DNA, 21 bp, Homo sapiens
aaagtggtcatctgtgtcatc

SEQ ID NO: 494 - DNA, 21 bp, Homo sapiens
actcaaccacagagaccatgc

SEQ ID NO: 495 - DNA, 21 bp, Homo sapiens
accacatctgtgtgactgtgc

SEQ ID NO: 496 - DNA, 21 bp, Homo sapiens
actgtgctgatctacttcctc

SEQ ID NO: 497 - DNA, 21 bp, Homo sapiens
acaccgtagtgggaatcacac

SEQ ID NO: 498 - DNA, 21 bp, Homo sapiens
acactatgggccagtgagatc

SEQ ID NO: 499 - DNA, 21 bp, Homo sapiens
actatgggccagtgagatccc

SEQ ID NO: 500 - DNA, 21 bp, Homo sapiens
acgagcaagtctctgccaagc

SEQ ID NO: 501 - DNA, 21 bp, Homo sapiens
acatcaacccagatctctacc

SEQ ID NO: 502 - DNA, 21 bp, Homo sapiens
acctgaagaagtttatccagc

SEQ ID NO: 503 - DNA, 21 bp, Homo sapiens
aagaagtttatccagcaggtc

SEQ ID NO: 504 - DNA, 21 bp, Homo sapiens
aagtttatccagcaggtctac

SEQ ID NO: 505 - DNA, 21 bp, Homo sapiens
aatgacaggttccgtctgggc

SEQ ID NO: 506 - DNA, 21 bp, Homo sapiens
aaatgaaatccacccggtatc

SEQ ID NO: 507 - DNA, 21 bp, Homo sapiens
aactgctcttcacgaagtgac

SEQ ID NO: 508 - DNA, 21 bp, Homo sapiens
aagtgactccaagaccatgac

SEQ ID NO: 509 - DNA, 21 bp, Homo sapiens
aagaccatgacagagagcttc

SEQ ID NO: 510 - DNA, 21 bp, Homo sapiens
accatgacagagagcttcagc

SEQ ID NO: 511 - DNA, 21 bp, Homo sapiens
acagagagcttcagcttctcc

SEQ ID NO: 512 - DNA, 21 bp, Homo sapiens
aaattcccttcatctggaacc

SEQ ID NO: 513 - DNA, 21 bp, Homo sapiens
aaccatcagaaacaccctcac

SEQ ID NO: 514 - DNA, 21 bp, Homo sapiens
aatcactgaactttgctgagc

SEQ ID NO: 515 - DNA, 21 bp, Homo sapiens
actttggctgcatgcgagtgc

SEQ ID NO: 516 - DNA, 21 bp, Homo sapiens
aaccagcctggcaaattgtcc

SEQ ID NO: 517 - DNA, 21 bp, Homo sapiens
aattgtcctttgggcagctgc

SEQ ID NO: 518 - DNA, 21 bp, Homo sapiens
aacgtggtagtgatgtggatc

SEQ ID NO: 519 - DNA, 21 bp, Homo sapiens
aatacagtggtgaacttcacc

SEQ ID NO: 520 - DNA, 21 bp, Homo sapiens
aacttcacctatgctgtccac

SEQ ID NO: 521 - DNA, 21 bp, Homo sapiens
acaacgaatggtactacggcc

SEQ ID NO: 522 - DNA, 21 bp, Homo sapiens
acagccaccaaagtggtcatc

SEQ ID NO: 523 - DNA, 21 bp, Homo sapiens
accaaagtggtcatctgtgtc

SEQ ID NO: 524 - DNA, 21 bp, Homo sapiens
aaagtggtcatctgtgtcatc

SEQ ID NO: 525 - DNA, 21 bp, Homo sapiens
actcaaccacagagaccatgc

SEQ ID NO: 526 - DNA, 21 bp, Homo sapiens
accacatctgtgtgactgtgc

SEQ ID NO: 527 - DNA, 21 bp, Homo sapiens
actgtgctgatctacttcctc

SEQ ID NO: 528 - DNA, 21 bp, Homo sapiens
acaccgtagtgggaatcacac

SEQ ID NO: 529 - DNA, 21 bp, Homo sapiens
acactatgggccagtgagatc

SEQ ID NO: 530 - DNA, 21 bp, Homo sapiens
actatgggccagtgagatccc

SEQ ID NO: 531 - DNA, 21 bp, Homo sapiens
acgagcaagtctctgccaagc

SEQ ID NO: 532 - DNA, 21 bp, Homo sapiens
acatcaacccagatctctacc

SEQ ID NO: 533 - DNA, 21 bp, Homo sapiens
acctgaagaagtttatccagc

SEQ ID NO: 534 - DNA, 21 bp, Homo sapiens
aagaagtttatccagcaggtc

SEQ ID NO: 535 - DNA, 21 bp, Homo sapiens
aagtttatccagcaggtctac

SEQ ID NO: 536 - DNA, 21 bp, Homo sapiens
accatctacatacacagtggc

SEQ ID NO: 537 - DNA, 21 bp, Homo sapiens
aactcagcctggctgattatc

SEQ ID NO: 538 - protein, 38 aa, Homo sapiens
MALNDCFLLNLEVDHFMHCNISSHSADLPVNDDWSHPG

SEQ ID NO: 539 - protein, 23 aa, Homo sapiens
ILYVIPAVYGVIILIGLIGNITL

SEQ ID NO: 540 - protein, 12 aa, Homo sapiens
IKIFCTVKSMRN

SEQ ID NO: 541 - protein, 23 aa, Homo sapiens
VPNLFISSLALGDLLLLITCAPV

SEQ ID NO: 542 - protein, 19 aa, Homo sapiens
DASRYLADRWLFGRIGCKL

SEQ ID NO: 543 - protein, 21 aa, Homo sapiens
IPFIQLTSVGVSVFTLTALSA

SEQ ID NO: 544 - protein, 20 aa, Homo sapiens
DRYKAIVRPMDIQASHALMK

SEQ ID NO: 545 - protein, 23 aa, Homo sapiens
ICLKAAFIWIISMLLAIPEAVFS

SEQ ID NO: 546 - protein, 31 aa, Homo sapiens
DLHPFHEESTNQTFISCAPYPHSNELHPKIH

SEQ ID NO: 547 - protein, 23 aa, Homo sapiens
SMASFLVFYVIPLSIISVYYYFI

SEQ ID NO: 548 - protein, 28 aa, Homo sapiens
AKNLIQSAYNLPVEGNIHVKKQIESRKR

SEQ ID NO: 549 - protein, 23 aa, Homo sapiens
LAKTVLVFVGLFAFCWLPNHVIY

SEQ ID NO: 550 - protein, 19 aa, Homo sapiens
LYRSYHYSEVDTSMLHFVT

SEQ ID NO: 551 - protein, 23 aa, Homo sapiens
SICARLLAFTNSCVNPFALYLLS

SEQ ID NO: 552 - protein, 58 aa, Homo sapiens
KSFRKQFNTQLLCCQPGLIIRSHSTGRSTTCMTSLKSTNPSVATFSLINGNICHERYV

SEQ ID NO: 553 - protein, 27 aa, Homo sapiens
MVFLSGNASDSSNCTQPPAPVNISKAI

SEQ ID NO: 554 - protein, 23 aa, Homo sapiens
LLGVILGGLILFGVLGNILVILS

SEQ ID NO: 555 - protein, 12 aa, Homo sapiens
VACHRHLHSVTH

SEQ ID NO: 556 - protein, 23 aa, Homo sapiens
YYIVNLAVADLLLTSTVLPFSAI

SEQ ID NO: 557 - protein, 14 aa, Homo sapiens
FEVLGYWAFGRVFC

SEQ ID NO: 558 - protein, 23 aa, Homo sapiens
NIWAAVDVLCCTASIMGLCIISI

SEQ ID NO: 559 - protein, 20 aa, Homo sapiens
DRYIGVSYPLRYPTIVTQRR

SEQ ID NO: 560 - protein, 23 aa, Homo sapiens
GLMALLCVWALSLVISIGPLFGW

SEQ ID NO: 561 - protein, 19 aa, Homo sapiens
RQPAPEDETICQINEEPGY

SEQ ID NO: 562 - protein, 23 aa, Homo sapiens
VLFSALGSFYLPLAIILVMYCRV

SEQ ID NO: 563 - protein, 65 aa, Homo sapiens
YVVAKRESRGLKSGLKTDKSDSEQVTLRIHRKNAPAGGSGMASAKTKTHFSVRLLKFSREKKAAK

SEQ ID NO: 564 - protein, 23 aa, Homo sapiens
TLGIVVGCFVLCWLPFFLVMPIG

SEQ ID NO: 565 - protein, 14 aa, Homo sapiens
SFFPDFKPSETVFK

SEQ ID NO: 566 - protein, 20 aa, Homo sapiens
IVFWLGYLNSCINPIIYPCS

SEQ ID NO: 567 - protein, 100 aa, Homo sapiens
SQEFKKAFQNVLRIQCLRRKQSSKHALGYTLHPPSQAVEGQHKDMVRIPVGSRETFYRISKTDGVCEWKFFSSMPRGSARITVSKDQSSCTTARGHTPMT

SEQ ID NO: 568 - protein, 34 aa, Homo sapiens
MDNVLPVDSDLSPNISTNTSEPNQFVQPAWQIVL

SEQ ID NO: 569 - protein, 23 aa, Homo sapiens
WAAAYTVIVVTSVVGNVVVMWII

SEQ ID NO: 570 - protein, 12 aa, Homo sapiens
LAHKRMRTVTNY

SEQ ID NO: 571 - protein, 23 aa, Homo sapiens
FLVNLAFAEASMAAFNTVVNFTY

SEQ ID NO: 572 - protein, 14 aa, Homo sapiens
AVHNEWYYGLFYCK

SEQ ID NO: 573 - protein, 22 aa, Homo sapiens
FHNFFPIAAVFASIYSMTAVAF

SEQ ID NO: 574 - protein, 20 aa, Homo sapiens
DRYMAIIHPLQPRLSATATK

SEQ ID NO: 575 - protein, 20 aa, Homo sapiens
VVICVIWVLALLLAFPQGYY

SEQ ID NO: 576 - protein, 34 aa, Homo sapiens
STTETMPSRVVCMIEWPEHPNKIYEKVYHICVTV

SEQ ID NO: 577 - protein, 23 aa, Homo sapiens
LIYFLPLLVIGYAYTVVGITLWA

SEQ ID NO: 578 - protein, 20 aa, Homo sapiens
SEIPGDSSDRYHEQVSAKRK

SEQ ID NO: 579 - protein, 23 aa, Homo sapiens
VVKMMIVVVCTFAICWLPFHIFF

SEQ ID NO: 580 - protein, 14 aa, Homo sapiens
LLPYINPDLYLKKF

SEQ ID NO: 581 - protein, 23 aa, Homo sapiens
IQQVYLAIMWLAMSSTMYNPIIY

SEQ ID NO: 582 - protein, 6 aa, Homo sapiens
CCLNDR

SEQ ID NO: 583 - DNA, 18 bp, Homo sapiens
catgctgctggccattcc

SEQ ID NO: 584 - DNA, 21 bp, Homo sapiens
aggtctggttggtgctttcct

SEQ ID NO: 585 - DNA, 23 bp, Mus musculus
tgtcttcacacttacggcactgt

SEQ ID NO: 586 - DNA, 20 bp, Mus musculus
gcatgggatgcctggatatc

SEQ ID NO: 587 - DNA, 18 bp, Homo sapiens
caaaacgctgggcatcgt

SEQ ID NO: 588 - DNA, 22 bp, Homo sapiens
gacccaatgggcatgactaaga

SEQ ID NO: 589 - DNA, 19 bp, Mus musculus
tgcccattgggtccttctt

SEQ ID NO: 590 - DNA, 26 bp, Mus musculus
ggtacccaagccaaaatactattttg

(19) United States
(12) Patent Application Publication
(10) Pub. No.: US 2005/0287565 A1
(43) Pub. Date: Dec. 29, 2005

(54) Methods, compositions and compound assays for inhibiting amyloid-beta protein production

(76) Inventors: Pascal Gerard Merchiers, Tielen (BE); Marcel Hoffmann, Uithoorn (NL); Koenraad Frederik Florentina Spittaels, Puurs (BE); Wendy Laenen, Lier (BE)

Correspondence Address: SYNNESTVEDT & LECHNER, LLP, 2600 ARAMARK TOWER, 1101 MARKET STREET, PHILADELPHIA, PA, 191072950, US

(21) Appl. No.: 11/127,877

(22) Filed: May 12, 2005

Related U.S. Application Data
(60) Provisional application No. 60/570,352, filed on May 12, 2004.
(60) Provisional application No. 60/603,948, filed on Aug. 24, 2004.

(51) Int. Cl. C12Q 1/68; G01N 33/53; A61K 48/00

(52) U.S. Cl. 435/6; 435/7.1; 514/44

(57) ABSTRACT

A method for identifying compounds that inhibit amyloid-beta precursor protein processing in cells, comprising contacting a test compound with a GPCR polypeptide, or fragment thereof, and measuring a compound-GPCR property related to the production of amyloid-beta peptide. Cellular assays of the method measure indicators including second messenger and/or amyloid beta peptide levels. Therapeutic methods, and pharmaceutical compositions including effective amyloid-beta precursor processing-inhibiting amounts of GPCR expression inhibitors, are useful for treating conditions involving cognitive impairment such as Alzheimers Disease.

### CROSS-REFERENCE TO RELATED APPLICATIONS

This application claims priority to U.S. Provisional Application No. 60/570,352, filed May 12, 2004, and U.S. Provisional Application No. 60/603,948, filed Aug. 24, 2004, the disclosures of which are incorporated herein by reference.

### FIELD OF THE INVENTION

This invention relates to the field of mammalian neuronal cell disorders, and in particular, to methods for identifying effective compounds, and therapies and compositions using such compounds, useful for the prevention and treatment of diseases associated with progressive loss of intellectual capacities in humans.

The neurological disorder that is most widely known for its progressive loss of intellectual capacities is Alzheimer's disease (AD). Worldwide, about 20 million people suffer from Alzheimer's disease. AD is clinically characterized by the initial loss of memory, followed by disorientation, impairment of judgment and reasoning, which is commonly referred to as cognitive impairment, and ultimately by full dementia. AD patients finally lapse into a severely debilitated, immobile state between four and twelve years after onset of the disease.

The key pathological evidence for AD is the presence of extracellular amyloid plaques and intracellular tau tangles in the brain, which are associated with neuronal degeneration (Ritchie and Lovestone (2002)). The extracellular amyloid plaques are believed to result from an increase in the insoluble amyloid beta peptide 1-42 produced by the metabolism of amyloid-beta precursor protein (APP). Following secretion, these amyloid beta 1-42 peptides form amyloid fibrils more readily than the amyloid beta 1-40 peptides, which are predominantly produced in healthy people. It appears that the amyloid beta peptide is on top of the neurotoxic cascade: experiments show that amyloid beta fibrils, when injected into the brains of P301L tau transgenic mice, enhance the formation of neurofibrillary tangles (Gotz et al. (2001)). In fact, a variety of amyloid beta peptides have been identified as amyloid beta peptides 1-42, 1-40, A-C9, A-C8, A-C7, which can be found in plaques and are often seen in cerebral spinal fluid.

The amyloid beta peptides are generated (or processed) from the membrane anchored APP, after cleavage by beta secretase and gamma secretase at position 1 and 40 or 42, respectively (FIG. 1A)(Annaert and De Strooper (2002)). In addition, high activity of beta secretase results in a shift of the cleavage at position 1 to position 11. Cleavage of amyloid-beta precursor protein by alpha secretase activity at position 17 and gamma secretase activity at 40 or 42 generates the non-pathological p3 peptide. Beta secretase was identified as the membrane anchored aspartyl protease BACE, while gamma secretase is a protein complex comprising presenilin 1 (PS1) or presenilin 2 (PS2), nicastrin, Anterior Pharynx Defective 1 (APH1) and Presenilin Enhancer 2 (PEN2). Of these proteins, the presenilins are widely thought to constitute the catalytic activity of the gamma secretase, while the other components play a role in the maturation and localization of the complex. The identity of the alpha secretase is still illustrious, although some results point towards the proteases ADAM 10 and TACE, which could have redundant functions.

A small fraction of AD cases (mostly early onset AD) are caused by autosomal dominant mutations in the genes encoding presenilin 1 and 2 (PS1; PS2) and the amyloid-beta precursor protein (APP), and it has been shown that mutations in APP, PS1 and PS2 alter the metabolism of amyloid-beta precursor protein leading to such increased levels of amyloid beta 1-42 produced in the brain. Although no mutations in PS1, PS2 and amyloid-beta precursor protein have been identified in late onset AD patients, the pathological characteristics are highly similar to the early onset AD patients. These increased levels of amyloid beta peptide could originate progressively with age from disturbed amyloid-beta precursor protein processing (e.g. high cholesterol levels enhance amyloid beta peptide production) or from decreased amyloid beta peptide catabolism. Therefore, it is generally accepted that AD in late onset AD patients is also caused by aberrant increased amyloid peptide levels in the brains. The level of these amyloid beta peptides, and more particularly amyloid-beta peptide 1-42, is increased in Alzheimer patients compared to the levels of these peptides in healthy persons. Thus, reducing the levels of these amyloid beta peptides is likely to be beneficial for patients with cognitive impairment.

### Reported Developments

The major current AD therapies are limited to delaying progressive memory loss by inhibiting the acetylcholinesterase enzyme, which increases acetylcholine neurotransmitter levels, which fall because the cholinergic neurons are the first neurons to degenerate during AD. This therapy does not halt the progression of the disease.

Therapies aimed at decreasing the levels of amyloid beta peptides in the brain, are increasingly being investigated and focus on the perturbed amyloid-beta precursor protein processing involving the beta- or gamma secretase enzymes.

The present invention is based on the discovery that certain known polypeptides are factors in the up-regulation and/or induction of amyloid beta precursor processing in neuronal cells, and that the inhibition of the function of such polypeptides are effective in reducing levels of amyloid beta peptides.

### SUMMARY OF THE INVENTION

The present invention relates to the relationship between the function of the G-protein coupled receptor(s) (“GPCR(s)”) and amyloid-beta precursor protein processing in mammalian cells.

One aspect of the present invention is a method for identifying a compound that inhibits the processing of amyloid-beta precursor protein in a mammalian cell, comprising
(a) contacting a compound with a GRPR, ADRA1A and TACR1 polypeptide; and
(b) measuring a compound-polypeptide property related to the production of amyloid-beta protein.

Aspects of the present method include the in vitro assay of compounds using polypeptide domains comprising an amino acid sequence selected from the group consisting of SEQ ID NO: 44, 50, 51, and 56, and cellular assays wherein GPCR inhibition is followed by observing indicators of efficacy, including second messenger levels and/or amyloid beta peptide levels.

Another aspect of the invention is a method of treatment or prevention of a condition involving cognitive impairment, or a susceptibility to the condition, in a subject suffering or susceptible thereto, by administering a pharmaceutical composition comprising an effective amyloid-beta precursor processing-inhibiting amount of a GPCR antagonist or inverse agonist.

A further aspect of the present invention is a pharmaceutical composition for use in said method wherein said inhibitor comprises a polynucleotide selected from the group of an antisense polynucleotide, a ribozyme, and a small interfering RNA (siRNA), wherein said agent comprises a nucleic acid sequence complementary to, or engineered from, a naturally occurring polynucleotide sequence encoding a polypeptide comprising an amino acid sequence selected from the group consisting of SEQ ID NO: 98-100, 122-133, 153-156, and 232-537.

Another further aspect of the present invention is a pharmaceutical composition comprising a therapeutically effective amyloid-beta precursor processing-inhibiting amount of a GPCR antagonist or inverse agonist or its pharmaceutically acceptable salt, hydrate, solvate, or prodrug thereof in admixture with a pharmaceutically acceptable carrier. The present polynucleotides and GPCR antagonist and inverse agonist compounds are also useful for the manufacturing of a medicament for the treatment of Alzheimer's disease.

### BRIEF DESCRIPTION OF THE DRAWINGS

FIG. 1A. APP processing: The membrane anchored amyloid precursor protein (APP) is processed by two pathways: the amyloidogenic and non-amyloidogenic pathway.

In the latter pathway, APP is cleaved first by alpha secretase and then by gamma secretase, yielding the p3 peptides (17-40 or 17-42). The amyloidogenic pathway generates the pathogenic amyloid beta peptides (A beta) after cleavage by beta- and gamma-secretase respectively. The numbers depicted are the positions of the amino acids comprising the A beta sequences.

FIG. 1B. Pictorial representation of transmembrane structure of GPCR proteins.

FIG. 2. Evaluation of the APP processing assay: Positive (PSIG384L; PS1L392V and BACE1) and negative (eGFP, LacZ and empty) control viruses are infected in Hek293APPwt at random MOI, mimicking a screening. A and B: Transduction is performed respectively with 1 and 0.2 μl of virus and amyloid beta 1-42 levels are performed. Data are represented as relative light units and correlate to pM of amyloid beta 1-42.

FIG. 3. Evaluation of the APP processing assay: Positive (PSIG384L; PSIL392V and BACE1) and negative (eGFP, LacZ and empty) control viruses are infected in SH-SY-5Y APPwt at random MOI, mimicking a screening. Transduction is performed with 1 μl of virus and amyloid beta 1-42 levels (A) or amyloid beta x-42 levels (B) are determined. Data are represented as relative light units and correlate to pM of amyloid beta 1-42 and x-42.

FIG. 4. Positive (PS1G384L and BACE1) and negative (eGFP, LacZ and empty) control viruses are infected in Hek293APPwt at random MOI. Transduction is performed respectively with 0.2 μl of virus and amyloid beta 1-42 levels are determined. Data are represented as single relative light units data points. The average and standard deviation of all negative controls is calculated and the cut off is determined using the AVERAGE+(3*STDEV) formula. The cut off is depicted as a line. All positive controls are clearly positioned above the cut-off.

FIG. 5A. Hek293 APPwt cells are infected with either empty adenovirus or adenovirus expressing GRPR. The cells are stimulated with known agonist, GRP, and amyloid beta 1-42 (A) and x-42 (B) levels are measured with the corresponding amyloid beta ELISA.

FIG. 5B. SH-SY5Y APPwt cells are infected with either empty adenovirus or adenovirus expressing GRPR. The cells are stimulated with known agonist, GRP, and amyloid beta 1-42 (A) and x-42 (B) levels are measured with the corresponding amyloid beta ELISA.

FIG. 6A. Hek293 APPwt cells are infected with either empty adenovirus or adenovirus expressing TACR1. The cells are stimulated with the agonist, substance P, and amyloid beta 1-42 (A) and x-42 (B) levels are measured with the corresponding amyloid beta ELISA. In panel C, the cells are infected with adenovirus expressing CAR1 and are treated with increasing amount of substance P and fixed concentrations of known antagonist, L733,060 hydrobromide. Amyloid beta x-42 levels are determined with the corresponding ELISA.

FIG. 6B. SH-SY5Y APPwt cells are infected with either empty adenovirus or adenovirus expressing TACR1. The cells are stimulated with the known agonist, substance P and amyloid beta 1-42 (A) and x-42 (B) levels are measured with the corresponding amyloid beta ELISA.

FIG. 7A. Hek293 APPwt cells are infected with either empty adenovirus or adenovirus expressing ADRA1A. The cells are stimulated with the known agonist, A61603 hydrobromide, and amyloid beta 1-42 (A) and x-42 (B) levels are measured with the corresponding amyloid beta ELISA. In panel C, the cells are treated with increasing amount of A61603 hydrobromide and fixed concentrations of known antagonist, RS17053. Amyloid beta x-42 levels are determined with the corresponding ELISA.

FIG. 7B. SH-SY5Y APPwt cells are infected with either empty adenovirus or adenovirus expressing ADRA1A. The cells are stimulated with A61603 hydrobromide and amyloid beta 1-42 (A) and x-42 (B) levels are measured with the corresponding amyloid beta ELISA. In panel C, the cells are infected with adenovirus expressing ADRA1A and are treated with increasing amount of A61603 hydrobromide and fixed concentrations of known antagonist, RS17053. Amyloid beta x-42 levels are determined with the corresponding ELISA.

FIG. 8. SH-SY5Y APPwt cells are infected with the indicated adenoviral knock down constructs with increasing MOI. Amyloid beta 1-42 levels are determined with the corresponding ELISA. Resulting amyloid beta 1-42 levels are normalized for cell number based upon ATP levels.

### DETAILED DESCRIPTION

The following terms are intended to have the meanings presented therewith below and are useful in understanding the description of and intended scope of the present invention.

Definitions:

The term “agonist” refers to a ligand that activates the intracellular response of the receptor to which the agonist binds.

The term “amyloid beta peptide” means amyloid beta peptides processed from the amyloid beta precursor protein (APP). The most common peptides include amyloid beta peptides 1-40, 1-42, 11-40 and 11-42. Other less prevalent amyloid beta peptide species are described as x-42, whereby x ranges from 2-10 and 12-17, and 1-y whereby y ranges from 24-39 and 41. For descriptive and technical purposes hereinbelow, “x” has a value of 2-17, and “y” has a value of 24 to 41.

The term “antagonist” means a moiety that bind competitively to the receptor at the same site as the agonists but which do not activate the intracellular response initiated by the active form of the receptor, and can thereby inhibit the intracellular responses by agonists. Antagonists do not diminish the baseline intracellular response in the absence of an agonist or partial agonist.

The term “carrier” means a non-toxic material used in the formulation of pharmaceutical compositions to provide a medium, bulk and/or useable form to a pharmaceutical composition. A carrier may comprise one or more of such materials such as an excipient, stabilizer, or an aqueous pH buffered solution. Examples of physiologically acceptable carriers include aqueous or solid buffer ingredients including phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as TWEEN™, polyethylene glycol (PEG), and PLURONICS™.

The term “compound” is used herein in the context of a “test compound” or a “drug candidate compound” described in connection with the assays of the present invention. As such, these compounds comprise organic or inorganic compounds, derived synthetically or from natural sources. The compounds include inorganic or organic compounds such as polynucleotides, lipids or hormone analogs that are characterized by relatively low molecular weights. Other biopolymeric organic test compounds include peptides comprising from about 2 to about 40 amino acids and larger polypeptides comprising from about 40 to about 500 amino acids, such as antibodies or antibody conjugates.

The term “constitutive receptor activation” means stabilization of a receptor in the active state by means other than binding of the receptor with its endogenous ligand or a chemical equivalent thereof.

The term “contact” or “contacting” means bringing at least two moieties together, whether in an in vitro system or an in vivo system.

The term “condition” or “disease” means the overt presentation of symptoms (i.e., illness) or the manifestation of abnormal clinical indicators (e.g., biochemical indicators), resulting from defects in one amyloid beta protein precursor processing. Alternatively, the term “disease” refers to a genetic or environmental risk of or propensity for developing such symptoms or abnormal clinical indicators.

The term “endogenous” shall mean a material that a mammal naturally produces. Endogenous in reference to, for example and not limitation, the term “receptor” shall mean that which is naturally produced by a mammal (for example, and not limitation, a human) or a virus. In contrast, the term non-endogenous in this context shall mean that which is not naturally produced by a mammal (for example, and not limitation, a human) or a virus. For example, and not limitation, a receptor which is not constitutively active in its endogenous form, but when manipulated becomes constitutively active, is most preferably referred to herein as a “non-endogenous, constitutively activated receptor.” Both terms can be utilized to describe both “in vivo” and “in vitro” systems. For example, and not a limitation, in a screening approach, the endogenous or non-endogenous receptor may be in reference to an in vitro screening system. As a further example and not limitation, where the genome of a mammal has been manipulated to include a non-endogenous constitutively activated receptor, screening of a candidate compound by means of an in vivo system is viable.

The term “expression” comprises both endogenous expression and overexpression by transduction.

The term “expressible nucleic acid” means a nucleic acid coding for a proteinaceous molecule, an RNA molecule, or a DNA molecule.

The term “hybridization” means any process by which a strand of nucleic acid binds with a complementary strand through base pairing. The term “hybridization complex” refers to a complex formed between two nucleic acid sequences by virtue of the formation of hydrogen bonds between complementary bases. A hybridization complex may be formed in solution (e.g., C₀ₜ or R₀ₜ analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed). The term “stringent conditions” refers to conditions that permit hybridization between polynucleotides and the claimed polynucleotides. Stringent conditions can be defined by salt concentration, the concentration of organic solvent, e.g., formamide, temperature, and other conditions well known in the art. In particular, reducing the concentration of salt, increasing the concentration of formamide, or raising the hybridization temperature can increase stringency.

The term “inhibit” or “inhibiting”, in relationship to the term “response” means that a response is decreased or prevented in the presence of a compound as opposed to in the absence of the compound.

The term “inverse agonist” mean a moiety that binds the endogenous form of the receptor, and which inhibits the baseline intracellular response initiated by the active endogenous form of the receptor below the normal base level of activity that is observed in the absence of the endogenous ligand, or agonists, or decrease GTP binding to membranes. Preferably, the baseline intracellular response is decreased in the presence of the inverse agonist by at least 30%, more preferably by at least 50%, and most preferably by at least 75%, as compared with the baseline response in the absence of the inverse agonist.

The term “ligand” means an endogenous, naturally occurring molecule specific for an endogenous, naturally occurring receptor.

The term “pharmaceutically acceptable prodrugs” as used herein means the prodrugs of the compounds useful in the present invention, which are, within the scope of sound medical judgment, suitable for use in contact with the tissues of patients with undue toxicity, irritation, allergic response commensurate with a reasonable benefit/risk ratio, and effective for their intended use of the compounds of the invention. The term “prodrug” means a compound that is transformed in vivo to yield an effective compound useful in the present invention or a pharmaceutically acceptable salt, hydrate or solvate thereof. The transformation may occur by various mechanisms, such as through hydrolysis in blood.

The compounds bearing metabolically cleavable groups have the advantage that they may exhibit improved bioavailability as a result of enhanced solubility and/or rate of absorption conferred upon the parent compound by virtue of the presence of the metabolically cleavable group, thus, such compounds act as pro-drugs. A thorough discussion is provided in Design of Prodrugs, H. Bundgaard, ed., Elsevier (1985); Methods in Enzymology; K. Widder et al, Ed., Academic Press, 42, 309-396 (1985); A Textbook of Drug Design and Development, Krogsgaard-Larsen and H. Bandaged, ed., Chapter 5; “Design and Applications of Prodrugs” 113-191 (1991); Advanced Drug Delivery Reviews, H. Bundgard, 8, A-C8, (1992); J. Pharm. Sci., 77, 285 (1988); Chem. Pharm. Bull., N. Nakeya et al, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, which are incorporated herein by reference. An example of the prodrugs is an ester prodrug. “Ester prodrug” means a compound that is convertible in vivo by metabolic means (e.g., by hydrolysis) to an inhibitor compound according to the present invention. For example an ester prodrug of a compound containing a carboxy group may be convertible by hydrolysis in vivo to the corresponding carboxy group.

The term “pharmaceutically acceptable salts” refers to the non-toxic, inorganic and organic acid addition salts, and base addition salts, of compounds of the present invention. These salts can be prepared in situ during the final isolation and purification of compounds useful in the present invention.

The term “polynucleotide” means a polynucleic acid, in single or double stranded form, and in the sense or antisense orientation, complementary polynucleic acids that hybridize to a particular polynucleic acid under stringent conditions, and polynucleotides that are homologous in at least about 60 percent of its base pairs, and more preferably 70 percent of its base pairs are in common, most preferably 90 percent, and in a special embodiment 100 percent of its base pairs. The polynucleotides include polyribonucleic acids, polydeoxyribonucleic acids, and synthetic analogues thereof. The polynucleotides are described by sequences that vary in length, that range from about 10 to about 5000 bases, preferably about 100 to about 4000 bases, more preferably about 250 to about 2500 bases. A preferred polynucleotide embodiment comprises from about 10 to about 30 bases in length. A special embodiment of polynucleotide is the polyribonucleotide of from about 10 to about 22 nucleotides, more commonly described as small interfering RNAs (siRNAs). Another special embodiment are nucleic acids with modified backbones such as peptide nucleic acid (PNA), polysiloxane, and 2′-O-(2-methoxy)ethylphosphorothioate, or including non-naturally occurring nucleic acid residues, or one or more nucleic acid substituents, such as methyl-, thio-, sulphate, benzoyl-, phenyl-, amino-, propyl-, chloro-, and methanocarbanucleosides, or a reporter molecule to facilitate its detection.

The term “polypeptide” relates to proteins, proteinaceous molecules, fractions of proteins (such as kinases, proteases, GPCRs), peptides and oligopeptides.

The term “solvate” means a physical association of a compound useful in this invention with one or more solvent molecules. This physical association includes hydrogen bonding. In certain instances the solvate is capable of isolation, for example when one or more solvent molecules are incorporated in the crystal lattice of the crystalline solid. “Solvate” encompasses both solution-phase and isolable solvates. Representative solvates include hydrates, ethanolates and methanolates.

The term “subject” includes humans and other mammals.

The term “effective amount” or “therapeutically effective amount” means that amount of a compound or agent that will elicit the biological or medical response of a subject that is being sought by a medical doctor or other clinician. In particular, with regard to treating an neuronal disorder, the term “effective amount” is intended to mean that effective amyloid-beta precursor processing inhibiting amount of an compound or agent that will bring about a biologically meaningful decrease in the levels of amyloid beta peptide in the subject's brain tissue.

The term “treating” means an intervention performed with the intention of preventing the development or altering the pathology of, and thereby alleviating a disorder, disease or condition, including one or more symptoms of such disorder or condition. Accordingly, “treating” refers to both therapeutic treatment and prophylactic or preventative measures. Those in need of treating include those already with the disorder as well as those in which the disorder is to be prevented. The related term “treatment,” as used herein, refers to the act of treating a disorder, symptom, disease or condition, as the term “treating” is defined above.

The background of the present inventors' discovery is described briefly below.

Background of the G-Protein Couple Receptors

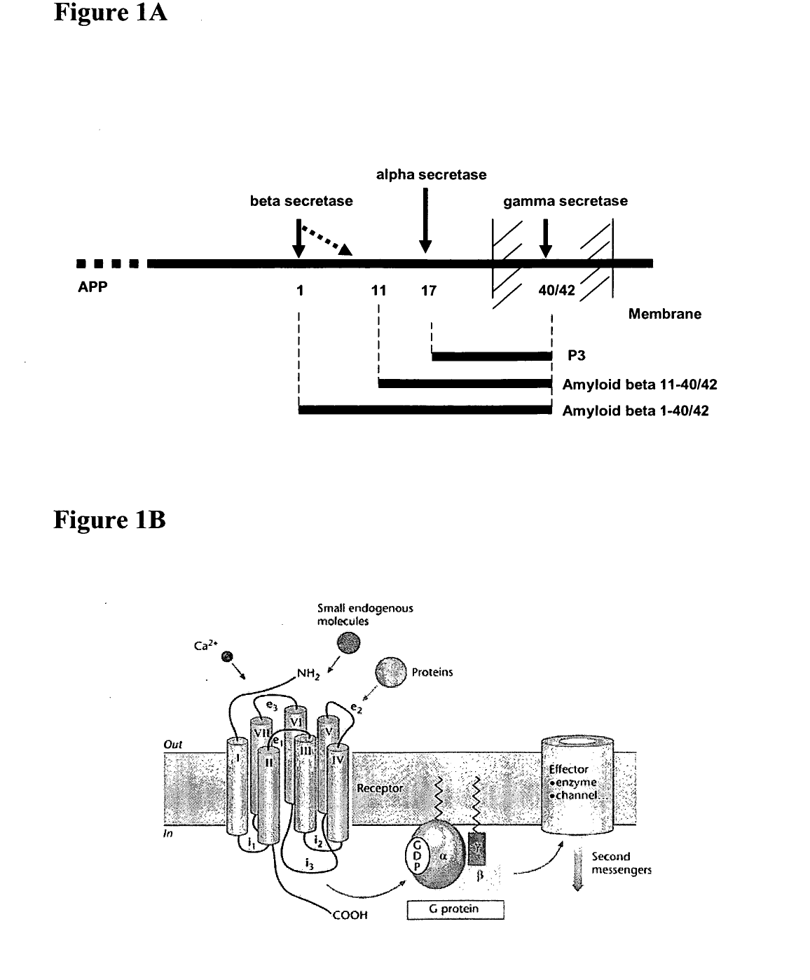

G protein-coupled receptors (GPCR) share a common structural motif. All these receptors have seven sequences of between 22 to 24 hydrophobic amino acids that form seven alpha helices, each of which spans the membrane forming 7 transmembrane domains, an extracellular N-terminus and an intracellular C-terminus. The transmembrane helices are joined by strands of amino acids having a larger loop between the fourth and fifth transmembrane helix on the extracellular side of the membrane. Another larger loop, composed primarily of hydrophilic amino acids, joins transmembrane helices five and six on the intracellular side of the membrane. See FIG. 1B.

Under physiological conditions, GPCRs exist in the cell membrane in equilibrium between two different states or conformations: an “inactive” state and an “active” state. A receptor in an inactive state is unable to link to the intracellular transduction pathway to produce a biological response. Changing the receptor conformation to the active state allows linkage to the transduction pathway and produces a biological response. A receptor may be stabilized in an active state by an endogenous ligand or an exogenous agonist ligand. Recent discoveries, including but not exclusively limited to, modifications to the amino acid sequence of the receptor, provide alternative mechanisms other than ligands to stabilize the active state conformation. These approaches effectively stabilize the receptor in an active state by simulating the effect of a ligand binding to the receptor. Stabilization by such ligand-independent approaches is termed “constitutive receptor activation.”

The major signal transduction cascades activated by GPCRs are initiated by the activation of heterotrimeric G-proteins, built from three different proteins; the G_{α}, G_{β} and G_{γ} subunits. It is believed that the loop joining helices five and six, as well as the carboxy terminus, interact with the G protein.

The signal transduction cascade starts with the activation of the receptor by an agonist. Transformational changes in the receptor are then translated down to the G-protein. The G-protein dissociates into the G_{α} subunit and the G_{βγ} subunit. Both subunits dissociate from the receptor and are both capable of initiating different cellular responses. Best known are the cellular effects that are initiated by the G_{α} subunit. It is for this reason that G-proteins are categorized by their G_{α} subunit. The G-proteins are divided into four groups: Gₛ, G_{i/o}, G_{q} and G_{12/13}. Each of these G-proteins is capable of activating an effector protein, which results in changes in second messenger levels in the cell. The changes in second messenger level are the triggers that make the cell respond to the extracellular signal in a specific manner. The activity of a GPCR can be measured by measuring the activity level of the second messenger.

The two most important second messengers in the cell are cAMP and Ca²⁺. The α-subunit of the Gₛ class of G-proteins is able to activate adenylyl cyclase, resulting in an increased turnover from ATP to cAMP. The α-subunit of G_{i/o} G-proteins does exactly the opposite and inhibits adenylyl cyclase activity resulting in a decrease of cellular cAMP levels. Together, these two classes of G-proteins regulate the second messenger cAMP. Ca²⁺ is regulated by the a-subunit of the G_{q} class of G-proteins. Through the activation of phospholipase C phosphatidylinositol 4,5-bisphosphate (PIP2) from the cell membrane are hydrolyzed to inositol 1,4,5-trisphosphate and 1,2-diacylglycerol, both these molecules act as second messengers. Inositol 1,4,5-trisphosphate binds specific receptors in the endoplasmatic reticulum, resulting in the opening of Ca²⁺ channels and release of Ca²⁺ in the cytoplasm.

References: Annaert, W. and B. De Strooper (2002). “A cell biological perspective on Alzheimer's disease.” Annu Rev Cell Dev Biol 18: 25-51.

Gotz, J., F. Chen, et al. (2001). “Formation of neurofibrillary tangles in P3011 tau transgenic mice induced by Abeta 42 fibrils.” Science 293(5534): 1491-5.
Lipinski, C. A., Lombardo, F., Dominy, B. W., and Feeney, P. J. Adv. Drug. Deliv. Rev., 23, 3-25, 1997
Marchese, A.; Docherty, J M.; Nguyen, T.; Heiber, M.; Cheng, R.; Heng, H H.; Tsui, L C.; Shi, X.; George S R. and O'Dowd, B F. (1994). Cloning of human genes encoding novel G protein-coupled receptors. Genomics, 23, 3: 609-618.
Marinissen, M. J. and J. S. Gutkind (2001). “G-protein-coupled receptors and signaling networks: emerging paradigms.” Trends Pharmacol Sci 22(7): 368-76.
Ritchie, K. and S. Lovestone (2002). “The dementias.” Lancet 360(9347): 1759-66.
Wess, J. (1998). “Molecular basis of receptor/G-protein-coupling selectivity.” Pharmacol Ther 80(3): 231-64.
   Applicants' Invention Based on GPCR Relationship to Amyloid Beta Peptides

As noted above, the present invention is based on the present inventors' discovery that the G-protein coupled receptor(s) (“GPCR(s)”) are factors in the up-regulation and/or induction of amyloid beta precursor processing in mammalian, and principally, neuronal cells, and that the inhibition of the function of such polypeptides is effective in reducing levels of amyloid beta protein peptides.

The present inventors are unaware of any prior knowledge linking GPCRs, and more particularly GRPR, ADRA1A and TACR1, and amyloid beta peptide formation and secretion. As discussed in more detail in the Experimental section below, the present inventors demonstrate that the increased expression of GRPR, ADRA1A and TACR1 increases, and the knockdown of GRPR, ADRA1A and TACR1 reduces, amyloid beta 1-42 in the conditioned medium of transduced cells. The present invention is based on these findings and the recognition that these GPCRs are putative drug targets for Alzheimer's disease. This is particularly the case for TACR1 since this protein is known to be present in the tissue of the central nervous system.

One aspect of the present invention is a method based on the aforesaid discovery for identifying a compound that inhibits the processing of amyloid-beta precursor protein in a mammalian cell, and may therefore be useful in reducing amyloid beta peptide levels in a subject. The present method comprises contacting a drug candidate compound with a GPCR polypeptide, or a fragment of said polypeptide, and measuring a compound-polypeptide property related to the production of amyloid-beta protein. The “compound-polypeptide property” is a measurable phenomenon chosen by the person of ordinary skill in the art, and based on the recognition that GPCR activation and deactivation is a causative factor in the activation and deactivation, respectively, of amyloid beta protein precursor processing, and an increase and decrease, respectively, of amyloid beta peptide levels. The measurable property may range from the binding affinity for a peptide domain of the GPCR polypeptide, to the level of any one of a number of “second messenger” levels resulting from the activation or deactivation of the GPCR, to a reporter molecule property directly linked to the aforesaid second messenger, and finally to the level of amyloid beta peptide secreted by the mammalian cell contacted with the compound.

Depending on the choice of the skilled artisan, the present assay method may be designed to function as a series of measurements, each of which is designed to determine whether the drug candidate compound is indeed acting on the GPCR to amyloid beta peptide pathway. For example, an assay designed to determine the binding affinity of a compound to the GPCR, or fragment thereof, may be necessary, but not sufficient, to ascertain whether the test compound would be useful for reducing amyloid beta peptide levels when administered to a subject. Nonetheless, such binding information would be useful in identifying a set of test compounds for use in an assay that would measure a different property, further down the biochemical pathway. Such second assay may be designed to confirm that the test compound, having binding affinity for a GPCR peptide, actually down-regulates or inhibits, as an agonist or inverse agonist, GPCR function in a mammalian cell. This further assay may measure a second messenger that is a direct consequence of the activation or deactivation of the GPCR, or a synthetic reporter system responding to the messenger. Measuring a different second messenger, and/or confirming that the assay system itself is not being affected directly and not the GPCR pathway may further validate the assay. In this latter regard, suitable controls should always be in place to insure against false positive readings.

The order of taking these measurements is not believed to be critical to the practice of the present invention, which may be practiced in any order. For example, one may first perform a screening assay of a set of compounds for which no information is known respecting the compounds' binding affinity for GPCR. Alternatively, one may screen a set of compounds identified as having binding affinity for a GPCR peptide domain, or a class of compounds identified as being agonist or inverse agonists of a GPCR. It is not essential to know the binding affinity for GPCR due to the possible compound interaction in the intra-membrane domain of the GPCR polypeptide, which domain conformation may not be possible to reproduce in an affinity experiment. However, for the present assay to be meaningful to the ultimate use of the drug candidate compounds, a measurement of the second messenger(s), or the ultimate amyloid beta peptide levels, is necessary. Validation studies including controls, and measurements of binding affinity to GPCR are nonetheless useful in identifying a compound useful in any therapeutic or diagnostic application.

The present assay method may be practiced in vitro, using one or more of the GPCR proteins, or fragments thereof, or membrane preparations made from cells transduced with vectors over-expressing the GPCR polypeptides. The amino acid sequences of the GPCRs, and useful fragments thereof are found in SEQ ID NO: 44, 50, 51 and 56, and 538-582. The binding affinity of the compound with the polypeptide can be measured by methods known in the art, such as using surface plasmon resonance biosensors (Biacore), by saturation binding analysis with a labeled compound (e.g. Scatchard and Lindmo analysis), by differential UV spectrophotometer, fluorescence polarization assay, Fluorometric Imaging Plate Reader (FLIPR®) system, Fluorescence resonance energy transfer, and Bioluminescence resonance energy transfer. The binding affinity of compounds can also be expressed in dissociation constant (Kd) or as IC50 or EC50. The IC50 represents the concentration of a compound that is required for 50% inhibition of binding of another ligand to the polypeptide. The EC50 represents the concentration required for obtaining 50% of the maximum effect in any assay that measures receptor function. The dissociation constant, Kd, is a measure of how well a ligand binds to the polypeptide, it is equivalent to the ligand concentration required to saturate exactly half of the binding-sites on the polypeptide. Compounds with a high affinity binding have low Kd, IC50 and EC50 values, i.e. in the range of 100 nM to 1 pM; a moderate to low affinity binding relates to a high Kd, IC50 and EC50 values, i.e. in the micromolar range.

The present assay method may also be practiced in a cellular assay, A host cell expressing a GPCR polypeptide can be a cell with endogenous expression of the polypeptide or a cell over-expressing the polypeptide e.g. by transduction. When the endogenous expression of the polypeptide is not sufficient to determine a baseline that can easily be measured, one may use using host cells that over express GPCR. Overexpression has the advantage that the level of the second messenger is higher than the activity level by endogenous expression. Accordingly, measuring such levels using presently available techniques is easier. In such cellular assay, the biological activity of the GPCR may be measured using a second messenger, such as cyclic AMP or Ca²⁺, cyclic GMP, inositol triphosphate (IP₃) and/or diacylglycerol (DAG). Cyclic AMP or Ca²⁺ are preferred second messengers to measure. Second messenger activation may be measured by several different techniques, either directly by ELISA or radioactive technologies or indirectly by reporter gene analysis, discussed below. Preferably the method further comprises contacting the host cell with an agonist for GPCR before determining the baseline level. The addition of an agonist further stimulates GPCR, thereby further increasing the activity level of the second messenger. Several such agonists (ligands) are known in the art; preferentially the agonist is GRP, Substance P or A61603. The GPCR polypeptides, when over expressed or activated, modulate the level of secreted amyloid beta peptides.

The present invention further relates to a method for identifying a compound that inhibits amyloid-beta precursor protein processing in a mammalian cell comprising:
(a) contacting a compound with a polypeptide comprising an amino acid sequence selected from the group consisting of SEQ ID NO: 44, 50, 51 and 56,
(b) determining the binding affinity of the compound to the polypeptide,
(c) contacting a population of mammalian cells expressing said polypeptide with the compound that exhibits a binding affinity of at least 10 micromolar, and
(d) identifying the compound that inhibits the amyloid-beta precursor protein processing in the cells.

A further embodiment of the present invention relates a method to identify a compound that inhibits the amyloid-beta precursor protein processing in a cell, wherein the activity level of the GPCR polypeptide is measured by determining the level of one or more second messengers, wherein the level of the one or second messenger is determined with a reporter controlled by a promoter, which is responsive to the second messenger. The reporter is a reporter gene under the regulation of a promoter that responds to the cellular level of second messengers. Such preferred second messengers are Cyclic AMP or Ca²⁺. The reporter gene should have a gene product that is easily detected, and that may be stably infected in the host cell. Such methods are well known by any person with ordinary skill in the art.

The reporter gene may be selected from alkaline phosphatase, green fluorescent protein (GFP), enhanced green fluorescent protein (eGFP), destabilized green fluorescent protein (dGFP), luciferase, and beta-galactosidase among others. The reporter is preferably luciferase or beta-galactosidase, which are readily available and easy to measure over a large range The promoter in the reporter construct is preferably a cyclic AMP-responsive promoter, an NF-KB responsive promoter, or a NF-AT responsive promoter. The cyclic-AMP responsive promoter is responsive to the cyclic-AMP levels in the cell. The NF-AT responsive promoter is sensitive to cytoplasmic Ca²⁺-levels in the cell. The NF-KB responsive promoter is sensitive for activated NF-KB levels in the cell.

A further embodiment of the present invention relates a method to identify a compound that inhibits the amyloid-beta precursor protein processing in a cell, wherein the activity level of the GPCR polypeptide is measured by determining the level of amyloid beta peptides. The levels of these peptides may be measured with specific ELISAs using antibodies specifically recognizing the different amyloid beta peptide species (see e.g. EXAMPLE 1). Secretion of the various amyloid beta peptides may also be measured using antibodies that bind all peptides. Levels of amyloid beta peptides can also be measured by Mass spectrometry analysis.

For high-throughput purposes, libraries of compounds may be used such as antibody fragment libraries, peptide phage display libraries, peptide libraries (e.g. LOPAP™, Sigma Aldrich), lipid libraries (BioMol), synthetic compound libraries (e.g. LOPAC™, Sigma Aldrich) or natural compound libraries (Specs, TimTec).

Preferred drug candidate compounds are low molecular weight compounds. Low molecular weight compounds, i.e. with a molecular weight of 500 Dalton or less, are likely to have good absorption and permeation in biological systems and are consequently more likely to be successful drug candidates than compounds with a molecular weight above 500 Dalton (Lipinski et al. (1997)). Peptides comprise another preferred class of drug candidate compounds, since peptides are known GPCRs antagonists. Peptides may be excellent drug candidates and there are multiple examples of commercially valuable peptides such as fertility hormones and platelet aggregation inhibitors. Natural compounds are another preferred class of drug candidate compound. Such compounds are found in and extracted from natural sources, and which may thereafter be synthesized.

Another preferred class of drug candidate compounds is an antibody. The present invention also provides antibodies directed against the extracellular domains of the GPCR. These antibodies should specifically bind to one or more of the extra-cellular domains of the GPCRs, or as described further below, engineered to be endogenously produced to bind to the intra-cellular GPCR domain. These antibodies may be monoclonal antibodies or polyclonal antibodies. The present invention includes chimeric, single chain, and humanized antibodies, as well as FAb fragments and the products of a FAb expression library, and Fv fragments and the products of an Fv expression library.

In certain embodiments, polyclonal antibodies may be used in the practice of the invention. The skilled artisan knows methods of preparing polyclonal antibodies. Polyclonal antibodies can be raised in a mammal, for example, by one or more injections of an immunizing agent and, if desired, an adjuvant. Typically, the immunizing agent and/or adjuvant are injected in the mammal by multiple subcutaneous or intraperitoneal injections. Antibodies may also be generated against the intact GPCR protein or polypeptide, or against a fragment such as its extracellular domain peptides, derivatives including conjugates, or other epitope of the GPCR protein or polypeptide, such as the GPCR embedded in a cellular membrane, or a library of antibody variable regions, such as a phage display library.

It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate). One skilled in the art without undue experimentation may select the immunization protocol.

In some embodiments, the antibodies may be monoclonal antibodies. Monoclonal antibodies may be prepared using methods known in the art. The monoclonal antibodies of the present invention may be “humanized” to prevent the host from mounting an immune response to the antibodies. A “humanized antibody” is one in which the complementarity determining regions (CDRs) and/or other portions of the light and/or heavy variable domain framework are derived from a non-human immunoglobulin, but the remaining portions of the molecule are derived from one or more human immunoglobulins. Humanized antibodies also include antibodies characterized by a humanized heavy chain associated with a donor or acceptor unmodified light chain or a chimeric light chain, or vice versa. The humanization of antibodies may be accomplished by methods known in the art (see, e.g. Mark and Padlan, (1994) “Chapter 4. Humanization of Monoclonal Antibodies”, The Handbook of Experimental Pharmacology Vol. 113, Springer-Verlag, New York). Transgenic animals may be used to express humanized antibodies.

Human antibodies can also be produced using various techniques known in the art, including phage display libraries (Hoogenboom and Winter, (1991) J. Mol. Biol. 227:381-8; Marks et al. (1991). J. Mol. Biol. 222:581-97). The techniques of Cole, et al. and Boerner, et al. are also available for the preparation of human monoclonal antibodies (Cole, et al. (1985) Monoclonal Antibodies and Cancer Therapy, Alan R. Liss, p. 77; Boerner, et al (1991). J. Immunol., 147(1):86-95).

Techniques known in the art for the production of single chain antibodies can be adapted to produce single chain antibodies to the GPCR polypeptides and proteins of the present invention. The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain cross-linking. Alternatively; the relevant cysteine residues are substituted with another amino acid residue or are deleted so as to prevent cross-linking.

Bispecific antibodies are monoclonal, preferably human or humanized, antibodies that have binding specificities for at least two different antigens and preferably for a cell-surface protein or receptor or receptor subunit. In the present case, one of the binding specificities is for one extracellular domain of the GPCR, the other one is for another extracellular domain of the same or different GPCR.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, (1983) Nature 305:537-9). Because of the random assortment of immunoglobulin heavy and light chains, these hybridomas (quadromas) produce a potential mixture of ten different antibody molecules, of which only one has the correct bispecific structure. Affinity chromatography steps usually accomplish the purification of the correct molecule. Similar procedures are disclosed in Trauneeker, et al. (1991) EMBO J. 10:3655-9.

According to another preferred embodiment, the assay method comprise using a drug candidate compound identified as having a binding affinity for GPCRs, and/or has already been identified as having down-regulating activity such as antagonist or inverse agonist activity vis-á-vis one or more GPCR. Examples of such compounds are the selective tachykinin NK1 receptor antagonists, subtype selective a1A-adrenoceptor antagonists, GRP receptor antagonists, identified in Table 8 below.

Another aspect of the present invention relates to a method for reducing amyloid-beta precursor protein processing in a mammalian cell, comprising by contacting said cell with an expression-inhibiting agent that inhibits the translation in the cell of a polyribonucleotide encoding a GPCR polypeptide. A particular embodiment relates to a composition comprising an polynucleotide including at least one antisense strand that functions to pair the agent with the target GPCR mRNA, and thereby down-regulate or block the expression of GPCR polypeptide. The inhibitory agent preferably comprises antisense polynucleotide, a ribozyme, and a small interfering RNA (siRNA), wherein said agent comprises a nucleic acid sequence complementary to, or engineered from, a naturally occurring polynucleotide sequence encoding a polypeptide comprising an amino acid sequence selected from the group consisting of SEQ ID NO: 44, 50, 51 and 56.

A special embodiment of the present invention relates to a method wherein the expression-inhibiting agent is selected from the group consisting of antisense RNA, antisense oligodeoxynucleotide (ODN), a ribozyme that cleaves the polyribonucleotide coding for SEQ ID NO: 44, 50, 51 and 56, a small interfering RNA (siRNA) that is sufficiently homologous to a portion of the polyribonucleotide corresponding to SEQ ID NO: 7, 13, 14 and 19 such that the siRNA interferes with the translation of the GPCR polyribonucleotide to the GPCR polypeptide.

Another embodiment of the present invention relates to a method wherein the expression-inhibiting agent is a nucleic acid expressing the antisense RNA, antisense oligodeoxynucleotide (ODN), a ribozyme that cleaves the polyribonucleotide coding for SEQ ID NO: 44, 50, 51 and 56, a small interfering RNA (siRNA) that is sufficiently homologous to a portion of the polyribonucleotide corresponding to SEQ ID NO: 7, 13, 14 and 19 such that the siRNA interferes with the translation of the GPCR polyribonucleotide to the GPCR polypeptide. Preferably the expression-inhibiting agent is an antisense RNA, ribozyme, antisense oligodeoxynucleotide, or siRNA comprising a nucleotide sequence selected from the group consisting of SEQ ID NO: 98-100, 122-133, 153-156 and 232-537.

The down regulation of gene expression using antisense nucleic acids can be achieved at the translational or transcriptional level. Antisense nucleic acids of the invention are preferably nucleic acid fragments capable of specifically hybridizing with all or part of a nucleic acid encoding a GPCR polypeptide or the corresponding messenger RNA. In addition, antisense nucleic acids may be designed which decrease expression of the nucleic acid sequence capable of encoding a GPCR polypeptide by inhibiting splicing of its primary transcript. Any length of antisense sequence is suitable for practice of the invention so long as it is capable of down-regulating or blocking expression of a nucleic acid coding for a GPCR. Preferably, the antisense sequence is at least about 17 nucleotides in length. The preparation and use of antisense nucleic acids, DNA encoding antisense RNAs and the use of oligo and genetic antisense is known in the art.

One embodiment of expression-inhibitory agent is a nucleic acid that is antisense to a nucleic acid comprising SEQ ID NO: 98-100, 122-133, 153-156 and 232-537. For example, an antisense nucleic acid (e.g. DNA) may be introduced into cells in vitro, or administered to a subject in vivo, as gene therapy to inhibit cellular expression of nucleic acids comprising SEQ ID NO: 98-100, 122-133, 153-156 and 232-537. Antisense oligonucleotides preferably comprise a sequence containing from about 17 to about 100 nucleotides and more preferably the antisense oligonucleotides comprise from about 18 to about 30 nucleotides. Antisense nucleic acids may be prepared from about 10 to about 30 contiguous nucleotides selected from the sequences of SEQ ID NO: 7, 13, 14 and 19, expressed in the opposite orientation.

The antisense nucleic acids are preferably oligonucleotides and may consist entirely of deoxyribo-nucleotides, modified deoxyribonucleotides, or some combination of both. The antisense nucleic acids can be synthetic oligonucleotides. The oligonucleotides may be chemically modified, if desired, to improve stability and/or selectivity. Since oligonucleotides are susceptible to degradation by intracellular nucleases, the modifications can include, for example, the use of a sulfur group to replace the free oxygen of the phosphodiester bond. This modification is called a phosphorothioate linkage. Phosphorothioate antisense oligonucleotides are water soluble, polyanionic, and resistant to endogenous nucleases. In addition, when a phosphorothioate antisense oligonucleotide hybridizes to its target site, the RNA-DNA duplex activates the endogenous enzyme ribonuclease (RNase) H, which cleaves the mRNA component of the hybrid molecule.

In addition, antisense oligonucleotides with phosphoramidite and polyamide (peptide) linkages can be synthesized. These molecules should be very resistant to nuclease degradation. Furthermore, chemical groups can be added to the 2′ carbon of the sugar moiety and the 5 carbon (C-5) of pyrimidines to enhance stability and facilitate the binding of the antisense oligonucleotide to its target site. Modifications may include 2′-deoxy, O-pentoxy, O-propoxy, O-methoxy, fluoro, methoxyethoxy phosphorothioates, modified bases, as well as other modifications known to those of skill in the art.

Another type of expression-inhibitory agent that reduces the levels of GPCRs is ribozymes. Ribozymes are catalytic RNA molecules (RNA enzymes) that have separate catalytic and substrate binding domains. The substrate binding sequence combines by nucleotide complementarity and, possibly, non-hydrogen bond interactions with its target sequence. The catalytic portion cleaves the target RNA at a specific site. The substrate domain of a ribozyme can be engineered to direct it to a specified mRNA sequence. The ribozyme recognizes and then binds a target mRNA through complementary base pairing. Once it is bound to the correct target site, the ribozyme acts enzymatically to cut the target mRNA. Cleavage of the mRNA by a ribozyme destroys its ability to direct synthesis of the corresponding polypeptide. Once the ribozyme has cleaved its target sequence, it is released and can repeatedly bind and cleave at other mRNAs.

Ribozyme forms include a hammerhead motif, a hairpin motif, a hepatitis delta virus, group I intron or RNaseP RNA (in association with an RNA guide sequence) motif or Neurospora VS RNA motif. Ribozymes possessing a hammerhead or hairpin structure are readily prepared since these catalytic RNA molecules can be expressed within cells from eukaryotic promoters (Chen, et al. (1992) Nucleic Acids Res. 20:4581-9). A ribozyme of the present invention can be expressed in eukaryotic cells from the appropriate DNA vector. If desired, the activity of the ribozyme may be augmented by its release from the primary transcript by a second ribozyme (Ventura, et al. (1993) Nucleic Acids Res. 21:3249-55).

Ribozymes may be chemically synthesized by combining an oligodeoxyribonucleotide with a ribozyme catalytic domain (20 nucleotides) flanked by sequences that hybridize to the target mRNA after transcription. The oligodeoxyribonucleotide is amplified by using the substrate binding sequences as primers. The amplification product is cloned into a eukaryotic expression vector.

Ribozymes are expressed from transcription units inserted into DNA, RNA, or viral vectors. Transcription of the ribozyme sequences are driven from a promoter for eukaryotic RNA polymerase I (pol (I), RNA polymerase II (pol II), or RNA polymerase III (pol III). Transcripts from pol II or pol III promoters are expressed at high levels in all cells; the levels of a given pol II promoter in a given cell type will depend on nearby gene regulatory sequences. Prokaryotic RNA polymerase promoters are also used, providing that the prokaryotic RNA polymerase enzyme is expressed in the appropriate cells (Gao and Huang, (1993) Nucleic Acids Res. 21:2867-72). It has been demonstrated that ribozymes expressed from these promoters can function in mammalian cells (Kashani-Sabet, et al. (1992) Antisense Res. Dev. 2:3-15).

A particularly preferred inhibitory agent is a small interfering RNA (siRNA). siRNAs mediate the post-transcriptional process of gene silencing by double stranded RNA (dsRNA) that is homologous in sequence to the silenced RNA. siRNA according to the present invention comprises a sense strand of 17-25 nucleotides complementary or homologous to a contiguous 17-25 nucleotide sequence selected from the group of sequences described in SEQ ID NO: 7, 13, 14 and 19 and an antisense strand of 17-23 nucleotides complementary to the sense strand. Exemplary sequences are identified as SEQ ID NOS: 232-537. The most preferred siRNA comprises sense and anti-sense strands that are 100 percent complementary to each other and the target polynucleotide sequence. Preferably the siRNA further comprises a loop region linking the sense and the antisense strand.

A self-complementing single stranded siRNA molecule polynucleotide according to the present invention comprises a sense portion and an antisense portion connected by a loop region linker. Preferably, the loop region sequence is 4-30 nucleotides long, more preferably 5-15 nucleotides long and most preferably 8 nucleotides long. In a most preferred embodiment the linker sequence is UUGCUAUA (SEQ ID NO: 231). Self-complementary single stranded siRNAs form hairpin loops and are more stable than ordinary dsRNA. In addition, they are more easily produced from vectors.

Analogous to antisense RNA, the siRNA can be modified to confirm resistance to nucleolytic degradation, or to enhance activity, or to enhance cellular distribution, or to enhance cellular uptake, such modifications may consist of modified internucleoside linkages, modified nucleic acid bases, modified sugars and/or chemical linkage the SiRNA to one or more moieties or conjugates. The nucleotide sequences are selected according to siRNA designing rules that give an improved reduction of the target sequences compared to nucleotide sequences that do not comply with these siRNA designing rules (For a discussion of these rules and examples of the preparation of siRNA, WO2004094636, published Nov. 4, 2004, and UA20030198627, are hereby incorporated by reference.

The present invention also relates to compositions, and methods using said compositions, comprising a DNA expression vector capable of expressing a polynucleotide capable of inhibiting amyloid beta protein precursor processing and described hereinabove as an expression inhibition agent.

A special aspect of these compositions and methods relates to the down-regulation or blocking of the expression of a GPCR polypeptide by the induced expression of a polynucleotide encoding an intracellular binding protein that is capable of selectively interacting with the GPCR polypeptide. An intracellular binding protein includes any protein capable of selectively interacting, or binding, with the polypeptide in the cell in which it is expressed and neutralizing the function of the polypeptide. Preferably, the intracellular binding protein is a neutralizing antibody or a fragment of a neutralizing antibody having binding affinity to an intra-cellular domain of the GPCR polypeptide of SEQ ID NO: 44, 50, 51 and 56. More preferably, the intracellular binding protein is a single chain antibody.

A special embodiment of this composition comprises the expression-inhibiting agent selected from the group consisting of antisense RNA, antisense oligodeoxynucleotide (ODN), a ribozyme that cleaves the polyribonucleotide coding for SEQ ID NO: 44, 50, 51 and 56, and a small interfering RNA (siRNA) that is sufficiently homologous to a portion of the polyribonucleotide corresponding to SEQ ID NO: 7, 13, 14 ad 19 such that the siRNA interferes with the translation of the GPCR polyribonucleotide to the GPCR polypeptide.

The polynucleotide expressing the expression-inhibiting agent or the encoding an intracellular binding protein is preferably included within a vector. The polynucleic acid is operably linked to signals enabling expression of the nucleic acid sequence and is introduced into a cell utilizing, preferably, recombinant vector constructs, which will express the antisense nucleic acid once the vector is introduced into the cell. A variety of viral-based systems are available, including adenoviral, retroviral, adeno-associated viral, lentiviral, herpes simplex viral or a sendaviral vector systems, and all may be used to introduce and express polynucleotide sequence for the expression-inhibiting agents in target cells.

Preferably, the viral vectors used in the methods of the present invention are replication defective. Such replication defective vectors will usually lack at least one region that is necessary for the replication of the virus in the infected cell. These regions can either be eliminated (in whole or in part), or be rendered non-functional by any technique known to a person skilled in the art. These techniques include the total removal, substitution, partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome, which are necessary for encapsidating, the viral particles.

In a preferred embodiment, the viral element is derived from an adenovirus. Preferably, the vehicle includes an adenoviral vector packaged into an adenoviral capsid, or a functional part, derivative, and/or analogue thereof. Adenovirus biology is also comparatively well known on the molecular level. Many tools for adenoviral vectors have been and continue to be developed, thus making an adenoviral capsid a preferred vehicle for incorporating in a library of the invention. An adenovirus is capable of infecting a wide variety of cells. However, different adenoviral serotypes have different preferences for cells. To combine and widen the target cell population that an adenoviral capsid of the invention can enter in a preferred embodiment, the vehicle includes adenoviral fiber proteins from at least two adenoviruses. Preferred adenoviral fiber protein sequences are serotype 17, 45 and 51. Techniques or construction and expression of these chimeric vectors are disclosed in US Published Patent Applications 20030180258 and 20040071660, hereby incorporated by reference.

In a preferred embodiment, the nucleic acid derived from an adenovirus includes the nucleic acid encoding an adenoviral late protein or a functional part, derivative, and/or analogue thereof. An adenoviral late protein, for instance an adenoviral fiber protein, may be favorably used to target the vehicle to a certain cell or to induce enhanced delivery of the vehicle to the cell. Preferably, the nucleic acid derived from an adenovirus encodes for essentially all adenoviral late proteins, enabling the formation of entire adenoviral capsids or functional parts, analogues, and/or derivatives thereof. Preferably, the nucleic acid derived from an adenovirus includes the nucleic acid encoding adenovirus E2A or a functional part, derivative, and/or analogue thereof. Preferably, the nucleic acid derived from an adenovirus includes the nucleic acid encoding at least one E4-region protein or a functional part, derivative, and/or analogue thereof, which facilitates, at least in part, replication of an adenoviral derived nucleic acid in a cell. The adenoviral vectors used in the examples of this application are exemplary of the vectors useful in the present method of treatment invention.

Certain embodiments of the present invention use retroviral vector systems. Retroviruses are integrating viruses that infect dividing cells, and their construction is known in the art. Retroviral vectors can be constructed from different types of retrovirus, such as, MoMuLV (“murine Moloney leukemia virus” MSV (“murine Moloney sarcoma virus”), HaSV (“Harvey sarcoma virus”); SNV (“spleen necrosis virus”); RSV (“Rous sarcoma virus”) and Friend virus. Lentiviral vector systems may also be used in the practice of the present invention. Retroviral systems and herpes virus system may be preferred vehicles for transfection of neuronal cells.

In other embodiments of the present invention, adeno-associated viruses (“AAV”) are utilized. The AAV viruses are DNA viruses of relatively small size that integrate, in a stable and site-specific manner, into the genome of the infected cells. They are able to infect a wide spectrum of cells without inducing any effects on cellular growth, morphology or differentiation, and they do not appear to be involved in human pathologies.

In the vector construction, the polynucleotide agents of the present invention may be linked to one or more regulatory regions. Selection of the appropriate regulatory region or regions is a routine matter, within the level of ordinary skill in the art. Regulatory regions include promoters, and may include enhancers, suppressors, etc.

Promoters that may be used in the expression vectors of the present invention include both constitutive promoters and regulated (inducible) promoters. The promoters may be prokaryotic or eukaryotic depending on the host. Among the prokaryotic (including bacteriophage) promoters useful for practice of this invention are lac, lacZ, T3, T7, lambda Pᵣ, P₁, and trp promoters. Among the eukaryotic (including viral) promoters useful for practice of this invention are ubiquitous promoters (e.g. HPRT, vimentin, actin, tubulin), intermediate filament promoters (e.g. desmin, neurofilaments, keratin, GFAP), therapeutic gene promoters (e.g. MDR type, CFTR, factor VIII), tissue-specific promoters (e.g. actin promoter in smooth muscle cells, or Flt and Flk promoters active in endothelial cells), including animal transcriptional control regions, which exhibit tissue specificity and have been utilized in transgenic animals: elastase I gene control region which is active in pancreatic acinar cells (Swift, et al. (1984) Cell 38:639-46; Omitz, et al. (1986) Cold Spring Harbor Symp. Quant. Biol. 50:399-409; MacDonald, (1987) Hepatology 7:425-515); insulin gene control region which is active in pancreatic beta cells (Hanahan, (1985) Nature 315:115-22), immunoglobulin gene control region which is active in lymphoid cells (Grosschedl, et al. (1984) Cell 38:647-58; Adames, et al. (1985) Nature 318:533-8; Alexander, et al. (1987) Mol. Cell. Biol. 7:1436-44), mouse mammary tumor virus control region which is active in testicular, breast, lymphoid and mast cells (Leder, et al. (1986) Cell 45:485-95), albumin gene control region which is active in liver (Pinkert, et al. (1987) Genes and Devel. 1:268-76), alpha-fetoprotein gene control region which is active in liver (Krumlauf, et al. (1985) Mol. Cell. Biol., 5:1639-48; Hammer, et al. (1987) Science 235:53-8), alpha 1-antitrypsin gene control region which is active in the liver (Kelsey, et al. (1987) Genes and Devel., 1: 161-71), beta-globin gene control region which is active in myeloid cells (Mogram, et al. (1985) Nature 315:338-40; Kollias, et al. (1986) Cell 46:89-94), myelin basic protein gene control region which is active in oligodendrocyte cells in the brain (Readhead, et al. (1987) Cell 48:703-12), myosin light chain-2 gene control region which is active in skeletal muscle (Sani, (1985) Nature 314.283-6), and gonadotropic releasing hormone gene control region which is active in the hypothalamus (Mason, et al. (1986) Science 234:1372-8).

Other promoters which may be used in the practice of the invention include promoters which are preferentially activated in dividing cells, promoters which respond to a stimulus (e.g. steroid hormone receptor, retinoic acid receptor), tetracycline-regulated transcriptional modulators, cytomegalovirus immediate-early, retroviral LTR, metallothionein, SV-40, E1 a, and MLP promoters.

The vectors may also include other elements, such as enhancers, repressor systems, and localization signals. A membrane localization signal is a preferred element when expressing a sequence encoding an intracellular binding protein, which functions by contacting the intracellular domain of the GPCR and is most effective when the vector product is directed to the inner surface of the cellular membrane, where its target resides. Membrane localization signals are well known to persons skilled in the art. For example, a membrane localization domain suitable for localizing a polypeptide to the plasma membrane is the C-terminal sequence CaaX for farnesylation (where “a” is an aliphatic amino acid residue, and “X” is any amino acid residue, generally leucine), for example, Cysteine-Alanine-Alanine-Leucine, or Cysteine-Isoleucine-Valine-Methionine. Other membrane localization signals include the putative membrane localization sequence from the C-terminus of Bcl-2 or the C-terminus of other members of the Bcl-2 family of proteins.

Additional vector systems include the non-viral systems that facilitate introduction of polynucleotide agents into a patient. For example, a DNA vector encoding a desired sequence can be introduced in vivo by lipofection. Synthetic cationic lipids designed to limit the difficulties encountered with liposome-mediated transfection can be used to prepare liposomes for in vivo transfection of a gene encoding a marker (Feigner, et. al. (1987) Proc. Natl. Acad. Sci. USA 84:7413-7); see Mackey, et al. (1988) Proc. Natl. Acad. Sci. USA 85:8027-31; Ulmer, et al. (1993) Science 259:1745-8). The use of cationic lipids may promote encapsulation of negatively charged nucleic acids, and also promote fusion with negatively charged cell membranes (Felgner and Ringold, (1989) Nature 337:387-8). Particularly useful lipid compounds and compositions for transfer of nucleic acids are described in International Patent Publications WO 95/18863 and WO 96/17823, and in U.S. Pat. No. 5,459,127. The use of lipofection to introduce exogenous genes into the specific organs in vivo has certain practical advantages and directing transfection to particular cell types would be particularly advantageous in a tissue with cellular heterogeneity, for example, pancreas, liver, kidney, and the brain. Lipids may be chemically coupled to other molecules for the purpose of targeting. Targeted peptides, e.g., hormones or neurotransmitters, and proteins for example, antibodies, or non-peptide molecules could be coupled to liposomes chemically. Other molecules are also useful for facilitating transfection of a nucleic acid in vivo, for example, a cationic oligopeptide (e.g., International Patent Publication WO 95/21931), peptides derived from DNA binding proteins (e.g., International Patent Publication WO 96/25508), or a cationic polymer (e.g., International Patent Publication WO 95/21931).

It is also possible to introduce a DNA vector in vivo as a naked DNA plasmid (see U.S. Pat. Nos. 5,693,622, 5,589,466 and 5,580,859). Naked DNA vectors for therapeutic purposes can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wilson, et al. (1992) J. Biol. Chem. 267:963-7; Wu and Wu, (1988) J. Biol. Chem. 263:14621-4; Hartmut, et al. Canadian Patent Application No. 2,012,311, filed Mar. 15, 1990; Williams, et al (1991). Proc. Natl. Acad. Sci. USA 88:2726-30). Receptor-mediated DNA delivery approaches can also be used (Curiel, et al. (1992) Hum. Gene Ther. 3:147-54; Wu and Wu, (1987) J. Biol. Chem. 262:4429-32).

The present invention also provides biologically compatible compositions comprising the compounds identified as antagonists and/or inverse agonists of GPCR, and the expression-inhibiting agents as described hereinabove.

A biologically compatible composition is a composition, that may be solid, liquid, gel, or other form, in which the compound, polynucleotide, vector, and antibody of the invention is maintained in an active form, e.g., in a form able to effect a biological activity. For example, a compound of the invention would have inverse agonist or antagonist activity on the GPCR; a nucleic acid would be able to replicate, translate a message, or hybridize to a complementary mRNA of a GPCR; a vector would be able to transfect a target cell and expression the antisense, antibody, ribozyme or siRNA as described hereinabove; an antibody would bind a GPCR polypeptide domain.

A preferred biologically compatible composition is an aqueous solution that is buffered using, e.g., Tris, phosphate, or HEPES buffer, containing salt ions. Usually the concentration of salt ions is similar to physiological levels. Biologically compatible solutions may include stabilizing agents and preservatives. In a more preferred embodiment, the biocompatible composition is a pharmaceutically acceptable composition. Such compositions can be formulated for administration by topical, oral, parenteral, intranasal, subcutaneous, and intraocular, routes. Parenteral administration is meant to include intravenous injection, intramuscular injection, and intraarterial injection or infusion techniques. The composition may be administered parenterally in dosage unit formulations containing standard, well-known non-toxic physiologically acceptable carriers, adjuvants and vehicles as desired.

A particularly preferred embodiment of the present composition invention is a cognitive-enhancing pharmaceutical composition comprising a therapeutically effective amount of an expression-inhibiting agent as described hereinabove, in admixture with a pharmaceutically acceptable carrier. Another preferred embodiment is a pharmaceutical composition for the treatment or prevention of a condition involving cognitive impairment or a susceptibility to the condition, comprising an effective amyloid beta peptide inhibiting amount of a GPCR antagonist or inverse agonist its pharmaceutically acceptable salts, hydrates, solvates, or prodrugs thereof in admixture with a pharmaceutically acceptable carrier. A particularly preferred class of such compositions comprises the selective tachykinin NK1 receptor antagonists, subtype selective a1A-adrenoceptor antagonists, and GRP receptor antagonist compounds identified in Table 8 below.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical compositions for oral use can be prepared by combining active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethyl-cellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate. Dragee cores may be used in conjunction with suitable coatings, such as concentrated sugar solutions, which may also contain gum arabic, talc, polyvinyl-pyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Preferred sterile injectable preparations can be a solution or suspension in a non-toxic parenterally acceptable solvent or diluent. Examples of pharmaceutically acceptable carriers are saline, buffered saline, isotonic saline (e.g. monosodium or disodium phosphate, sodium, potassium; calcium or magnesium chloride, or mixtures of such salts), Ringer's solution, dextrose, water, sterile water, glycerol, ethanol, and combinations thereof 1,3-butanediol and sterile fixed oils are conveniently employed as solvents or suspending media. Any bland fixed oil can be employed including synthetic mono- or di-glycerides. Fatty acids such as oleic acid also find use in the preparation of injectables.

The composition medium can also be a hydrogel, which is prepared from any biocompatible or non-cytotoxic homo- or hetero-polymer, such as a hydrophilic polyacrylic acid polymer that can act as a drug absorbing sponge. Certain of them, such as, in particular, those obtained from ethylene and/or propylene oxide are commercially available. A hydrogel can be deposited directly onto the surface of the tissue to be treated, for example during surgical intervention.

Embodiments of pharmaceutical compositions of the present invention comprise a replication defective recombinant viral vector encoding the polynucleotide inhibitory agent of the present invention and a transfection enhancer, such as poloxamer. An example of a poloxamer is Poloxamer 407, which is commercially available (BASF, Parsippany, N.J.) and is a non-toxic, biocompatible polyol. A poloxamer impregnated with recombinant viruses may be deposited directly on the surface of the tissue to be treated, for example during a surgical intervention. Poloxamer possesses essentially the same advantages as hydrogel while having a lower viscosity.

The active expression-inhibiting agents may also be entrapped in microcapsules prepared, for example, by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1980) 16th edition, Osol, A. Ed.

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™. (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The present invention also provides methods of inhibiting the processing of amyloid-beta precursor protein in a subject suffering or susceptible to the abnormal processing of said protein, which comprise the administration to said subject a therapeutically effective amount of an expression-inhibiting agent of the invention. Another aspect of the present method invention is the treatment or prevention of a condition involving cognitive impairment or a susceptibility to the condition. A special embodiment of this invention is a method wherein the condition is Alzheimer's disease.

As defined above, therapeutically effective dose means that amount of protein, polynucleotide, peptide, or its antibodies, agonists or antagonists, which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. The exact dosage is chosen by the individual physician in view of the patient to be treated. Dosage and administration are adjusted to provide sufficient levels of the active moiety or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

The pharmaceutical compositions according to this invention may be administered to a subject by a variety of methods. They may be added directly to target tissues, complexed with cationic lipids, packaged within liposomes, or delivered to target cells by other methods known in the art. Localized administration to the desired tissues may be done by catheter, infusion pump or stent. The DNA, DNA/vehicle complexes, or the recombinant virus particles are locally administered to the site of treatment. Alternative routes of delivery include, but are not limited to, intravenous injection, intramuscular injection, subcutaneous injection, aerosol inhalation, oral (tablet or pill form), topical, systemic, ocular, intraperitoneal and/or intrathecal delivery. Examples of ribozyme delivery and administration are provided in Sullivan et al. WO 94/02595.

Antibodies according to the invention may be delivered as a bolus only, infused over time or both administered as a bolus and infused over time. Those skilled in the art may employ different formulations for polynucleotides than for proteins. Similarly, delivery of polynucleotides or polypeptides is specific to particular cells, conditions, locations, etc.

As discussed hereinabove, recombinant viruses may be used to introduce DNA encoding polynucleotide agents useful in the present invention. Recombinant viruses according to the invention are generally formulated and administered in the form of doses of between about 10⁴ and about 10¹⁴ pfu. In the case of AAVs and adenoviruses, doses of from about 10⁶ to about 10¹¹ pfu are preferably used. The term pfu (“plaque-forming unit”) corresponds to the infective power of a suspension of virions and is determined by infecting an appropriate cell culture and measuring the number of plaques formed. The techniques for determining the pfu titre of a viral solution are well documented in the prior art.

Still another aspect or the invention relates to a method for diagnosing a pathological condition involving cognitive impairment or a susceptibility to the condition in a subject, comprising determining the amount of polypeptide comprising an amino acid sequence selected from the group consisting of SEQ ID NO: 44, 50, 51 and 56 in a biological sample, and comparing the amount with the amount of the polypeptide in a healthy subject, wherein an increase of the amount of polypeptide compared to the healthy subject is indicative of the presence of the pathological condition.

### Experimental Section

### EXAMPLE 1

### Screening for GPCRs that Modulate Amyloid Beta 1-42 Levels

To identify novel drug targets that change the APP processing, stable cell lines over expressing APP are made by transfecting Hek293 or SH-SY5Y cells with APP770 wt cDNA cloned into pcDNA3.1, followed by selection with G418 for 3 weeks. At this time point colonies are picked and stable clones are expanded and tested for their secreted amyloid-beta peptide levels. The cell lines designated as “Hek293 APPwt” and “SH-SY5Y APPwt” are used in the assays.

Hek293 APPwt Assay: Cells seeded in collagen-coated plates at a cell density of 15000 cells/well (384 well plate) in DMEM (10% FBS), are infected 24 h later with 1 μl or 0.2 μl of adenovirus (corresponding to an average multiplicity of infection (MOI) of 120 and 24 respectively). The following day, the virus is washed away and DMEM (25 mM Hepes; 10% FBS) is added to the cells. Amyloid-beta peptides are allowed to accumulate during 24 h.

SH-SY5Y APPwt Assay: Cells are seeded in collagen-coated plates at a cell density of 15000 cells/well (384 well plate) in Dulbecco's MEM with Glutamax I+15% FBS HI+non-essential amino acids+Geneticin 500 μg/ml. The cells are differentiated towards the neuronal phenotype by adding 9-cis retinoic acid to a final concentration of 1 μM on day 1, day 3, day 5 and day 8. On day 9, the cells are infected with 1 μl of adenovirus (corresponding to an average multiplicity of infection (MOI) of 120 respectively). The following day, the virus is washed away and DMEM 25 mM Hepes 10% FBS is added to the cells. Amyloid beta peptides are allowed to accumulate for 24 h.

ELISA: The ELISA plate is prepared by coating with a capture antibody (JRF/cAbeta42/26) (the antibody recognizes a specific epitope on the C-terminus of Abeta 1-42; obtained from M Mercken, Johnson and Johnson Pharmaceutical Research and Development, B-2340 Beerse, Belgium) overnight in buffer 42 (Table 2) at a concentration of 2.5 μg/ml. The excess capture antibody is washed away the next morning with PBS and the ELISA plate is then blocked overnight with casein buffer (see Table 2) at 4° C. Upon removal of the blocking buffer, 30 μl of the sample is transferred to the ELISA plate and incubated overnight at 4° C. After extensive washing with PBS-Tween20 and PBS, 30 μl of the horseradish peroxidase (HRP) labeled detection antibody (Peroxidase Labeling Kit, Roche), JRF/AbetaN/25-HRP (obtained from M Mercken, Johnson and Johnson Pharmaceutical Research and Development, B-2340 Beerse, Belgium) is diluted 1/5000 in buffer C (see Table 2) and added to the wells for another 2 h. Following the removal of excess detection antibody by a wash with PBS-Tween20 and PBS, HRP activity is detected via addition of luminol substrate (Roche), which is converted into a chemiluminescent signal by the HRP enzyme.

In addition, for the SH-SY5Y APPwt assay, the samples are also analyzed in an amyloid beta x-42 ELISA. This ELISA detects all amyloid beta peptide species ending at position 42, comprising 1-42, 11-42 and 17-42 (p3), which originate respectively from BACE activity at position 1 and 11, and alpha secretase activity at position 17. Thus, in addition to the amyloidogenic pathway, the non-amyloidogenic pathway is also monitored. The protocol for the Abeta x-42 ELISA is identical to the protocol for the Abeta 1-42 ELISA, except that a HRP labeled 4G8 antibody (Signet; the antibody recognizes a specific epitope in the center of the Abeta peptides) is used as detection antibody.

TABLE 1
Buffers and Solutions Used for ELISA

| | |
|---|---|
| Buffer 42 | 30 mM NaHCO₃, 70 mM Na₂CO₃, 0.05% NaN₃, pH9.6 |
| Casein | 0.1% casein in PBS 1x |
| buffer | |
| EC Buffer | 20 mM sodium phosphate, 2 mM EDTA, 400 mM NaCl, |
| | 0.2% BSA, 0.05% CHAPS, 0.4% casein, 0.05% NaN₃, pH7 |
| Buffer C | 20 mM sodium phosphate, 2 mM EDTA, 400 mM NaCl, 1% |
| | BSA, pH7 |
| PBS 10x | 80 g NaCl + 2 g KCl + 11.5 g Na₂HPO₄.7H₂O + 2 g KH₂PO₄ |
| | in 1 1 milli Q, pH 7.4 |
| PBST | PBS 1x with 0.05% Tween 20 |

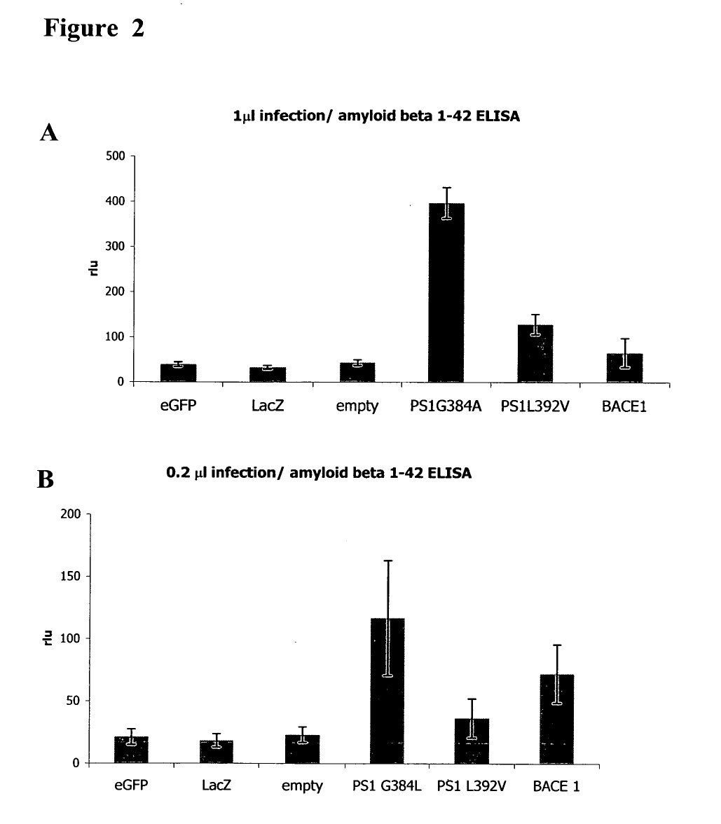

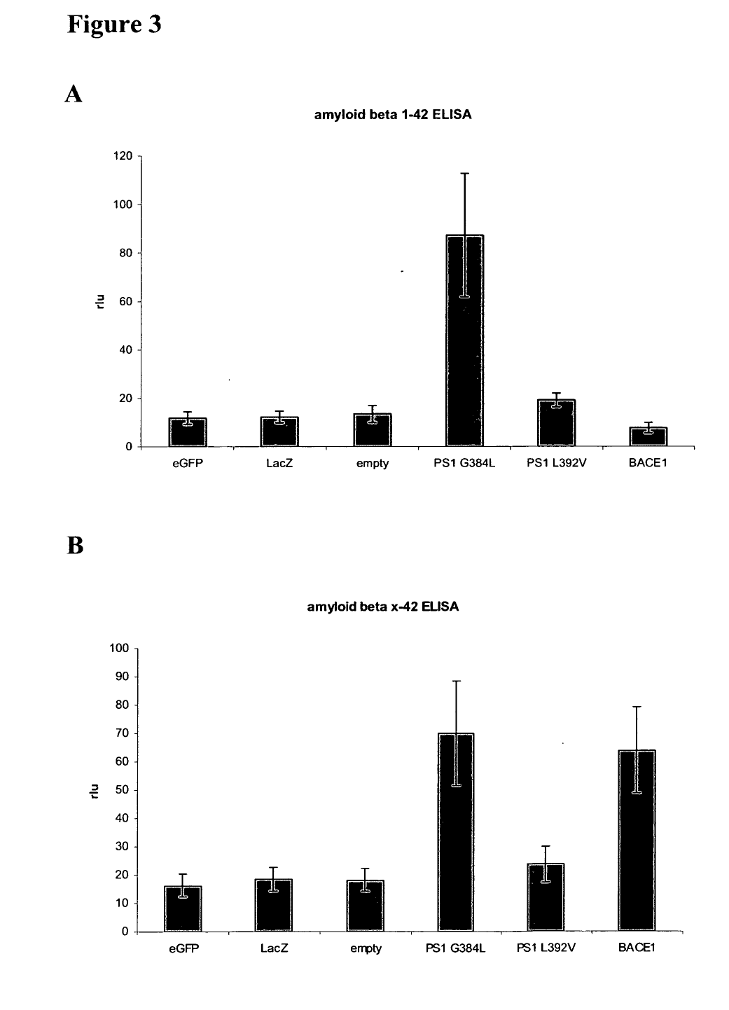

To validate the assay, the effect of adenoviral over expression with random titer of two clinical PS1 mutants and BACE on amyloid beta 1-42 production is evaluated in the Hek293 APPwt cells. As is shown in FIG. 2, all PS1 and BACE constructs induce amyloid beta 1-42 levels as expected. As is shown in FIG. 3, adenoviral overexpression of the clinical PS1 mutants in the SH-SY5Y APPwt cells also yield a significant induction of amyloid beta 1-42 levels. However, since overexpression of BACE in the SH-SY5Y APPwt cells do not result in an induction of amyloid beta 1-42 levels, amyloid beta x-42 levels are determined and show a clear induction.

An adenoviral GPCR cDNA library was constructed as follows. DNA fragments covering the full coding region of the GPCRs, are amplified by PCR from a pooled placental and fetal liver cDNA library (InvitroGen). All fragments are cloned into an adenoviral vector as described in U.S. Pat. No. 6,340,595, the contents of which are herein incorporated by reference, and subsequently adenoviruses are made harboring the corresponding cDNAs. The screen types using these libraries are presented in Table 1A.

TABLE 1A

| Screen | | | |
|---|---|---|---|
| number | Cell type | ELISA | Adenoviral library |
| H35 | Hek293 APPwt | Abeta 1-42 | GPCR library + agonists |
| H37 | SH-SY5Y APPwt | Abeta 1-42 | GPCR library + agonists |
| H38 | SH-SY5Y APPwt | Abeta x-42 | GPCR library + agonists |

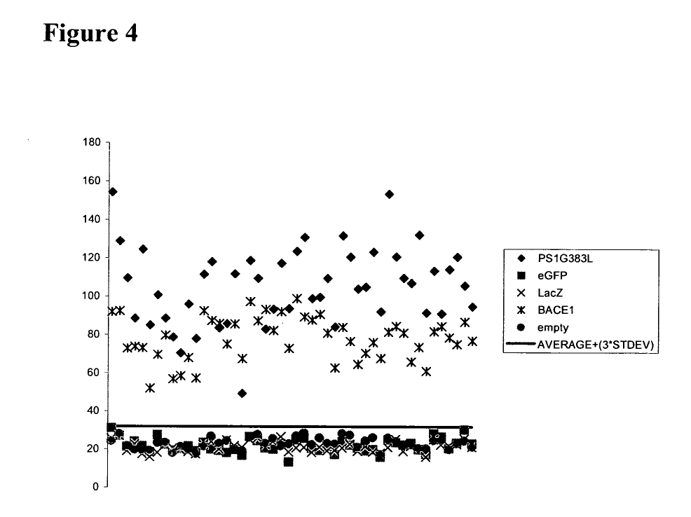

Activators of amyloid beta production are selected by calculating the average and standard deviation of all data points during the screening run (i.e. all plates processed in one week) and applying the formula AVERAGE+(N×STDEV) to calculate the cut off value (N is determined individually for every screen and is indicated in Tables 1B, C, D, E, F, which present the results of the screenings). All cDNAs scoring higher then the cut off value are considered as positives and thus modulate amyloid beta 1-42 levels. This is validated by infecting Hek293APPwt cells with a control plate containing PSIG384L, BACEI and eGFP, empty and LacZ adenoviruses. The average and standard deviation are calculated based upon the negative controls. Applying the cut off (AVERAGE+(3×STDEV)) reveals that all positive controls are identified as positive data points (FIG. 4). Repressors of the amyloid beta production are selected in a similar way, except that the cDNAs have to score lower than the cut off value determined by the formula AVERAGE—(N×STDEV). The same procedure applies for the SH-SY5Y APPwt cells. One of the selected activators during the screen was APP, underscoring the relevance of the identified hits.

Tables 1B-1F below present the results of the screening studies, measuring amyloid beta (Abeta) 1-42 and x-42 levels in Hek293 APPwt and SH-SY5Y APPwt cells infected with adenoviral cDNA library described above. The data is analyzed using four data points for every screen. A cDNA is considered a hit when at least 2 data points out of 4 score positive. Blank boxes indicate that the screen was not performed for that specific cDNA. [Act means activator, Rep means repressor. A hit is indicated as the number 1. A negative data point is indicated as “−”. PS and RS represent respectively primary screen and rescreen].

TABLE 1B

| Screening | H35 | | | | | | | |
|---|---|---|---|---|---|---|---|---|
| Agonist | 2.5 nM | | | | 25 nM | | | |
| Infection | 1 μl | | | | 1 μl | | | |
| N for Act | 1.5 | | | | 1.5 | | | |
| N for Rep | −1.1 | | | | −1.1 | | | |
| cDNA | PS | | RS | | PS | | RS | |
| LTB4R | −1.305 | −1.358 | −1.5466 | −1.5503 | −1.406 | −1.292 | −1.5323 | −1.5456 |
| | 1 | 1 | 1 | 1 | 1 | 1 | 1 | 1 |
| EDG1 | −1.131 | −1.155 | −1.108 | −1.0086 | −1.162 | −0.987 | −1.3999 | −1.1465 |
| | 1 | 1 | 1 | — | 1 | — | 1 | 1 |
| P2RY1 | 0.432 | 0.228 | 1.2527 | 0.1881 | 0.099 | −0.099 | 1.7015 | 1.7586 |
| | — | — | — | — | — | — | 1 | 1 |
| TA3 | 0.913 | 1.674 | 1.6889 | 1.2775 | 0.566 | 1.208 | 0.9497 | 1.5363 |
| | — | 1 | 1 | — | — | — | — | 1 |
| CHRM5 | 1.916 | 2.364 | 0.2957 | 2.1227 | 0.565 | 1.262 | 0.7642 | 1.5492 |
| | 1 | 1 | — | 1 | — | — | — | 1 |
| GABBR1 | 1.153 | 1.113 | 1.6997 | 1.9085 | 2.908 | 0.793 | 0.4846 | 1.316 |
| | — | — | 1 | 1 | 1 | — | — | — |
| PTGER2 | 1.815 | 1.592 | 3.227 | 2.0915 | 2.217 | 1.606 | 2.5595 | 3.0714 |
| | 1 | 1 | 1 | 1 | 1 | 1 | 1 | 1 |
| Tar1 | 1.268 | 0.979 | 1.7755 | 1.8376 | 0.579 | 1.071 | 0.9903 | 0.33 |
| | — | — | 1 | 1 | — | — | — | — |

TABLE 1C

| Screening | H37 | | | | | | | |
|---|---|---|---|---|---|---|---|---|
| Agonist | 2.5 nM | | | | 25 nM | | | |
| Infection | 1 μl | | | | 1 μl | | | |
| N for Act | 2 | | | | 2 | | | |
| N for Rep | −1.5 | | | | −1.5 | | | |
| cDNA | PS | | RS | | PS | | RS | |
| LTB4R | −1.932 | −1.402 | −1.186 | −1.503 | −1.715 | 0.031 | −1.235 | −1.765 |
| | 1 | — | — | 1 | 1 | — | — | 1 |
| EDG1 | −1.059 | −0.857 | −1.019 | −0.893 | 0.243 | 0.343 | −0.727 | −1.867 |
| | — | — | — | — | — | — | — | 1 |
| CNR1 | 3.661 | 3.477 | 3.352 | 3.444 | 2.745 | 3.167 | 2.714 | 3.019 |
| | 1 | 1 | 1 | 1 | 1 | 1 | 1 | 1 |
| AGTR1 | −0.744 | −0.591 | −0.356 | −0.855 | −0.014 | −0.149 | 1.629 | −0.915 |
| | — | — | — | — | — | — | — | — |
| CHRM2 | 0.774 | 0.818 | 0.197 | 1.384 | −0.413 | 2.044 | 2.06 | 0.312 |
| | — | — | — | — | — | 1 | 1 | — |
| ADRA1A | 0.548 | 0.708 | −0.039 | 0.429 | 0.927 | 0.39 | 1.082 | −1.001 |
| | — | — | — | — | — | — | — | — |
| CHRM3 | −1.539 | −0.728 | −1.602 | −0.568 | −0.527 | −0.608 | −0.671 | −1.333 |
| | 1 | — | 1 | — | — | — | — | — |
| ADORA3 | −0.46 | 0.263 | 0.367 | 1.265 | 1.073 | 2.476 | 2.789 | −1.037 |
| | — | — | — | — | — | 1 | 1 | — |
| OPRM1 | −1.659 | −1.338 | −1.632 | −1.055 | −0.61 | −0.193 | −0.765 | −1.79 |
| | 1 | 0 | 1 | — | — | — | — | 1 |
| PTGER2 | 1.022 | 3.147 | −0.068 | 0.369 | 1.382 | 1.686 | 3.954 | −0.09 |
| | — | 1 | — | — | — | — | 1 | — |
| Tar1 | 2.808 | 1.727 | 0.582 | 1.237 | 2.144 | 4.218 | 1.918 | −0.515 |
| | 1 | — | — | — | 1 | 1 | — | — |
| Screening | H38 | | | | | | | |
| Agonist | 2.5 nM | | | | 25 nM | | | |
| Infection | 1 μl | | | | 1 μl | | | |
| N for Act | 2 | | | | 2 | | | |
| N for Rep | −1.5 | | | | −1.5 | | | |
| cDNA | PS | | RS | | PS | | RS | |
| LTB4R | −1.557 | −1.473 | −2.431 | −1.58 | −1.691 | −1.876 | −1.778 | −1.486 |
| | 1 | — | 1 | 1 | 1 | 1 | 1 | — |
| EDG1 | −0.801 | −1.468 | −1.991 | −2 | −1.479 | −1.88 | −1.657 | −1.255 |
| | — | — | 1 | 1 | — | 1 | 1 | — |
| CNR1 | 3.542 | 2.769 | 1.107 | 2.06 | 0.936 | 1.731 | 2.325 | 3.059 |
| | 1 | 1 | — | 1 | — | — | 1 | 1 |
| AGTR1 | −0.222 | −1.693 | −0.906 | −1.596 | 0.372 | −0.642 | 0.046 | −0.544 |
| | 0 | 1 | — | 1 | 0 | — | — | — |
| CHRM2 | 0.632 | 0.199 | −0.618 | 0.257 | −1.265 | 0.973 | 1.19 | 0.892 |
| | — | — | — | — | — | — | — | — |
| ADRA1A | 2.393 | 2.975 | 0.849 | 1.335 | 1.34 | 3.609 | 2.849 | 2.79 |
| | 1 | 1 | — | — | — | 1 | 1 | 1 |
| CHRM3 | −0.375 | −0.89 | −1.558 | −1.279 | −1.091 | −1.618 | −1.018 | −0.954 |
| | — | — | 1 | — | — | 1 | — | — |
| ADORA3 | 1.838 | −0.553 | −0.706 | 1.417 | 0.261 | 1.033 | 2.14 | 0.462 |
| | 0 | 0 | 0 | 0 | — | — | 1 | — |
| OPRM1 | −0.733 | −1.882 | −2.001 | −1.565 | −1.174 | −0.914 | −0.242 | −1.17 |
| | — | 1 | 1 | 1 | — | — | — | — |
| PTGER2 | 2.137 | 0.862 | −0.074 | 1.877 | 0.264 | 1.003 | 2.752 | 2.029 |
| | 1 | — | — | — | — | — | 1 | 1 |
| Tar1 | 3.075 | 1.353 | −0.018 | 1.301 | 0.603 | 2.663 | 1.644 | 1.109 |
| | 1 | — | — | — | — | 1 | — | — |

TABLE 1D

| Screening | H35 | | | | | | | |
|---|---|---|---|---|---|---|---|---|
| infection | 0.2 μl | | | | 1 μl | | | |
| N for Act | 2 | | | | 2 | | | |
| N for Rep | −1 | | | | −1 | | | |
| cDNA | PS | | RS | | PS | | RS | |
| CCR2 | −1.15 | −0.696 | −0.591 | −0.461 | −1.045 | −0.909 | −1.114 | −0.66 |
| | 1 | — | — | — | 1 | — | 1 | — |
| CCR5 | −0.787 | −0.812 | −0.761 | −0.816 | −0.776 | −0.7 | −1.123 | −1.015 |
| | — | — | — | — | — | — | 1 | 1 |
| CCR6 | −1.329 | −1.186 | −1.053 | −0.456 | −1.152 | −1.026 | −0.995 | −0.828 |
| | 1 | 1 | 1 | — | 1 | 1 | — | — |
| TACR1 | −0.808 | −0.229 | −0.454 | −0.47 | −1.131 | −1.079 | −0.555 | −1.025 |
| | — | — | — | — | 1 | 1 | — | 1 |
| GRPR | 1.691 | 3.039 | 3.601 | 2.413 | 0.925 | 1.954 | 1.418 | 1.421 |
| | — | 1 | 1 | 1 | — | — | — | — |
| NMU2R | 0.501 | 2.337 | 1.136 | 2.063 | 1.021 | 1.719 | 1.317 | 1.472 |
| | — | 1 | — | 1 | — | — | — | — |

TABLE 1E

| Screening | H37 | | | | H38 | | | |
|---|---|---|---|---|---|---|---|---|
| Infection | 1 μl | | | | 1 μl | | | |
| N for Act | 2 | | | | 2 | | | |
| N for Rep | −1.5 | | | | −1.5 | | | |
| cDNA | PS | | RS | | PS | | RS | |
| CCXCR1 | −1.547 | −1.715 | −1.092 | −0.096 | −1.28 | −1.758 | −0.673 | −0.881 |
| | 1 | 1 | — | — | — | 1 | — | — |
| CCR3 | −1.272 | −1.633 | −1.569 | −1.834 | −0.89 | −0.846 | −1.105 | −1.785 |
| | — | 1 | 1 | 1 | — | — | — | 1 |
| SLT | 0.919 | 0.692 | −0.022 | −0.293 | 3.032 | 2.055 | 0.414 | −0.149 |
| | — | — | — | — | 1 | 1 | — | 0 |
| BDKRB2 | 0.138 | 0.27 | 2.132 | 0.69 | 2.107 | 1.192 | 2.209 | 0.682 |
| | — | — | 1 | — | 1 | — | 1 | — |
| FZD5 | −1.682 | −0.947 | −1.516 | −1.543 | −1.269 | −1.264 | −1.311 | −1.912 |
| | 1 | — | 1 | 1 | — | — | — | 1 |
| CCR5 | −2.097 | −1.933 | −1.356 | −0.832 | −1.518 | −1.466 | −1.268 | −1.254 |
| | 1 | 1 | — | — | 1 | — | — | — |
| CCR6 | −2.184 | −2.15 | −1.187 | −1.687 | −1.673 | −1.582 | −0.898 | −1.73 |
| | 1 | 1 | — | 1 | 1 | 1 | — | 1 |
| CD97 | 2.395 | 1.337 | 1.216 | 2.408 | 1.477 | 0.427 | 0.89 | 0.733 |
| | 1 | — | — | 1 | — | — | — | — |
| GRPR | 1.182 | 0.772 | 1.211 | 0.507 | 2.31 | 2.289 | 2.641 | 1.834 |
| | — | — | — | — | 1 | 1 | 1 | — |

This initial screening work provided GPCR-related leads for the up-regulation and down-regulation of amyloid beta protein processing targets. These leads are presented in Tables 2A and 2B below.

TABLE 2A
G-Protein Coupled Receptors Related to Amyloid Beta Up-Regulation

| | | | SEQ ID NO's: | | |
|---|---|---|---|---|---|
| | | | | | Corresponding |
| | | | | | inhibitory agents |
| Accession | Description | Code | DNA | Protein | (compounds) |
| NM_000677 | adenosine A3 | ADORA3 | 1 | 38 | 75-77 |
| | receptor | | | | |
| NM_001840 | cannabinoid receptor 1 | CNR1 | 2 | 39 | 78-82 |
| NM_021903 | gamma-aminobutyric | GABBR1 | 3 | 40 | 83-85 |
| | acid B receptor 1 | | | | |
| NM_000739 | cholinergic receptor, | CHRM2 | 4 | 41 | 86-89 |
| | muscarinic 2 | | | | |
| NM_012125 | cholinergic receptor, | CHRM5 | 5 | 42 | 90-94 |
| | muscarinic 5 | | | | |
| NM_001784 | CD97 antigen | CD97 | 6 | 43 | 95-97 |
| NM_005314 | gastrin-releasing | GRPR | 7 | 44 | 98-100 |
| | peptide receptor | | | | |
| NM_020167 | neuromedin U | NMU2R | 8 | 45 | 101-103 |
| | receptor 2 | | | | |
| NM_002563 | purinergic receptor | P2RY1 | 9 | 46 | 104-108 |
| | P2Y, G-protein | | | | |
| | coupled, 1 | | | | |
| NM_000956 | prostaglandin E | PTGER2 | 10 | 47 | 109-111 |
| | receptor 2 | | | | |
| NM_175057 | trace amine receptor 3 | TAR3 | 11 | 48 | 112-118 |
| NM_138327 | trace amine receptor 1 | Tar1 | 12 | 49 | 119-121 |
| NM_000680 | adrenergic, alpha-1A-, | ADRA1A_{—} | 13 | 50 | 122-127 |
| | receptor | TV1 | | | |
| NM_033302 | adrenergic, alpha-1A-, | ADRA1A_{—} | 14 | 51 | 128-133 |
| | receptor | TV3 | | | |
| NM_000623 | bradykinin receptor | BDKRB2 | 15 | 52 | 134-140 |
| | B2 | | | | |
| NM_032503 | G protein-coupled | GPR145 | 16 | 53 | 141-143 |
| | receptor 145 | | | | |

TABLE 2B
G-Protein Coupled Receptors Related to Amyloid Beta Peptide Down-regulation

| | | | SEQ ID NO's: | | |
|---|---|---|---|---|---|
| | | | | | Corresponding |
| | | | | | inhibitory agents |
| Accession | Code | Description | DNA | Protein | (compounds) |
| NM_000740 | cholinergic receptor, | CHRM3 | 17 | 54 | 144-147 |
| | muscarinic 3 | | | | |
| NM_000914 | opioid receptor, mu 1 | OPRM1 | 18 | 55 | 148-152 |
| NM_001058 | tachykinin receptor 1 | TACR1 | 19 | 56 | 153-156 |
| NM_005283 | chemokine (C motif) | CCXCR1 | 20 | 57 | 157-161 |
| | receptor 1 | | | | |
| NM_001400 | endothelial | EDG1 | 21 | 58 | 162-166 |
| | differentiation, | | | | |
| | sphingolipid G- | | | | |
| | protein-coupled | | | | |
| | receptor, 1 | | | | |
| NM_181657 | leukotriene B4 | LTB4R | 22 | 59 | 167-169 |
| | receptor | | | | |
| NM_000647 | chemokine (C—C | CCR2 | 23 | 60 | 170-172 |
| | motif) receptor 2 | | | | |
| NM_000579 | chemokine (C—C | CCR5 | 24 | 61 | 173-177 |
| | motif) receptor 5 | | | | |
| NM_004367 | chemokine (C—C | CCR6 | 25 | 62 | 178-182 |
| | motif) receptor 6 | | | | |
| NM_003468 | frizzled homolog 5 | FZD5 | 26 | 63 | 183-186 |
| NM_001837 | chemokine (C—C | CCR3 | 27 | 64 | 187-189 |
| | motif) receptor 3 | | | | |
| NM_000685 | angiotensin II | AGTR1 | 28 | 65 | 190-192 |
| NM_178329 | receptor, type 1 | | | | |
| NM_005293 | G protein-coupled | GPR20 | 29 | 66 | 193-195 |
| | receptor 20 | | | | |
| NM_003485 | G protein-coupled | GPR68 | 30 | 67 | 196-200 |
| | receptor 68 | | | | |

The experimental work following this initial screening of GPCRs indicates that the GCPRs identified as GRPR, ADRA1A, and TACR1 [SEQ ID NO: 7, 13, 14, 19 (DNA sequence); and 44, 50, 51, and 56 (amino acid sequence)] are involved in APP processing.

Following the initial screening work, additional screening of these GPCRs in Hek293 APPwt cells and SH-SY5Y APPwt cells demonstrate that increased expression thereof leads to (a) increased levels of amyloid beta x-42 peptides in the conditioned medium of Hek293 APPwt cells, and (b) increased levels of amyloid beta 1-42 and x-42 peptides in the conditioned medium of SH-SY5Y APPwt cells. These results indicate that GRPR, ADRA1A, and TACR1 expression is involved in aberrant APP processing.

The sequence information for these GCPRs, exemplary derivative sequences for expression-inhibiting agents (SEQ ID NO: 98-100, 122-133, 153-156 and 232-537), and the proteins domains of GRPR, ADRA1A, and TACR1 (SEQ ID NO: 538-582) are provided in Table 3 below.:

TABLE 3

DNA and Amino Acid Sequences for GPCRs involved in APP processing, DNA
Sequences for expression-inhibiting agent, and the hairpin loop sequence
of the shRNA, and the various domains of GRPR, ADRA1A, and TACR1.

| | | | | | |
|---|---|---|---|---|---|
| SEQ | | Oligo or | | | |
| ID | | Protein | | | |
| No. | Accession | Segment Name | Sequence | Name | Type |

| | | | | | |
|---|---|---|---|---|---|
| 7 | NM_005314 | | | GRPR | DNA |
| 13 | NM_000680 | | | ADRA1A_TV1 | DNA |
| 14 | NM_033302 | | | ADRA1A_TV3 | DNA |
| 19 | NM_001056 | | | TACR1 | DNA |
| 44 | | | | GRPR | Protein |
| 50 | | | | ADRA1A_TV1 | Protein |
| 51 | | | | ADRA1A_TV3 | Protein |
| 56 | | | | TACR1 | Protein |
| 231 | | | UUGCUAU | loop | DNA |
| 232 | NM_005314 | NM_005314_idx597 | ACAGTCAAGTCCATGCGAAAC | GRPR (BB2) | DNA |
| 233 | NM_005314 | NM_005314_idx671 | AACGTGTGCTCCAGTGGATGC | GRPR (BB2) | DNA |
| 234 | NM_005314 | NM_008177_idx683 | AATTGGCTGCAAACTGATCCC | GRPR (BB2) | DNA |
| 235 | NM_005314 | NM_005314_idx808 | ACAGATACAAAGCCATTGTCC | GRPR (BB2) | DNA |
| 236 | NM_005314 | NM_008177_idx782 | ACGGCCAATGGATATCCAGGC | GRPR (BB2) | DNA |
| 237 | NM_005314 | NM_008177_idx804 | TCCCATGCCCTGATGAAGATC | GRPR (BB2) | DNA |
| 238 | NM_005314 | NM_005314_idx864 | AAGATCTGCCTCAAAGCCGCC | GRPR (BB2) | DNA |
| 239 | NM_005314 | NM_005314_idx876 | AAAGCCGCCTTTATCTGGATC | GRPR (BB2) | DNA |
| 240 | NM_005314 | NM_005314_idx963 | ACCAACCAGACCTTCATTAGC | GRPR (BB2) | DNA |
| 241 | NM_005314 | NM_005314_idx994 | ACCCACACTCTAATGAGCTTC | GRPR (BB2) | DNA |
| 242 | NM_005314 | NM_005314_idx998 | ACACTCTAATGAGCTTCACCC | GRPR (BB2) | DNA |
| 243 | NM_005314 | NM_005314_idx1104 | AATCTGATCCAGAGTGCTTAC | GRPR (BB2) | DNA |
| 244 | NM_005314 | NM_005314_idx1191 | ACAGTGCTGGTGTTTGTGGGC | GRPR (BB2) | DNA |
| 245 | NM_005314 | NM_008177_idx1192 | ACCATGTCATCTACCTGTACC | GRPR (BB2) | DNA |
| 246 | NM_005314 | NM_008177_idx1231 | AAGTGGACACCTCCATGCTCC | GRPR (BB2) | DNA |
| 247 | NM_005314 | NM_005314_idx1284 | ACCTCCATGCTCCACTTTGTC | GRPR (BB2) | DNA |
| 248 | NM_005314 | NM_005314_idx1390 | AACAGTTCAACACTCAGCTGC | GRPR (BB2) | DNA |
| 249 | NM_005314 | NM_005314_idx1398 | AACACTCAGCTGCTCTGTTGC | GRPR (BB2) | DNA |
| 250 | NM_005314 | NM_005314_idx1399 | ACACTCAGCTGCTCTGTTGCC | GRPR (BB2) | DNA |
| 251 | NM_005314 | NM_005314_idx1449 | ACTGGAAGGAGTACAACCTGC | GRPR (BB2) | DNA |
| 252 | NM_005314 | NM_005314_idx1454 | AAGGAGTACAACCTGCATGAC | GRPR (BB2) | DNA |
| 253 | NM_005314 | NM_005314_idx1461 | ACAACCTGCATGACCTCCCTC | GRPR (BB2) | DNA |
| 254 | NM_005314 | NM_005314_idx1473 | ACCTCCCTCAAGAGTACCAAC | GRPR (BB2) | DNA |
| 255 | NM_005314 | NM_005314_idx1642 | AAAGAGCCTTCAGAATGCTCC | GRPR (BB2) | DNA |
| 256 | NM_005314 | NM_005314_idx597 | AGTCAAGTCCATGCGAAAC | GRPR (BB2) | DNA |
| 257 | NM_005314 | NM_005314_idx671 | CGTGTGCTCCAGTGGATGC | GRPR (BB2) | DNA |
| 258 | NM_005314 | NM_008177_idx683 | TTGGCTGCAAACTGATCCC | GRPR (BB2) | DNA |
| 259 | NM_005314 | NM_005314_idx808 | AGATACAAAGCCATTGTCC | GRPR (BB2) | DNA |
| 260 | NM_005314 | NM_008177_idx782 | GGCCAATGGATATCCAGGC | GRPR (BB2) | DNA |
| 261 | NM_005314 | NM_008177_idx804 | CCATGCCCTGATGAAGATC | GRPR (BB2) | DNA |
| 262 | NM_005314 | NM_005314_idx864 | GATCTGCCTCAAAGCCGCC | GRPR (BB2) | DNA |
| 263 | NM_005314 | NM_005314_idx876 | AGCCGCCTTTATCTGGATC | GRPR (BB2) | DNA |
| 264 | NM_005314 | NM_005314_idx963 | CAACCAGACCTTCATTAGC | GRPR (BB2) | DNA |
| 265 | NM_005314 | NM_005314_idx994 | CCACACTCTAATGAGCTTC | GRPR (BB2) | DNA |
| 266 | NM_005314 | NM_005314_idx998 | ACTCTAATGAGCTTGACCC | GRPR (BB2) | DNA |
| 267 | NM_005314 | NM_005314_idx1104 | TCTGATCCAGAGTGCTTAC | GRPR (BB2) | DNA |
| 268 | NM_005314 | NM_005314_idx1191 | AGTGCTGGTGTTTGTGGGC | GRPR (BB2) | DNA |
| 269 | NM_005314 | NM_008177_idx1192 | CATGTCATCTACCTGTACC | GRPR (BB2) | DNA |
| 270 | NM_005314 | NM_008177_idx1231 | GTGGACACCTCCATGCTCC | GRPR (BB2) | DNA |
| 271 | NM_005314 | NM_005314_idx1284 | CTCCATGCTCCACTTTGTC | GRPR (BB2) | DNA |
| 272 | NM_005314 | NM_005314_idx1390 | CAGTTCAACACTCAGCTGC | GRPR (BB2) | DNA |
| 273 | NM_005314 | NM_005314_idx1398 | CACTCAGCTGCTCTGGTGC | GRPR (BB2) | DNA |
| 274 | NM_005314 | NM_005314_idx1399 | ACTCAGCTGCTCTGTTGCC | GRPR (BB2) | DNA |
| 275 | NM_005314 | NM_005314_idx1449 | TGGAAGGAGTACAACCTGC | GRPR (BB2) | DNA |
| 276 | NM_005314 | NM_005314_idx1454 | GGAGTACAACCTGCATGAC | GRPR (BB2) | DNA |
| 277 | NM_005314 | NM_005314_idx1461 | AACCTGCATGACCTCCCTC | GRPR (BB2) | DNA |
| 278 | NM_005314 | NM_005314_idx1473 | CTCCCTCAAGAGTACCAAC | GRPR (BB2) | DNA |
| 279 | NM_005314 | NM_005314_idx1642 | AGAGCCTTCAGAATGCTCC | GRPR (BB2) | DNA |
| 280 | NM_000680 | NM_000680_idx566 | AACATCCTAGTGATCCTCTCC | ADRA1A_TV | DNA |
| 281 | NM_000680 | NM_000680_idx617 | ACGCACTACTACATCGTCAAC | ADRA1A_TV | DNA |
| 282 | NM_000680 | NM_000680_idx1091 | AAGTCTGGCCTCAAGACCGAC | ADRA1A_TV | DNA |
| 283 | NM_000680 | NM_000680_idx1128 | AAGTGACGCTCCGCATCCATC | ADRA1A_TV | DNA |
| 284 | NM_000680 | NM_000680_idx1199 | ACGCACTTCTCAGTGAGGCTC | ADRA1A_TV | DNA |
| 285 | NM_000680 | NM_000680_idx1480 | AAAGCAGTCTTCCAAACATGC | ADRA1A_TV | DNA |
| 286 | NM_000680 | NM_000680_idx1481 | AAGCAGTCTTCCAAACATGCC | ADRA1A_TV | DNA |
| 287 | NM_000680 | NM_000680_idx1493 | AAACATGCCCTGGGCTACACC | ADRA1A_TV | DNA |
| 288 | NM_000680 | NM_000680_idx1548 | ACAAGGACATGGTGCGCATCC | ADRA1A_TV | DNA |
| 289 | NM_000680 | NM_000680_idx1667 | ACAGTGTCCAAAGACCAATCC | ADRA1A_TV | DNA |
| 290 | NM_000680 | NM_000680_idx1676 | AAAGACCAATCCTCCTGTACC | ADRA1A_TV | DNA |
| 291 | NM_000680 | NM_000680_idx1683 | AATCCTCCTGTACCACAGCCC | ADRA1A_TV | DNA |
| 292 | NM_000680 | NM_000680_idx1769 | AAGAACCATCAAGTTCCAACC | ADRA1A_TV | DNA |
| 293 | NM_000680 | NM_000680_idx1779 | AAGTTCCAACCATTAAGGTCC | ADRA1A_TV | DNA |
| 294 | NM_000680 | NM_000680_idx1787 | ACCATTAAGGTCCACACCATC | ADRA1A_TV | DNA |
| 295 | NM_000680 | NM_000680_idx1793 | AAGGTCCACACCATCTCCCTC | ADRA1A_TV | DNA |
| 296 | NM_000680 | NM_000680_idx1802 | ACCATCTCCCTCAGTGAGAAC | ADRA1A_TV | DNA |
| 297 | NM_000680 | NM_000680_idx1864 | AATAATCTTAGGTACCCACCC | ADRA1A_TV | DNA |
| 298 | NM_033303 | NM_000680_idx566 | AACATCCTAGTGATCCTCTCC | ADRA1A_TV | DNA |
| 299 | NM_033303 | NM_000680_idx617 | ACGCACTACTACATCGTCAAC | ADRA1A_TV | DNA |
| 300 | NM_033303 | NM_000680_idx1091 | AAGTCTGGCCTCAAGACCGAC | ADRA1A_TV | DNA |
| 301 | NM_033303 | NM_000680_idx1228 | AAGTGACGCTCCGCATCCATC | ADRA1A_TV | DNA |
| 302 | NM_033303 | NM_000680_idx1199 | ACGCACTTCTCAGTGAGGCTC | ADRA1A_TV | DNA |
| 303 | NM_033303 | NM_000680_idx1480 | AAAGCAGTCTTCCAAACATGC | ADRA1A_TV | DNA |
| 304 | NM_033303 | NM_000680_idx1481 | AAGCAGTCTTCCAAACATGCC | ADRA1A_TV | DNA |
| 305 | NM_033303 | NM_000680_idx1493 | AAACATGCCCTGGGCTACACC | ADRA1A_TV | DNA |
| 306 | NM_033303 | NM_000680_idx1548 | ACAAGGACATGGTGCGCATCC | ADRA1A_TV | DNA |
| 307 | NM_033303 | NM_000680_idx1667 | ACAGTGTCCAAAGACCAATCC | ADRA1A_TV | DNA |
| 308 | NM_033303 | NM_000680_idx1676 | AAAGACCAATCCTCCTGTACC | ADRA1A_TV | DNA |
| 309 | NM_033303 | NM_000680_idx1683 | AATCCTCCTGTACCACAGCCC | ADRA1A_TV | DNA |
| 310 | NM_033303 | NM_033303_idx1706 | ACGAAGTCTCGCTCTGTCACC | ADRA1A_TV | DNA |
| 311 | NM_033303 | ENSG00000 | AATGGCATGATCTTGGCTCAC | ADRA1A_TV | DNA |
| | | 171556_idx1607 | | | |
| 312 | NM_033303 | ENSG00000 | ACGATCTTGGCTCACTGCAAC | ADRA1A_TV | DNA |
| | | 116032_idx5773 | | | |
| 313 | NM_033303 | NM_033303_idx1783 | ACGATCTTGGCTCACTGCAAC | ADRA1A_TV | DNA |
| 314 | NM_033303 | NM_033303_idx1896 | ACCATGTTGGCCAGGATGATC | ADRA1A_TV | DNA |
| 315 | NM_033303 | NM_033303_idx1928 | ACCTCATGATCTGCCTGCCTC | ADRA1A_TV | DNA |
| 316 | NM_033303 | NM_033303_idx2152 | AACACACACACACATTCTCTC | ADRA1A_TV | DNA |
| 317 | NM_033303 | NM_033303_idx2153 | ACACACACACACATTCTCTCC | ADRA1A_TV | DNA |
| 318 | NM_033303 | NM_033303_idx2161 | ACACATTCTCTCCATGGTGAC | ADRA1A_TV | DNA |
| 319 | NM_033303 | NM_033303_idx2204 | ACATAGTACACCATGGAGCAC | ADRA1A_TV | DNA |
| 320 | NM_033303 | NM_033303_idx2213 | ACCATGGAGCACGGTTTAAGC | ADRA1A_TV | DNA |
| 321 | NM_033303 | NM_033303_idx2223 | ACGGTTTAAGCACCACTGGAC | ADRA1A_TV | DNA |
| 322 | NM_033303 | NM_033303_idx2271 | ACCTTCCCATAGACACCCAGC | ADRA1A_TV | DNA |
| 323 | NM_033302 | NM_000680_idx566 | AACATCCTAGTGATCCTCTCC | ADRA1A_TV | DNA |
| 324 | NM_033302 | NM_000680_idx617 | ACGCACTACTACATCGTCAAC | ADRA1A_TV | DNA |
| 325 | NM_033302 | NM_000680_idx1091 | AAGTCTGGCCTCAAGACCGAC | ADRA1A_TV | DNA |
| 326 | NM_033302 | NM_000680_idx1128 | AAGTGACGCTCCGCATCCATC | ADRA1A_TV | DNA |
| 327 | NM_033302 | NM_000680_idx1199 | ACGCACTTCTCAGTGAGGCTC | ADRA1A_TV | DNA |
| 328 | NM_033302 | NM_000680_idx1480 | AAAGCAGTCTTCCAAACATGC | ADRA1A_TV | DNA |
| 329 | NM_033302 | NM_000680_idx1481 | AAGCAGTCTTCCAAACATGCC | ADRA1A_TV | DNA |
| 330 | NM_033302 | NM_000680_idx1493 | AAACATGCCCTGGGCTACACC | ADRA1A_TV | DNA |
| 331 | NM_033302 | NM_000680_idx1548 | ACAAGGACATGGTGCGCATCC | ADRA1A_TV | DNA |
| 332 | NM_033302 | NM_000680_idx1667 | ACAGTGTCCAAAGACCAATCC | ADRA1A_TV | DNA |
| 333 | NM_033302 | NM_000680_idx1676 | AAAGACCAATCCTCCTGTACC | ADRA1A_TV | DNA |
| 334 | NM_033302 | NM_000680_idx1683 | AATCCTCCTGTACCACAGCCC | ADRA1A_TV | DNA |
| 335 | NM_033302 | NM_033302_idx1710 | ACACACCCATGACATGAAGCC | ADRA1A_TV | DNA |
| 336 | NM_033302 | NM_033302_idx1721 | ACATGAAGCCAGCTTCCCGTC | ADRA1A_TV | DNA |
| 337 | NM_033302 | NM_033302_idx1743 | ACGACTGTTGTCCTTACTGCC | ADRA1A_TV | DNA |
| 338 | NM_033302 | NM_033302_idx1872 | AAGCATCCATCTGACTAAGGC | ADRA1A_TV | DNA |
| 339 | NM_033304 | NM_000680_idx566 | AACATCCTAGTGATCCTCTCC | ADRA1A_TV | DNA |
| 340 | NM_033304 | NM_000680_idx617 | ACGCACTACTACATCGTCAAC | ADRA1A_TV | DNA |
| 341 | NM_033304 | NM_000680_idx1091 | AAGTCTGGCCTCAAGACCGAC | ADRA1A_TV | DNA |
| 342 | NM_033304 | NM_000680_idx1128 | AAGTGACGCTCCGCATCCATC | ADRA1A_TV | DNA |
| 343 | NM_033304 | NM_000680_idx1199 | ACGCACTTCTCAGTGAGGCTC | ADRA1A_TV | DNA |
| 344 | NM_033304 | NM_000680_idx1480 | AAAGCAGTCTTCCAAACATGC | ADRA1A_TV | DNA |
| 345 | NM_033304 | NM_000680_idx1481 | AAGCAGTCTTCCAAACATGCC | ADRA1A_TV | DNA |
| 346 | NM_033304 | NM_000680_idx1493 | AAACATGCCCTGGGCTACACC | ADRA1A_TV | DNA |
| 347 | NM_033304 | NM_000680_idx1548 | ACAAGGACATGGTGCGCATCC | ADRA1A_TV | DNA |
| 348 | NM_033304 | NM_000680_idx1667 | ACAGTGTCCAAAGACCAATCC | ADRA1A_TV | DNA |
| 349 | NM_033304 | NM_000680_idx1676 | AAAGACCAATCCTCCTGTACC | ADRA1A_TV | DNA |
| 350 | NM_033304 | NM_000680_idx1683 | AATCCTCCTGTACCACAGCCC | ADRA1A_TV | DNA |
| 351 | NM_033304 | NM_033304_idx1550 | AAAGGGTCTAGAATGCTGATC | ADRA1A_TV | DNA |
| 352 | NM_033304 | NM_033304_idx1602 | AATGAGGAGTCAGCTGGAAGC | ADRA1A_TV | DNA |
| 353 | NM_033304 | NM_033304_idx1663 | AAACTGGATATCCCAACCTTC | ADRA1A_TV | DNA |
| 354 | NM_033304 | NM_033304_idx1687 | ACCAGTAGGTTTCATGGTTAC | ADRA1A_TV | DNA |
| 355 | NM_000680 | NM_000680_idx566 | CATCCTAGTGATCCTCTCC | ADRA1A_TV | DNA |
| 356 | NM_000680 | NM_000680_idx617 | GCACTACTACATCGTCAAC | ADRA1A_TV | DNA |
| 357 | NM_000680 | NM_000680_idx1091 | GTCTGGCCTCAAGACCGAC | ADRA1A_TV | DNA |
| 358 | NM_000680 | NM_000680_idx1128 | GTCACGCTCCGCATCCATC | ADRA1A_TV | DNA |
| 359 | NM_000680 | NM_000680_idx1199 | GCACTTCTCAGTGAGGCTC | ADRA1A_TV | DNA |
| 360 | NM_000680 | NM_000680_idx1480 | AGCAGTCTTCCAAACATGC | ADRA1A_TV | DNA |
| 361 | NM_000680 | NM_000680_idx1481 | GCAGTCTTCCAAACATGCC | ADRA1A_TV | DNA |
| 362 | NM_000680 | NM_000680_idx1493 | ACATGCCCTGGGCTACACC | ADRA1A_TV | DNA |
| 363 | NM_000680 | NM_000680_idx1548 | AAGGACATGGTGCGCATCC | ADRA1A_TV | DNA |
| 364 | NM_000680 | NM_000680_idx1667 | AGTGTCCAAAGACCAATCC | ADRA1A_TV | DNA |
| 365 | NM_000680 | NM_000680_idx1676 | AGACCAATCCTCCTGTACC | ADRA1A_TV | DNA |
| 366 | NM_000680 | NM_000680_idx1683 | TCCTCCTGTACCACAGCCC | ADRA1A_TV | DNA |
| 367 | NM_000680 | NM_000680_idx1769 | GAACCATCAAGTTCCAACC | ADRA1A_TV | DNA |
| 368 | NM_000680 | NM_000680_idx1779 | GTTCCAACCATTAAGGTCC | ADRA1A_TV | DNA |
| 369 | NM_000680 | NM_000680_idx1787 | CATTAAGGTCCACACCATC | ADRA1A_TV | DNA |
| 370 | NM_000680 | NM_000680_idx1793 | GGTCCACACCATCTCCCTC | ADRA1A_TV | DNA |
| 371 | NM_000680 | NM_000680_idx1802 | CATCTCCCTCAGTGAGAAC | ADRA1A_TV | DNA |
| 372 | NM_000680 | NM_000680_idx1864 | TAATCTTAGGTACCCACCC | ADRA1A_TV | DNA |
| 373 | NM_033303 | NM_000680_idx566 | CATCCTAGTGATCCTCTCC | ADRA1A_TV | DNA |
| 374 | NM_033303 | NM_000680_idx617 | GCACTACTACATCGTCAAC | ADRA1A_TV | DNA |
| 375 | NM_033303 | NM_000680_idx1091 | GTCTGGCCTCAAGACCGAC | ADRA1A_TV | DNA |
| 376 | NM_033303 | NM_000680_idx1128 | GTGACGCTCCGCATCCATC | ADRA1A_TV | DNA |
| 377 | NM_033303 | NM_000680_idx1199 | GCACTTCTCAGTGAGGCTC | ADRA1A_TV | DNA |
| 378 | NM_033303 | NM_000680_idx1480 | AGCAGTCTTCCAAACATGC | ADRA1A_TV | DNA |
| 379 | NM_033303 | NM_000680_idx1481 | GCAGTCTTCCAAACATGCC | ADRA1A_TV | DNA |
| 380 | NM_033303 | NM_000680_idx1493 | ACATGCCCTGGGCTACACC | ADRA1A_TV | DNA |
| 381 | NM_033303 | NM_000680_idx1548 | AAGGACATGGTGCGCATCC | ADRA1A_TV | DNA |
| 382 | NM_033303 | NM_000680_idx1667 | AGTGTCCAAAGACCAATCC | ADRA1A_TV | DNA |
| 383 | NM_033303 | NM_000680_idx1676 | AGACCAATCCTCCTGTACC | ADRA1A_TV | DNA |
| 384 | NM_033303 | NM_000680_idx1683 | TCCTCCTGTACCACAGCCC | ADRA1A_TV | DNA |
| 385 | NM_033303 | NM_033303_idx1706 | GAAGTCTCGCTCTGTCACC | ADRA1A_TV | DNA |
| 386 | NM_033303 | ENSG00000 | TGGCATGATCTTGGCTCAC | ADRA1A_TV | DNA |
| | | 171556_idx1607 | | | |
| 387 | NM_033303 | ENSG00000 | GATCTTGGCTCACTGCAAC | ADRA1A_TV | DNA |
| | | 116032_idx5773 | | | |
| 388 | NM_033303 | NM_033303_idx1783 | GAGATTCTCCTGCCTCAGC | ADRA1A_TV | DNA |
| 389 | NM_033303 | NM_033303_idx1896 | CATGTTGGCCAGGATGATC | ADRA1A_TV | DNA |
| 390 | NM_033303 | NM_033303_idx1928 | CTCATGATCTGCCTGCCTC | ADRA1A_TV | DNA |
| 391 | NM_033303 | NM_033303_idx2152 | CACACACACACATTCTCTC | ADRA1A_TV | DNA |
| 392 | NM_033303 | NM_033303_idx2153 | ACACACACACATTCTCTCC | ADRA1A_TV | DNA |
| 393 | NM_033303 | NM_033303_idx2161 | ACATTCTCTCCATGGTGAC | ADRA1A_TV | DNA |
| 394 | NM_033303 | NM_033303_idx2204 | ATAGTACACCATGGAGCAC | ADRA1A_TV | DNA |
| 395 | NM_033303 | NM_033303_idx2213 | CATGGAGCACGGTTTAAGC | ADRA1A_TV | DNA |
| 396 | NM_033303 | NM_033303_idx2223 | GGTTTAAGCACCACTGGAC | ADRA1A_TV | DNA |
| 397 | NM_033303 | NM_033303_idx2271 | CTTCCCATAGACACCCAGC | ADRA1A_TV | DNA |
| 398 | NM_033302 | NM_000680_idx566 | CATCCTAGTGATCCTCTCC | ADRA1A_TV | DNA |
| 399 | NM_033302 | NM_000680_idx617 | GCACTACTACATCGTCAAC | ADRA1A_TV | DNA |
| 400 | NM_033302 | NM_000680_idx1091 | GTCTGGCCTCAAGACCGAC | ADRA1A_TV | DNA |
| 401 | NM_033302 | NM_000680_idx1128 | GTGACGCTCCGCATCCATC | ADRA1A_TV | DNA |
| 402 | NM_033302 | NM_000680_idx1199 | GCACTTCTCAGTGAGGCTC | ADRA1A_TV | DNA |
| 403 | NM_033302 | NM_000680_idx1480 | AGCAGTCTTCCAAACATGC | ADRA1A_TV | DNA |
| 404 | NM_033302 | NM_000680_idx1481 | GCAGTCTTCCAAACATGCC | ADRA1A_TV | DNA |
| 405 | NM_033302 | NM_000680_idx1493 | ACATGCCCTGGGCTACACC | ADRA1A_TV | DNA |
| 406 | NM_033302 | NM_000680_idx1548 | AAGGACATGGTGCGCATCC | ADRA1A_TV | DNA |
| 407 | NM_033302 | NM_000680_idx1667 | AGTGTCCAAAGACCAATCC | ADRA1A_TV | DNA |
| 408 | NM_033302 | NM_000680_idx1676 | AGACCAATCCTCCTGTACC | ADRA1A_TV | DNA |
| 409 | NM_033302 | NM_000680_idx1683 | TCCTCCTGTACCACAGCCC | ADRA1A_TV | DNA |
| 410 | NM_033302 | NM_033302_idx1710 | ACACCCATGACATGAAGCC | ADRA1A_TV | DNA |
| 411 | NM_033302 | NM_033302_idx1721 | ATGAAGCCAGCTTCCCGTC | ADRA1A_TV | DNA |
| 412 | NM_033302 | NM_033302_idx1743 | GACTGTTGTCCTTACTGCC | ADRA1A_TV | DNA |
| 413 | NM_033302 | NM_033302_idx1872 | GCATCCATCTGACTAAGGC | ADRA1A_TV | DNA |
| 414 | NM_033304 | NM_000680_idx566 | CATCCTAGTGATCCTCTCC | ADRA1A_TV | DNA |
| 415 | NM_033304 | NM_000680_idx617 | GCACTACTACATCGTCAAC | ADRA1A_TV | DNA |
| 416 | NM_033304 | NM_000680_idx1091 | GTCTGGCCTCAAGACCGAC | ADRA1A_TV | DNA |
| 417 | NM_033304 | NM_000680_idx1128 | GTGACGCTCCGCATCCATC | ADRA1A_TV | DNA |
| 418 | NM_033304 | NM_000680_idx1199 | GCACTTCTCAGTGAGGCTC | ADRA1A_TV | DNA |
| 419 | NM_033304 | NM_000680_idx1480 | AGCAGTCTTCCAAACATGC | ADRA1A_TV | DNA |
| 420 | NM_033304 | NM_000680_idx1481 | GCAGTCTTCCAAACATGCC | ADRA1A_TV | DNA |
| 421 | NM_033304 | NM_000680_idx1493 | ACATGCCCTGGGCTACACC | ADRA1A_TV | DNA |
| 422 | NM_033304 | NM_000680_idx1548 | AAGGACATGGTGCGCATCC | ADRA1A_TV | DNA |
| 423 | NM_033304 | NM_000680_idx1667 | AGTGTCCAAAGACCAATCC | ADRA1A_TV | DNA |
| 424 | NM_033304 | NM_000680_idx1676 | AGACCAATCCTCCTGTACC | ADRA1A_TV | DNA |
| 425 | NM_033304 | NM_000680_idx1683 | TCCTCCTGTACCACAGCCC | ADRA1A_TV | DNA |
| 426 | NM_033304 | NM_033304_idx1550 | AGGGTCTAGAATGCTGATC | ADRA1A_TV | DNA |
| 427 | NM_033304 | NM_033304_idx1602 | TGAGGAGTCAGCTGGAAGC | ADRA1A_TV | DNA |
| 428 | NM_033304 | NM_033304_idx1663 | ACTGGATATCCCAACCTTC | ADRA1A_TV | DNA |
| 429 | NM_033304 | NM_033304_idx1687 | CAGTAGGTTTCATGGTTAC | ADRA1A_TV | DNA |
| 430 | NM_001058 | NM_001058_idx290 | AACCAGCCTGGCAAATTGTCC | TACR1 | DNA |
| 431 | NM_001058 | NM_001058_idx303 | AATTGTCCTTTGGGCAGCTGC | TACR1 | DNA |
| 432 | NM_001058 | NM_001058_idx358 | AACGTGGTAGTGATGTGGATC | TACR1 | DNA |
| 433 | NM_001058 | NM_001058_idx463 | AATACAGTGGTGAACTTCACC | TACR1 | DNA |
| 434 | NM_001058 | NM_001058_idx475 | AACTTCACCTATGCTGTCCAC | TACR1 | DNA |
| 435 | NM_001058 | NM_001058_idx494 | ACAACGAATGGTACTACGGCC | TACR1 | DNA |
| 436 | NM_001058 | NM_001058_idx526 | AAGTTCCACAACTTCTTTCCC | TACR1 | DNA |
| 437 | NM_001058 | NM_001058_idx643 | ACAGCCACCAAAGTGGTCATC | TACR1 | DNA |
| 438 | NM_001058 | NM_001058_idx649 | ACCAAAGTGGTCATCTGTGTC | TACR1 | DNA |
| 439 | NM_001058 | NM_001058_idx652 | AAAGTGGTCATCTGTGTCATC | TACR1 | DNA |
| 440 | NM_001058 | NM_001058_idx713 | ACTCAACCACAGAGACCATGC | TACR1 | DNA |
| 441 | NM_001058 | NM_001058_idx797 | ACCACATCTGTGTGACTGTGC | TACR1 | DNA |
| 442 | NM_001058 | NM_001058_idx811 | ACTGTGCTGATCTACTTCCTC | TACR1 | DNA |
| 443 | NM_001058 | NM_001058_idx857 | ACACCGTAGTGGGAATCACAC | TACR1 | DNA |
| 444 | NM_001058 | NM_001058_idx874 | ACACTATGGGCCAGTGAGATC | TACR1 | DNA |
| 445 | NM_001058 | NM_001058_idx876 | ACTATGGGCCAGTGAGATCCC | TACR1 | DNA |
| 446 | NM_001058 | NM_001058_idx920 | ACGAGCAAGTCTCTGCCAAGC | TACR1 | DNA |
| 447 | NM_001058 | NM_001058_idx1025 | ACATCAACCCAGATCTCTACC | TACR1 | DNA |
| 448 | NM_001058 | NM_001058_idx1043 | ACCTGAAGAAGTTTATCCAGC | TACR1 | DNA |
| 449 | NM_001058 | NM_001058_idx1048 | AAGAAGTTTATCCAGCAGGTC | TACR1 | DNA |
| 450 | NM_001058 | NM_001058_idx1051 | AAGTTTATCCAGCAGGTCTAC | TACR1 | DNA |
| 451 | NM_001058 | NM_001058_idx1135 | AATGACAGGTTCCGTCTGGGC | TACR1 | DNA |
| 452 | NM_001058 | NM_001058_idx1214 | AAATGAAATCCACCCGGTATC | TACR1 | DNA |
| 453 | NM_001058 | NM_001058_idx1363 | AACTGCTCTTCACGAACTGAC | TACR1 | DNA |
| 454 | NM_001058 | NM_001058_idx1377 | AAGTGACTCCAAGACCATGAC | TACR1 | DNA |
| 455 | NM_001058 | NM_001058_idx1387 | AAGACCATGACAGAGAGCTTC | TACR1 | DNA |
| 456 | NM_001058 | NM_001058_idx1390 | ACCATGACAGAGAGCTTCAGC | TACR1 | DNA |
| 457 | NM_001058 | NM_001058_idx1396 | ACAGAGAGCTTCAGCTTCTCC | TACR1 | DNA |
| 458 | NM_001058 | NM_001058_idx1497 | AAATTCCCTTCATCTGGAACC | TACR1 | DNA |
| 459 | NM_001058 | NM_001058_idx1514 | AACCATCAGAAACACCCTCAC | TACR1 | DNA |
| 460 | NM_001058 | NM_001058_idx1648 | AATCACTGAACTTTGCTGAGC | TACR1 | DNA |
| 461 | NM_001058 | NM_001058_idx1733 | ACTTTGGCTGCATGCGAGTGC | TACR1 | DNA |
| 462 | NM_015727 | NM_001058_idx290 | AACCAGCCTGGCAAATTGTCC | TACR1 | DNA |
| 463 | NM_015727 | NM_001058_idx303 | AATTGTCCTTTGGGCAGCTGC | TACR1 | DNA |
| 464 | NM_015727 | NM_001058_idx358 | AACGTGGTAGTGATGTGGATC | TACR1 | DNA |
| 465 | NM_015727 | NM_001058_idx463 | AATACAGTGGTGAACTTCACC | TACR1 | DNA |
| 466 | NM_015727 | NM_001058_idx475 | AACTTCACCTATGCTGTCCAC | TACR1 | DNA |
| 467 | NM_015727 | NM_001058_idx494 | ACAACGAATGGTACTACGGCC | TACR1 | DNA |
| 468 | NM_015727 | NM_001058_idx643 | ACAGCCACCAAAGTGGTCATC | TACR1 | DNA |
| 469 | NM_015727 | NM_001058_idx649 | ACCAAAGTGGTCATCTGTGTC | TACR1 | DNA |
| 470 | NM_015727 | NM_001058_idx652 | AAAGTGGTCATCTGTGTCATC | TACR1 | DNA |
| 471 | NM_015727 | NM_001058_idx713 | ACTCAACCACAGAGACCATGC | TACR1 | DNA |
| 472 | NM_015727 | NM_001058_idx797 | ACCACATCTGTGTGACTGTGC | TACR1 | DNA |
| 473 | NM_015727 | NM_001058_idx811 | ACTGTGCTGATCTACTTCCTC | TACR1 | DNA |
| 474 | NM_015727 | NM_001058_idx857 | ACACCGTAGTGGGAATCACAC | TACR1 | DNA |
| 475 | NM_015727 | NM_001058_idx874 | ACACTATGGGCCAGTGAGATC | TACR1 | DNA |
| 476 | NM_015727 | NM_001058_idx876 | ACTATGGGCCAGTGAGATCCC | TACR1 | DNA |
| 477 | NM_015727 | NM_001058_idx920 | ACGAGCAAGTCTCTGCCAAGC | TACR1 | DNA |
| 478 | NM_015727 | NM_001058_idx1025 | ACATCAACCCAGATCTCTACC | TACR1 | DNA |
| 479 | NM_015727 | NM_001058_idx1043 | ACCTGAAGAAGTTTATCCAGC | TACR1 | DNA |
| 480 | NM_015727 | NM_001058_idx1048 | AAGAAGTTTATCCAGCAGGTC | TACR1 | DNA |
| 481 | NM_015727 | NM_001058_idx1051 | AAGTTTATCCAGCAGGTCTAC | TACR1 | DNA |
| 482 | NM_015727 | NM_015727_idx1095 | ACCATCTACATACACAGTGGC | TACR1 | DNA |
| 483 | NM_015727 | NM_015727_idx1195 | AACTCAGCCTGGCTGATTATC | TACR1 | DNA |
| 484 | NM_001058 | NM_001058_idx290 | AACCAGCCTGGCAAATTGTCC | TACR1 | DNA |
| 485 | NM_001058 | NM_001058_idx303 | AATTGTCCTTTGGGCAGCTGC | TACR1 | DNA |
| 486 | NM_001058 | NM_001058_idx358 | AACGTGGTAGTGATGTGGATC | TACR1 | DNA |
| 487 | NM_001058 | NM_001058_idx463 | AATACAGTGGTGAACTTCACC | TACR1 | DNA |
| 488 | NM_001058 | NM_001058_idx475 | AACTTCACCTATGCTGTCCAC | TACR1 | DNA |
| 489 | NM_001058 | NM_001058_idx494 | ACAACGAATGGTACTACGGCC | TACR1 | DNA |
| 490 | NM_001058 | NM_001058_idx526 | AAGTTCCACAACTTCTTTCCC | TACR1 | DNA |
| 491 | NM_001058 | NM_001058_idx643 | ACAGCCACCAAAGTGGTCATC | TACR1 | DNA |
| 492 | NM_001058 | NM_001058_idx649 | ACCAAAGTGGTCATCTGTGTC | TACR1 | DNA |
| 493 | NM_001058 | NM_001058_idx652 | AAAGTGGTCATCTGTGTCATC | TACR1 | DNA |
| 494 | NM_001058 | NM_001058_idx713 | ACTCAACCACAGAGACCATGC | TACR1 | DNA |
| 495 | NM_001058 | NM_001058_idx797 | ACCACATCTGTGTGACTGTGC | TACR1 | DNA |
| 496 | NM_001058 | NM_001058_idx811 | ACTGTGCTGATCTACTTCCTC | TACR1 | DNA |
| 497 | NM_001058 | NM_001058_idx857 | ACACCGTAGTGGGAATCACAC | TACR1 | DNA |
| 498 | NM_001058 | NM_001058_idx874 | ACACTATGGGCCAGTGAGATC | TACR1 | DNA |
| 499 | NM_001058 | NM_001058_idx876 | ACTATGGGCCAGTGAGATCCC | TACR1 | DNA |
| 500 | NM_001058 | NM_001058_idx920 | ACGAGCAAGTCTCTGCCAAGC | TACR1 | DNA |
| 501 | NM_001058 | NM_001058_idx1025 | ACATCAACCCAGATCTCTACC | TACR1 | DNA |
| 502 | NM_001058 | NM_001058_idx1043 | ACCTGAAGAAGTTTATCCAGC | TACR1 | DNA |
| 503 | NM_001058 | NM_001058_idx1048 | AAGAAGTTTATCCAGCAGGTC | TACR1 | DNA |
| 504 | NM_001058 | NM_001058_idx1051 | AAGTTTATCCAGCAGGTCTAC | TACR1 | DNA |
| 505 | NM_001058 | NM_001058_idx1135 | AATGACAGGTTCCGTCTGGGC | TACR1 | DNA |
| 506 | NM_001058 | NM_001058_idx1214 | AAATGAAATCCACCCGGTATC | TACR1 | DNA |
| 507 | NM_001058 | NM_001058_idx1363 | AACTGCTCTTCACGAAGTGAC | TACR1 | DNA |
| 508 | NM_001058 | NM_001058_idx1377 | AAGTGACTCCAAGACCATGAC | TACR1 | DNA |
| 509 | NM_001058 | NM_001058_idx1387 | AAGACCATGACAGAGAGCTTC | TACR1 | DNA |
| 510 | NM_001058 | NM_001058_idx1390 | ACCATGACAGAGAGCTTCAGC | TACR1 | DNA |
| 511 | NM_001058 | NM_001058_idx1396 | ACAGAGAGCTTCAGCTTCTCC | TACR1 | DNA |
| 512 | NM_001058 | NM_001058_idx1497 | AAATTCCCTTCATCTGGAACC | TACR1 | DNA |
| 513 | NM_001058 | NM_001058_idx1514 | AACCATCAGAAACACCCTCAC | TACR1 | DNA |
| 514 | NM_001058 | NM_001058_idx1648 | AATCACTGAACTTTGCTGAGC | TACR1 | DNA |
| 515 | NM_001058 | NM_001058_idx1733 | ACTTTGGCTGCATGCGAGTGC | TACR1 | DNA |
| 516 | NM_015727 | NM_001058_idx290 | AACCAGCCTGGCAAATTGTCC | TACR1 | DNA |
| 517 | NM_015727 | NM_001058_idx303 | AATTGTCCTTTGGGCAGCTGC | TACR1 | DNA |
| 518 | NM_015727 | NM_001058_idx358 | AACGTGGTAGTGATGTGGATC | TACR1 | DNA |
| 519 | NM_015727 | NM_001058_idx463 | AATACAGTGGTGAACTTCACC | TACR1 | DNA |
| 520 | NM_015727 | NM_001058_idx475 | AACTTCACCTATGCTGTCCAC | TACR1 | DNA |
| 521 | NM_015727 | NM_001058_idx494 | ACAACGAATGGTACTACGGCC | TACR1 | DNA |
| 522 | NM_015727 | NM_001058_idx643 | ACAGCCACCAAAGTGGTCATC | TACR1 | DNA |
| 523 | NM_015727 | NM_001058_idx649 | ACCAAAGTGGTCATCTGTGTC | TACR1 | DNA |
| 524 | NM_015727 | NM_001058_idx652 | AAAGTGGTCATCTGTGTCATC | TACR1 | DNA |
| 525 | NM_015727 | NM_001058_idx713 | ACTCAACCACAGAGACCATGC | TACR1 | DNA |
| 526 | NM_015727 | NM_001058_idx797 | ACCACATCTGTGTGACTGTGC | TACR1 | DNA |
| 527 | NM_015727 | NM_001058_idx811 | ACTGTGCTGATCTACTTCCTC | TACR1 | DNA |
| 528 | NM_015727 | NM_001058_idx857 | ACACCGTAGTGGGAATCACAC | TACR1 | DNA |
| 529 | NM_015727 | NM_001058_idx874 | ACACTATGGGCCAGTGAGATC | TACR1 | DNA |
| 530 | NM_015727 | NM_001058_idx876 | ACTATGGGCCAGTGAGATCCC | TACR1 | DNA |
| 531 | NM_015727 | NM_001058_idx920 | ACGAGCAAGTCTCTGCCAAGC | TACR1 | DNA |
| 532 | NM_015727 | NM_001058_idx1025 | ACATCAACCCAGATCTCTACC | TACR1 | DNA |
| 533 | NM_015727 | NM_001058_idx1043 | ACCTGAAGAAGTTTATCCAGC | TACR1 | DNA |
| 534 | NM_015727 | NM_001058_idx1048 | AAGAAGTTTATCCAGCAGGTC | TACR1 | DNA |
| 535 | NM_015727 | NM_001058_idx1051 | AAGTTTATCCAGCAGGTCTAC | TACR1 | DNA |
| 536 | NM_015727 | NM_015727_idx1095 | ACCATCTACATACACAGTGGC | TACR1 | DNA |
| 537 | NM_015727 | NM_015727_idx1195 | AACTCAGCCTGGCTGATTATC | TACR1 | DNA |
| 538 | | N-term | MALNDCFLLNLEVDHFMHCNI | GRPR | Protein |
| | | | SSHSADLPVNDDWSHPG | | |
| 539 | | TM1 | ILYVIPAVYGVIILIGLIGNITL | GRPR | Protein |
| 540 | | IL1 | IKIFCTVKSMRN | GRPR | Protein |
| 541 | | TM2 | VPNLFISSLALGDLLLLITCAPV | GRPR | Protein |
| 542 | | EL1 | DASRYLADRWLFGRIGCKL | GRPR | Protein |
| 543 | | TM3 | IPFIQLTSVGVSVFTLTALSA | GRPR | Protein |
| 544 | | IL2 | DRYKAIVRPMDIQASHALMK | GRPR | Protein |
| 545 | | TM4 | ICLKAAFIWIISMLLAIPEAVFS | GRPR | Protein |
| 546 | | EL2 | DLHPFHEESTNQTFISCAPYPHS | GRPR | Protein |
| | | | NELHPKIH | | |
| 547 | | TM5 | SMASFLVFYVIPLSIISVYYYFI | GRPR | Protein |
| 548 | | IL3 | AKNLIQSAYNLPVEGNIHVKKQ | GRPR | Protein |
| | | | IESRKR | | |
| 549 | | TM6 | LAKTVLVFVGLFAFCWLPNHVIY | GRPR | Protein |
| 550 | | EL3 | LYRSYHYSEVDTSMLHFVT | GRPR | Protein |
| 551 | | TM7 | SICARLLAFTNSCVNPFALYLLS | GRPR | Protein |
| 552 | | C-term | KSFRKQFNTQLLCCQPGLIIRSH | GRPR | Protein |
| | | | STGRSTTCMTSLKSTNPSVATFS | | |
| | | | LINGNICHERYV | | |
| 553 | | N-term | MVFLSGNASDSSNCTQPPAPVN | ADRA1A | Protein |
| | | | ISKAI | | |
| 554 | | TM1 | LLGVILGGLILFGVLGNILVILS | ADRA1A | Protein |
| 555 | | IL1 | VACHRHLHSVTH | ADRA1A | Protein |
| 556 | | TM2 | YYIVNLAVADLLLTSTVLPFSAI | ADRA1A | Protein |
| 557 | | EL1 | FEVLGYWAFGRVFC | ADRA1A | Protein |
| 558 | | TM3 | NIWAAVDVLCCTASIMGLCIISI | ADRA1A | Protein |
| 559 | | IL2 | DRYIGVSYPLRYPTIVTQRR | ADRA1A | Protein |
| 560 | | TM4 | GLMALLCVWALSLVISIGPLFGW | ADRA1A | Protein |
| 561 | | EL2 | RQPAPEDETICQINEEPGY | ADRA1A | Protein |
| 562 | | TM5 | VLFSALGSFYLPLAIILVMYCRV | ADRA1A | Protein |
| 563 | | IL3 | YVVAKRESRGLKSGLKTDKSD | ADRA1A | Protein |
| | | | SEQVTLRIHRKNAPAGGSGMAS | | |
| | | | AKTKTHFSVRLLKFSREKKAAK | | |
| 564 | | TM6 | TLGIVVGCFVLCWLPFFLVMPIG | ADRA1A | Protein |
| 565 | | EL3 | SFFPDFKPSETVFK | ADRA1A | Protein |
| 566 | | TM7 | IVFWLGYLNSCINPIIYPCS | ADRA1A | Protein |
| 567 | | C-term | SQEFKKAFQNVLRLIQCLRRKQS | ADRA1A | Protein |
| | | | SKHALGYTLHPPSQAVEGQHK | | |
| | | | DMVRIPVGSRETFYRISKTDGV | | |
| | | | CEWKFFSSMPRGSARITVSKDQ | | |
| | | | SSCTTARGHTPMT | | |
| 568 | | N-term | MDNVLPVDSDLSPNISTNTSEP | TACR1 | Protein |
| | | | NQFVQPAWQIVL | | |
| 569 | | TM1 | WAAAYTVIVVTSVVGNVVVMWII | TACR1 | Protein |
| 570 | | IL1 | LAHKRMRTVTNY | TACR1 | Protein |
| 571 | | TM2 | FLVNLAFAEASMAAFNTVVNFTY | TACR1 | Protein |
| 572 | | EL1 | AVHNEWYYGLFYCK | TACR1 | Protein |
| 573 | | TM3 | FHNFFPIAAVFASIYSMTAVAF | TACR1 | Protein |
| 574 | | IL2 | DRYMAIIHPLQPRLSATATK | TACR1 | Protein |
| 575 | | TM4 | VVICVIWVLALLLAFPQGYY | TACR1 | Protein |
| 576 | | EL2 | STTETMPSRVVCMIEWPEHPNK | TACR1 | Protein |
| | | | IYEKVYHICVTV | | |
| 577 | | TM5 | LIYFLPLLVIGYAYTVVGITLWA | TACR1 | Protein |
| 578 | | IL3 | SEIPGDSSDRYHEQVSAKRK | TACR1 | Protein |
| 579 | | TM6 | VVKMMIVVVCTFAICWLPFHIFF | TACR1 | Protein |
| 580 | | EL3 | LLPYINPDLYLKKF | TACR1 | Protein |
| 581 | | TM7 | IQQVYLAIMWLAMSSTMYNPIIY | TACR1 | Protein |
| 582 | | C-term | CCLNPR | TACR1 | Protein |

### EXAMPLE 2

### Expression Of GPCRs In The Human Brain

Upon identification of a modulator of APP processing, it is important to evaluate whether the modulator is expressed in the tissue and the cells of interest. This can be achieved by measuring the RNA and/or protein levels in the tissue and cells. In recent years, RNA levels are being quantified through real time PCR technologies, whereby the RNA is first transcribed to cDNA and then the amplification of the cDNA of interest is monitored during a PCR reaction. The amplification plot and the resulting Ct value are indicators for the amount of RNA present in the sample. Ct values are determined in the presence or absence of the reverse transcriptase step (+RT versus −RT). An amplification signal in the −RT condition indicates the occurrence of non-specific PCR products originating from the genomic DNA. If the +RT Ct value is 3 Ct values higher than the —RT Ct value, then the investigated RNA is present in the sample.

To assess whether the identified GPCRs are expressed in the human brain, real time PCR with specific primers for each GPCR of the invention is performed on human total brain, human cerebral cortex, and human hippocampal total RNA (BD Biosciences)(see Table 3). In addition, to assess the neuronal expression, the expression analysis was also performed on RNA samples prepared from mouse or rat primary neuron cell cultures using PCR primers for the murine or rat homolog of the polypeptide of the invention.

TABLE 3

Primers used in the quantitative real time
PCR analysis for GPCR-expression.

| | | | | SEQ |
|---|---|---|---|---|
| | | | | ID |
| Gene | Species | Primer name | Sequence | NO. |
| GRPR | H. Sapiens | GRPR_Hs_For | CATGCTGCTGGCCATITCC | 583 |
| | H. Sapiens | GRPR_Hs_Rev | AGGTCTGGTTGGTGCTTTCCT | 584 |
| GRPR | Mus Musculus | GRPR_Mm_For | TGTCTTCACACTTACGGCACTGT | 585 |
| | Mus Musculus | GRPR_Mm_Rev | GCATGGGATGCCTGGATATC | 586 |
| ADRA1A | H. Sapiens | ADRA1A_Hs_For | CAAAACGCTGGGCATCGT | 587 |
| | H. Sapiens | ADRA1A_Hs_Rev | GACCCAATGGGCATGACTAAGA | 588 |
| ADRA1A | Mus Musculus | ADRA1A_Mm_For | TGCCCATTGGGTCCTTCTT | 589 |
| | Mus Musculus | ADRA1A_Mm_Rev | GGTACCCAAGCCAAAATACTATTTTG | 590 |
| TACR1 | H. Sapiens | | Hs00185530_m1 (Applied Biosystems) | 591 |
| | H. Sapiens | | Hs00185530_m1 (Applied Biosystems) | 592 |

Forty ng of RNA are reverse-transcribed to DNA using the MultiScribe Reverse Transcriptase (50 U/μl) enzyme (Applied BioSystems). The resulting cDNA is amplified with AmpliTaq Gold DNA polymerase (Applied BioSystems) during 40 cycles using an ABI PRISM® 7000 Sequence Detection System. Amplification of the transcript is detected via SybrGreen which results in a fluorescent signal upon intercalation in double stranded DNA.

Total RNA isolated from mouse primary neurons and human total brain, cerebral cortex and hippocampal are analyzed for the presence of the GPCR transcripts via quantitative real time PCR. The Ct values for the genes listed in Table 2 indicate that they are detected in all RNA samples (Table 4).

To gain more insight into the specific cellular expression, immunohistochemistry (protein level) and/or in situ hybridization (RNA level) are carried out on sections from human normal and Alzheimer's brain hippocampal, cortical and subcortical structures. These results indicate whether expression occurs in neurons, microglia cells, or astrocytes. The comparison of diseased tissue with healthy tissue indicates whether the GPCR is expressed in the diseased tissue and whether its expression level is changed compared to the non-pathological situation.

TABLE 4

| Total RNA isolated from human brain, | | | |
|---|---|---|---|
| human cerebral cortex, human hippocampus | | | |
| and mouse or rat primary | | Ct values obtained | |
| hippocampal neuronal cultures is tested for | | during quantitative | |
| the presence of the respective RNA | | real time PCR | |
| via quantitative realtime PCR. | | Ct | |
| Gene | Tissue | RT+ | RT− |
| GRPR | Human Brain Hippocampus | 25, 25 | 40 |
| | Human Brain Cerebral Cortex | 25, 04 | 40 |
| | Mus Musculus Primary Hippocampal | 30, 46 | 40 |
| | Neurons | | |
| ADRA1A | Human Brain Hippocampus | 22, 26 | 40 |
| | Human Brain Cerebral Cortex | 21, 58 | 38, 94 |
| | Mus Musculus Primary Hippocampal | 29, 54 | 40 |
| | Neurons | | |
| TACR1 | Human Brain Hippocampus | 29, 96 | 40 |
| | Human Brain Cerebral Cortex | 29, 45 | 40 |

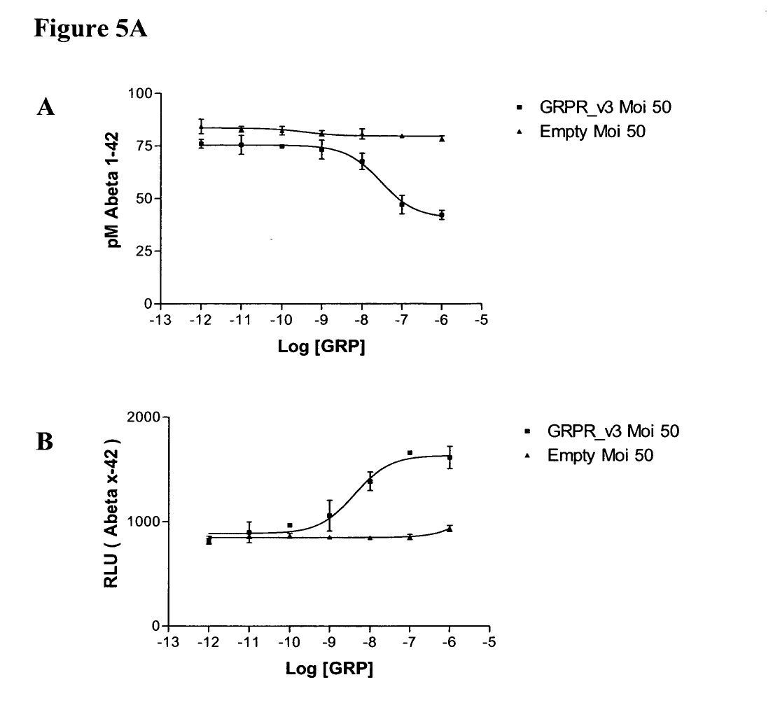

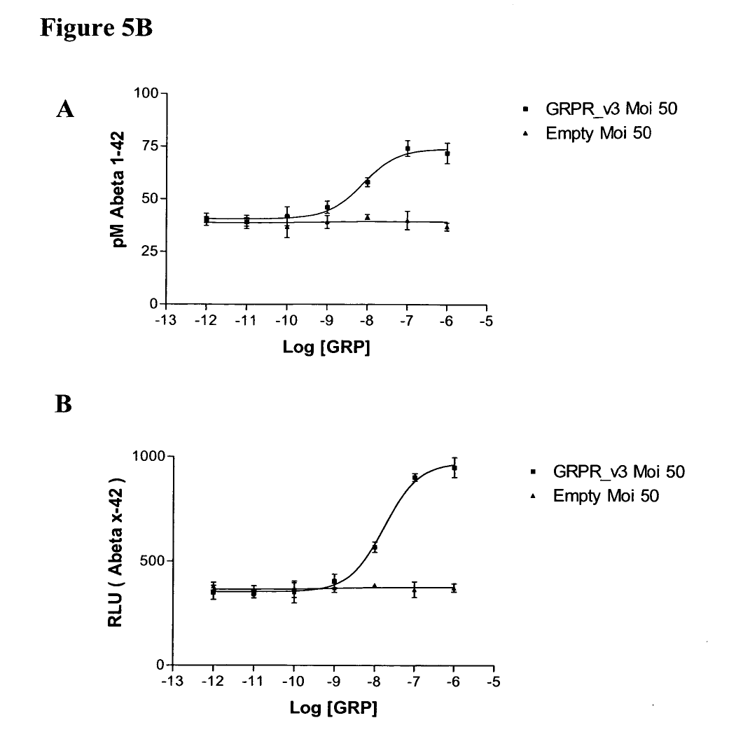

The stimulatory effect of GRPR, ADRA1A and TACR1 is confirmed upon re-screening of the viruses with a known titer (viral particles/ml), as determined by quantitative real time PCR. GRPR, ADRA1A and TACR1 virus is infected at MOIs ranging from 2 to 1250 and the experiment is performed as described above. In addition, the effect of GRPR, ADR1A and TACR1 on amyloid beta 1-42 and x-42 levels are checked under similar conditions as above (FIGS. 5-7). The respective ELISAs are performed as described above, except that the following antibodies were used: for the amyloid beta 1-40 ELISA, the capture and detection antibody are respectively JRF/cAbeta40/10 and JRF/AbetaN/25-HRP (obtained from M Mercken, Johnson and Johnson Pharmaceutical Research and Development, B-2340 Beerse, Belgium), for the amyloid beta 11-42 ELISA, the capture and detection antibody are respectively JRF/cAbeta42/26 and JRF/hAb11/1 (obtained from M Mercken, Johnson and Johnson Pharmaceutical Research and Development, B-2340 Beerse, Belgium), for the amyloid beta x-42 ELISA (x ranges from I-17), the capture and detection antibody are respectively JRF/cAbeta42/26 and 4G8-HRP (obtained respectively from M Mercken, Johnson and Johnson Pharmaceutical Research and Development, B-2340 Beerse, Belgium and from Signet, USA) while for the amyloid beta 1-y ELISA (y ranges from 24-42) the capture and detection antibodies are JRF/AbetaN/25 and 4G8-HRP, respectively (obtained respectively from M Mercken, Johnson and Johnson Pharmaceutical Research and Development, B-2340 Beerse, Belgium and from Signet, USA). The amyloid beta 1-y ELISA is used for the detection of amyloid peptides with a variable C-terminus (amyloid beta 1-37; 1-38; 1-39; 1-40; 1-42).

### EXAMPLE 3

### GRPR Agonist Validation (A) Hek293 APPwt and (B) SYSY APPwt Cells

Agonists for GRPR are tested to evaluate whether inducing GRPR activity results in an reduction of the amyloid beta 1-42 levels. For this, Hek293 APPwt and SH-SY5Y APPwt cells are seeded in 96 well plates at a cell density of 30,000 cells/well and are infected respectively with Ad5/empty, and Ad5/GRPR_v3 over a 24 hours period at a MOI of 50. Viruses are washed away and fresh medium containing increasing amounts of agonist (GRP; gastrin related peptide) is added to the cells. 24 h later, the conditioned medium is assayed in the amyloid beta 1-42 and amyloid beta x-42 ELISA as described in EXAMPLE 1. FIGS. 5A and 5B show the changes in amyloid beta 1-42 and amyloid beta x-42 levels as a function of concentration.

### EXAMPLE 4

### TACR1 Specific Agonist and Antagonist Validation in (A) Hek293 APPwt and (B) SH-SY5y APPwt Cells

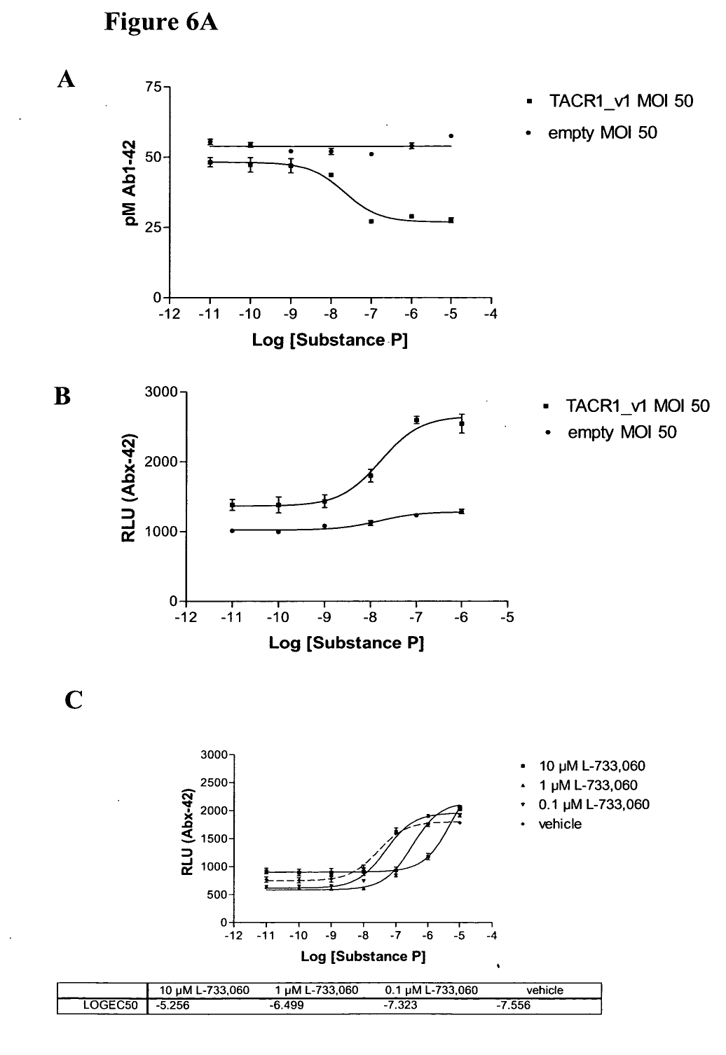

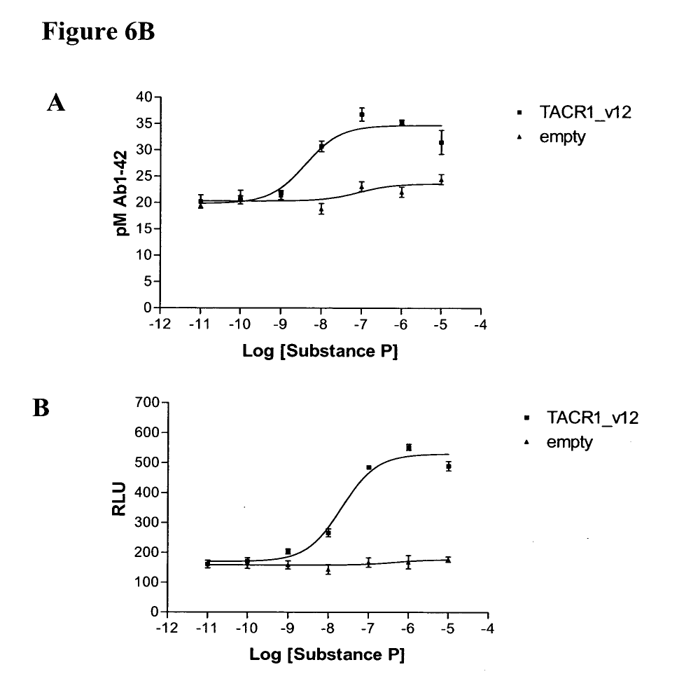

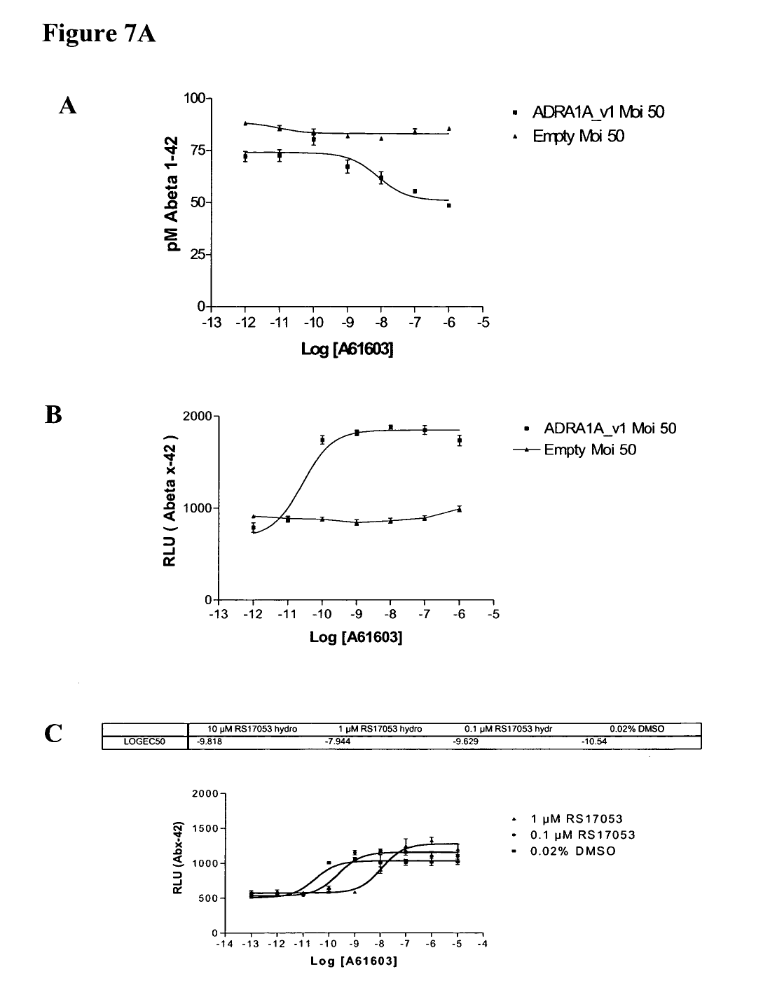

Agonists for TACR1 are tested to evaluate whether inducing TACR1 activity increases or decreases amyloid beta 1-42 levels. Hek293 APPwt cells and SH-SY5Y APPwt cells are infected respectively with Ad5/empty, Ad5/TACR1_v1, or Ad5/TACR1_v12 over a 24 hours period. Viruses are washed away and fresh medium containing increasing amounts of agonist (substance P) is added to the cells. 24 h later, the conditioned medium is assayed in the amyloid beta 1-42 and amyloid beta x-42 ELISA as described in EXAMPLE 1. As shown in FIG. 6A, substance P decreased the amount of amyloid beta 1-42 secreted in the Hek293 APPwt cells medium in a concentration dependent manner. In contrast, as shown in FIG. 6A-B and FIG. 6B, substance P increased the amount of amyloid beta x-42 secreted in the Hek293 APPwt cells medium, as well as the amount of amyloid beta 1-42 and amyloid beta x-42 secreted in the SH-SY5Y APPwt cells medium, all in a concentration dependent manner,

An antagonist for TACR1 is tested to evaluate whether inhibiting TACR1 results in a decrease of the amyloid beta x-42 levels. Hek293 APPwt cells are infected with Ad5/TACR1_v1 over a 24 hours period. Viruses are washed away and fresh medium containing increasing amounts of agonist (substance P) in the absence and presence of fixed (0.1, 1 and 10 μM) concentrations of L-733,060 hydrochloride is added to the cells. 24 h later, the conditioned medium is assayed in the amyloid beta x-42 ELISA as described in EXAMPLE 1. As shown in FIG. 6A-C, the observed Substance P EC50 values are increased with increasing concentration of the antagonist, L-733,060 hydrochloride, which reduces the amount of amyloid beta x-42 secreted in the medium in a concentration dependent manner.

### EXAMPLE 5

### ADRA1A Agonists and Antagonist Validation in (A) Hek293 APPwt and (B) SH-SY5y APPwt Cells

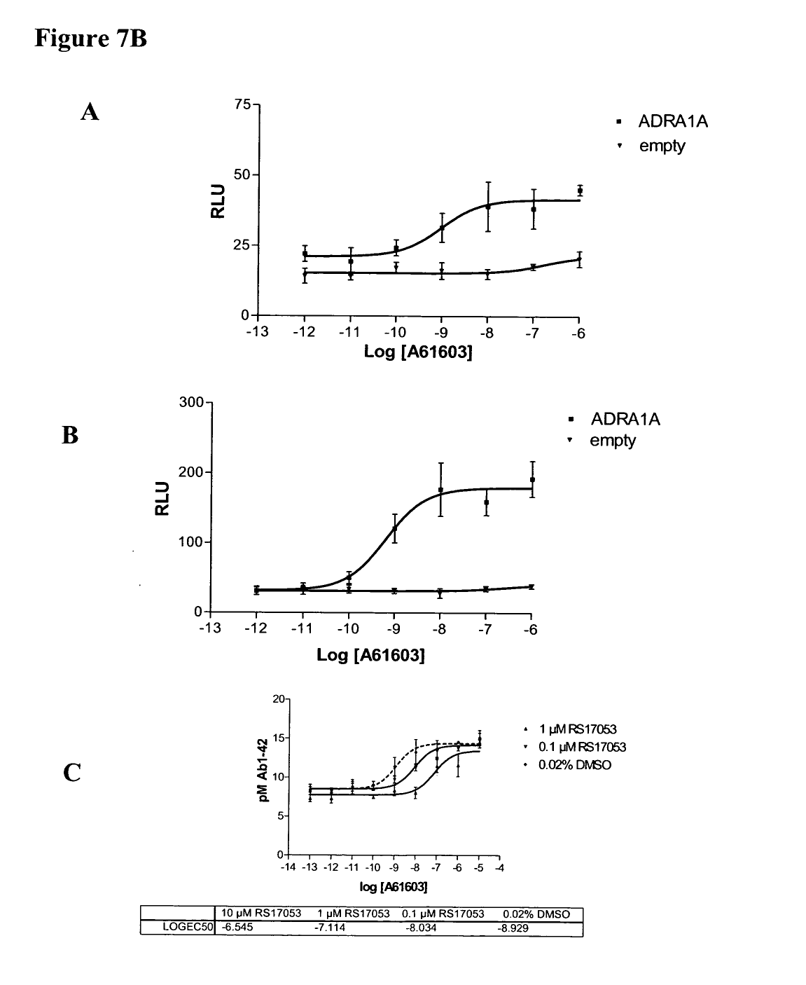

Agonists for ADRA1A are tested to evaluate whether inducing ADRA1A activity results in a decrease of amyloid beta 1-42 levels. Hek293 APPwt cells and SH-SY5Y APPwt cells are infected respectively with Ad5/empty and Ad5/ADRA1A_v1 over a 24-hour period. Viruses are washed away and fresh medium containing increasing amounts of agonist (A61603) added to the cells. 24 h later, the conditioned medium is assayed in the amyloid beta 1-42 and amyloid beta x-42 ELISA as described in EXAMPLE 1. As shown in FIG. 7A, A61603 decreased the amount of amyloid beta 1-42 secreted in the Hek293 APPwt cells medium in a concentration dependent manner. In contrast, as shown in FIG. 7A-B and FIG. 7B, A61603 increased the amount of amyloid beta x-42 secreted in the Hek293 APPwt cells medium, as well as the amount of amyloid beta 1-42 and amyloid beta x-42 secreted in the SH-SY5Y APPwt cells medium, all in a concentration dependent manner.

An antagonist for ADRA1A is tested to evaluate whether inhibiting the ADRA1A receptor results in a decrease of the amyloid beta 1-42 levels. SH-SY5Y APPwt cells and Hek293 APPwt cells are infected with Ad5/ADRAlA_v1 over a 24 hours period. Viruses are washed away and fresh medium containing increasing amounts of agonist (A61603) in the absence and presence of fixed (0.1 and 1 μM) concentrations of RS-17053 hydrochloride is added to the cells. 24 h later, the conditioned medium is assayed in the amyloid beta x-42 ELISA (Hek293 APPwt cells) and amyloid beta 1-42 ELISA (SH-SY5Y APPwt cells) as described in EXAMPLE 1. As shown in FIG. 7A-C, RS-17053 hydrochloride reduced the amount of amyloid beta x-42 secreted in Hek293 APPwt cell medium, and amyloid beta 1-42 secreted in the SH-SY5Y APPwt cell medium, both in a concentration dependent manner. The observed EC50 values increased with increasing concentration of the antagonist.

### EXAMPLE 6

### Amyloid Beta Peptide Reduction Via Knock Down of GPCR Expression

The effect of an antagonist may be mimicked through the use of siRNA-based strategies, which result in decreased expression levels of the targeted protein. For example, transfection with shRNA including a 17-25 nt mRNA targeting sequence coding for a portion of GRPR and TACR1 reduces amyloid beta 1-42.

The knock-down assay is performed as follows: Cells are seeded in collagen-coated plates in 50 μl, at a cell density of 15000 cells/well (384 well plate) in DMEM 10% FBS containing 1 μM 9 cis-retinoic acid. 48 h later, 10 μl of fresh DMEM 10% FBS containing 1 μM 9 cis-retinoic acid is added and the cells are infected with adenovirus containing the knock down sequences at an MOI ranging from 50 to 1250 and an adenovirus harboring the APPsw cDNA at an MOI of 500. The following day, the virus is washed away with 80 μl DMEM 10% FBS containing 1 μM 9 cis-retinoic acid and 80 μl DMEM 10% FBS containing 1 μM 9 cis-retinoic acid is added to the cells. After 96 h, the medium is refreshed with 80 μl DMEM 10% FBS containing 1 μM 9 cis-retinoic acid and 0.025 mM Hepes. Amyloid beta peptides are allowed to accumulate during 48 h. The amyloid beta 1-42 ELISA is performed as described in EXAMPLE 1.

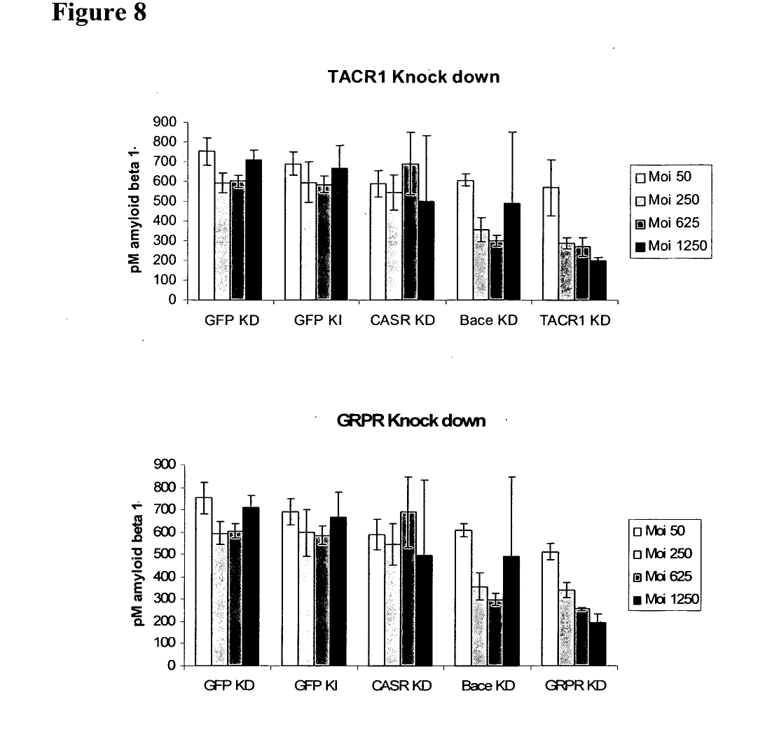

Adenoviruses carrying knock down sequences targeting TACR1 and GRPR reduce amyloid beta 1-42 levels compared to adenoviruses either over expressing eGFP or containing knock down sequences targeting eGFP and CASR (FIG. 8). The reduction in amyloid beta 1-42 levels is similar as observed with a knock down sequence targeting BACEI. These data show that TACR1 and GRPR modulate amyloid beta 1-42 levels.

### EXAMPLE 7

### Amyloid Beta Production In Rat Primary Neuronal Cells

To investigate whether GRPR, ADR1A and TACR1 affects amyloid beta production in a primary neuron, human or rat primary hippocampal or cortical neurons are transduced with adenovirus containing the GRPR, ADR1A and TACR1 cDNA. Amyloid beta levels are determined by ELISA (see EXAMPLE 1). Since rodent APP genes carry a number of mutations in APP compared to the human sequence, they produce less amyloid beta 1-40 and 1-42. To achieve higher amyloid beta levels, the neurons are co-transduced with adenovirus containing cDNA for GRPR, ADRIA and TACR1 and with cDNA coding for human wild type APP or human Swedish mutant APP (which enhances amyloid beta production).

Rat primary neuron cultures are prepared from brain of E18-E19-day-old fetal Sprague Dawley rats according to Goslin and Banker (Culturing Nerve cells, second edition, 1998 ISBN 0-262-02438-1). Single cell suspensions obtained from the hippocampus or cortices are prepared. The number of viable cells is determined and plated on poly-L-lysine-coated plastic 96-well plates in minimal essential medium (MEM) supplemented with 10% horse serum. The cells are seeded at a density of 50,000 cells per well (i.e. about 166,000 cells/cm²). After 3-4 h, culture medium is replaced by 160 μl serum-free neurobasal medium with B27 supplement (GIBCO BRL). Cytosine arabinoside (5 μM) is added 24 h after plating to prevent non-neuronal (glial) cell proliferation.

Neurons are used at day 5 after plating. Before adenoviral transduction, 150 μl conditioned medium of these cultures is transferred to the corresponding wells in an empty 96-well plate and 50 μl of the conditioned medium is returned to the cells. The remaining 100 μl/well is stored at 37° C. and 5% CO₂. Hippocampal primary neuron cultures are infected with the crude lysate of Ad5C09Att00/A011200-GRPR, -ADR1A and -TACR1_v3, Ad5C09Att00/A010801-LacZ_v1, Ad5C09Att00/A010800-eGFP_v1 and Ad5C09Att00/A010800-luc_v17 viruses containing the cDNA of GRPR, ADR1A and TACR1, LacZ, eGFP and luciferase respectively at different MOIs, ranging from 250 to 2000. In addition the cells are co-infected with the purified adenovirus Ad5C01Att01/A010800 APP_v6 expressing human wild type APP695 at an MOI of 2000. Sixteen to twenty-four hours after transduction, virus is removed and cultures are washed with 100 μl pre-warmed fresh neurobasal medium. After removal of the wash solution, new medium, containing 50 μl of the stored conditioned medium and 50 μl of fresh neurobasal medium, is transferred to the corresponding cells. Medium is harvested after 48 and 72 hours. The cell number in the wells is determined by assessing the ATP levels. Amyloid beta concentration is determined by amyloid beta 1-42 specific ELISA (see EXAMPLE 1). Amyloid beta 1-42 levels are normalized for cell number.

### EXAMPLE 10

### Ligand Screens For GPCRs

Reporter Gene Screen.

Mammalian cells such as Hek293 or CHO-K1 cells are either stably transfected with a plasmid harboring the luciferase gene under the control of a cAMP dependent promoter (CRE elements) or transduced with an adenovirus harboring a luciferase gene under the control of a cAMP dependent promoter. In addition reporter constructs can be used with the luciferase gene under the control of a Ca²⁺ dependent promoter (NF-AT elements) or a promoter that is controlled by activated NF-κB. These cells, expressing the reporter construct, are then transduced with an adenovirus harboring the cDNA of GRPR, ADRA1A or TACR1. Forty (40) hours after transduction the cells are treated with the following:
a) an agonist for the receptor (e.g. GRP, Substance P or A61603) and screened against a large collection of reference compounds comprising peptides (LOPAP, Sigma Aldrich), lipids (Biomol, TimTech), carbohydrates (Specs), natural compounds (Specs, TimTech), small chemical compounds (Tocris), commercially available screening libraries, and compounds that have been demonstrated to have binding affinity for a polypeptide comprising an amino acid sequence selected from the group consisting of SEQ ID NO: 44, 50, 51, 56, and 538-582; or
b) a large collection of reference compounds comprising peptides (LOPAP, Sigma Aldrich), lipids (Biomol, TimTech), carbohydrates (Specs), natural compounds (Specs, TimTech), small chemical compounds (Tocris), commercially available screening libraries, and compounds that have been demonstrated to have binding affinity for a polypeptide comprising an amino acid sequence selected from the group consisting of SEQ ID NO: 44, 50, 51, 56, and 538-582, including selective tachykinin NK1 receptor antagonist, subtype selective a1A-adrenoceptor antagonist, and GRP receptor antagonist compounds identified in Table 8 below, and salts, hydrates, or solvates, only, as GRPR, ADRA1A and TACR1 are considered to be a constitutively active GPCR.

Compounds, which decrease the agonist induced increase in luciferase activity or the constitutive activity, are considered to be antagonists or inverse agonists for GRPR, ADRA1A or TACR1. These compounds are screened again for verification and screened against their effect on secreted amyloid beta peptide levels. The compounds are also screened to verify binding to the GPCR. The binding, amyloid-beta peptide and reporter activity assays can be performed in essentially any order to screen compounds.

In addition, cells expressing the NF-AT reporter gene can be transduced with an adenovirus harboring the cDNA encoding the α-subunit of G₁₅ or chimerical Gα subunits. G₁₅ is a promiscuous G protein of the G_{q} class that couples to many different GPCRs and as such re-directs their signaling towards the release of intracellular Ca²⁺ stores. The chimerical G alpha subunits are members of the Gₛ and G_{i/o} family by which the last 5 C-S&L terminal residues are replaced by those of G_{αq}, these chimerical G-proteins also redirect cAMP signaling to Ca²⁺ signaling.

FLIPR screen.

Mammalian cells such as Hek293 or CHO-KI cells are stably transfected with an expression plasmid construct harboring the cDNA of GRPR, ADRA1A or TACR1. Cells are seeded, grown, and selected until sufficient stable cells can be obtained. Cells are loaded with a Ca²⁺ dependent fluorophore such as Fura3 or Fura4. After washing away the excess of fluorophore the cells are screened against a large collection of reference compounds comprising peptides (LOPAP, Sigma Aldrich), lipids (Biomol, TimTech), 10 carbohydrates (Specs), natural compounds (Specs, TimTech), small chemical compounds (Tocris), commercially available screening libraries, and compounds that have been demonstrated to have binding affinity for a polypeptide comprising an amino acid sequence selected from the group consisting of SEQ ID NO: 44, 50, 51, 56, and 538-582, including the selective tachykinin NK1 receptor antagonists, subtype selective a1A-adrenoceptor antagonists, and GRP receptor antagonists identified in Table 8 below, by simultaneously adding an agonist (alternatively no agonist need be added if the constitutive activity of the receptor is used) and a compound to the cells. Activation of the receptor is measured as an almost instantaneously increase in fluorescence due to the interaction of the fluorophore and the Ca²⁺ that is released. Compounds that reduce or inhibit the agonist induced increase in fluorescence (or constitutive fluorescence) are considered to be antagonists or inverse agonists for the receptor they are screened against. These compounds are screened again to measure the amount of secreted amyloid beta peptide as well as binding to GRPR, ADRA1A or TACR1.

AequoScreen.

CHO cells, stably expressing Apoaequorin are stably transfected with a plasmid construct harboring the cDNA of GRPR, ADRA1A or TACR1. Cells are seeded, grown, and selected until sufficient stable cells can be obtained. The cells are loaded with coelenterazine, a cofactor for apoaequorin. Upon receptor activation intracellular Ca²⁺ stores are emptied and the aequorin will react with the coelenterazine in a light emitting process. The emitted light is a measure for receptor activation. The CHO, stable expressing both the apoaequorin and the receptor are screened against a large collection of reference compounds comprising peptides (LOPAP, Sigma Aldrich), lipids (Biomol, TimTech), carbohydrates (Specs), natural compounds (Specs, TimTech), small chemical compounds (Tocris), commercially available screening libraries, and compounds that have been demonstrated to have binding affinity for a polypeptide comprising an amino acid sequence selected from the group consisting of SEQ ID NO: 44, 50, 51, 56, and 538-582, including the selective tachykinin NK1 receptor antagonists, subtype selective a1A-adrenoceptor antagonists, and GRP receptor antagonists identified in Table 8 below, its salts, hydrates, or solvates, by simultaneously adding an agonist (alternatively no agonist need be added if the constitutive activity of the receptor is used) and a compound to the cells. Activation of the receptor is measured as an almost instantaneously light flash due to the interaction of the apoaequorin, coelenterazine, and the Ca²⁺ that is released. Compounds that reduce or inhibit the agonist induced increase in light or the constitutive activity are considered to be antagonists or inverse agonists for the receptor they are screened against. These compounds are screened again to measure the amount of secreted amyloid beta peptide as well as binding to GRPR, ADRA1A or TACR1.

In addition, CHO cells stable expressing the apoaequorin gene are stably transfected with a plasmid construct harboring the cDNA encoding the α-subunit of G₁₅ or chimerical G_{α} subunits. G₁₅ is a promiscuous G protein of the G_{q} class that couples to many different GPCRs and as such redirects their signaling towards the release of intracellular Ca²⁺ stores. The chimerical G alpha subunits are members of the Gₛ and G_{i/o} family by which the last 5 C-terminal residues are replaced by those of G_{αq}, these chimerical G-proteins also redirect cAMP signaling to Ca²⁺ signaling.

Screening for Compounds that Bind to the GPCR Polypeptides (Displacement Experiment)

Compounds are screened for binding to the GRPR, ADRA1A or TACR1 polypeptides. The affinity of the compounds to the polypeptides is determined in a displacement experiment. In brief, the GPCR polypeptides are incubated with a labeled (radiolabeled, fluorescent labeled) ligand that is known to bind to the polypeptide (e.g., GRP, Substance P or A61603) and with an unlabeled compound. The displacement of the labeled ligand from the polypeptide is determined by measuring the amount of labeled ligand that is still associated with the polypeptide. The amount associated with the polypeptide is plotted against the concentration of the compound to calculate IC₅₀ values. This value reflects the binding affinity of the compound to its target, i.e. the GRPR, ADRA1A or TACR1 polypeptides. Strong binders have an IC₅₀ in the nanomolar and even picomolar range. Compounds that have an IC₅₀ of at least 10 micromol or better (nmol to pmol) are applied in beta amyloid secretion assay to check for their effect on the beta amyloid secretion and processing. The GRPR, ADRA1A or TACR1 polypeptides can be prepared in a number of ways depending on whether the assay are run on cells, cell fractions or biochemically, on purified proteins.

Screening for Compounds that Bind to GRPR, ADRA1A or TACR1 (Generic GPCR Screening Assay)

When a G protein receptor becomes constitutively active, it binds to a G protein (G_{q}, Gₛ, Gᵢ, Gₒ) and stimulates the binding of GTP to the G protein. The G protein alpha subunit then acts as a GTPase and slowly hydrolyses the GTP to GDP, whereby the receptor, under normal conditions, becomes deactivated. However, constitutively activated receptors continue to exchange GDP to GTP. A non-hydrolyzable analog of GTP, [³⁵S]GTPγS, can be used to monitor enhanced binding to membranes which express constitutively activated receptors. It is reported that [³⁵S]GTPγS can be used to monitor G protein coupling to membranes in the absence and presence of ligand. Moreover, a preferred approach is the use of a GPCR-G protein fusion protein. The strategy to generate a GRPR-, ADRA1A- and/or TACR1-G protein fusion protein is well known for those known in the art. Membranes expressing GRPR-, ADRA1A- and TACR1-G protein fusion protein are prepared for use in the direct identification of candidate compounds such as inverse agonist. Homogenized membranes with GRPR-, ADRA1A- and TACR1-G protein fusion protein are transferred in a 96-well plate. A pin-tool is used to transfer a candidate compound in each well plus [³⁵S]GTPγS, followed by incubation on a shaker for 60 minutes at room temperature. The assay is stopped by spinning of the plates at 4000 RPM for 15 minutes at 22° C. The plates are then aspirated and radioactivity is then read.

Receptor Ligand Binding Study On Cell Surface

The receptor is expressed in mammalian cells (Hek293, CHO, COS7) by adenoviral transducing the cells (see U.S. Pat. No. 6,340,595). The cells are incubated with both labeled ligand (iodinated, tritiated, or fluorescent) and the unlabeled compound at various concentrations, ranging from 10 μM to 10 μM (3 hours at 4° C.: 25 mM HEPES, 140 mM NaCl, 1 mM CaCl₂, 5 mM MgCl₂ and 0.2% BSA, adjusted to pH 7.4). Reactions mixtures are aspirated onto PEI-treated GF/B glass filters using a cell harvester (Packard). The filters are washed twice with ice cold wash buffer (25 mM HEPES, 500 mM NaCl, 1 mM CaCl₂, 5 mM MgCl₂, adjusted to pH 7.4). Scintillant (MicroScint-10; 35 μl) is added to dried filters and the filters counted in a (Packard Topcount) scintillation counter. Data are analyzed and plotted using Prism software (GraphPad Software, San Diego, Calif.). Competition curves are analyzed and IC₅₀ values calculated. If one or more data points do not fall within the sigmoidal range of the competition curve or close to the sigmoidal range the assay is repeated and concentrations of labeled ligand and unlabeled compound adapted to have more data points close to or in the sigmoidal range of the curve.

Receptor Ligand Binding Studies On Membrane Preparations

Membranes preparations are isolated from mammalian cells (Hek293, CHO, COS7) cells over expressing the receptor is done as follows: Medium is aspirated from the transduced cells and cells are harvested in 1×PBS by gentle scraping. Cells are pelleted (2500 rpm 5 min) and resuspended in 50 mM Tris pH 7.4 (10×10⁶ cells/ml). The cell pellet is homogenized by sonicating 3×5 sec (UP50H; sonotrode MSI; max amplitude: 140 μm; max Sonic Power Density: 125W/cm²). Membrane fractions are prepared by centrifuging 20 min at maximal speed (13000 rpm˜15000 to 20000 g or rcf). The resulting pellet is resuspended in 500 μl 50 mM Tris pH 7.4 and sonicated again for 3×5 sec. The membrane fraction is isolated by centrifugation and finally resuspended in PBS. Binding competition and derivation of IC₅₀ values are determined as described above.

Internalization Screen (1)

Activation of a GPCR-associated signal transduction pathway commonly leads to translocation of specific signal transduction molecules from the cytoplasm to the plasma membrane or from the cytoplasm to the nucleus. Norak has developed their transfluor assay based on agonist-induced translocation of receptor-β-arrestin-GFP complex from the cytosol to the plasma membrane and subsequent internalization of this complex, which occurs during receptor desensitization. A similar assay uses GFP tagged receptor instead of β-arrestin. Hek293 cells are transduced with a GRPR-, ADRA1A- or TACR1-eGFP vector that translates for a GRPR-, ADRA1A- and TACR1-eGFP fusion protein. 48 hours after transduction, the cells are set to fresh serum-free medium for 60 minutes and treated with a ligand (e.g. 100 nM GRP, Substance P or A61603) for 15, 30, 60 or 120 minutes at 37° C. and 5% CO₂. After indicated exposure times, cells are washed with PBS and fixed with 5% paraformaldehyde for 20 minutes at RT. GFP fluorescence is visualized with a Zeiss microscope with a digital camera. This method aims for the identification of compounds that inhibit a ligand-mediated (constitutive activity-mediated) translocation of the fusion protein to intracellular compartments.

Internalization Screen (2)

Various variations on translocation assays exists using β-arrestin and β-galactosidase enzyme complementation and BRET based assays with receptor as energy donor and β-arrestin as energy acceptor. Also the use of specific receptor antibodies labeled with pH sensitive dyes are used to detect agonist induced receptor translocation to acidic lysosomes. All of he translocation assays are used for screening for both agonistic and antagonistic acting ligands.

Melanophore Assay (Arena Pharmaceutical)

The melanophore assay is based on the ability of GPCRs to alter the distribution of melanin containing melanosomes in Xenopus melanophores. The distribution of the melanosomes depends on the exogenous receptor that is either G_{i/o} or G_{s/q} coupled. The distribution of the melanosomes (dispersed or aggregated) is easily detected by measuring light absorption. This type of assay is used for both agonist as well as antagonist compound screens.

The following Table identifies known, agonists and antagonists of GPCRs tested by the present inventors, and includes information respecting the manufacturers of the agonist and/or antagonist.

TABLE 8

| | SEQ | | |
|---|---|---|---|
| Target | ID | agonist | antagonist |
| OPRM1 | 55 | Endomorphin-1 (sigma E3273) | β-FNA (Tocris 0926) |
| | | Endomorphin-2 (sigma E3148) | M-CAM (Tocris 0898) |
| | | Ohmefentanyl | CTAP |
| | | DAMGO (sigma E7384) | CTOP (Tocris 1578) |
| | | PL017 | Cyprodime (sigma C153) |
| | | DALDA (sigma D144) | Naloxonazine* (Sigma |
| | | Sufentanil* | N176) |
| | | Morphiceptin (sigma M4264) | |
| CCXCR1 | 57 | SCM-1/lymphotactin a(sigma L9788) | none |
| | | SCM-1/lymphotactin b | |
| LTB4R | 59 | No selective agonists available | U75302 (sigma U1508) |
| | | | LY2931111 |
| | | | CP195543 |
| | | | CP105696 |
| | | | SB209247 |
| | | | SC53228 |
| | | | CGS25019C |
| CCR5 | 61 | MIP-1beta (peprotech EC 300-09) | TAK779 |
| | | R5-hiv GP120 | MCP-3 (peprotech EC 300- |
| | | Non-selective | 17) |
| | | RANTES (sigma R6267) | MAb 2D7 |
| | | MCP-2 (peprotech EC 300-15) | Non-selective |
| | | | NSC651016 |
| | | | VMIP-II |
| | | | AOP-RANTES |
| | | | Met-RANTES |
| FZD5 | 63 | Wnt5 | None |
| AGTR1 | 65 | L163491 (partial agonist) | Valsartan* |
| | | | Losartan* |
| | | | Irbesartan* |
| | | | Candesartan* |
| | | | Eprosartan* |
| | | | Telmisartan* |
| | | | ZD7155 (Tocris 1211) |
| GABBR1 | 40 | Baclofen* (Tocris 0796) | CGP 35348 (tocris 1245) |
| | | SKF 97541 (Tocris 0379) | CGP 46381 (tocris 1247) |
| | | GABApentin* | CGP 52432 (tocris 1246) |
| | | | CGP 54626 hydrochloride |
| | | | (tocris 1088) |
| | | | CGP 55845 (tocris 1248) |
| | | | 2-Hydroxysaclofen (tocris |
| | | | 245) |
| | | | Phaclofen (tocris 178) |
| | | | Saclofen (tocris 246) |
| | | | SCH 50911 (tocris 984) |
| CHRM5 | 42 | Non-specific only | Non specific only |
| GRPR | 44 | GRP (Sigma G-8022) | D-Phe⁶,Cpa¹⁴-? 13-14]- |
| (BB2) | | Bombesin (Sigma B4272) | Bombesin(6-14) |
| | | Neuromedin B (Sigma N-3762) | D-Phe⁶-Bombesin(6-13) |
| | | | ethyl ester |
| | | | BW 1023U90 |
| P2RY1 | 46 | 2-Methylthio-ADP trisodium salt | MRS2279 |
| | | (tocris 1624) | MRS2269 |
| | | 2-Methylthioadenosine triphosphate | MRS2286 |
| | | tetrasodium salt (tocris 1062) | MRS 2179 tetraammonium |
| | | | salt (tocris 900) |
| Tar1 | 49 | β-phenylethylamine. | |
| | | tyramine | |
| ADRA1A_TV3& | 51 | Oxymetazoline* (Tocris 1142) | 5-methylurapidil (Sigma |
| TV1 | | A61603 (Tocris 1052) | U-101) |
| | | SKF-89748 | +)-Niguldipine (Sigma N- |
| | | | 135) |
| | | | Indoramin |
| | | | RS17053 (Tocris 0985) |
| | | | RS 100329 (Tocris 1325) |
| | | | WB 4101 (Tocris 0946) |
| | | | Aldomet ® |
| | | | (Methyldopa)(as disclosed |
| | | | in US Patent No. |
| | | | 2,868,818, hereby |
| | | | incorporated by reference) |
| | | | Cardura ® (Doxazosin) |
| | | | Catapres ®; Catapres- |
| | | | TTS ®; Duraclon ™ |
| | | | (Clonidine) |
| | | | Dibenzyline ® |
| | | | (Phenoxybenzamine) |
| | | | Hylorel ® (Guanadrel) |
| | | | Hytrin ® (Terazosin) |
| | | | Minipress ® (Prazosin) |
| | | | Tenex ® (Guanfacine) |
| | | | (disclosed in US Patent No. |
| | | | 5,686,612, hereby |
| | | | incorporated by reference) |
| GPR145 | 53 | | |
| ADORA3 | 38 | 2-Cl-IB-MECA (tocris 1104) | I-ABOPX |
| | | HEMADO (tocris 1579) | MRS 1191 (Sigma M-227) |
| | | IB-MECA (tocris 1066) | MRS 1220 (Sigma M-228) |
| | | | MRS 1523 (Sigma M- |
| | | | 1809) |
| | | | MRS 1220 (tocris 1217) |
| | | | MRS 1334 (tocris 1385) |
| | | | VUF 5574 (tocris 1359) |
| BDKRB2 | 52 | Peptide: | Peptide: |
| | | BK (Sigma B 3259) | HOE 140 |
| | | Lys-BK (1-8) | NPC 17731 |
| | | Ile-Ser-BK (Sigma B1643) | D- |
| | | | Arg[Hyp³,Thi⁵,HypE(trans- |
| | | | propyl)⁷,Oic⁸]-BK |
| | | Non-peptide: | Non-peptide: |
| | | FR 190997 | Bradyzide* (Sigma B 1680) |
| | | FR-191413 | FR 167344 |
| | | | FR 173657 |
| | | | WIN 64,338 (Tocris 1057) |
| CCR2 | 60 | MCP-1 (peprotech EC 300-04) | Eotaxin-3 (CCL26) |
| | | MCP-2 (peprotech EC 300-15) | (peprotech EC 300-48) |
| | | MCP-3 (peprotech EC 300-17) | RS-504393 |
| | | MCP-4 (peprotech EC 300-24) | |
| | | muMCP-5 (peprotech EC 250-04) | |
| CCR3 | 64 | Eotaxin (CCL11) (peprotech EC 300- | SB-297006 |
| | | 21) | SB-328437 |
| | | Eotaxin-2 (CCL24) (peprotech EC | 4-benzylpiperidin-1-yl-n- |
| | | 300-33) | propylureas |
| | | Eotaxin-3 (CCL26) (peprotech EC | erythro-3-(4-benzyl-2- |
| | | 300-48) | (alpha-hydroxyalkyl) |
| | | RANTES (peprotech EC 300-06) | piperidin-1-yl)-n-propyl |
| | | MCP-2 (peprotech EC 300-15) | ureas |
| | | MCP-3 (peprotech EC 300-17) | Compound X |
| | | MCP-4 (peprotech EC 300-24) | xanthene-9-carboxamide |
| | | vMIP-II | Banyu (I) |
| CCR6 | 62 | MIP-3alpha (peprotech EC 300-29A) | |
| CD97 | 43 | CD55 (Decay accelerating factor: | |
| | | DAF) | |
| CHRM2 | 41 | Bethanechol* (Sigma C 5259) | AF-DX 116 (tocris 1105) |
| | | Aceclidine hydrochloride (tocris 689) | AF-DX 384 (tocris 1345) |
| | | Arecaidine but-2-ynyl ester tosylate | (S)-(+)-Dimethindene |
| | | (tocris 382) | maleate (tocris 1425) |
| | | Arecaidine propargyl ester tosylate | Nitrocaramiphen |
| | | (tocris 383) | hydrochloride (tocris 469) |
| | | 5-Methylfurmethiodide (tocris 588) | |
| | | Oxotremorine sesquifumarate (tocris | |
| | | 843) | |
| | | Oxotremorine M (tocris 1067) | |
| | | Pilocarpine hydrochloride (tocris 694) | |
| CHRM3 | 54 | L689,660 | Hexahydro-sila-difenidol |
| | | | p-Fluorohexahydro- |
| | | | siladifenidol (Sigma-127) |
| | | | 4-DAMP (Darifenacin*) |
| | | | (tocris 482) |
| CNR1 | 39 | arachidonyl-2′-Cl-ethylamide (Tocris | SR141716A |
| | | 1319) | LY-320135 |
| | | arachidonoylcyclopropylamide (Tocris | AM251 (Tocris 1117) |
| | | 1318) | AM 281 (Tocris 1115) |
| | | (R)-(+)-Methanandamide (Tocris | |
| | | 1121) | |
| EDG1 | 58 | S1P (avantipolar lipids 860492) | |
| (S1P₁) | | | |
| NMU2R | 45 | GRP (Sigma 8022) | PD 168368 |
| | | Bombesin (Sigma B 4272) | PD 165929 |
| | | Neuromedin B (Sigma N 3762) | |
| PTGER2 | 47 | Prostaglandin E2 | AH 6809 (tocris 671) |
| | | (R)-Butaprost (Sigma B 6309) | |
| | | Alprostadil* (Tocris 1620) | |
| | | 11-deoxy PGE1 | |
| | | Misoprostol (Sigma M 6932) | |
| | | PGE2 (Sigma P5640) | |
| | | AH-13205 | |
| | | AY 23626 | |
| TACR1 | 56 | Substance P (Tocris 1156) | Peptide: |
| | | [Sar⁹,Met(O₂)¹¹]-Substance P (Tocris | GR 82334 (Tocris 1670) |
| | | 1178) | GR 71251 (Sigma 2421) |
| | | Hemokinin (Tocris 1535) | L-668,169 (Sigma L 116) |
| | | GR73632 (Tocris 1669) | Non-peptide: |
| | | | L-732,138 (Tocris 0868) |
| | | | L-733,060 (Tocris 1145) |
| | | | L-703,606 (Sigma L-119) |
| | | | CP-99,994 |
| | | | RP 67580 (Tocris 1635) |
| | | | SR 140333 |
| | | | PD 154075 |
| | | | LY-303870 |
| | | | MK-869 108 |
| | | | MPC-4505 |
| | | | Aprepitant (Emend ™) |

1. A method for identifying a compound that inhibits the processing of amyloid-beta precursor protein in a mammalian cell, comprising
(a) contacting a compound with a polypeptide comprising an amino acid sequence selected from the group consisting of SEQ ID NO: 44, 50, 51, 56, and 538-582; and
(b) measuring a compound-polypeptide property related to the production of amyloid-beta protein.

2. The method according to claim 1, wherein said polypeptide comprises SEQ ID NO: 538-582 in an in vitro cell-free preparation.

3. The method according to claim 1, wherein said polypeptide is membrane-bound.

4. The method according to claim 2, wherein said polypeptide is present as a transmembrane cell receptor in a mammalian cell.

5. The method of claim 1, wherein said property is a binding affinity of said compound to said polypeptide.

6. The method of claim 4, wherein said property is activation of a biological pathway producing an indicator of the processing of amyloid-beta precursor protein.

7. The method of claim 6 wherein said indicator is a second messenger.

8. The method of claim 7 wherein said second messenger is cyclic AMP or Ca²⁺.

9. The method of claim 6 wherein said indicator is amyloid-beta peptide.

10. The method of claim 9 wherein said amyloid-beta protein is selected from the group consisting of one or more of amyloid-beta peptide 1-42, 1-40, 11-42 and 11-40.

11. The method of claim 10 wherein said amyloid-beta protein is amyloid-beta peptide 1-42.

12. The method according to claim 6 wherein said indicator induces the expression of a reporter in said mammalian cell.

13. The method according to claim 12 wherein the reporter is selected from the group consisting of alkaline phosphatase, GFP, eGFP, dGFP, luciferase and β-galactosidase.

14. The method according to claim 1, wherein said compound is selected from the group consisting of compounds of a commercially available screening library and compounds that have been demonstrated to have binding affinity for a polypeptide comprising an amino acid sequence selected from the group consisting of SEQ ID NO: 44, 50, 51, 56, and 538-582.

15. The method according to claim 2, wherein said compound is a peptide in a phage display library or an antibody fragment library.

16. The method according to claim 1, wherein said compound is an selective tachykinin NK1 receptor antagonist, subtype selective a1A-adrenoceptor antagonist, or a GRP receptor antagonists, or the pharmaceutically acceptable salts, hydrates, or solvents thereof.

17. An agent for the inhibition of amyloid-beta precursor processing selected from the group consisting of an antisense polynucleotide, a ribozyme, and a small interfering RNA (siRNA), wherein said agent comprises a nucleic acid sequence complementary to, or engineered from, a naturally occurring polynucleotide sequence encoding a polypeptide comprising an amino acid sequence selected from the group consisting of SEQ ID NO: 44, 50, 51, 56, and 538-582.

18. The agent according to claim 17, wherein a vector in a mammalian cell expresses said agent.

19. The agent according to claim 18, wherein said vector is an adenoviral, retroviral, adeno-associated viral, lentiviral, a herpes simplex viral or a sendaiviral vector.

20. The agent according to claim 19, wherein said antisense polynucleotide and said siRNA comprise an antisense strand of 17-25 nucleotides complementary to a sense strand, wherein said sense strand is selected from 17-25 continuous nucleotides of a naturally occurring nucleic acid sequence encoding a polypeptide comprising an amino acid sequence selected from the group consisting of SEQ ID NO: 44, 50, 51 and 56.

21. The agent according to claim 20, wherein said siRNA further comprises said sense strand.

22. The agent according to claim 20, wherein said sense strand is selected from 17-25 continuous nucleotides of a nucleic acid sequence selected from the group consisting of SEQ ID NO: 7,13,14 and 19.

23. The agent according to claim 17, wherein said siRNA further comprises a loop region connecting said sense and said antisense strand.

24. The agent according to claim 23 wherein said loop region comprises a nucleic acid sequence defined of SEQ ID NO: 231.

25. The agent according to claim 17, wherein said agent is an antisense polynucleotide, ribozyme, or siRNA comprising a nucleic acid sequence selected from the group consisting of SEQ ID NO: 98-100, 122-133, 153-156 and 232-537.

26. A cognitive enhancing pharmaceutical composition comprising a therapeutically effective amount of an agent of claim 17 in admixture with a pharmaceutically acceptable carrier.

27. The cognitive enhancing pharmaceutical composition according to claim 26 wherein said agent comprises a polynucleotide comprising a nucleic acid sequence selected from the group consisting of SEQ ID NO: 98-100, 122-133, 153-156 and 232-537, a polynucleotide complementary to said nucleic acid sequence, and a combination thereof.

28. A method of inhibiting the processing of amyloid-beta precursor protein in a subject suffering or susceptible to the abnormal processing of said protein, comprising administering to said subject a pharmaceutical composition according to claim 26.

29. A method according to claim 28 for treatment or prevention of a condition involving cognitive impairment or a susceptibility to the condition.

30. The method according to claim 29 wherein the condition is Alzheimer's disease.

31. A pharmaceutical composition for the treatment or prevention of a condition involving cognitive impairment or a susceptibility to the condition, comprising an effective amyloid-beta precursor processing-inhibiting amount of a GPCR antagonist or inverse agonist.

32. A composition according to claim 31, wherein said GPCR antagonist or inverse agonist is a selective tachykinin NK1 receptor antagonist, subtype selective a1A-adrenoceptor antagonist, or a GRP receptor antagonist, its pharmaceutically acceptable salts, hydrates, solvates, or prodrugs thereof in admixture with a pharmaceutically acceptable carrier.

33. A composition according to claim 32, further comprising labeling indicating use of said composition for the treatment or prevention of a condition involving cognitive impairment or a susceptibility to said condition.
